(19) United States
(12) Patent Application Publication
(10) Pub. No.: US 2026/0327267 A1
(43) Pub. Date: Oct. 22, 2026

(54) ENGINEERED BLOOD BRAIN BARRIER PENETRANT AAV CAPSIDS

(71) Applicant: Sangamo Therapeutics, Inc., Richmond, CA (US)

(72) Inventors: Matthew R. Tiffany, San Mateo, CA (US); David S. Ojala, Richmond, CA (US)

(73) Assignee: Sangamo Therapeutics, Inc., Richmond, CA (US)

(21) Appl. No.: 19/483,086

(22) PCT Filed: May 15, 2024
(86) PCT No.: PCT/US2024/029507

§ 371 Date: Nov. 10, 2025

Related U.S. Application Data
(60) Provisional application No. 63/466,598, filed on May 15, 2023.
(60) Provisional application No. 63/606,012, filed on Dec. 4, 2023.

(51) Int. Cl. C07K 14/005; C12N 15/86

(52) U.S. Cl. CPC C07K 14/005; C12N 15/86; C12N 2750/14122; C12N 2750/14143; C12N 2750/14145

(57) ABSTRACT

This application relates to engineering AAV capsids. In some embodiments, the engineered AAV capsids are capable of penetrating the blood brain barrier and transducing cells in the central nervous system.

### CROSS REFERENCE TO RELATED APPLICATIONS

This application claims the benefit of U.S. Provisional Patent Application No. 63/466,598 filed on May 15, 2023 and entitled “Engineered Blood Brain Barrier Penetrant AAV Capsids,” and U.S. Provisional Patent Application No. 63/606,012 filed on Dec. 4, 2023 and entitled, “Engineered Blood Brain Barrier Penetrant AAV Capsids,” the entire contents of each of which are incorporated by reference herein.

### FIELD

This application relates to engineering AAV capsids.

### BACKGROUND

The clinical translation of genomic medicines to treat disorders of the central nervous system (CNS) has been limited by inefficient gene delivery. AAV capsids that cross the blood brain barrier (BBB) in rodents exhibit widespread CNS transduction and efficacy; however, capsids that cross the BBB in non-human primates have been challenging to engineer.

Attempts at providing AAV capsids with improved properties, e.g., improved tropism to a target cell or tissue upon systemic administration, have met with limited success. As such, there is a need for improved methods of producing AAV capsids and resulting AAV capsids for delivery of genetic material of interest to a target cell or tissue, e.g., a CNS cell or tissue, including delivery across the BBB.

### SUMMARY

In an aspect, CNS-targeting molecules, i.e., targeting peptides, are provided. In another aspect, an engineered AAV capsid protein is provided wherein a peptide sequence is inserted into a parent capsid at a peptide insertion site. In embodiments, the peptide sequence, the parent capsid, and the peptide insertion site are as indicated in a single row of Table 1.

In an aspect, an adeno-associated virus (AAV) capsid protein is provided comprising, consisting of, or consisting essentially of at least 3, 4, 5, 6, 7, 8, 9, or all contiguous amino acids of any amino acid sequence set forth in any one of SEQ ID NO: 1-1232.

In embodiments, the amino acid sequence comprises, consists of, or consists essentially of at least 3, 4, 5, 6, 7, 8, 9, or all contiguous amino acids of the amino acid sequence set forth in SEQ ID NO: 71.

In embodiments, the AAV capsid protein comprises, consists of, or consists essentially of serotype AAV2. In embodiments, the serotype AAV2 comprises, consists of, or consists essentially of a sequence having at least 80%, 85%, 90%, 95%, or 99% sequence identity to the sequence set forth in SEQ ID NO: 1233. In embodiments, the amino acid sequence is inserted into the AAV2 capsid protein between amino acids 588 and 589. In embodiments, the AAV capsid protein having SEQ ID NO 71 inserted therein comprises, consists of, or consists essentially of a sequence having at least 80%, 85%, 90%, 95%, or 99% sequence identity to the sequence set forth in SEQ ID NO: 1233. In embodiments, the AAV capsid having SEQ ID NO 71 inserted therein protein comprises, consists of, or consists essentially of the sequence set forth in SEQ ID NO: 1233.

In embodiments, the AAV capsid protein comprises, consists of, or consists essentially of serotype AAV9. In embodiments, the serotype AAV9 comprises, consists of, or consists essentially of a sequence having at least 80%, 85%, 90%, 95%, or 99% sequence identity to the sequence set forth in SEQ ID NO: 1234. In embodiments, the amino acid sequence is inserted into the AAV9 capsid protein between amino acids 587 and 590. In embodiments, the AAV capsid protein having SEQ ID NO 71 inserted therein comprises, consists of, or consists essentially of a sequence having at least 80%, 85%, 90%, 95%, or 99% sequence identity to the sequence set forth in SEQ ID NO: 1235. In embodiments, the AAV capsid having SEQ ID NO 71 inserted therein protein comprises, consists of, or consists essentially of the sequence set forth in SEQ ID NO: 1235.

In embodiments, the AAV capsid protein comprises, consists of, or consists essentially of an amino acid sequence set forth in any one of SEQ ID NO: 1-1232. In embodiments, the AAV capsid protein comprises, consists of, or consists essentially of an amino acid sequence set forth in SEQ ID NO: 71.

In embodiments, the AAV capsid protein comprises, consists of, or consists essentially of AAV1, AAV2, AAV3B, AAV5, AAV6, AAV8, AAV9, AAV3, AAV4, AAV7, AAV11, AAVrh10, AAVrh39, or AAVrh74. In embodiments, the AAV capsid protein comprises, consists of, or consists essentially of AAV2 or AAV9.

In embodiments, the amino acid sequence is inserted into a surface exposed region of the AAV capsid.

In an aspect, an AAV capsid protein is provided comprising, consisting of, or consisting essentially of at least 3, 4, 5, 6, 7, 8, 9 or all contiguous amino acids of an amino acid sequence set forth in any of SEQ ID NO: 1-1232, wherein the amino acid sequence is inserted between amino acid positions 450 and 600 of the AAV capsid protein.

In embodiments, the amino acid sequence is inserted between amino acid positions 588 and 589 of the AAV capsid protein. In embodiments, the AAV capsid protein comprises, consists of, or consists essentially of the sequence forth in SEQ ID NO: 1233. In embodiments, the amino acid sequence comprises the sequence set forth in SEQ ID NO: 71.

In embodiments, the amino acid sequence is inserted between amino acid positions 587 and 590 of the AAV capsid protein. In embodiments, the AAV capsid protein comprises consists of, or consists essentially of a sequence having at least 80%, 85%, 90%, 95%, or 99% sequence identity to the sequence set forth in SEQ ID NO: 1234. In embodiments, the amino acid sequence comprises, consists of, or consists essentially of SEQ ID NO: 71. In embodiments, the AAV capsid protein having SEQ ID NO 71 inserted therein comprises, consists of, or consists essentially of a sequence having at least 80%, 85%, 90%, 95%, or 99% sequence identity to the sequence set forth in SEQ ID NO: 1235. In embodiments, the AAV capsid protein comprises a sequence having at least 80%, 85%, 90%, 95%, or 99% sequence identity to the sequence set forth in SEQ ID NO: 1235, and at least 3, 4, 5, 6, 7, 8, 9, or all contiguous amino acids of SEQ ID NO: 71. In embodiments, the AAV capsid protein comprises, consists of, or consists essentially of the sequence set forth in SEQ ID NO: 1235.

In an aspect, a CNS-targeting molecule is provided comprising, consisting of, or consisting essentially of at least 3, 4, 5, 6, 7, 8, 9, or all contiguous amino acids of an amino acid set forth in any of SEQ ID NO: 1-1232.

In embodiments, the amino acid sequence comprises, consists of, or consists essentially of at least 3, 4, 5, 6, 7, 8, 9, or all contiguous amino acids of the amino acid sequence set forth in SEQ ID NO: 71. In embodiments, the amino acid sequence is fused or conjugated to a small molecule, antibody, scFV, ASO, siRNA, lipid, polymer, or recombinant protein.

### BRIEF DESCRIPTION OF THE DRAWINGS

FIG. 1 illustrates an example schematic of the barriers and selective pressure to identify AAV variants that exhibited enrichment in CNS tissue.

FIG. 2 illustrates examples of SIFTER (Selecting in vivo for transduction and expression of RNA) capsid libraries linked to barcodes. Each capsid is linked to multiple barcodes defined in a look-up table and created by pooled oligo synthesis. Different oligo pool designs allow for multiplexing libraries varying parental capsid identity or mutational strategy. Hundreds to thousands of unique molecular identifiers (UMIs) are cloned per barcode enabling detection of individual transduction events. Barcodes and UMIs are expressed under the control of either a neuron specific human synapsin I or ubiquitous CMV promoter, allowing the read out of capsid performance in multiple cell types.

FIG. 3 shows the diversities of the five round 2 sub-libraries (Library A-E) that were multiplexed and cloned under expression of a neuron specific human synapsin 1 (hSyn1) promoter or a ubiquitous CMV promoter. The resulting 10 libraries were administered via intravenous injection, and an endpoint analysis to recover library transcripts was performed.

FIG. 4 provides data showing that the production of libraries is relatively uniform with most variants found +/−32-fold change from the cloned library. As expected, variants that produce well are associated with more UMIs recovered. Bubbles circled in black outlines are variants that were selected for a third-round evaluation from NHP tissue samples. The position of the AAV9 in the library is highlighted.

FIGS. 5 and 6. The library was administered to two cynomolgus macaques and barcodes were recovered from CNS and peripheral tissues to assess biodistribution and expression of the library transcript.

FIG. 5 shows data from in vitro induced pluripotent stem cell (iPS) neuron transduction. Overall, the performance of the library in vitro is not predictive of the performance in vivo as many recovered variants from the NHP samples are less enriched in cultured neurons.

FIG. 6 shows data of in vivo library recovery from NHP samples. After filtering out variants that were poorly recovered, a total of 6,728 variants were identified from the human synapsin I library, and 4,005 variants from the CMV library. Based on consistency in recovery across animals and fold enrichment, 810 variants were selected from the hSyn1 library and 426 variants from the CMV library, for a final evaluation in NHP.

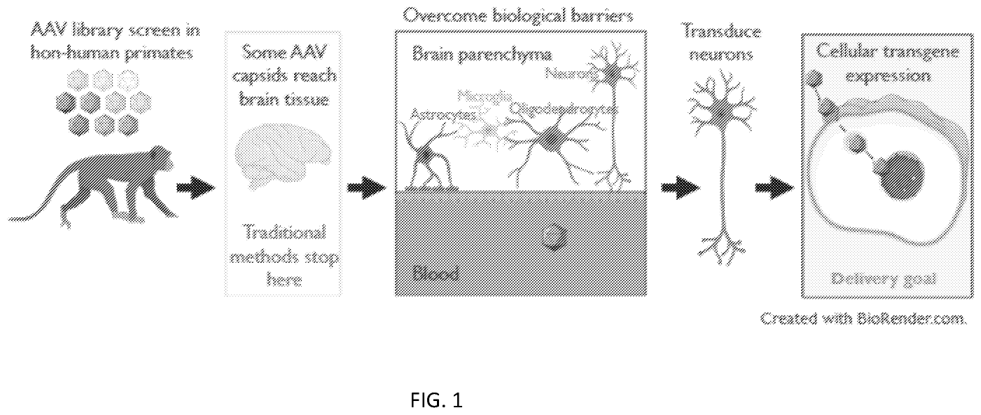

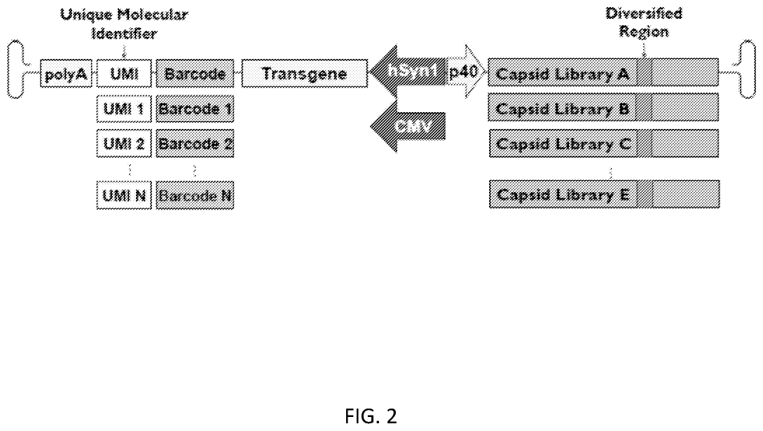

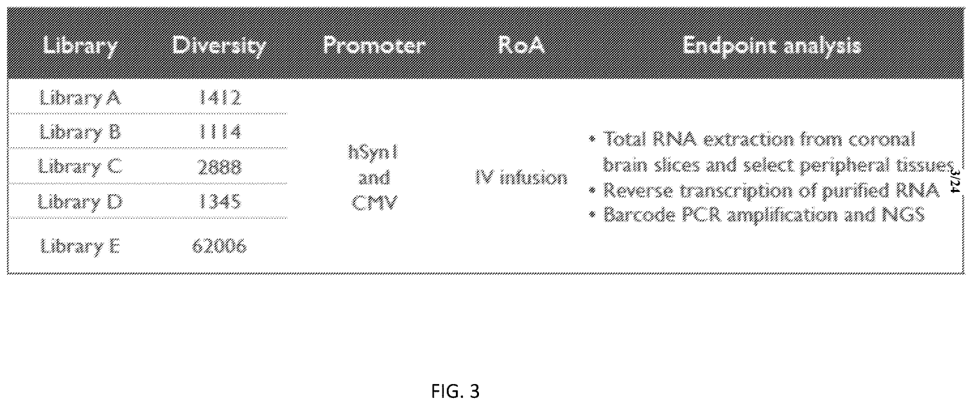

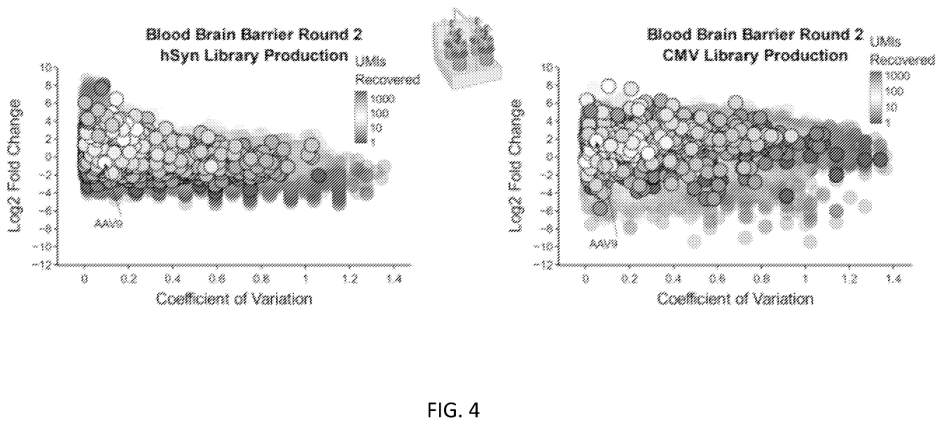

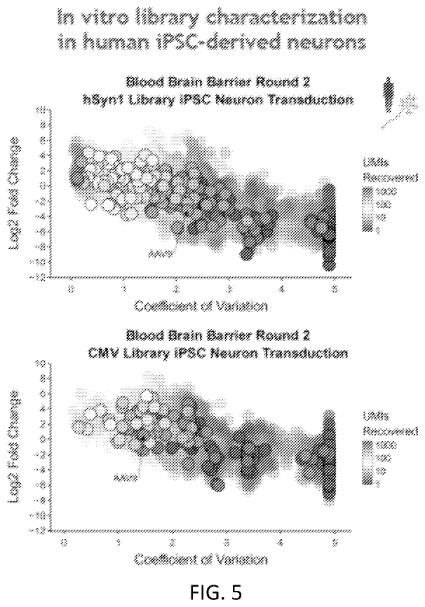

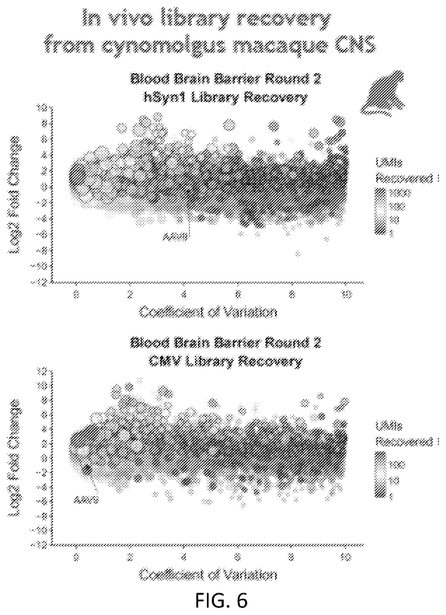

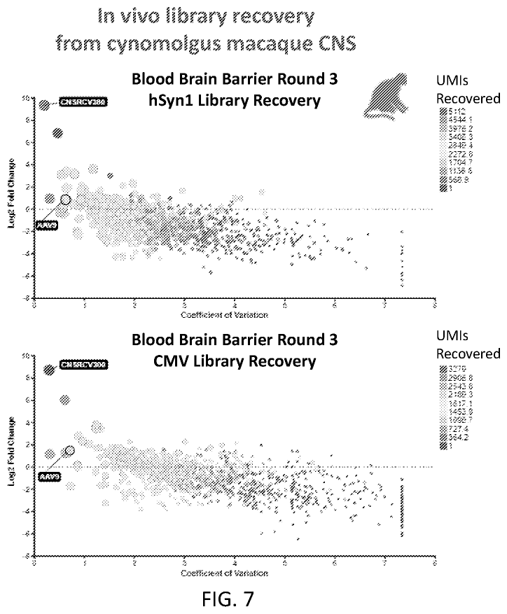

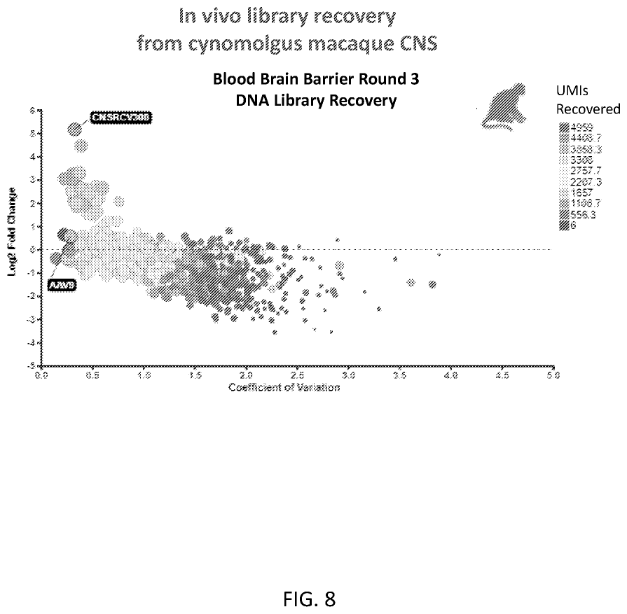

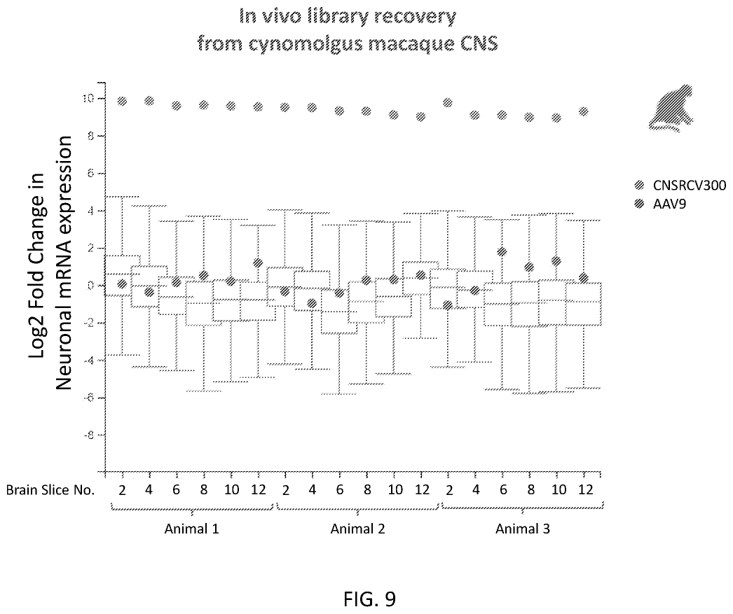

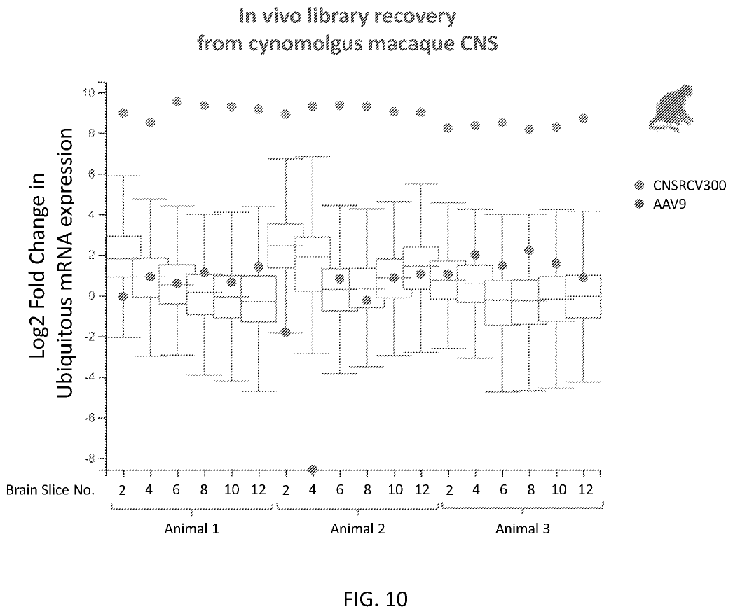

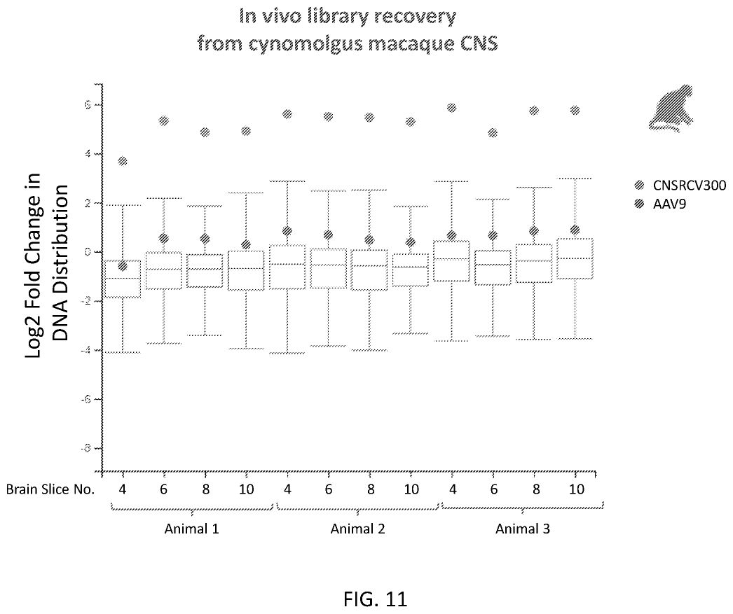

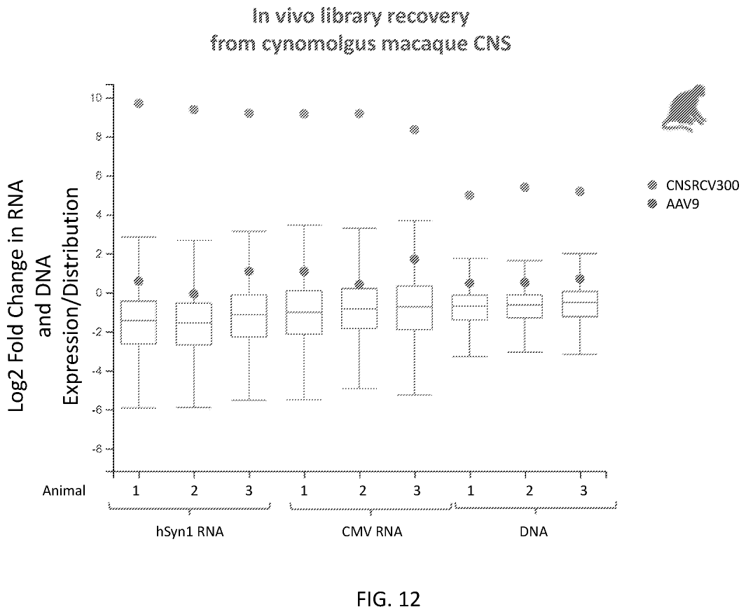

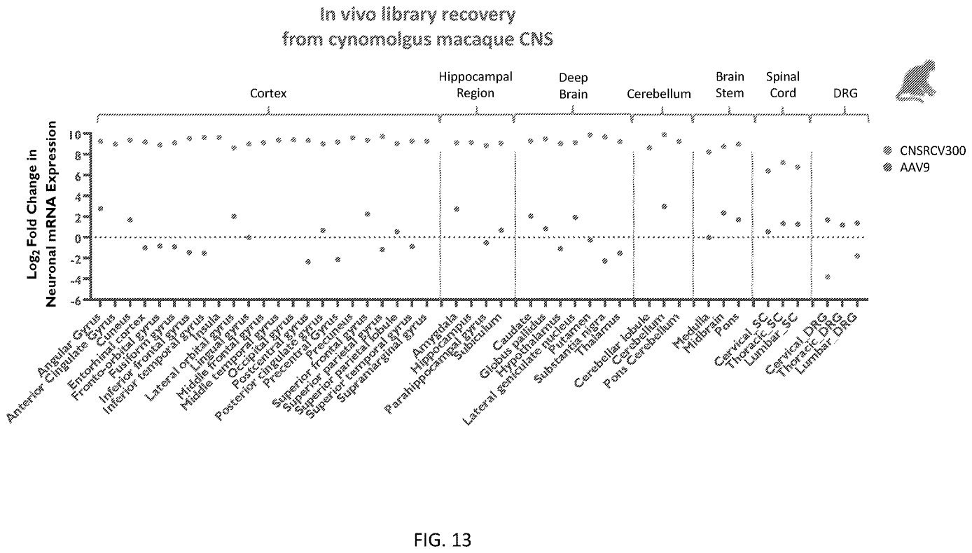

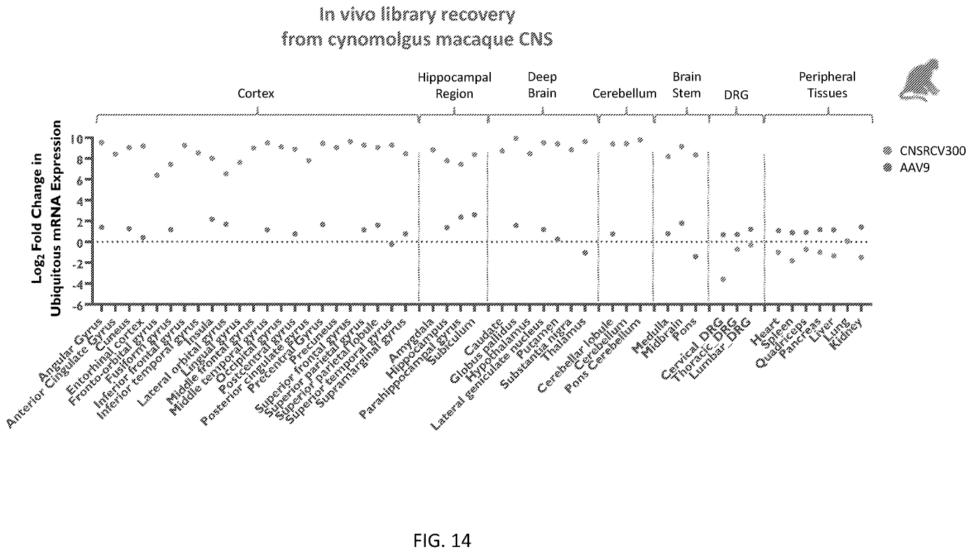

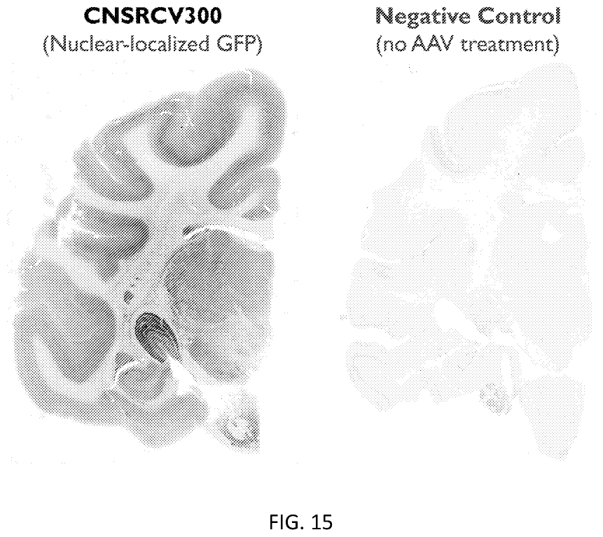

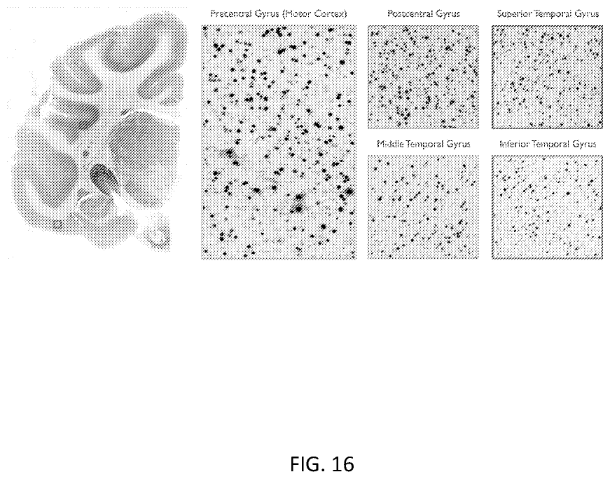

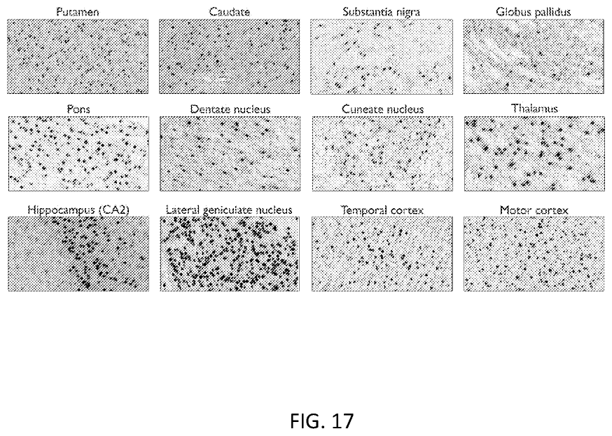

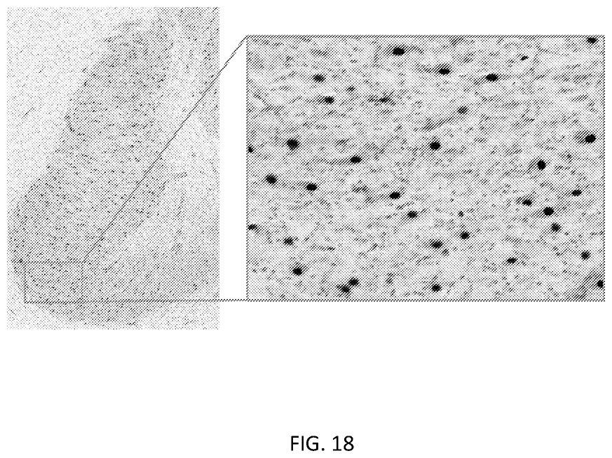

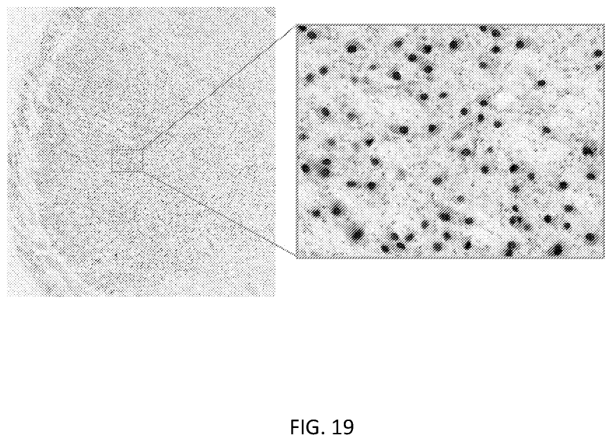

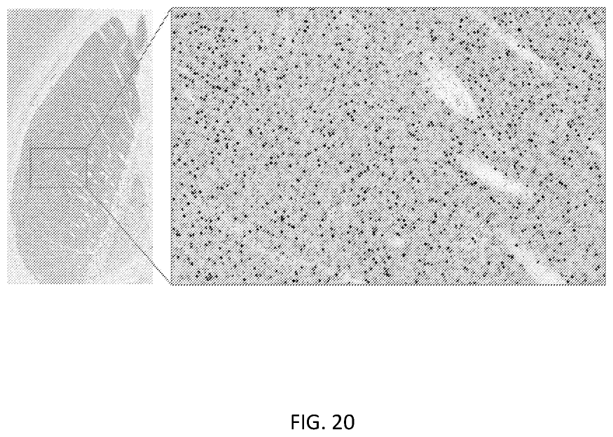

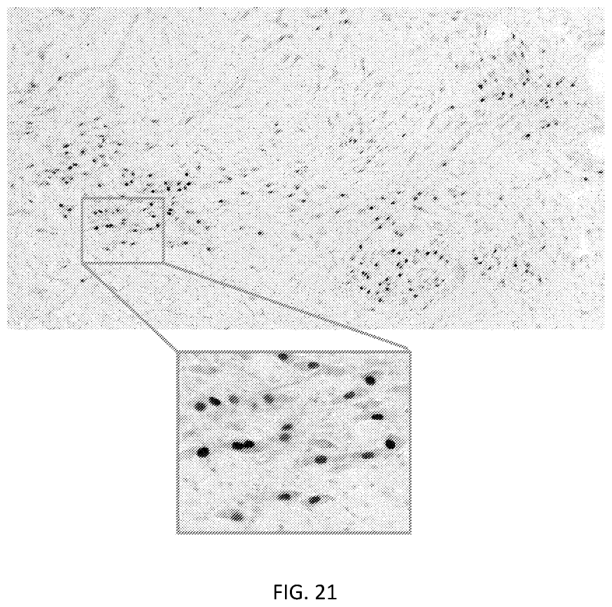

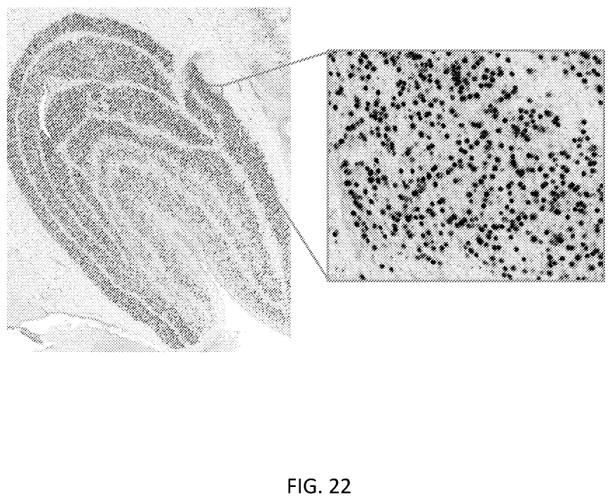

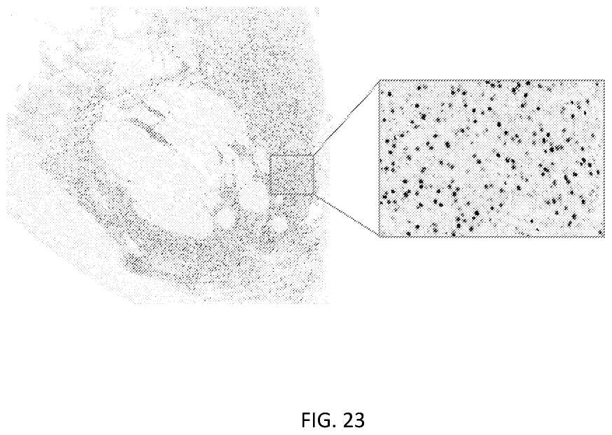

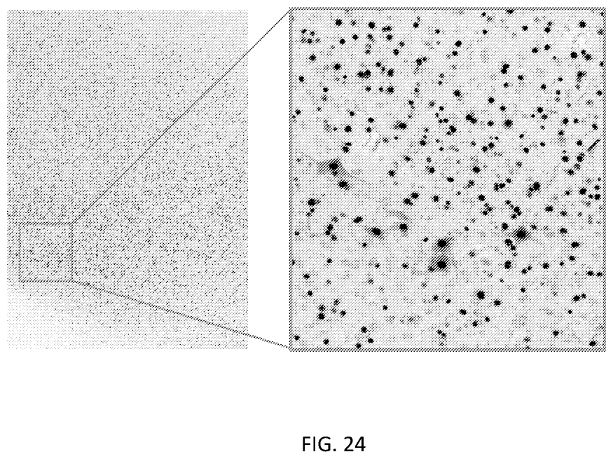

Bubble Plot Legend for FIGS. 5 and 6. The graphs show the fold-change in variant enrichment normalized to the administered viral library on the y-axis and the coefficient of variation in the detection on the x-axis. The bubble size corresponds to the fraction of replicates the variant was recovered from. The color scale indicates the number of unique molecular identifiers (UMI) associated with each variant. The top graphs are the variants' performance under the control of the neuron specific hSyn1 promoter and the bottom graphs are under the control of the ubiquitous CMV promoter. Bubbled circles in black outlines are variants that were selected for a third-round evaluation from NHP tissue samples.

FIG. 7. The round 3 library was administered to three cynomolgus macaques and barcodes were recovered from CNS tissues to assess on-target biodistribution and expression of the library transcript. The figure shows the performance of the 1236 variants in the round 3 library evaluation. The log 2 fold change (log 2FC) in expression of each variant from the hSyn1 and CMV promoters is shown on the y-axis and the coefficient of variation of the log 2 fold change measurement is shown on the x-axis. The data represents assessment of whole brain transduction and mRNA expression mediated by each capsid in the library.

FIG. 8. The figure shows the performance of the 1236 variants in the round 3 library evaluation. The vector genome biodistribution of each variant is shown. The data represents assessment of whole brain transduction mediated by each capsid in the library.

FIG. 9. The graph shows the log 2 fold-change in neuronal (hSyn1) mRNA expression of CNSRCV300 (also referred to as SEQ ID NO: 1235, STAC-BBB, STAC/BBB, or STACBBB) and AAV9 across six brain slices analyzed per animal. The box and whisker plots contains the rest of the round 3 library performance.

FIG. 10. The graph shows the log 2 fold-change in ubiquitous (CMV) mRNA expression of CNSRCV300 and AAV9 across six brain slices analyzed per animal. The box and whisker plots contains the rest of the round 3 library performance.

FIG. 11. The graph shows the log 2 fold-change in vector genome biodistribution of CNSRCV300 and AAV9 across four brain slices analyzed per animal. The box and whisker plots contains the rest of the round 3 library performance.

FIG. 12. The graph shows the log 2 fold-change in mRNA expression and vector genome biodistribution of CNSRCV300 and AAV9 across all animals included in the study. The box and whisker plots contains the rest of the round 3 library performance.

FIG. 13. The graph shows the log 2 fold change in neuronal (hSyn1) mRNA expression of CNSRCV300 and AAV9 in all tissues punches analyzed across the cortex, hippocampal region, deep brain regions, cerebellum and brain stem. The neuronal (hSyn1) mRNA expression in cervical, thoracic and lumbar spinal cord and dorsal root ganglia levels is also shown for CNSRCV300 and AAV9.

FIG. 14. The graph shows the log 2 fold change in ubiquitous (CMV) mRNA expression of CNSRCV300 and AAV9 in all tissues punches analyzed across the cortex, hippocampal region, deep brain regions, cerebellum and brain stem. The ubiquitous (CMV) mRNA expression in cervical, thoracic and lumbar dorsal root ganglia levels is also shown for CNSRCV300 and AAV9. Lastly, peripheral tissues mRNA expression in peripheral tissues is also shown for CNSRCV300 and AAV9.

FIG. 15. The image shows that CNSRCV300 drives widespread and robust transgene expression throughout the brain at a dose of 2e13 vg/kg. Negative control tissue without AAV treatment shows no signal.

FIG. 16. The images show that CNSRCV300 exhibits widespread transduction across all cortical regions.

FIG. 17. The images show that CNSRCV300 mediates efficient transduction of neurons in a variety of cortical and subcortical regions.

FIG. 18. The images show that CNSRCV300 mediates efficient transduction of neurons in the dentate nucleus.

FIG. 19. The images show that CNSRCV300 mediates efficient transduction of neurons in the Thalamus.

FIG. 20. The images show that CNSRCV300 mediates efficient transduction of neurons in the Putamen.

FIG. 21. The images show that CNSRCV300 mediates efficient transduction of neurons in the substantia nigra.

FIG. 22. The images show that CNSRCV300 mediates efficient transduction of neurons in the lateral geniculate nucleus.

FIG. 23. The images show that CNSRCV300 mediates efficient transduction of neurons in the pons.

FIG. 24. The images show that CNSRCV300 mediates efficient transduction of neurons in the precentral gyrus.

### DETAILED DESCRIPTION

In an aspect, CNS (central nervous system)-targeting molecules, i.e., targeting peptides, are provided. In embodiments, CNS-targeting molecules comprising a targeting peptide seq indicated in Table 1 are provided. In another aspect, engineered AAV capsid proteins are provided. In embodiments, a targeting peptide is inserted into an AAV capsid, for example an AAV9 capsid protein or an AAV2 capsid protein. In embodiments, the targeting peptide is any of the targeting peptide disclosed in Table 1. In embodiments, the peptide sequence is inserted into the AAV9 or AAV2 capsid at any of the peptide insertion sites disclosed in Table 1. In some embodiments, the targeting peptide comprises a targeting peptide that functions to target the CNS-targeting molecule to a specific target tissue (e.g., CNS tissue).

### Libraries of AAV Capsid Proteins

In one aspect, disclosed herein is the development of libraries encoding AAV capsid proteins with a desired characteristic compared to a natural/wild-type AAV serotype. Thus, described herein are libraries of AAV capsid proteins with a desired characteristic compared to a natural/wild-type AAV serotype. In some embodiments, the desired characteristic is enhanced cell or tissue tropism as compared to the natural/wild-type AAV serotype, for example, enhanced cell or tissue tropism to the central nervous system (CNS) as compared to the natural/wild-type AAV serotype. In some embodiments, the desired characteristic is increased penetrance through the blood brain barrier following administration to a subject. In some embodiments, the desired characteristic is wider distribution throughout the multiple brain regions, e.g., frontal cortex, sensory cortex, motor cortex, putamen, thalamus, cerebellar cortex, dentate nucleus, caudate, and/or hippocampus. In some embodiments, the desired characteristic is elevated genetic material expression in multiple brain regions. In some embodiments, the desired characteristic is delivery of genetic material of interest to a desired tissue, cell, or organelle.

In some embodiments, each member of a library comprises one or more of a) a nucleic acid sequence encoding an AAV capsid protein comprising a targeting peptide inserted into a hypervariable and/or surface exposed loop of the capsid protein: b) a nucleic acid sequence encoding barcode: c) one or more nucleic acid sequences encoding one or more promoters; and d) a nucleic acid sequence encoding a unique molecular identifier (UMI). In some embodiments, each member of the library also includes genetic material to be delivered to a cell or tissue of interest. In some embodiments, each member of the library also includes a polyA sequence.

In some embodiments, the genetic material encodes one or more peptides or a polypeptides. In some embodiments, the genetic material encodes one or more antibodies or antibody fragments. In some embodiments the genetic material encodes one or more regulatory RNA, such as RNAi agents or microRNA.

In some embodiments, the genetic material can include sequences that are coding sequences. In some embodiments, the genetic material can include sequences that are non-coding sequences. In some embodiments, the genetic material can include sequences that are both coding sequences and non-coding sequences. In some embodiments, the expression of the genetic material is capable of being regulated. In some embodiments, the genetic material comprises elements that are regulatable.

In some embodiments, mRNA is encoded in the genetic material. In some embodiments, the mRNA is codon optimized.

In some embodiments, the genetic material encodes a gene therapy product. A gene therapy product can include a peptide, a polypeptide, a recombination donor template, or an RNA molecule that when expressed carries out a desired therapeutic effect. In some embodiments, the therapeutic effect is treating any one more diseases or disorders described herein.

In some embodiments, each member of the library is fused or coupled to an active agent. In some embodiments, each member of the library is fused or coupled to an active agent through conjugation. In some embodiments, the active agent comprises a therapeutic agent. In some embodiments, the therapeutic agent comprises a DNA-binding and/or targeting composition, for example, a zinc finger protein (ZFP), TAL-effector domain, and/or a sgRNA of CRISPR/Cas system. In some embodiments, the therapeutic agent comprises an antibody or a portion of an antibody (e.g., Fc region). In some embodiments, the peptide is fused to a Fc region of an antibody. In some embodiments, the peptide is fused to the C-terminus of the Fc region. In some embodiments, the peptide is fused to the N-terminus of the Fc region. In some embodiments, the therapeutic agent comprises an RNAi agent (e.g., siRNA, shRNA, lncRNA, piRNA, snoRNA, or miRNA). In some embodiments, the peptide is fused or coupled directly to at least one strand of the RNAi. In some embodiments, the peptide the peptide is fused or coupled to at least one strand of RNAi using a linker. In some embodiments, the peptide is fused or coupled to the sense strand of RNAi. In some embodiments, the peptide is fused or coupled to the antisense strand of RNAi. In some embodiments the active agent comprises a diagnostic agent. In some embodiments, the diagnostic agent comprises a detectable moiety such as a fluorophore. In some embodiments, the active agent is a small molecule.

In some embodiments, the promoter is operably linked to the genetic material to be delivered to the cell. In some embodiments, the one or more promoters comprises a tissue and/or cell specific promoter. In some embodiments, the one more promoters comprise a ubiquitous promoter. Examples of ubiquitous promoters include cytomegalovirus (CMV), chicken β-actin (CBA), ubiquitin C (UBC), and elongation factor 1α-subunit (EF1-α), amongst others.

In some embodiments, the one or more promoters comprise a cell type and/or tissue specific type promoter. Exemplary cell type and/or tissue specific promoters include the human synapsin promoter (hSyn1), only expressed in neurons, or the transthyretin promoter (TTR), expressed in hepatocytes. Other non-limiting cell type and/or tissue specific promoters for use in the methods and compositions of the invention include cytokeratin 18 and 19 (epithelial cell specific. Other cell-specific promoters include GFAP promoter (astrocytes), TBG promoter (liver), CAMK promoter (skeletal muscle), MYH6 promoter (cardiomyocytes).

In embodiments, tissue specific or cell specific promoters can restrict expression to tissues or cells of the CNS or PNS. In embodiments, tissue specific or cell specific promoters can be used to restrict expression to neurons of the sympathetic system, the parasympathetic system, astrocytes, microglia, oligodendrocytes, and/or Schwann cells.

In some embodiments, the one or more promoters are naturally occurring promoters. In some embodiments, the one or more promoters are synthetic. In some embodiments, the one or more promoters is derived from mammals, humans, viruses, or plants. In some embodiments, the one or more promoters is truncated. In some embodiments, the one or more promoters is mutated.

In some embodiments, a nucleic acid comprising a barcode is added to the genome of each AAV capsid proteins in a library. In some embodiments, the barcode is bioinformatically linked to the targeting peptide introduced into the capsid protein. In some embodiments, the DNA sequences encoding the targeting peptide are synthesized to further comprise a random or specified barcode. The barcode may comprise 5, 6, 7, 8, 9, 10, 11, 12, 13, 14, 15, 16, 17, 18 or more nucleotides. In some embodiments, each targeting peptide is linked to at least 2 distinct barcodes. In some embodiments, each barcode is linked to one or more UMI.

In some embodiments, each member of the library comprises a nucleic acid comprising more than one barcode sequences. In some embodiments, each member of the library comprises two or more nucleic acids each comprising a barcode sequence. In some embodiments, each member of the library comprises a first nucleic acid comprising a first barcode and a second nucleic acid comprising a second barcode. In some embodiments, the first nucleic acid comprising the first barcode and the second nucleic acid comprising the second barcode are different. In some embodiments, each of the first nucleic acid comprising the first barcode and the second nucleic acid comprising the second barcode is independently operatively linked to a promoter.

In some embodiments, the AAV capsid proteins are derived from AAV1, AAV2, AAV3B, AAV5, AAV6, AAV8 and AAV9 serotypes. In some embodiments, the AAV variant capsid proteins are derived from less well characterized AAV serotypes, including but not limited to AAV4, AAV7, AAVrh10, AAVrh39, and AAVrh74. In some embodiments, a library of AAV variants comprises AAV variant capsid proteins derived from a single AAV serotype. In some embodiments, a library of AAV variants comprises AAV variant capsid proteins derived from 2, 3, 4, 5, 6, 7, 8, 9, 10, 11, 12, 13, 14, 15, or more AAV serotypes. In some embodiments, the AAV variant capsid proteins derived from 2, 3, 4, 5, 6, 7, 8, 9, 10, 11, 12, 13, 14, 15, or more AAV serotypes are combined once individual serotype libraries are developed. In some embodiments, combinatorial libraries are generated by modifying nucleic acids encoding AAV capsid proteins from 2, 3, 4, 5, 6, 7, 8, 9, 10, 11, 12, 13, 14, 15, or more serotypes in the same pool.

In some embodiments, the AAV serotype includes the AAV2 serotype. In some embodiments, the AAV2 serotype includes a mutation of arginine to alanine at amino acid position 588. In some embodiments, the AAV2 serotype includes the sequence SEQ ID NO: 1233. In some embodiments, the AAV serotype includes the AAV9 serotype. In some embodiments, AAV9 serotype includes the sequence of SEQ ID NO: 1234.

In some embodiments, targeting peptides are introduced into a DNA sequence encoding an exposed loop in the capsid protein. In some embodiments, the targeting peptide is inserted into exposed loops (e.g. hypervariable regions) in the AAV capsid. In some embodiments, the targeting peptide comprises 6, 7, 8, 9, 10, 11, 12, 13, 14, 15, 16, 17, 18, 19, 20, or more amino acids. In some embodiments, the targeting peptide comprises between 9 and 16 amino acids. In some embodiments, the targeting peptide is 9 amino acids in length. In some embodiments, the targeting peptide is 16 amino acids in length. In some embodiments, the HI loop is targeted (mutated) while in others, the DE loop is targeted (mutated). In some embodiments, mutations (e.g., insertions, deletions and/or substitutions) are made in both loops. In further embodiments, targeting peptides are introduced into the VR region of a surface loop, including into VR-I, VR-II, VR-III, VR-IV, VR-V, VR-VI, VR-VII, VR-VIII and or VR-IX. In yet other embodiments, targeting peptides are made in VR-I, VR-IV, and or VR-VIII. In some embodiments, targeting peptides are introduced into the AAV capsid proteins VP1, VP2 or VP3, or in two of the capsid proteins in any combination, or in all three. In some embodiments, the targeting peptides are introduced into VP1. In some embodiments, the targeting peptides are introduced into VP2. In some embodiments, the targeting peptides are introduced into VP3. In some embodiments, the targeting peptides are introduced into VP1 and VP2. In some embodiments, the targeting peptides are introduced into VP1 and VP3. In some embodiments, the mutations are introduced into VP2 and VP3. In some embodiments, the targeting peptides are introduced into VP1, VP2, and VP3. In some embodiments, the targeting peptide is introduced at a single site in a gene encoding a capsid protein.

In some exemplary embodiments, a targeting peptide is introduced into the variable regions VR-I, VR-IV, or VR-VIII of the capsid protein. In some embodiments, the targeting peptide is introduced at a location between positions 450 and 600 of the capsid protein. In some embodiments, the targeting peptide is inserted between positions 587 and 590 of the capsid protein. In some embodiments, the targeting peptide is inserted between positions 587 and 590 of the AAV9 capsid protein. In some embodiments, the targeting peptide is inserted between positions 588 and 589 of the capsid protein. In some embodiments, position 588 has been altered from an arginine to an alanine. In some embodiments, the targeting peptide is inserted between positions 588 and 589 of the AAV2 capsid protein.

In some embodiments, the targeting peptide comprises any one of SEQ ID NO: 1-1232. In some embodiments, the targeting peptide comprises at least 5, 6, 7, 8, or 9 contiguous amino acids of any one of SEQ ID NO: 1-1232. In some embodiments, the targeting peptide comprises a sequence that is at least 80% identical to any one of SEQ ID NO: 1-1232. In some embodiments, the targeting peptide comprises a sequence that is at least 80% identical, least 85% identical, at least 90% identical, at least 95%, or at least 99% identical to or comprises any one of SEQ ID NO: 1-1232. In some embodiments, the targeting peptide comprises SEQ ID NO: 71. In some embodiments, the targeting peptide comprises at least 3, 4, 5, 6, 7, 8, or 9 contiguous amino acids of SEQ ID NO: 71.

### Engineered AAV Capsid Proteins

### Development of Engineered AAV Capsid Proteins

Described are compositions comprising an engineered AAV capsid proteins and methods of making and using the same.

Disclosed are methods and compositions to develop engineered AAV capsid proteins with a desired characteristic compared to a natural/wild-type AAV serotype. For example, capsid proteins are useful in delivering peptides (e.g., targeting peptides) across the blood brain barrier following administration to a subject. In some embodiments, the engineered AAV capsid proteins are useful in achieving is wider distribution of the genetic material throughout the multiple brain regions, e.g., frontal cortex, sensory cortex, motor cortex, putamen, thalamus, cerebellar cortex, dentate nucleus, caudate, and/or hippocampus. In some embodiments, the engineered AAV capsid proteins are useful in elevating genetic material expression in multiple brain regions. In some embodiments, the engineered AAV capsid proteins are used to deliver genetic material of interest to a desired tissue, cell, or organelle.

### Gene Editing System

In some embodiments, the genetic material of interest comprises a gene editing system or portions of a gene editing system. In some embodiments, the gene editing system is capable of inducing single or double-stranded breaks into nucleic acid sequences. In some embodiments, the gene editing system is capable of inserting, substituting, or deleting a base or a sequence of bases into nucleic acid sequences. In some embodiments, the gene editing system includes a CRISPR-Cas system. In some embodiments, the gene editing system includes a TALEN. In some embodiments, the gene editing system includes a zinc finger nuclease.

Engineered AAV Capsid Proteins within a Cell

In some embodiments, engineered AAV capsid proteins are contained within a cell. In some embodiments, the cell is derived from the CNS. In some embodiments, the cell is derived from the PNS. In some embodiments, the cell is derived from the brain. In some embodiments, the cell is derived from the spinal cord. In some embodiments, the cell is derived from any of the frontal cortex, the sensory cortex, the motor cortex, the cerebellar cortex, the cerebral cortex, the brain stem, the hippocampus, or the thalamus, amongst others.

### Engineered AAV Capsid Proteins Delivered to a Target Cell

The engineered AAV capsid proteins may be delivered to one or more target cells, tissues, organs, or organisms. In some embodiments, the engineered AAV capsid proteins demonstrate enhanced tropism for a target cell type, tissue or organ. As a non-limiting example, the engineered AAV capsid proteins may have enhanced tropism for cells and tissues of the central or peripheral nervous systems (PNS), or cells and tissues of a muscle. The engineered AAV capsid proteins may, in addition, or alternatively, have decreased tropism for an undesired target cell-type, tissue or organ. As a non-limiting example, the engineered AAV capsid proteins may have enhanced tropism for B cells, hematopoietic cells, leukocytes, platelets, macrophages, megakaryocytes, monocytes and/or T cells.

### Methods of Detecting Engineered AAV Capsid Proteins

In some embodiments, a method of identifying an engineered AAV capsid protein with a desired characteristic compared to a natural/wild-type AAV serotype is provided comprising: (i) contacting a cell, cell line, or tissue in vitro or in vivo with any one of the libraries of engineered AAV capsid proteins, (ii) allowing the engineered AAV capsid proteins in said library to transduce the cell, cell line, or tissue; (iii) recovering from the cell, cell line, or tissue the AAV variant; and (iv) identifying the engineered AAV capsid protein with the desired characteristic.

In another aspect, disclosed herein are methods for directed evolution of engineered AAV capsid proteins and identification of an engineered AAV capsid protein with a desired characteristic compared to a natural/wild-type AAV serotype. In some embodiments, the steps for directed evolution of engineered AAV capsid proteins to identify engineered AAV capsid proteins with a desired characteristic compared to a natural/wild-type AAV serotype comprise (i) insertion of targeting peptides into hypervariable and/or surface-exposed loops in capsid proteins from one or more AAV serotypes creating libraries of modified variant capsids for each AAV serotype; (ii) packaging of the variant AAVs in producer cells wherein adenovirus helper and AAV rep functions are supplied in trans; (iii) purification of viral capsid library pools; (iv) administration of the pools in vitro or in vivo; (v) recovery of engineered AAV capsid proteins from target tissues or cell lines; (vi) next-generation sequencing to determine the identity of the engineered variant capsid sequences; (vii) repeated rounds of in vitro or in vivo selection where variants are isolated from a target tissue or cell line; and (viii) full evaluation of enriched variants. In some embodiments, the desired characteristic includes enhanced tissue tropism as compared to the natural/wild-type AAV serotype. In some embodiments, the desired characteristic includes enhanced tissue tropism for tissues of the peripheral nervous system as compared to the natural/wild-type AAV serotype. In some embodiments, the desired characteristic includes enhanced tissue tropism of the central nervous system as compared to the natural/wild-type AAV serotype. In some embodiments, the desired characteristic includes enhanced ability to cross the blood brain barrier as compared to the natural/wild-type AAV serotype. In some embodiments, the targeting peptides that are inserted into the hypervariable and/or surface-exposed loops include any of the sequences set forth in SEQ ID NO: 1-1232. In some embodiments, SEQ ID NO: 71 is inserted into the hypervariable and/or surface exposed loop.

### Capsid Proteins

In some embodiments, capsid proteins, for example AAV1, AAV2, AAV3B, AAV5, AAV6, AAV8 and AAV9 are chosen for starting points. In some embodiments, capsid proteins from less well characterized AAV serotypes are chosen, including but not limited to AAV4, AAV7, AAV11, AAVrh10, AAVrh39, and AAVrh74. In some embodiments, a library of AAV variants comprises AAV variant capsid proteins derived from a single AAV serotype. In some embodiments, a library of AAV variants comprises AAV variant capsid proteins derived from 2, 3, 4, 5, 6, 7, 8, 9, 10, 11, 12, 13, 14, 15, or more AAV serotypes. In some embodiments, the AAV variant capsid proteins derived from 2, 3, 4, 5, 6, 7, 8, 9, 10, 11, 12, 13, 14, 15, or more AAV serotypes are combined once individual serotype libraries are developed. In some embodiments, combinatorial libraries are generated by modifying nucleic acids encoding AAV capsid proteins from 2, 3, 4, 5, 6, 7, 8, 9, 10, 11, 12, 13, 14, 15, or more serotypes in the same pool.

In some embodiments, the libraries are packaged in HEK293 cells where the helper functions (e.g. E2A, E4, VA, E1A and E1B) are supplied in trans. In some embodiments, the AA V rep function comprises rep78, rep68, rep52, and rep40 genes. In some embodiments, the rep genes are supplied in trans. In some embodiments, the start codon of the rep78 and/or the rep68 gene is altered from ACG to ATG to increase replication of the capsid library construct containing inverted terminal repeats (ITRs), thereby improving AAV library manufacturing yield. In some embodiments, the cap genes are supplied as genetic material to the manufactured AAVs. In some embodiments, the capsid gene is controlled by the p40 promoter such that it is only expressed during manufacturing in HEK293 cells in the presence of helper virus functions.

### CNS (Central Nervous System)-Targeting Molecules (Targeting Peptides)

Described herein are CNS-targeting molecules, i.e, targeting peptides. In some embodiments, the CNS-targeting molecules have enhanced tropism for a cell or tissue, such as the delivery of genetic material of interest to said cell or tissue, for example a CNS tissue or PNS tissue or a CNS cell or PNS cell.

In some embodiments, the CNS-targeting molecule comprises a sequence set forth in SEQ ID NO: 1-1232. In some embodiments, the targeting peptide comprises at least 5, 6, 7, 8, or 9 contiguous amino acids of a sequence set forth in SEQ ID NO: 1-1232. In some embodiments, the targeting peptide comprises the sequence set forth in SEQ ID NO: 71. In some embodiments, the targeting peptide comprises at least 3, 4, 5, 6, 7, 8, or 9 contiguous amino acids of the sequence set forth in SEQ ID NO: 71.

In some embodiments, the CNS-targeting molecule comprises variants of the amino acid sequences set forth in SEQ ID NO: 1-1232. In embodiments, a variant refers to any one or more of a substitution, deletion, or addition to any of the amino acids of any of the amino acid sequences set forth in SEQ ID NO: 1-1232. In some embodiments, the variant comprises 1, 2, 3, or 4 substitutions to any of the amino acids of any of the amino acid sequences set forth in SEQ ID NO: 1-1232. In some embodiments, the variant comprises 1, 2, 3, or 4 deletions to any of the amino acids of any of the amino acid sequences set forth in SEQ ID NO: 1-1232. In some embodiments, the variant comprises 1, 2, 3, or 4 insertions to any of the amino acids of any of the amino acid sequences set forth in SEQ ID NO: 1-1232. In some embodiments, the variant comprises any combination of the substitutions, deletions, or insertions described above.

In embodiments, a variant refers to a variant in the nucleotide sequence that encodes any of the amino acid sequences set forth in SEQ ID NO: 1-1232. In embodiments, the variant in the nucleotide sequence results in encoding any one or more of a substitution, deletion, or addition to any of the amino acids of any of the amino acid sequences set forth in SEQ ID NO: 1-1232. In embodiments, the variant in the nucleotide sequence encodes 1, 2, 3, or 4 substitutions to any of the amino acids of any of the amino acid sequences set forth in SEQ ID NO: 1-1232. In embodiments, the variant in the nucleotide sequence encodes 1, 2, 3, or 4 deletions to any of the amino acids of any of the amino acid sequences set forth in SEQ ID NO: 1-1232. In embodiments, the variant in the nucleotide sequence encodes 1, 2, 3, or 4 insertions to any of the amino acids of any of the amino acid sequences set forth in SEQ ID NO: 1-1232. In embodiments, the variant in the nucleotide sequence encodes any combination of the substitutions, deletions, or insertions described above.

In some embodiments, the CNS-targeting molecule is fused or conjugated to a small molecule, an antibody, scFV, ASO (antisense oligonucleotide), siRNA, lipid, polymer or recombinant protein. In some embodiments, any of SEQ ID NO: 1-1232 are fused or conjugated to a small molecule, an antibody, scFV, ASO (antisense oligonucleotide), siRNA, lipid, polymer or recombinant protein. In some embodiments, SEQ ID NO: 71 is fused or conjugated to a small molecule, an antibody, scFV, ASO (antisense oligonucleotide), siRNA, lipid, polymer or recombinant protein. In some embodiments, CNS-targeting molecules may be utilized to enable a small molecule, an antibody, scFV, ASO (antisense oligonucleotide), siRNA, lipid, polymer or recombinant protein to cross the blood brain barrier.

In some embodiments, the CNS-targeting molecules are part of an engineered AAV capsid protein. In some embodiments the engineered capsid protein comprises any of the serotypes of AAV1, AAV2, AAV3B, AAV5, AAV6, AAV8 and AAV9. In embodiments, the capsid protein comprises the serotype AAV2 or AAV9. In some embodiments, the AAV2 serotype comprises SEQ ID NO: 1233. In some embodiments, the AAV2 serotype comprises a variant of SEQ ID NO: 1233. In some embodiments, the AAV9 serotype comprises SEQ ID NO: 1234. In some embodiments, the AAV9 serotype comprises a variant of SEQ ID NO: 1234. In embodiments, a variant refers to any one or more of a substitution, deletion, or addition to any of the amino acids in either of the amino acid sequences set forth in SEQ ID NO: 1233 or SEQ ID NO: 1234. In embodiments, the variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 substitutions of any of the amino acids in either of the amino acid sequences of SEQ ID NO: 1233 or SEQ ID NO: 1234. In embodiments, the variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 deletions of any of the amino acids in either of the amino acid sequences set forth of SEQ ID NO: 1233 or SEQ ID NO: 1234. In embodiments, the variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 insertions of any of the amino acids in either of the amino acid sequences set forth SEQ ID NO: 1233 or SEQ ID NO: 1234.

In some embodiments, the variant comprises an amino acid sequence that comprises at least 80% sequence identity to the sequence set forth in SEQ ID NO: 1234 or SEQ ID NO: 1233, and at least 3, 4, 5, 6, 7, 8, 9 or all contiguous amino acids of any of SEQ ID NOS: 1-1232.

In some embodiments, the AAV2 serotype comprises a sequence that is at least 80% identical, at least 85% identical, at least 90% identical, or at least 95% identical to SEQ ID NO: 1233. In some embodiments, the AAV9 serotype comprises a sequence that is at least 80% identical, at least 85% identical, at least 90% identical, or at least 95% identical to SEQ ID NO: 1234.

In embodiments, a variant refers to a variant in the nucleotide sequence that encodes the amino acid sequences set forth in SEQ ID NO: 1233 or SEQ ID NO: 1234. In some embodiments, the variant in the nucleotide sequences results in encoding any one or more of a substitution, deletion, or addition to any of the amino acids of either of the amino acid sequences set forth in SEQ ID NO: 1233 or SEQ ID NO: 1234. In embodiments, the variant in the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 substitutions of any of the amino acids in either of the amino acid sequences of SEQ ID NO: 1233 or SEQ ID NO: 1234. In embodiments, the variant in the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 deletions of any of the amino acids in either of the amino acid sequences of SEQ ID NO: 1233 or SEQ ID NO: 1234. In some embodiments, the variant in the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 insertions of any of the amino acids in either of the amino acid sequences of SEQ ID NO: 1233 or SEQ ID NO: 1234.

In some embodiments, the variant in the nucleotide sequence encodes an amino acid sequence that comprises at least 80% sequence identity to the sequence set forth in SEQ ID NO: 1234 or SEQ ID NO: 1233, and at least 3, 4, 5, 6, 7, 8, 9 or all contiguous amino acids of any of SEQ ID NOS: 1-1232

In some embodiments, a nucleotide sequence encodes an amino acid sequence that is at least 80% identical, at least 85% identical, at least 90% identical, or at least 95% identical to SEQ ID NO: 1233. In some embodiments, a nucleotide sequence encodes an amino acid sequence that is at least 80% identical, at least 85% identical, at least 90% identical, or at least 95% identical to SEQ ID NO: 1234.

In some embodiments, any of the CNS-targeting molecules set forth in any of SEQ ID NO: 1-1232 is inserted into an AAV. In some embodiments, any of the CNS-targeting molecules set forth in any of SEQ ID NO: 1-1232 is inserted into AAV2. In some embodiments, AAV2 comprises the sequence set forth in SEQ ID NO: 1233. In some embodiments, SEQ ID NO: 71 is inserted into SEQ ID NO: 1233. SEQ ID NO: 1233 is AAV2 with an amino acid substitution. In some embodiments, any of the CNS-targeting molecules set forth in any of SEQ ID NO: 1-1232 is inserted into AAV9. In some embodiments, AAV9 comprises the sequence set forth in SEQ ID NO: 1234. In some embodiments, SEQ ID NO: 71 is inserted into SEQ ID NO: 1234. SEQ ID NO: 1234 is AAV9. SEQ ID NO: 71 inserted into SEQ ID NO: 1234 can result in SEQ ID NO: 1235.

In some embodiments, insertion of SEQ ID NO: 71 into AAV9 results in the sequence set forth in SEQ ID NO: 1235 (CNSRCV300). In some embodiments, insertion of SEQ ID NO: 71 into AAV9 results in a variant sequence of SEQ ID NO: 1235. In embodiments, the variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 substitutions, insertions, and/or deletions of any of the amino acids in the amino acid sequence of SEQ ID NO: 1235. In some embodiments, the variant comprises at least 80% sequence identity, at least 85% sequence identity, at least 90% sequence identity, at least 95% sequence identity, or at least 99% sequence identity with SEQ ID NO: 1235 or comprises SEQ ID NO: 1235. In embodiments, the variant comprises at least 80%, 85%, 90%, 95%, or 99% sequence identity to the sequence set forth in SEQ ID NO: 1235, and at least 3, 4, 5, 6, 7, 8, or 9 contiguous amino acids of SEQ ID NO: 71.

In some embodiments, the variant refers to a variant in the nucleotide sequence that encodes the amino acid sequences set forth in SEQ ID NO: 1235. In embodiments, the variant in the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 substitutions, insertions, and/or deletions of any of the amino acids in the amino acid sequence of SEQ ID NO: 1235. In some embodiments, the variant in the nucleotide sequence encodes an amino acid sequence that comprises at least 80% sequence identity, at least 85% sequence identity, at least 90% sequence identity, at least 95% sequence identity, or at least 99% sequence identity with SEQ ID NO: 1235. In embodiments, the variant in the nucleotide sequence encodes an amino acid sequence that comprises at least 80%, 85%, 90%, 95%, or 99% sequence identity to the sequence set forth in SEQ ID NO: 1235, and at least 3, 4, 5, 6, 7, 8, or 9 contiguous amino acids of SEQ ID NO: 71.

### AAV Particles

In some embodiments, the CNS-targeting molecules are part of an engineered AAV capsid protein, and the engineered AAV capsid proteins are packaged into AAV particles. In some embodiments, the AAV particles that have enhanced tropism for a target tissue (e.g., CNS and PNS) are provided. CNS-targeting molecules may be inserted into an AAV capsid protein sequence to alter tropism to a particular cell-type, tissue, organ or organism, in vivo, ex vivo or in vitro. In some embodiments, the AAV particles are capable of penetrating the blood brain barrier.

### Delivery of AAV Particles

The AAV particles may be delivered to one or more target cells, tissues, organs, or organisms. In some embodiments, the AAV particles demonstrate enhanced tropism for a target cell type, tissue or organ. As a non-limiting example, the AAV particle may have enhanced tropism for cells and tissues of the central or peripheral nervous systems (CNS and PNS, respectively), or cells and tissues of a muscle. The AAV particles may, in addition, or alternatively, have decreased tropism for an undesired target cell-type, tissue or organ.

In some embodiments, the AAV particles are used to deliver a viral genome to a tissue or cells such as CNS or PNS cell or tissue.

The delivered viral genome may include genetic material of interest, such as, for example, an antibody, an enzyme, or regulatory RNA, amongst others. In some embodiments, the viral genome includes at least one ITR sequence. In some embodiments, the viral genome includes 2 ITR sequences. In some embodiments, the ITR sequences flank the genetic material of interest. In some embodiments, the ITR sequences are complementary to each other. In some embodiments, the ITR regions are derived from the same serotype as the capsid protein. ITR regions may be between 100 and 150 nucleotides in length.

In some embodiments, the AAV particles can be used to infect a wide range of cells (including quiescent and dividing cells) without integration into the host genome and without replicating. In some embodiments, the genome of the virus contains the components required for the assembly of a functional recombinant virus, or viral particle, which is loaded with or engineered to target a particular tissue and express or deliver genetic material of interest to the particular tissue.

### AAV Capsid Proteins Comprising CNS-Targeting Molecules (Targeting Peptides)

In some embodiments, the CNS-targeting molecules, i.e., targeting peptides, are part of a recombinant AAV capsid protein. In some embodiments, AAV capsid proteins described herein may be produced recombinantly and may be based on adeno-associated virus (AAV) wild type sequence.

CNS-targeting molecules may be inserted into an AAV capsid protein sequence to alter tropism relative to the natural AAV capsid protein, to a particular cell-type, tissue, organ or organism, in vivo, ex vivo or in vitro. Stated another way, CNS-targeting molecules, which refer to the targeting peptides, that are inserted into the capsid protein, allow the capsid protein to penetrate the blood brain barrier.

In some embodiments, the targeting peptide is used for enhanced or improved transduction of a target cell or tissue (e.g., cells or tissues of the central nervous system (CNS) or peripheral nervous system (PNS)). In some embodiments, the targeting peptide is used to facilitate the AAV capsid protein across the blood brain barrier following administration to a subject. In some embodiments, the targeting peptide is used for enhanced or improved distribution of the genetic material throughout the multiple brain regions, e.g., frontal cortex, sensory cortex, motor cortex, putamen, thalamus, cerebellar cortex, dentate nucleus, caudate, and/or hippocampus. In some embodiments, the targeting peptide is used for enhanced or improved genetic material expression in multiple brain regions. In some embodiments, the targeting peptide is used for enhanced or improved delivery of genetic material of interest to a desired tissue, cell, or organelle.

In some embodiments, the targeting peptide increases tropism of the AAV capsid to a cell, region, or tissue of the CNS. Examples of CNS cells include but are not limited to neurons (e.g., excitatory neurons, inhibitory neurons, and motor neurons) and glial cells (e.g., ependymal cells, astrocytes, oligodendrocytes. Examples of CNS tissue include but are not limited to the cortex (e.g., frontal cortex, parietal cortex, occipital cortex, temporal cortex), thalamus, hypothalamus, striatum, hippocampus, entorhinal cortex, and basal ganglia.

In some embodiments, the AAV capsid protein comprising a targeting peptide is capable of increased tropism by at least 1.1-, 1.2-, 1.3-, 1.4-, 1.5-fold, relative to an AAV capsid protein that lacks a targeting peptide. In some embodiments, the AAV capsid protein comprising a targeting peptide is capable of increased tropism by over 1.5-fold, relative to an AAV capsid protein that lacks a targeting peptide.

In some embodiments, the AAV capsid protein comprising the targeting peptide facilitates increased expression of delivered genetic material (e.g., a therapeutic cargo) by at least 1.1-, 1.2-, 1.3-, 1.4-, 1.5-fold in a specific cell, region, or tissue, relative to an AAV capsid protein that lacks a targeting peptide. In some embodiments, the AAV capsid protein comprising the targeting peptide facilitates increased expression of delivered genetic material (e.g., a therapeutic cargo) by more than 1.5-fold in a specific cell, region, or tissue, relative to an AAV capsid protein that lacks a targeting peptide.

In some examples, the targeting peptide is between 6 amino acids and 20 amino acids in length, for example 6, 7, 8, 9, 10, 11, 12, 13, 14, 15, 16, 17, 18, 19, or 20 amino acids in length. In some examples, the targeting peptide is between 9 and 16 amino acids in length. In some examples, the targeting peptide is 9 amino acids in length. In some examples, the targeting peptide is 16 amino acids in length.

In some embodiments, the targeting peptide comprises an amino acid sequence of any sequence set forth in SEQ ID NO: 1-1232. In some embodiments, the targeting peptide comprises the amino acid sequence set forth in SEQ ID NO: 71. In some embodiments, the targeting peptide comprises at least 3, 4, 5, 6, 7, 8, 9, or all contiguous amino acids of any sequence set forth in SEQ ID NO: 1-1232. In some embodiments, the targeting peptide comprises at least 3, 4, 5, 6, 7, 8, or 9 contiguous amino acids of the sequence set forth in SEQ ID NO: 71. In some embodiments, the targeting peptide is part of an AAV vector. In some embodiments, the targeting peptide is part of a capsid protein of the AAV vector. In some embodiments, nucleic acid sequences encode targeting peptides.

In some embodiments, the AAV capsid protein comprise a nucleic acid sequence encoding a peptide that comprises an amino acid sequence of any sequence set forth in SEQ ID NO: 1-1232. In some embodiments, the AAV capsid protein comprise a nucleic acid sequence encoding a peptide that comprises an amino acid sequence of SEQ ID NO: 71. In some embodiments, the AAV capsid protein comprise a nucleic acid sequence encoding a peptide that comprises at least 3, 4, 5, 6, 7, 8, 9, or all contiguous amino acids of any sequence set forth in SEQ ID NO: 1-1232. In some embodiments, the AAV capsid protein comprise a nucleic acid sequence encoding a peptide that comprises at least 3, 4, 5, 6, 7, 8, or 9 contiguous amino acids of the sequence set forth in SEQ ID NO: 71.

Insertion of Targeting Peptides into Capsid Proteins

In some embodiments, a targeting peptide is a part of a capsid protein, and the targeting peptide is inserted at a location between amino acid residues 450 and 600 of the capsid protein. In some embodiments, the amino acid sequence is inserted at a location between amino acid residues 587 and 590 of the AAV9 capsid protein. In some embodiments, the amino acid sequence is inserted at location between amino acid residues 588 and 589 of the AAV2 capsid protein.

In some embodiments, a peptide sequence that comprises any of the sequences set forth in SEQ ID NO: 1-1232 is inserted into the capsid protein. In some embodiments, the peptide sequence comprises 3, 4, 5, 6, 7, 8, 9 or all contiguous amino acids of any of the sequences set forth in SEQ ID NO: 1-1232. In some embodiments, disclosed is a peptide sequence that comprises the sequence set forth in SEQ ID NO: 71. In some embodiments, the peptide sequence comprises 3, 4, 5, 6, 7, 8, or 9 contiguous amino acids of the sequence set forth in SEQ ID NO: 71.

In some embodiments, a targeting peptide is inserted into any of the AAV capsid protein comprises any of the AAV serotypes AAV1, AAV2, AAV3B, AAV5, AAV6, AAV8 and AAV9. In some embodiments, the AAV capsid protein comprises the AAV2 serotype. In some embodiments, the AAV2 serotype includes a mutation at position 588 from arginine to alanine. In some embodiments, the AAV2 serotype comprises the sequence of SEQ ID NO: 1233. In some embodiments, an amino acid sequence is inserted between positions 450 and 600 of SEQ ID NO: 1233. In some embodiments, an amino acid sequence is inserted between positions 588 and 589 of SEQ ID NO: 1233. In some embodiments, the AAV capsid protein comprises the AAV9 serotype. In some embodiments, the AAV9 serotype includes the sequence of SEQ ID NO: 1234. In some embodiments, an amino acid sequence is inserted between positions 450 and 600 of SEQ ID NO: 1234. In some embodiments, the amino acid sequence is inserted between positions 587 and 590 of SEQ ID NO: 1234. In some embodiments, the amino acid sequence inserted into the AAV2 or AAV9 serotypes comprises an amino acid sequence set forth in SEQ ID NO: 1-1232. In some embodiments, the amino acid sequence inserted into the AAV2 or AAV9 serotypes comprises an amino acid sequence set forth in SEQ ID NO: 71. In some embodiments, insertion of the amino acid sequence set forth in SEQ ID NO: 71 into an AAV9 serotype results in the sequence set forth in SEQ ID NO: 1235 (CNSRCV300).

In some embodiments, a targeting peptide is inserted into an AAV capsid protein. Any targeting peptide described herein may be inserted into a parent AAV capsid protein in any location that results in fully functional AAV particles. The targeting peptide may be inserted into capsid proteins VP1, VP2 and/or VP3. In some embodiments, a targeting peptide, is inserted in a hypervariable region of the AAV capsid protein. Non-limiting examples of such hypervariable and/or surface exposed loop of the AAV capsid protein. In some embodiments, the targeting peptide is inserted into the H1 loop. In some embodiments, the targeting peptide is inserted into the DE loop. In some embodiments, the targeting sequencing is inserted into the variable region of the surface exposed loop, for example any of VR-I, VR-II, VR-III, VR-IV, VR-V, VR-VI, VR-VII, VR-VIII and VR-IX. In some embodiments, the targeting peptide comprises any of SEQ ID NO: 1-1232. In some embodiments, the targeting peptide comprises SEQ ID NO: 71.

In some embodiments, the AAV capsid proteins described herein have enhanced tropism for a specific cell or tissue, for example, a CNS or PNS cell or tissue. In some embodiments, the enhanced tropism for a specific cell or tissue is due to the insertion of any of the sequences set forth in SEQ ID NO: 1-1232 into the AAV capsid protein. In some embodiments, the enhanced tropism for a specific cell or tissue is due to the insertion of SEQ ID NO: 71 in to the AAV capsid protein. In some embodiments, the AAV capsid proteins are capable of penetrating the blood brain barrier. In some embodiments, the AAV capsid proteins described herein are capable of penetrating the blood brain barrier due to the insertion of any of the sequences set forth in SEQ ID NO: 1-1232 into the AAV capsid protein. In some embodiments, the AAV capsid proteins described herein are capable of penetrating the blood brain barrier due to the insertion of SEQ ID NO: 71 into the AAV capsid protein. In some embodiments, the AAV capsid proteins described herein are capable of distributing throughout multiple brain regions including, but not limited to the frontal cortex, sensory cortex, motor cortex, putamen, thalamus, cerebellar cortex, and hippocampus. In some embodiments, the AAV capsid proteins described herein are capable of distributing throughout multiple brain regions due to the insertion of any of the SEQ ID NO: 1-1232 into the AAV capsid protein. In some embodiments, the AAV capsid proteins described herein are capable of distributing throughout multiple brain regions due to the insertion of SEQ ID NO: 71 into the AAV capsid protein.

### Pharmaceutical Compositions and Dosage Forms

Compositions described herein including CNS-targeting molecules (targeting peptides), AAV capsid proteins, AAV particles, and AAV vectors can be included in pharmaceutical compositions. In some embodiments, the pharmaceutical compositions can include one or more excipients or diluents to (1) increase stability; (2) increase cell transfection or transduction; (3) permit the sustained or delayed release of the genetic material; (4) alter the biodistribution (e.g., target the composition to specific tissues or cell types); (5) increase the translation of encoded protein; (6) alter the release profile of encoded protein and/or (7) allow for regulatable expression of the genetic material.

The pharmaceutical compositions described herein can be administered periodically, such as once or twice a day, or any other suitable time period. For example, pharmaceutical compositions may be administered to a subject in need once a week, once every other week, once every three weeks, once a month, every other month, every three months, every six months, every nine months, once a year, every eighteen months, every two years, every thirty months, every three years, every five years, every 10 years, every 20 years, or a one-time administration.

In some embodiments, the compositions described herein (CNS-targeting (targeting peptides), AAV capsid proteins, AAV particles, and AAV vectors) can be formulated in a wide variety of dosage forms, including but not limited to nasal, pulmonary, oral, topical, or parenteral dosage forms for clinical. Each of the dosage forms can comprise various solubilizing agents, disintegrating agents, surfactants, fillers, thickeners, binders, diluents such as wetting agents or other pharmaceutically acceptable excipients. The compositions described herein can also be formulated for injection, insufflation, infusion, or intradermal exposure. For instance, an injectable formulation may comprise the disclosed compositions in an aqueous or non-aqueous solution at a suitable pH and tonicity. The compositions can be included liquid dosage form for oral administration, such as suspensions, emulsions, or syrups.

In some embodiments, the pharmaceutical compositions described herein function to increase the stability, increase transduction or transfection efficiency, impact biodistribution, increase expression of the protein, and/or alter the release profile.

### Methods of Delivery of and Treatment Using the CNS-Targeting Molecules

In some embodiments, methods for introducing the compositions described herein (CNS-targeting molecules (targeting peptides), AAV capsid proteins, AAV particles, and AAV vectors) into cells and/or tissues are provided. In some embodiments, the methods comprise introducing into cells and/or tissues any of the compositions described herein in an amount sufficient to modulate, e.g., increase, the production of a target mRNA and/or protein in the cells and/or tissues.

In some embodiments, the compositions described herein are delivered via a localized delivery route. In some embodiments, the localized delivery route includes any one or more of intramuscular administration, intraparenchymal administration, and intracerebral administration, amongst others. In some embodiments, the compositions described herein are administered via a localized delivery route through a bolus infusion.

In some embodiments, the compositions described herein are administered through systemic administration. In some embodiments, systemic administration includes intravenous administration. In some embodiments, intravenous administration includes subcutaneous administration. In some embodiments, the systemic administration includes intraventricular administration.

In some embodiments, the compositions described herein are administered to the central nervous system of via intraventricular administration and/or intravenous administration. In some embodiments, the compositions described herein are administered to the central nervous system via systemic administration. In some embodiments, the systemic administration is intravenous (IV) injection. In some embodiments, the CNS-targeting molecules described herein are administered to the central nervous system via intraventricular administration.

In some embodiments the compositions can be delivered to target cell or target tissue including, but not limited to, the CNS, heart, lung, trachea, esophagus, muscle, bone, cartilage, stomach, pancreas, intestine, liver, bladder, kidney, ureter, urethra, uterus, fallopian tube, ovary, testes, prostate, eye, blood, lymph, or oral mucosa. In some embodiments, the target cell or tissue includes, but is not limited to CNS, heart, lung, trachea, esophagus, muscle, bone, cartilage, stomach, pancreas, intestine, liver, bladder, kidney, ureter, urethra, uterus, fallopian tube, ovary, testes, prostate, eye, blood, lymph, or oral mucosa. In some embodiments, the target cell or target tissue is a CNS cell or tissue. In some embodiments, the target cell or tissue is liver cell or tissue.

In some embodiments, the target cell includes, but is not limited to, neurons, glial cells, astrocytes, oligodendroglia, microglia, Schwann cells, ependymal cells, hepatocytes, stellate fat storing cells, Kupffer cells, liver endothelial cells, epithelial cells, cardiomyocytes, smooth muscle cells, T-cells, B cells, hematopoietic stem cells, and embryonic stem cells.

In some embodiments, the compositions described herein are delivered to the central nervous system through the cerebral spinal fluid pathway. In some embodiments, compositions described herein are administered to the central nervous system via intraparenchymal delivery. In some embodiments, the compositions described herein are administered to the central nervous system via intracranial delivery. In some embodiments, the compositions described herein are delivered to the central nervous system via intraocular delivery. In some embodiments, the compositions described herein are administered to the brain. In some embodiments, the compositions described herein are administered to the brain via injection into the brain. In some embodiments, the compositions described herein are administered to the brain via intrahippocampal injection.

In some embodiments, disclosed is a method of delivering a nucleic acid to a target cell or tissue of a subject, comprising: administering a composition comprising an AAV vector, wherein the AAV vector further comprises a capsid protein comprising a targeting peptide comprising at least 5, 6, 7, 8, or 9 contiguous amino acids of an amino acid sequence set forth in SEQ ID NO: 1-1232. In some embodiments, the capsid protein comprises the targeting peptide set forth in any one of SEQ ID NO: 1-1232.

In some embodiments, the compositions described herein are administered as part of a composition that allows for extended release. In some embodiments, the compositions comprise a formulation that includes a depot.

Disclosed herein are methods of treatment using any of the compositions described herein (CNS-targeting molecules (targeting peptides), AAV capsid proteins, AAV vectors, and AAV particles). In embodiments, the disclosed compositions can be used to treat any one or more of muscular or neuromuscular disorders, neurooncological disorders, neurological diseases/disorders, and neurodegenerative disorders, amongst others. In embodiments, the disclosed compositions can be used to treat any one or more of Alzheimer's disease, Huntington's disease; autism; Parkinson's disease; Spinal muscular atrophy, Friedreich's ataxia. In embodiments, the disclosed compositions are used in treatments through any of the methods of delivery described herein.

In some embodiments, disclosed are methods for treating, or ameliorating a disease or condition associated with abnormal gene and/or protein in a subject in need of treatment, the methods comprising administering to the subject any effective amount of at least one of the compositions described herein (CNS-targeting molecules (targeting peptides), AAV capsid proteins, AAV vectors, and AAV particles), delivering the compositions described herein into targeted cells, inhibiting or activating the gene expression and protein production, and ameliorating symptoms of the disease or condition in the subject.

### EQUIVALENTS AND SCOPE

The disclosure includes many equivalents to the specific embodiments described herein. A person of skill in the art will be able to ascertain equivalents to the specific embodiments, through routine experimentation.

It is assumed that words of this disclosure are for the purpose of description and not limitation. Changes to words in the claims can be made, while still retaining the scope of the disclosure in its broad aspect. Specific embodiments of the disclosure have been described herein. However, these embodiments are not intended to be limiting of the broad scope of this disclosure. While some embodiments comprise/include the disclosed features and may therefore include additional features not specifically described, other embodiments may be essentially free of or completely free of non-disclosed elements—that is, non-disclosed elements may optionally be essentially omitted or completely omitted.

The disclosure is further illustrated by the following examples, which are intended to be purely illustrative and not limiting of the disclosure herein.

### EXAMPLES

### Example 1. Methods

### 1.1. AAV Capsid Library Generation

Capsid variants for library screening were synthesized as an oligo pool. Each capsid peptide was synthesized with unique nucleotide sequences encoding the peptide, and each peptide was linked to at least two distinct barcodes. The oligo pool was cloned into a linearized intermediate plasmid, followed by cloning of a constant donor sequence to separate the barcode and peptide region and generate the full AAV vector construct. Two separate constant donor sequences were used, generating the barcoded library transcript under the control of a neuron specific promoter or a ubiquitous promoter. This enables an assessment of which capsids drive the most functional mRNA expression in neurons as well as all cell types transduced. Moreover, each barcode is linked to a unique molecular identifier (UMI). Based on the overall size of the cloned library each barcode is appended to hundreds to thousands of UMIs. The utility of the UMI is to additionally assess how many distinct AAV transduction events give rise to the NGS read counts that are measured.

The peptide sequences listed in Table 1 were inserted into variable region 8 of AAV serotypes 2 and 9. Where indicated in Table 1, peptides are inserted into AAV9 (SEQ ID 1234) between 587/590, the insertions are after amino acid 587 and before 590 replacing amino acids 588 and 589. Other peptides inserted into AAV9 indicated in Table 1 are inserted between amino acids 588/589. Peptides inserted into AAV2, were inserted between positions 588/589 and the wildtype arginine at position 588 is altered to alanine (R588A) (Seq ID 1233).

These AAV plasmid libraries were manufactured in HEK293 cells. Briefly, libraries were produced by transient transfection including supplementation of Rep in trans, capsids were purified by cesium density centrifugation, and buffer exchanged into PBS by Amicon filtration. DNase-resistant viral genomic titers were measured by quantitative real time PCR.

### 1.2. Administration of AAV Libraries to Cynomolgus Macaques

The AAV library was administered into cynomolgus macaques as an intravenous bolus injection with a volume ≤5 mL per kg of animal body weight.

### 1.3. Tissue Collection and Processing

At the time of sacrifice, the brain was removed and placed in a coronal brain matrix in an ice-cold nuclease free PBS bath for approximately 10 minutes. The brain was sliced at a 4 mm coronal slice thickness. All slices were hemisected along the mid-sagittal plane. The brain slices were processed and stored according to the following table.

1.4. Brain Tissue Collection for Molecular Analysis (mRNA and DNA)

Brain slices collected for mRNA and DNA molecular analysis (NGS) were placed in chilled RNA Later and refrigerated (1 to 8° C.) for approximately 24 to 72 hours to preserve mRNA integrity. After storage, 2 mm punches were collected according to the Sponsor-provided brain template. The brain punch template is maintained in the raw data. Following removal from RNA Later and prior to obtaining the punches, the brain slices were placed on a flat surface and cross reference labels added to the brain slice and photographed (photographs are maintained with the raw data). All brain punch samples were placed into clean vials, frozen on dry ice and stored at −60° C. or below until shipment. The residual brain slices, following sample collection, were frozen and stored at −60° C. or below until shipment.

The residual brain slices, following sample collection, were frozen and stored at −60° C. or below until shipment.

### 1.5. Spinal Cord and Dorsal Root Ganglia

The spinal cord with dorsal root ganglia (DRG) attached were divided into four segments (cervical, thoracic, lumbar and sacral). From the central portion of each segment, a single 2-2.5 cm cross-sections were collected for potential evaluation. The cross-sections were placed in RNA Later and refrigerated (1 to 8° C.) for approximately 24 to 72 hours after which the samples were frozen at −60° C. or below until shipment. Right and left DRG pairs associated with the portion of each segment was removed and frozen (left and right saved together) on dry ice and stored at −60° C. or below until shipment.

### 1.6. Biodistribution—Other Tissues

An approximate 500-600 mg sample of tissue from the liver, testes, pancreas, lung, skeletal muscle (quadriceps), heart, kidney, lymph node, and spleen were collected for analysis. Samples were frozen on dry ice and stored at −60° C. or below until shipment.

1.7. Tissue Lysis and Total RNA Isolation for Analysis of Library Expression from Punches

Brain punches to be processed were placed on dry ice. For spinal cord, DRG and liver, approximately 35 mg of each tissue sample was excised on dry ice. Each tissue sample was then transferred to an Eppendorf tube pre-filled with 600 μL of TRIZOL and two 3.2 mm steel beads. Sample tubes were placed into a Retsch MM300 Tissue-Lyser and 8 rounds of homogenization were performed at a frequency of 25.1 Hz for 1½ minutes with a 2-minute pause between rounds to prevent overheating.

Isolation and purification of total RNA from homogenized tissue was performed using the MagMAX™-96 Total RNA Isolation Kit in conjunction with the KingFisher™ Flex Purification System according to the manufacturer's protocol. Briefly, bromochloropropane (BCP) was added and then centrifugation was performed to separate the homogenate into aqueous and organic phases. The aqueous phase containing partially purified RNA was then transferred to a 96 well KingFisher processing plate. Isopropanol (100%) was added to each well followed by addition of magnetic RNA binding beads. Subsequent processing was performed on the KingFisher™ Flex Purification System. Briefly, the RNA binding beads were magnetically captured and an on-bead DNase digestion and several washes were performed. Purified RNA was eluted in 100 μL of low salt elution buffer. The KingFisher processing plate was then transferred to a magnetic stand on the benchtop. The eluants (~90 μL) were transferred away from any residual beads into a 96-well PCR plate for downstream processing. The yield and purity of the RNA was determined using a NanoDrop 8000 spectrophotometer.

1.8. Tissue Lysis and Total RNA Isolation for Analysis of Library Expression from Brain Slices

Hemisected brain slices in cassettes to be processed were placed on dry ice. Each brain slice was removed from the cassette, weighted (up to 3 g), and placed into 50 mL Bigprep Lysing Matrix D tubes. The tubes were filled with TRIZOL Reagent at 10 mL per g of tissue. The samples were then placed in a CoolBigPrep adapter for 2×50 mL tubes and into a FastPrep-2 Classic bead beating grinder and lysis system. The tissue samples were homogenized at 4.0 meters/second for 30 seconds with 2 minutes pause. The tissue homogenization was repeated 4 times. The lysate was centrifuged at 4,300×g for 5 minutes at 4° C. The clarified lysate was removed to a new tube and a second round of centrifugation and removal of clarified lysate to a fresh tube was conducted. Two 5 mL aliquots were transferred to separate tubes. The remaining sample was frozen at −80° C. if present. The 5 mL aliquots were processed by adding 0.2 mL per mL Trizol of molecular grade chloroform. The sample was mixed by inversion and vortexing for 30 seconds then centrifuged at 4,300×g for 30 minutes at 4° C. The aqueous phase, approximately 50% of the lysate volume or ~2.5 mL was removed equally to 2 mL microcentrifuge tubes. 0.5 mL per mL Trizol of molecular grade isopropanol was added to the tubes and mixed by inversion. The samples were incubated for 10 minutes on ice followed by centrifugation at 12,000×g for 10 minutes at 4° C. The RNA pellet was resuspended in 75% ethanol at 1 mL per mL Trizol, vortexed, and centrifuged at 12,000×g for 5 minutes at 4° C. The RNA pellet was air dried for 5 minutes and dissolved in 0.2 mL per mL Trizol with RNAse free DEPC treated water. The yield and purity of the RNA was determined using a NanoDrop 8000 spectrophotometer. Samples were spot checked for RNA integrity using the Agilent RNA 6000 Nano kit and Bioanalyzer 2100.

1.9. Purification of mRNA from Total RNA

mRNA was purified from select brain slice derived total RNA using Dynabeads mRNA Purification Kit following the manufacturer's instructions. Samples were spot checked for RNA integrity using the Agilent RNA 6000 Nano kit and Bioanalyzer 2100.

1.10. Reverse Transcription of Total RNA and mRNA to cDNA

The maximum allowable RNA input per kit was used following the manufacturer's protocol. QuantiTect Reverse Transcription Kit was used with a library transcript specific RT oligo using total RNA or purified mRNA at template. The manufacturer's protocol was used.

### 1.11. PCR Amplification of Library Barcodes, Illumina NGS, and Bioinformatic Analysis

All cDNA from various sources were treated similarly. Vector genomes from the test articles were extracted using QIAamp MinElute Virus Spin Kit. Library barcodes were amplified using transcript specific oligos with KAPA HiFi HotStart ReadyMix. Amplification of library specific amplicons was performed with the following cycling conditions: 98° C. for 2:00 min; 25 cycles at 98° C. for 15 sec; 58° C. for 25 sec; 72° C. for 30 sec followed by 72° C. for 3 minutes. Amplification was qualitatively confirmed by agarose gel electrophoresis and relative apparent amplification was used to determine the dilution of amplicons needed for indexing. Illumina plate level i5 and well level i7 indices were added to the amplicons with 12 cycles of amplification: 98° C. for 30 sec; 12 cycles at 98° C. for 15 sec; 60° C. for 25 sec; 72° C. for 30 sec followed by 72° C. for 10 minutes. Finally, samples were pooled and purified using Qiagen GeneRead Size Selection Kit following the manufacturer's protocol. Samples were sequenced on Illumina MiSeq platform using MiSeq Reagent Kit v2.

Following NGS of library amplicons the reads were demultiplexed and features were extracted using a custom bioinformatic pipeline. Extracted barcodes were used to query a predetermined lookup table and return the identity of the corresponding variant. Finally, the log 2 fold change enrichment of each variant was normalized to its relative abundance in the administered test article.

### Example 2. Results from Round 2 Library Evaluation

Previously, a first-round SIFTER (Selecting in vivo for transduction and expression of RNA) library was conducted that identified ~65,000 AAV variants included in this round 2 library. The round 2 library of ~65,000 variants each under the control of neuron specific human synapsin 1 (hSyn1) promoter or a ubiquitous CMV promoter was administered to two NHPs. Library barcodes were recovered from cDNA reverse transcribed from total RNA extracted from CNS tissue. Approximately 14,000 variants from this round 2 SIFTER library were recovered. After filtering out variants that were poorly recovered, a total of 6,728 variants were identified from the hSyn1 library, and 4,005 variants from the CMV library. A total of 1,236 variants were selected for evaluation in a final selection to nominate lead capsids. Several of the variants exhibited substantial improvement over AAV9 in CNS transduction.

The fold change enrichment in each tissue sample collected was determined by NGS and normalized to the abundance in the administered test article. The average log 2FC across all the tissue samples analyzed from the animals per group is shown in Table 1.

TABLE 1

| | | Parent | Average | Peptide Insertion |
|---|---|---|---|---|
| Sequence ID | Peptide Sequence | Capsid | Log2FC | Site |
| Seq ID 1 | NFRQSEMLN | AAV9 | 9.99 | 587/590 |
| Seq ID 2 | QRNTDHITP | AAV9 | 9.33 | 587/590 |
| Seq ID 3 | MQFDPQKPD | AAV9 | 9.22 | 587/590 |
| Seq ID 4 | TMSSNDNMR | AAV9 | 9.10 | 587/590 |
| Seq ID 5 | VKESVQKAA | AAV9 | 8.82 | 587/590 |
| Seq ID 6 | ILNVSKPDN | AAV9 | 8.31 | 587/590 |
| Seq ID 7 | SQAAQLPTQ | AAV9 | 8.24 | 587/590 |
| Seq ID 8 | QVMAIDAAI | AAV9 | 8.17 | 587/590 |
| Seq ID 9 | NDTARERAI | AAV9 | 7.98 | 587/590 |
| Seq ID 10 | SMAAKVTAE | AAV9 | 7.94 | 587/590 |
| Seq ID 11 | TLGHQHSPI | AAV9 | 7.83 | 587/590 |
| Seq ID 12 | TVPSSGKQH | AAV9 | 7.49 | 587/590 |
| Seq ID 13 | AMVQISTPL | AAV9 | 7.47 | 587/590 |
| Seq ID 14 | VQVTGTVRF | AAV9 | 7.34 | 587/590 |
| Seq ID 15 | MIAQKTVVH | AAV9 | 7.32 | 587/590 |
| Seq ID 16 | NTHFQGQPP | AAV9 | 7.26 | 587/590 |
| Seq ID 17 | MDMQATGKN | AAV9 | 7.25 | 587/590 |
| Seq ID 18 | NLAHTRQPQ | AAV9 | 7.24 | 587/590 |
| Seq ID 19 | LDIPPMNSS | AAV9 | 7.22 | 587/590 |
| Seq ID 20 | QHSTPSWPS | AAV9 | 7.17 | 587/590 |
| Seq ID 21 | AFTRHNGTGGKSIYGA | AAV2 | 7.11 | R588A/589 |
| Seq ID 22 | ARGARDEFT | AAV9 | 7.07 | 587/590 |
| Seq ID 23 | RPSNDKAMM | AAV9 | 7.06 | 587/590 |
| Seq ID 24 | AQVHMTMPQ | AAV9 | 7.05 | 587/590 |
| Seq ID 25 | ARQTFQHHL | AAV9 | 7.00 | 587/590 |
| Seq ID 26 | HQTDAPTDW | AAV9 | 6.98 | 587/590 |
| Seq ID 27 | ANMISEPRISYGNDAA | AAV9 | 6.97 | 588/589 |
| Seq ID 28 | IERWQAEII | AAV9 | 6.96 | 587/590 |
| Seq ID 29 | VPHNGLHST | AAV9 | 6.93 | 587/590 |
| Seq ID 30 | RIHDPSLVS | AAV9 | 6.93 | 587/590 |
| Seq ID 31 | IPATHPQLM | AAV9 | 6.89 | 587/590 |
| Seq ID 32 | ITTVKNVTV | AAV9 | 6.89 | 587/590 |
| Seq ID 33 | DKSVKSTAI | AAV9 | 6.88 | 587/590 |
| Seq ID 34 | IRTHEVPLQ | AAV9 | 6.87 | 587/590 |
| Seq ID 35 | SPSSTSNNN | AAV9 | 6.74 | 587/590 |
| Seq ID 36 | LSTTSQVHN | AAV9 | 6.70 | 587/590 |
| Seq ID 37 | NMHNETSMT | AAV9 | 6.70 | 587/590 |
| Seq ID 38 | NDSRKANIG | AAV9 | 6.68 | 587/590 |
| Seq ID 39 | KPPNVQDAK | AAV9 | 6.65 | 587/590 |
| Seq ID 40 | FQHMTPKPS | AAV9 | 6.60 | 587/590 |
| Seq ID 41 | KQPSRLMEW | AAV9 | 6.54 | 587/590 |
| Seq ID 42 | VYDQVKATH | AAV9 | 6.51 | 587/590 |
| Seq ID 43 | SYRNQYDDQ | AAV9 | 6.45 | 587/590 |
| Seq ID 44 | VQSTKQSVQ | AAV9 | 6.42 | 587/590 |
| Seq ID 45 | HQGKVDAQN | AAV9 | 6.42 | 587/590 |
| Seq ID 46 | IQGEIRVHT | AAV9 | 6.41 | 587/590 |
| Seq ID 47 | TQTLKVEDR | AAV9 | 6.38 | 587/590 |
| Seq ID 48 | VQSPPGIMP | AAV9 | 6.37 | 587/590 |
| Seq ID 49 | VHYAPNYTT | AAV9 | 6.37 | 587/590 |
| Seq ID 50 | YGGSETNVR | AAV9 | 6.29 | 587/590 |
| Seq ID 51 | IRTQTHPQV | AAV9 | 6.28 | 587/590 |
| Seq ID 52 | ETQVQPIQH | AAV9 | 6.27 | 587/590 |
| Seq ID 53 | FPVSSVNDV | AAV9 | 6.27 | 587/590 |
| Seq ID 54 | IQKISQAMA | AAV9 | 6.26 | 587/590 |
| Seq ID 55 | SQTQIGQPY | AAV9 | 6.22 | 587/590 |
| Seq ID 56 | AIRTNVGPV | AAV9 | 6.19 | 587/590 |
| Seq ID 57 | TQMSGQTPH | AAV9 | 6.16 | 587/590 |
| Seq ID 58 | PAKSVMIPY | AAV9 | 6.15 | 587/590 |
| Seq ID 59 | QRVSQTNSF | AAV9 | 6.14 | 587/590 |
| Seq ID 60 | AFAGANYAAWAVNVAA | AAV2 | 6.10 | R588A/589 |
| Seq ID 61 | AVVRPQSTM | AAV9 | 6.09 | 587/590 |
| Seq ID 62 | PLQTPPSDN | AAV9 | 6.07 | 587/590 |
| Seq ID 63 | VTVSQQMTK | AAV9 | 6.07 | 587/590 |
| Seq ID 64 | DPFEGRIST | AAV9 | 6.05 | 587/590 |
| Seq ID 65 | NNPTGGIAL | AAV9 | 6.05 | 587/590 |
| Seq ID 66 | ARYTHLVAIQNKEEIA | AAV2 | 6.02 | R588A/589 |
| Seq ID 67 | IVTGGVLKP | AAV9 | 6.00 | 587/590 |
| Seq ID 68 | THMSTQASI | AAV9 | 6.00 | 587/590 |
| Seq ID 69 | AVPGEEEQKFEVEKYA | AAV2 | 5.97 | R588A/589 |
| Seq ID 70 | ATDTSHLVV | AAV9 | 5.96 | 587/590 |
| Seq ID 71 | YVNIMDDMD | AAV9 | 5.96 | 587/590 |
| Seq ID 72 | IRPDKQGGV | AAV9 | 5.95 | 587/590 |
| Seq ID 73 | NQTESQWKG | AAV9 | 5.94 | 587/590 |
| Seq ID 74 | TNEGLYKDN | AAV9 | 5.94 | 587/590 |
| Seq ID 75 | IMVGNQTVM | AAV9 | 5.92 | 587/590 |
| Seq ID 76 | IWAEKGLDT | AAV9 | 5.91 | 587/590 |
| Seq ID 77 | MPSQGYQQV | AAV9 | 5.90 | 587/590 |
| Seq ID 78 | NTFGHNVAP | AAV9 | 5.87 | 587/590 |
| Seq ID 79 | ASRTVSDSIKKLITSA | AAV2 | 5.87 | R588A/589 |
| Seq ID 80 | NHTSTPPQQ | AAV9 | 5.86 | 587/590 |
| Seq ID 81 | NAHAPIPVE | AAV9 | 5.83 | 587/590 |
| Seq ID 82 | KSTNTHEIG | AAV9 | 5.83 | 587/590 |
| Seq ID 83 | NTRVTNAHD | AAV9 | 5.82 | 587/590 |
| Seq ID 84 | AHRDACLTGSKIQVDA | AAV2 | 5.81 | R588A/589 |
| Seq ID 85 | AVAAETGQI | AAV9 | 5.80 | 587/590 |
| Seq ID 86 | NQPSMVNGH | AAV9 | 5.80 | 587/590 |
| Seq ID 87 | PMTQQHHSM | AAV9 | 5.80 | 587/590 |
| Seq ID 88 | YEKVDSLMM | AAV9 | 5.79 | 587/590 |
| Seq ID 89 | MDVGTHGKA | AAV9 | 5.78 | 587/590 |
| Seq ID 90 | THNSQVHPS | AAV9 | 5.77 | 587/590 |
| Seq ID 91 | PTQVRQVQQ | AAV9 | 5.73 | 587/590 |
| Seq ID 92 | ITRTVNTVE | AAV9 | 5.71 | 587/590 |
| Seq ID 93 | AMIMSPFGT | AAV9 | 5.70 | 587/590 |
| Seq ID 94 | HAFPSFPAA | AAV9 | 5.70 | 587/590 |
| Seq ID 95 | KMMDNTPFY | AAV9 | 5.68 | 587/590 |
| Seq ID 96 | PQRLSVDPN | AAV9 | 5.67 | 587/590 |
| Seq ID 97 | TWGTYQHSV | AAV9 | 5.66 | 587/590 |
| Seq ID 98 | HYQQRDMSQ | AAV9 | 5.66 | 587/590 |
| Seq ID 99 | SQGHVQERM | AAV9 | 5.64 | 587/590 |
| Seq ID 100 | QPRTTGNDF | AAV9 | 5.64 | 587/590 |
| Seq ID 101 | PKGTISSQQ | AAV9 | 5.62 | 587/590 |
| Seq ID 102 | REHTAAWFA | AAV9 | 5.62 | 587/590 |
| Seq ID 103 | QLTHSNTSQ | AAV9 | 5.62 | 587/590 |
| Seq ID 104 | KDMGAGHSM | AAV9 | 5.60 | 587/590 |
| Seq ID 105 | ARGAQRLAALGDTAWA | AAV9 | 5.60 | 588/589 |
| Seq ID 106 | KQAHREHEE | AAV9 | 5.58 | 587/590 |
| Seq ID 107 | KSSHPYTDE | AAV9 | 5.55 | 587/590 |
| Seq ID 108 | MPNHVAYKD | AAV9 | 5.53 | 587/590 |
| Seq ID 109 | PPTTQYPQT | AAV9 | 5.52 | 587/590 |
| Seq ID 110 | RSHTTGQDS | AAV9 | 5.51 | 587/590 |
| Seq ID 111 | SQLKASVTN | AAV9 | 5.51 | 587/590 |
| Seq ID 112 | TAPSTRFEV | AAV9 | 5.50 | 587/590 |
| Seq ID 113 | PSTLEPEVW | AAV9 | 5.49 | 587/590 |
| Seq ID 114 | YTSPPQMMV | AAV9 | 5.49 | 587/590 |
| Seq ID 115 | PVPSRQVST | AAV9 | 5.48 | 587/590 |
| Seq ID 116 | LEKVQVHQN | AAV9 | 5.47 | 587/590 |
| Seq ID 117 | NQIKLQSMS | AAV9 | 5.46 | 587/590 |
| Seq ID 118 | GVMSPTPMV | AAV9 | 5.46 | 587/590 |
| Seq ID 119 | PTQRAERYF | AAV9 | 5.45 | 587/590 |
| Seq ID 120 | PTAKVVDSV | AAV9 | 5.45 | 587/590 |
| Seq ID 121 | HRIHEVQGG | AAV9 | 5.45 | 587/590 |
| Seq ID 122 | KNTTPVGQQ | AAV9 | 5.44 | 587/590 |
| Seq ID 123 | AGPPLKGAGGKERPGA | AAV2 | 5.44 | R588A/589 |
| Seq ID 124 | IQPASGQNM | AAV9 | 5.42 | 587/590 |
| Seq ID 125 | YQNQGVNVP | AAV9 | 5.40 | 587/590 |
| Seq ID 126 | NASSQSNAN | AAV9 | 5.36 | 587/590 |
| Seq ID 127 | SNTVRGDAA | AAV9 | 5.36 | 587/590 |
| Seq ID 128 | SKKAEFGTE | AAV9 | 5.35 | 587/590 |
| Seq ID 129 | KVSSEMMVH | AAV9 | 5.34 | 587/590 |
| Seq ID 130 | TMIEQRMDQ | AAV9 | 5.34 | 587/590 |
| Seq ID 131 | PPHIYHHMS | AAV9 | 5.33 | 587/590 |
| Seq ID 132 | MVNKAPTGA | AAV9 | 5.31 | 587/590 |
| Seq ID 133 | KSPVARGDT | AAV9 | 5.31 | 587/590 |
| Seq ID 134 | LLKQRQLMD | AAV9 | 5.29 | 587/590 |
| Seq ID 135 | KMKQIELVE | AAV9 | 5.29 | 587/590 |
| Seq ID 136 | RAPNRVNDE | AAV9 | 5.28 | 587/590 |
| Seq ID 137 | TMNQAPPAQ | AAV9 | 5.27 | 587/590 |
| Seq ID 138 | LVKEDEHWI | AAV9 | 5.26 | 587/590 |
| Seq ID 139 | MIGQRDNNS | AAV9 | 5.25 | 587/590 |
| Seq ID 140 | TSHMVSHAV | AAV9 | 5.24 | 587/590 |
| Seq ID 141 | IQDEEAKYT | AAV9 | 5.23 | 587/590 |
| Seq ID 142 | TGGSKFPYM | AAV9 | 5.23 | 587/590 |
| Seq ID 143 | VEGSAQMIK | AAV9 | 5.21 | 587/590 |
| Seq ID 144 | FTDISKEPS | AAV9 | 5.21 | 587/590 |
| Seq ID 145 | TERPMMQQL | AAV9 | 5.20 | 587/590 |
| Seq ID 146 | VSLGHDKPV | AAV9 | 5.19 | 587/590 |
| Seq ID 147 | TQRTHISDM | AAV9 | 5.16 | 587/590 |
| Seq ID 148 | TSYSTHASS | AAV9 | 5.15 | 587/590 |
| Seq ID 149 | NTKSADHEY | AAV9 | 5.13 | 587/590 |
| Seq ID 150 | RDPLREQRP | AAV9 | 5.13 | 587/590 |
| Seq ID 151 | LGQRTVQAQ | AAV9 | 5.12 | 587/590 |
| Seq ID 152 | VHDMTHQVN | AAV9 | 5.12 | 587/590 |
| Seq ID 153 | DYPEYHDIW | AAV9 | 5.11 | 587/590 |
| Seq ID 154 | QTQQGNYPK | AAV9 | 5.11 | 587/590 |
| Seq ID 155 | QASVGTVRV | AAV9 | 5.10 | 587/590 |
| Seq ID 156 | SPAHQRGVD | AAV9 | 5.08 | 587/590 |
| Seq ID 157 | QMYGEKSVS | AAV9 | 5.06 | 587/590 |
| Seq ID 158 | NTAVQRGSV | AAV9 | 5.06 | 587/590 |
| Seq ID 159 | KTIQQTQDV | AAV9 | 5.06 | 587/590 |
| Seq ID 160 | QMQTLGDDI | AAV9 | 5.05 | 587/590 |
| Seq ID 161 | SHTGQTREL | AAV9 | 5.04 | 587/590 |
| Seq ID 162 | KRQNTESAF | AAV9 | 5.04 | 587/590 |
| Seq ID 163 | NFDPLSVAE | AAV9 | 5.02 | 587/590 |
| Seq ID 164 | KILQQEPLM | AAV9 | 4.99 | 587/590 |
| Seq ID 165 | IVHAVIQST | AAV9 | 4.99 | 587/590 |
| Seq ID 166 | TMDHNPIQT | AAV9 | 4.99 | 587/590 |
| Seq ID 167 | KQPTPTNLW | AAV9 | 4.98 | 587/590 |
| Seq ID 168 | EQGNPGVSI | AAV9 | 4.97 | 587/590 |
| Seq ID 169 | IHSRVQQVT | AAV9 | 4.96 | 587/590 |
| Seq ID 170 | PQSQYRPAN | AAV9 | 4.95 | 587/590 |
| Seq ID 171 | YRRDQENAI | AAV9 | 4.95 | 587/590 |
| Seq ID 172 | TANDAIVAT | AAV9 | 4.94 | 587/590 |
| Seq ID 173 | ALYVGSKTKEGVVHGA | AAV9 | 4.94 | 588/589 |
| Seq ID 174 | MASTNVGTS | AAV9 | 4.93 | 587/590 |
| Seq ID 175 | AIQASDTYI | AAV9 | 4.93 | 587/590 |
| Seq ID 176 | NMQPRIVNA | AAV9 | 4.93 | 587/590 |
| Seq ID 177 | QSDKEQFKH | AAV9 | 4.92 | 587/590 |
| Seq ID 178 | SVINQQAKA | AAV9 | 4.91 | 587/590 |
| Seq ID 179 | NVPGSLKIQ | AAV9 | 4.91 | 587/590 |
| Seq ID 180 | RKDNIPMMV | AAV9 | 4.91 | 587/590 |
| Seq ID 181 | NKVHGTIQD | AAV9 | 4.90 | 587/590 |
| Seq ID 182 | SRKDKSEAE | AAV9 | 4.90 | 587/590 |
| Seq ID 183 | HAQASQTQF | AAV9 | 4.89 | 587/590 |
| Seq ID 184 | HLGHHDGAA | AAV9 | 4.89 | 587/590 |
| Seq ID 185 | QPQMGVMVQ | AAV9 | 4.89 | 587/590 |
| Seq ID 186 | ANLVRNIDA | AAV9 | 4.88 | 588/589 |
| Seq ID 187 | SDIKKRNAV | AAV9 | 4.88 | 587/590 |
| Seq ID 188 | RVMTNSIDV | AAV9 | 4.88 | 587/590 |
| Seq ID 189 | KHQQQLAEK | AAV9 | 4.88 | 587/590 |
| Seq ID 190 | QQTPQTHPV | AAV9 | 4.87 | 587/590 |
| Seq ID 191 | IHNAHVPHQ | AAV9 | 4.86 | 587/590 |
| Seq ID 192 | VSRTPHTDF | AAV9 | 4.85 | 587/590 |
| Seq ID 193 | MQMKGQQPP | AAV9 | 4.84 | 587/590 |
| Seq ID 194 | HQSGQVPNI | AAV9 | 4.84 | 587/590 |
| Seq ID 195 | QLTGVASGH | AAV9 | 4.84 | 587/590 |
| Seq ID 196 | PEQSPQMMF | AAV9 | 4.83 | 587/590 |
| Seq ID 197 | NTTPQASLM | AAV9 | 4.82 | 587/590 |
| Seq ID 198 | DEFGAYMPI | AAV9 | 4.82 | 587/590 |
| Seq ID 199 | PKLIQQVNT | AAV9 | 4.81 | 587/590 |
| Seq ID 200 | KPGPAQVQW | AAV9 | 4.81 | 587/590 |
| Seq ID 201 | PLKDVQLSH | AAV9 | 4.80 | 587/590 |
| Seq ID 202 | KDPFYRSEN | AAV9 | 4.80 | 587/590 |
| Seq ID 203 | SQPGNQLKM | AAV9 | 4.79 | 587/590 |
| Seq ID 204 | MQNTMRLQD | AAV9 | 4.79 | 587/590 |
| Seq ID 205 | ILVKNKDEI | AAV9 | 4.79 | 587/590 |
| Seq ID 206 | PVHGQLQGQ | AAV9 | 4.79 | 587/590 |
| Seq ID 207 | PTLPGVGMR | AAV9 | 4.77 | 587/590 |
| Seq ID 208 | PPQAAQITT | AAV9 | 4.77 | 587/590 |
| Seq ID 209 | TKVQQQDSW | AAV9 | 4.77 | 587/590 |
| Seq ID 210 | IMTSHTVAV | AAV9 | 4.77 | 587/590 |
| Seq ID 211 | HPHMAGQQM | AAV9 | 4.76 | 587/590 |
| Seq ID 212 | PVQTQHAVV | AAV9 | 4.75 | 587/590 |
| Seq ID 213 | QIGHKQEYN | AAV9 | 4.75 | 587/590 |
| Seq ID 214 | HEQQIRRDT | AAV9 | 4.75 | 587/590 |
| Seq ID 215 | DRFKTVQNE | AAV9 | 4.75 | 587/590 |
| Seq ID 216 | AAFQQTDIT | AAV9 | 4.74 | 587/590 |
| Seq ID 217 | ERSAVVMIA | AAV9 | 4.73 | 587/590 |
| Seq ID 218 | QYIQGRQPI | AAV9 | 4.73 | 587/590 |
| Seq ID 219 | LREHAYPDV | AAV9 | 4.73 | 587/590 |
| Seq ID 220 | KLMDQGVTA | AAV9 | 4.72 | 587/590 |
| Seq ID 221 | TSQKQATHT | AAV9 | 4.72 | 587/590 |
| Seq ID 222 | VPNNKNPQV | AAV9 | 4.72 | 587/590 |
| Seq ID 223 | NVVIVMIFM | AAV9 | 4.72 | 587/590 |
| Seq ID 224 | AISYRGTWSTAESGAA | AAV2 | 4.72 | R588A/589 |
| Seq ID 225 | NTTSQTGNV | AAV9 | 4.71 | 587/590 |
| Seq ID 226 | PQESQPENE | AAV9 | 4.71 | 587/590 |
| Seq ID 227 | TMGSRQTPI | AAV9 | 4.71 | 587/590 |
| Seq ID 228 | TQVVSGQEI | AAV9 | 4.68 | 587/590 |
| Seq ID 229 | PEELKSKQT | AAV9 | 4.68 | 587/590 |
| Seq ID 230 | INKYSQYTT | AAV9 | 4.68 | 587/590 |
| Seq ID 231 | QPTQGTLRA | AAV9 | 4.68 | 587/590 |
| Seq ID 232 | NQVHTKDDS | AAV9 | 4.68 | 587/590 |
| Seq ID 233 | YITQIHEDD | AAV9 | 4.67 | 587/590 |
| Seq ID 234 | HIGGSGNTM | AAV9 | 4.67 | 587/590 |
| Seq ID 235 | HQNHPEHAV | AAV9 | 4.65 | 587/590 |
| Seq ID 236 | LKGSVQQAP | AAV9 | 4.65 | 587/590 |
| Seq ID 237 | PLIPRQEVI | AAV9 | 4.64 | 587/590 |
| Seq ID 238 | TLVTQSDSS | AAV9 | 4.64 | 587/590 |
| Seq ID 239 | LKIDVTKIT | AAV9 | 4.62 | 587/590 |
| Seq ID 240 | PSHVGFTSV | AAV9 | 4.62 | 587/590 |
| Seq ID 241 | LYRDQGNIT | AAV9 | 4.62 | 587/590 |
| Seq ID 242 | SVKQMDATH | AAV9 | 4.61 | 587/590 |
| Seq ID 243 | QFDRVAQDK | AAV9 | 4.61 | 587/590 |
| Seq ID 244 | YPTGTPPAT | AAV9 | 4.61 | 587/590 |
| Seq ID 245 | DRVQSTSPH | AAV9 | 4.61 | 587/590 |
| Seq ID 246 | KTQKPYQED | AAV9 | 4.61 | 587/590 |
| Seq ID 247 | SQVAEQRGI | AAV9 | 4.60 | 587/590 |
| Seq ID 248 | SESMHGSVS | AAV9 | 4.60 | 587/590 |
| Seq ID 249 | TITDQQSHG | AAV9 | 4.60 | 587/590 |
| Seq ID 250 | TRHVVTGGY | AAV9 | 4.59 | 587/590 |
| Seq ID 251 | QQNMKLQVY | AAV9 | 4.59 | 587/590 |
| Seq ID 252 | SKFTYYDPW | AAV9 | 4.59 | 587/590 |
| Seq ID 253 | MAVQHTTNQ | AAV9 | 4.58 | 587/590 |
| Seq ID 254 | SSMQGASGV | AAV9 | 4.58 | 587/590 |
| Seq ID 255 | MKYSTQTQS | AAV9 | 4.58 | 587/590 |
| Seq ID 256 | IKQPQSFAQ | AAV9 | 4.57 | 587/590 |
| Seq ID 257 | TPVAQANKN | AAV9 | 4.57 | 587/590 |
| Seq ID 258 | QDVMTSPMQ | AAV9 | 4.57 | 587/590 |
| Seq ID 259 | PQVLGQDEA | AAV9 | 4.56 | 587/590 |
| Seq ID 260 | TDTVDTGSM | AAV9 | 4.56 | 587/590 |
| Seq ID 261 | AQEGARQKLHELQEKA | AAV9 | 4.55 | 588/589 |
| Seq ID 262 | ATSEYGRAY | AAV9 | 4.54 | 587/590 |
| Seq ID 263 | LKGPQQQAI | AAV9 | 4.54 | 587/590 |
| Seq ID 264 | VFREAGNVH | AAV9 | 4.54 | 587/590 |
| Seq ID 265 | PKQVSDIMF | AAV9 | 4.53 | 587/590 |
| Seq ID 266 | HMMDGGVPY | AAV9 | 4.53 | 587/590 |
| Seq ID 267 | IRAASTGHM | AAV9 | 4.53 | 587/590 |
| Seq ID 268 | PDGYHVEMF | AAV9 | 4.52 | 587/590 |
| Seq ID 269 | QAQSNNIYF | AAV9 | 4.52 | 587/590 |
| Seq ID 270 | TMNGQSKNF | AAV9 | 4.52 | 587/590 |
| Seq ID 271 | HQARREVEY | AAV9 | 4.52 | 587/590 |
| Seq ID 272 | GDQGRVVSH | AAV9 | 4.52 | 587/590 |
| Seq ID 273 | ACCPLAEESDNVDSAA | AAV2 | 4.51 | R588A/589 |
| Seq ID 274 | QQNQSIFNI | AAV9 | 4.51 | 587/590 |
| Seq ID 275 | TSDAVKTDQ | AAV9 | 4.51 | 587/590 |
| Seq ID 276 | TQAPDVLPV | AAV9 | 4.51 | 587/590 |
| Seq ID 277 | TTDTAKATY | AAV9 | 4.50 | 587/590 |
| Seq ID 278 | IDEHSEHYV | AAV9 | 4.49 | 587/590 |
| Seq ID 279 | PINSRIVDP | AAV9 | 4.49 | 587/590 |
| Seq ID 280 | PDQRGHMYA | AAV9 | 4.49 | 587/590 |
| Seq ID 281 | FLMSHSEVQ | AAV9 | 4.49 | 587/590 |
| Seq ID 282 | FIQQSNERN | AAV9 | 4.48 | 587/590 |
| Seq ID 283 | NPTRDAKPP | AAV9 | 4.48 | 587/590 |
| Seq ID 284 | RWGRAQEDE | AAV9 | 4.47 | 587/590 |
| Seq ID 285 | PRNMQPMTD | AAV9 | 4.46 | 587/590 |
| Seq ID 286 | MVSQREINQ | AAV9 | 4.45 | 587/590 |
| Seq ID 287 | MSSSTRMAV | AAV9 | 4.45 | 587/590 |
| Seq ID 288 | PLNQHQKIS | AAV9 | 4.45 | 587/590 |
| Seq ID 289 | DNASHVHMQ | AAV9 | 4.44 | 587/590 |
| Seq ID 290 | DMKRVDYMQ | AAV9 | 4.44 | 587/590 |
| Seq ID 291 | NHLVDRTTQ | AAV9 | 4.43 | 587/590 |
| Seq ID 292 | VIQASSITA | AAV9 | 4.43 | 587/590 |
| Seq ID 293 | AQAMTNKNK | AAV9 | 4.43 | 587/590 |
| Seq ID 294 | INFKQEVHQ | AAV9 | 4.43 | 587/590 |
| Seq ID 295 | AYAQLPHEK | AAV9 | 4.43 | 587/590 |
| Seq ID 296 | AVTIQNWCKRGRKQCA | AAV2 | 4.42 | R588A/589 |
| Seq ID 297 | NPQNSGMAM | AAV9 | 4.42 | 587/590 |
| Seq ID 298 | SRDRDTAYA | AAV9 | 4.41 | 587/590 |
| Seq ID 299 | PHNSPGMTR | AAV9 | 4.41 | 587/590 |
| Seq ID 300 | KMMPSQMQD | AAV9 | 4.40 | 587/590 |
| Seq ID 301 | NHAPVHERM | AAV9 | 4.39 | 587/590 |
| Seq ID 302 | RALKQIDAD | AAV9 | 4.39 | 587/590 |
| Seq ID 303 | QRVEGFIQD | AAV9 | 4.38 | 587/590 |
| Seq ID 304 | AKTDSCQGDSGGPLVA | AAV2 | 4.37 | R588A/589 |
| Seq ID 305 | TSASVQSAM | AAV9 | 4.37 | 587/590 |
| Seq ID 306 | NVTTHAPTI | AAV9 | 4.37 | 587/590 |
| Seq ID 307 | RVQTPDHDH | AAV9 | 4.37 | 587/590 |
| Seq ID 308 | QVTKEFASF | AAV9 | 4.37 | 587/590 |
| Seq ID 309 | ASVTPQYLY | AAV9 | 4.37 | 587/590 |
| Seq ID 310 | VTQKTDNTL | AAV9 | 4.36 | 587/590 |
| Seq ID 311 | VKMGIQAIA | AAV9 | 4.35 | 587/590 |
| Seq ID 312 | PLDNYLASM | AAV9 | 4.35 | 587/590 |
| Seq ID 313 | DENGGVPRI | AAV9 | 4.35 | 587/590 |
| Seq ID 314 | HWQQPSIDG | AAV9 | 4.34 | 587/590 |
| Seq ID 315 | TPSQGSHGS | AAV9 | 4.33 | 587/590 |
| Seq ID 316 | HNGMNQVDK | AAV9 | 4.33 | 587/590 |
| Seq ID 317 | PHMHNMATP | AAV9 | 4.32 | 587/590 |
| Seq ID 318 | IQVRDRTPE | AAV9 | 4.31 | 587/590 |
| Seq ID 319 | APDAHTRMP | AAV9 | 4.31 | 587/590 |
| Seq ID 320 | GAVSHHQMI | AAV9 | 4.31 | 587/590 |
| Seq ID 321 | NVQRTGVGN | AAV9 | 4.31 | 587/590 |
| Seq ID 322 | PDMLQGKMY | AAV9 | 4.31 | 587/590 |
| Seq ID 323 | NYFASSDQM | AAV9 | 4.30 | 587/590 |
| Seq ID 324 | PRQGSLAHM | AAV9 | 4.30 | 587/590 |
| Seq ID 325 | SQQVSKQMQ | AAV9 | 4.30 | 587/590 |
| Seq ID 326 | KRNQEGNGV | AAV9 | 4.29 | 587/590 |
| Seq ID 327 | PMHSSHNDK | AAV9 | 4.28 | 587/590 |
| Seq ID 328 | TLQPQHIAK | AAV9 | 4.28 | 587/590 |
| Seq ID 329 | SAGVVISVV | AAV9 | 4.28 | 587/590 |
| Seq ID 330 | PLVPRNTPQ | AAV9 | 4.28 | 587/590 |
| Seq ID 331 | PNQTQMLNK | AAV9 | 4.27 | 587/590 |
| Seq ID 332 | PMSSEYRPS | AAV9 | 4.27 | 587/590 |
| Seq ID 333 | ESAHSEFKV | AAV9 | 4.27 | 587/590 |
| Seq ID 334 | PQRVQEQGT | AAV9 | 4.27 | 587/590 |
| Seq ID 335 | MVQQSLKPA | AAV9 | 4.26 | 587/590 |
| Seq ID 336 | EQQPAQYHG | AAV9 | 4.25 | 587/590 |
| Seq ID 337 | TRNAQTTPL | AAV9 | 4.25 | 587/590 |
| Seq ID 338 | PARSGVQIV | AAV9 | 4.25 | 587/590 |
| Seq ID 339 | DVFTRQSEG | AAV9 | 4.24 | 587/590 |
| Seq ID 340 | SMHTHQGGQ | AAV9 | 4.24 | 587/590 |
| Seq ID 341 | VNQNVRMED | AAV9 | 4.24 | 587/590 |
| Seq ID 342 | LPQAKMADM | AAV9 | 4.24 | 587/590 |
| Seq ID 343 | RMMDNSIHT | AAV9 | 4.24 | 587/590 |
| Seq ID 344 | MEATAEHRI | AAV9 | 4.24 | 587/590 |
| Seq ID 345 | DARAHTQQV | AAV9 | 4.24 | 587/590 |
| Seq ID 346 | IKDMEPQLH | AAV9 | 4.22 | 587/590 |
| Seq ID 347 | ALITILGTVKPNANRA | AAV9 | 4.22 | 588/589 |
| Seq ID 348 | NHQARTGEF | AAV9 | 4.21 | 587/590 |
| Seq ID 349 | PKNHPVSAV | AAV9 | 4.20 | 587/590 |
| Seq ID 350 | KFPTEDKYI | AAV9 | 4.20 | 587/590 |
| Seq ID 351 | SPVATQKMV | AAV9 | 4.20 | 587/590 |
| Seq ID 352 | DLAEHTRFK | AAV9 | 4.20 | 587/590 |
| Seq ID 353 | SNTQPRPQQ | AAV9 | 4.19 | 587/590 |
| Seq ID 354 | TIDITPRKL | AAV9 | 4.18 | 587/590 |
| Seq ID 355 | PAFVDENPP | AAV9 | 4.18 | 587/590 |
| Seq ID 356 | VSQETRTTI | AAV9 | 4.17 | 587/590 |
| Seq ID 357 | EHTGGQFRL | AAV9 | 4.17 | 587/590 |
| Seq ID 358 | MLGKSDQVW | AAV9 | 4.16 | 587/590 |
| Seq ID 359 | PQRDMVNMK | AAV9 | 4.15 | 587/590 |
| Seq ID 360 | NSRPGAAMA | AAV9 | 4.14 | 587/590 |
| Seq ID 361 | TQQAPHNAY | AAV9 | 4.14 | 587/590 |
| Seq ID 362 | PNITGGAIM | AAV9 | 4.12 | 587/590 |
| Seq ID 363 | PDQKMMFKL | AAV9 | 4.11 | 587/590 |
| Seq ID 364 | LKHATVEST | AAV9 | 4.11 | 587/590 |
| Seq ID 365 | QLSRDTRQD | AAV9 | 4.11 | 587/590 |
| Seq ID 366 | PDSKPHAYH | AAV9 | 4.11 | 587/590 |
| Seq ID 367 | MQPPAQQRT | AAV9 | 4.11 | 587/590 |
| Seq ID 368 | VTIFDMFDQ | AAV9 | 4.10 | 587/590 |
| Seq ID 369 | IMAYTGQPV | AAV9 | 4.10 | 587/590 |
| Seq ID 370 | RTEYEMSTR | AAV9 | 4.10 | 587/590 |
| Seq ID 371 | QAQRKTEFI | AAV9 | 4.10 | 587/590 |
| Seq ID 372 | QGNETKPNS | AAV9 | 4.10 | 587/590 |
| Seq ID 373 | RMASQGQDM | AAV9 | 4.10 | 587/590 |
| Seq ID 374 | PKDGVPQMQ | AAV9 | 4.08 | 587/590 |
| Seq ID 375 | TIAPAIANL | AAV9 | 4.08 | 587/590 |
| Seq ID 376 | RTHQSSPDS | AAV9 | 4.08 | 587/590 |
| Seq ID 377 | PTTDMQSNI | AAV9 | 4.08 | 587/590 |
| Seq ID 378 | HATSQKYET | AAV9 | 4.07 | 587/590 |
| Seq ID 379 | ALGQARSDA | AAV9 | 4.07 | 588/589 |
| Seq ID 380 | PIATAYKTG | AAV9 | 4.07 | 587/590 |
| Seq ID 381 | HFKPTQDTS | AAV9 | 4.06 | 587/590 |
| Seq ID 382 | RPNTKAMQE | AAV9 | 4.06 | 587/590 |
| Seq ID 383 | NMPQGRVAM | AAV9 | 4.06 | 587/590 |
| Seq ID 384 | KTINTPNIL | AAV9 | 4.05 | 587/590 |
| Seq ID 385 | SRIQHDGAQ | AAV9 | 4.05 | 587/590 |
| Seq ID 386 | HPPMPNNTK | AAV9 | 4.04 | 587/590 |
| Seq ID 387 | RIMPQMQHD | AAV9 | 4.04 | 587/590 |
| Seq ID 388 | AVILELDKTVEHLPTA | AAV2 | 4.03 | R588A/589 |
| Seq ID 389 | HSTARSGEF | AAV9 | 4.03 | 587/590 |
| Seq ID 390 | SQPQHMNVS | AAV9 | 4.03 | 587/590 |
| Seq ID 391 | PGGSYSHVV | AAV9 | 4.03 | 587/590 |
| Seq ID 392 | TVTHAHPTT | AAV9 | 4.02 | 587/590 |
| Seq ID 393 | IVATAPRVA | AAV9 | 4.02 | 587/590 |
| Seq ID 394 | VTRTSVAIE | AAV9 | 4.02 | 587/590 |
| Seq ID 395 | MIVPTPMNQ | AAV9 | 4.01 | 587/590 |
| Seq ID 396 | IQVMHSTTH | AAV9 | 4.01 | 587/590 |
| Seq ID 397 | QEPTYKNEV | AAV9 | 4.00 | 587/590 |
| Seq ID 398 | VVPTMNNTR | AAV9 | 4.00 | 587/590 |
| Seq ID 399 | RETTVYPLQ | AAV9 | 4.00 | 587/590 |
| Seq ID 400 | RITTESTIH | AAV9 | 4.00 | 587/590 |
| Seq ID 401 | AEEVAARRARVVWCAA | AAV2 | 3.99 | R588A/589 |
| Seq ID 402 | DPVRRKMQV | AAV9 | 3.99 | 587/590 |
| Seq ID 403 | PYSSKTQDP | AAV9 | 3.98 | 587/590 |
| Seq ID 404 | WPETKQEKS | AAV9 | 3.97 | 587/590 |
| Seq ID 405 | PNTQHLKAE | AAV9 | 3.97 | 587/590 |
| Seq ID 406 | WQSTGKQSI | AAV9 | 3.97 | 587/590 |
| Seq ID 407 | KASTDPHSR | AAV9 | 3.97 | 587/590 |
| Seq ID 408 | KPTGTIQGY | AAV9 | 3.95 | 587/590 |
| Seq ID 409 | HLGTAIVQP | AAV9 | 3.95 | 587/590 |
| Seq ID 410 | PKFSHATSE | AAV9 | 3.94 | 587/590 |
| Seq ID 411 | IRDGIQMHV | AAV9 | 3.94 | 587/590 |
| Seq ID 412 | TTNQERLQS | AAV9 | 3.94 | 587/590 |
| Seq ID 413 | QIKVPPLDQ | AAV9 | 3.94 | 587/590 |
| Seq ID 414 | LHHERGPSA | AAV9 | 3.92 | 587/590 |
| Seq ID 415 | KPLGQAQVQ | AAV9 | 3.91 | 587/590 |
| Seq ID 416 | TPASATRDS | AAV9 | 3.91 | 587/590 |
| Seq ID 417 | PQQTQVSGQ | AAV9 | 3.91 | 587/590 |
| Seq ID 418 | ERSSQSIIQ | AAV9 | 3.90 | 587/590 |
| Seq ID 419 | YHEWRHIPT | AAV9 | 3.89 | 587/590 |
| Seq ID 420 | PLQASGTAY | AAV9 | 3.88 | 587/590 |
| Seq ID 421 | TNNGVRSII | AAV9 | 3.88 | 587/590 |
| Seq ID 422 | VQPEITPGQ | AAV9 | 3.88 | 587/590 |
| Seq ID 423 | QDDHNKVQQ | AAV9 | 3.87 | 587/590 |
| Seq ID 424 | PIVKQNTDT | AAV9 | 3.86 | 587/590 |
| Seq ID 425 | HTLPRLQEE | AAV9 | 3.86 | 587/590 |
| Seq ID 426 | QPTQGTVRA | AAV9 | 3.86 | 587/590 |
| Seq ID 427 | TRPQGPLSP | AAV9 | 3.86 | 587/590 |
| Seq ID 428 | TYAQPHQNQ | AAV9 | 3.84 | 587/590 |
| Seq ID 429 | IQMEQKQQV | AAV9 | 3.84 | 587/590 |
| Seq ID 430 | TQTPPQDAS | AAV9 | 3.84 | 587/590 |
| Seq ID 431 | SGAQEKWEM | AAV9 | 3.84 | 587/590 |
| Seq ID 432 | ASDTEKEDRSPSAIFA | AAV9 | 3.83 | 588/589 |
| Seq ID 433 | PNYQTKMSF | AAV9 | 3.83 | 587/590 |
| Seq ID 434 | RPGAAVAPA | AAV9 | 3.82 | 587/590 |
| Seq ID 435 | LAGSHVSTQ | AAV9 | 3.82 | 587/590 |
| Seq ID 436 | LPPGNQTNN | AAV9 | 3.82 | 587/590 |
| Seq ID 437 | NHQHDIEWF | AAV9 | 3.82 | 587/590 |
| Seq ID 438 | ISEDGSNRK | AAV9 | 3.81 | 587/590 |
| Seq ID 439 | SMGIHPTNA | AAV9 | 3.81 | 587/590 |
| Seq ID 440 | TPLAMASQK | AAV9 | 3.81 | 587/590 |
| Seq ID 441 | NVTSNTIQF | AAV9 | 3.80 | 587/590 |
| Seq ID 442 | NYNTVPQPK | AAV9 | 3.80 | 587/590 |
| Seq ID 443 | PRVLQGVQV | AAV9 | 3.79 | 587/590 |
| Seq ID 444 | TGTRTSAQQ | AAV9 | 3.79 | 587/590 |
| Seq ID 445 | NHHRPDVFV | AAV9 | 3.79 | 587/590 |
| Seq ID 446 | PGPNERQIM | AAV9 | 3.78 | 587/590 |
| Seq ID 447 | PHDRLPQEV | AAV9 | 3.78 | 587/590 |
| Seq ID 448 | PNAGVMDEE | AAV9 | 3.78 | 587/590 |
| Seq ID 449 | TAHYRTAET | AAV9 | 3.78 | 587/590 |
| Seq ID 450 | RPQSSATTH | AAV9 | 3.77 | 587/590 |
| Seq ID 451 | IAHFQPKDT | AAV9 | 3.76 | 587/590 |
| Seq ID 452 | FAHPEKGFF | AAV9 | 3.76 | 587/590 |
| Seq ID 453 | QAYAKTAQL | AAV9 | 3.75 | 587/590 |
| Seq ID 454 | TTSVSVMPK | AAV9 | 3.75 | 587/590 |
| Seq ID 455 | ASVDTEQRR | AAV9 | 3.75 | 587/590 |
| Seq ID 456 | WHEAIIVTG | AAV9 | 3.74 | 587/590 |
| Seq ID 457 | VDNDIEWKF | AAV9 | 3.74 | 587/590 |
| Seq ID 458 | NRVGSQAPT | AAV9 | 3.73 | 587/590 |
| Seq ID 459 | AAVIVANPREEIIVKA | AAV2 | 3.72 | R588A/589 |
| Seq ID 460 | PTTPQMVSL | AAV9 | 3.72 | 587/590 |
| Seq ID 461 | ALKNFAKYFLHQSHEA | AAV2 | 3.71 | R588A/589 |
| Seq ID 462 | KYVNVEVQT | AAV9 | 3.71 | 587/590 |
| Seq ID 463 | LLDAGTSGK | AAV9 | 3.71 | 587/590 |
| Seq ID 464 | NAPAQQKMM | AAV9 | 3.70 | 587/590 |
| Seq ID 465 | VDDHAHRQQ | AAV9 | 3.70 | 587/590 |
| Seq ID 466 | PVKLANPTN | AAV9 | 3.70 | 587/590 |
| Seq ID 467 | HALVHQGAV | AAV9 | 3.70 | 587/590 |
| Seq ID 468 | VFQKSHSTE | AAV9 | 3.69 | 587/590 |
| Seq ID 469 | FKQRDSNET | AAV9 | 3.69 | 587/590 |
| Seq ID 470 | EQMSQRVGF | AAV9 | 3.69 | 587/590 |
| Seq ID 471 | RQAGQFPDS | AAV9 | 3.69 | 587/590 |
| Seq ID 472 | ESQANAHNA | AAV9 | 3.69 | 587/590 |
| Seq ID 473 | PPDSKSLNQ | AAV9 | 3.69 | 587/590 |
| Seq ID 474 | PEQPSQSQY | AAV9 | 3.69 | 587/590 |
| Seq ID 475 | DGIHTSVQM | AAV9 | 3.69 | 587/590 |
| Seq ID 476 | RFDLQEGQS | AAV9 | 3.69 | 587/590 |
| Seq ID 477 | DRDRAMMNI | AAV9 | 3.69 | 587/590 |
| Seq ID 478 | YPPEAQMEM | AAV9 | 3.68 | 587/590 |
| Seq ID 479 | TAVTTHNGS | AAV9 | 3.68 | 587/590 |
| Seq ID 480 | EWALDQYGA | AAV9 | 3.68 | 587/590 |
| Seq ID 481 | ITQSNERMT | AAV9 | 3.68 | 587/590 |
| Seq ID 482 | NITHQTMLQ | AAV9 | 3.67 | 587/590 |
| Seq ID 483 | MMNGSQQFQ | AAV9 | 3.67 | 587/590 |
| Seq ID 484 | QQTTGRVQQ | AAV9 | 3.66 | 587/590 |
| Seq ID 485 | LVNPDPMQY | AAV9 | 3.66 | 587/590 |
| Seq ID 486 | HTHDMDMTP | AAV9 | 3.66 | 587/590 |
| Seq ID 487 | PQTKNTMDD | AAV9 | 3.66 | 587/590 |
| Seq ID 488 | DPERQTRPY | AAV9 | 3.66 | 587/590 |
| Seq ID 489 | TKARSDPLE | AAV9 | 3.65 | 587/590 |
| Seq ID 490 | RVNDQPYQM | AAV9 | 3.65 | 587/590 |
| Seq ID 491 | RPSNVDSAT | AAV9 | 3.65 | 587/590 |
| Seq ID 492 | RMSNDQKSF | AAV9 | 3.64 | 587/590 |
| Seq ID 493 | SMTSAFDRI | AAV9 | 3.64 | 587/590 |
| Seq ID 494 | IHDKQMNNQ | AAV9 | 3.63 | 587/590 |
| Seq ID 495 | NEYFPYEDL | AAV9 | 3.63 | 587/590 |
| Seq ID 496 | SIVPTANQD | AAV9 | 3.63 | 587/590 |
| Seq ID 497 | IFEQAHTNK | AAV9 | 3.62 | 587/590 |
| Seq ID 498 | AGDTRSLQV | AAV9 | 3.62 | 587/590 |
| Seq ID 499 | LQNQTKPVI | AAV9 | 3.62 | 587/590 |
| Seq ID 500 | QGDIVQHHF | AAV9 | 3.61 | 587/590 |
| Seq ID 501 | EIQTYKSIV | AAV9 | 3.61 | 587/590 |
| Seq ID 502 | KAPSIVQGT | AAV9 | 3.61 | 587/590 |
| Seq ID 503 | QTPQHGTVL | AAV9 | 3.61 | 587/590 |
| Seq ID 504 | KLMTMYDGQ | AAV9 | 3.60 | 587/590 |
| Seq ID 505 | LWTNQPSAW | AAV9 | 3.60 | 587/590 |
| Seq ID 506 | HSDQMRQMF | AAV9 | 3.60 | 587/590 |
| Seq ID 507 | EWQNEATPA | AAV9 | 3.60 | 587/590 |
| Seq ID 508 | RPERDPMHI | AAV9 | 3.60 | 587/590 |
| Seq ID 509 | AVWQAANTH | AAV9 | 3.60 | 587/590 |
| Seq ID 510 | RTATTQGGF | AAV9 | 3.57 | 587/590 |
| Seq ID 511 | PTNLVEAGE | AAV9 | 3.57 | 587/590 |
| Seq ID 512 | TRMNQTPLQ | AAV9 | 3.57 | 587/590 |
| Seq ID 513 | PSFTQDRDR | AAV9 | 3.56 | 587/590 |
| Seq ID 514 | SSAAHTVTY | AAV9 | 3.56 | 587/590 |
| Seq ID 515 | SHPQPFMKW | AAV9 | 3.56 | 587/590 |
| Seq ID 516 | QQGVREAFI | AAV9 | 3.56 | 587/590 |
| Seq ID 517 | AQSPLVSSSDSPPRPA | AAV2 | 3.55 | R588A/589 |
| Seq ID 518 | DSAVRQNPF | AAV9 | 3.55 | 587/590 |
| Seq ID 519 | KSNSMQEAH | AAV9 | 3.55 | 587/590 |
| Seq ID 520 | PRQLSSIPS | AAV9 | 3.54 | 587/590 |
| Seq ID 521 | DPSAQKQNY | AAV9 | 3.54 | 587/590 |
| Seq ID 522 | TGAFDDHDI | AAV9 | 3.54 | 587/590 |
| Seq ID 523 | NGTHLVTRA | AAV9 | 3.54 | 587/590 |
| Seq ID 524 | TGGKVQLGS | AAV9 | 3.53 | 587/590 |
| Seq ID 525 | KQLKTMNDF | AAV9 | 3.53 | 587/590 |
| Seq ID 526 | PHDTRIAVD | AAV9 | 3.53 | 587/590 |
| Seq ID 527 | ERHIAHTWT | AAV9 | 3.53 | 587/590 |
| Seq ID 528 | YRPHPQQHP | AAV9 | 3.53 | 587/590 |
| Seq ID 529 | YNLPQPKEI | AAV9 | 3.53 | 587/590 |
| Seq ID 530 | DTQQQPPYK | AAV9 | 3.52 | 587/590 |
| Seq ID 531 | QSNQVKQMD | AAV9 | 3.52 | 587/590 |
| Seq ID 532 | IEVKQQQNG | AAV9 | 3.51 | 587/590 |
| Seq ID 533 | DQTHNIMQK | AAV9 | 3.51 | 587/590 |
| Seq ID 534 | VQEEGWLYA | AAV9 | 3.50 | 587/590 |
| Seq ID 535 | KQMVAQMSM | AAV9 | 3.50 | 587/590 |
| Seq ID 536 | SINGQQHDY | AAV9 | 3.50 | 587/590 |
| Seq ID 537 | TPDMFKSPN | AAV9 | 3.50 | 587/590 |
| Seq ID 538 | GNHSVHHQQ | AAV9 | 3.50 | 587/590 |
| Seq ID 539 | ENSRTTTVG | AAV9 | 3.49 | 587/590 |
| Seq ID 540 | TFVVGQSGK | AAV9 | 3.49 | 587/590 |
| Seq ID 541 | APLRPQTGE | AAV9 | 3.49 | 587/590 |
| Seq ID 542 | QTSSSTHHA | AAV9 | 3.49 | 587/590 |
| Seq ID 543 | KIAATALSS | AAV9 | 3.49 | 587/590 |
| Seq ID 544 | PESGQSKMH | AAV9 | 3.48 | 587/590 |
| Seq ID 545 | VISVGQMAK | AAV9 | 3.48 | 587/590 |
| Seq ID 546 | AERPQQGMH | AAV9 | 3.48 | 587/590 |
| Seq ID 547 | TQQNQIIQR | AAV9 | 3.47 | 587/590 |
| Seq ID 548 | ARQAQEKFGKDKSPKA | AAV2 | 3.47 | R588A/589 |
| Seq ID 549 | VTAVTTPSH | AAV9 | 3.47 | 587/590 |
| Seq ID 550 | SQVRGPETT | AAV9 | 3.47 | 587/590 |
| Seq ID 551 | PLNHQQIQR | AAV9 | 3.46 | 587/590 |
| Seq ID 552 | AVVPRPHEI | AAV9 | 3.46 | 587/590 |
| Seq ID 553 | TPNMEDNRQ | AAV9 | 3.46 | 587/590 |
| Seq ID 554 | HRVTGVNDV | AAV9 | 3.46 | 587/590 |
| Seq ID 555 | FTGNQGAQN | AAV9 | 3.46 | 587/590 |
| Seq ID 556 | PRLAETSSQ | AAV9 | 3.45 | 587/590 |
| Seq ID 557 | SPRQSDPAH | AAV9 | 3.45 | 587/590 |
| Seq ID 558 | MNDDQQPPP | AAV9 | 3.44 | 587/590 |
| Seq ID 559 | QMKPLPQAM | AAV9 | 3.43 | 587/590 |
| Seq ID 560 | YSRHENISY | AAV9 | 3.43 | 587/590 |
| Seq ID 561 | TEGERVGML | AAV9 | 3.43 | 587/590 |
| Seq ID 562 | NHVPVLWAD | AAV9 | 3.43 | 587/590 |
| Seq ID 563 | TLAPLATTS | AAV9 | 3.43 | 587/590 |
| Seq ID 564 | NQRDVTNLT | AAV9 | 3.43 | 587/590 |
| Seq ID 565 | QQNNTVPNQ | AAV9 | 3.43 | 587/590 |
| Seq ID 566 | EKNMHTGVH | AAV9 | 3.42 | 587/590 |
| Seq ID 567 | VTLQGATPN | AAV9 | 3.42 | 587/590 |
| Seq ID 568 | PTKTSTGQM | AAV9 | 3.42 | 587/590 |
| Seq ID 569 | FNVDQHRNS | AAV9 | 3.42 | 587/590 |
| Seq ID 570 | PMTLLKDIQ | AAV9 | 3.42 | 587/590 |
| Seq ID 571 | DPTRPIETV | AAV9 | 3.42 | 587/590 |
| Seq ID 572 | ERKQQLSEM | AAV9 | 3.42 | 587/590 |
| Seq ID 573 | NQEQFGMIV | AAV9 | 3.42 | 587/590 |
| Seq ID 574 | TTQIMVGTK | AAV9 | 3.41 | 587/590 |
| Seq ID 575 | MYAKTEVQW | AAV9 | 3.41 | 587/590 |
| Seq ID 576 | PVGHQMDVY | AAV9 | 3.41 | 587/590 |
| Seq ID 577 | QVFQDRTSL | AAV9 | 3.40 | 587/590 |
| Seq ID 578 | LKDNQQATF | AAV9 | 3.40 | 587/590 |
| Seq ID 579 | AGNTHTLIN | AAV9 | 3.40 | 587/590 |
| Seq ID 580 | KVPPQHLTA | AAV9 | 3.40 | 587/590 |
| Seq ID 581 | GIVTATRVQ | AAV9 | 3.40 | 587/590 |
| Seq ID 582 | NIAHHQGSN | AAV9 | 3.39 | 587/590 |
| Seq ID 583 | PAMPGAAAR | AAV9 | 3.39 | 587/590 |
| Seq ID 584 | VQSITPPQS | AAV9 | 3.39 | 587/590 |
| Seq ID 585 | QATHQGQNH | AAV9 | 3.39 | 587/590 |
| Seq ID 586 | PTNAPGVTE | AAV9 | 3.39 | 587/590 |
| Seq ID 587 | MHRAQAIAE | AAV9 | 3.39 | 587/590 |
| Seq ID 588 | AQASEKMMK | AAV9 | 3.39 | 587/590 |
| Seq ID 589 | RMNIPPNGT | AAV9 | 3.38 | 587/590 |
| Seq ID 590 | RPSDINSGD | AAV9 | 3.38 | 587/590 |
| Seq ID 591 | MQPKSAQNV | AAV9 | 3.38 | 587/590 |
| Seq ID 592 | LAASMQGAH | AAV9 | 3.38 | 587/590 |
| Seq ID 593 | TSIITQAAN | AAV9 | 3.37 | 587/590 |
| Seq ID 594 | SDREETFNM | AAV9 | 3.37 | 587/590 |
| Seq ID 595 | NQYNMQPSK | AAV9 | 3.37 | 587/590 |
| Seq ID 596 | WVSEEHFLT | AAV9 | 3.37 | 587/590 |
| Seq ID 597 | QSATSVTSQ | AAV9 | 3.37 | 587/590 |
| Seq ID 598 | HARQSYNDP | AAV9 | 3.36 | 587/590 |
| Seq ID 599 | AMNVISHDGTPA | AAV2 | 3.36 | R588A/589 |
| Seq ID 600 | STIMSEAEF | AAV9 | 3.36 | 587/590 |
| Seq ID 601 | HANTNVLEK | AAV9 | 3.36 | 587/590 |
| Seq ID 602 | KPQVTPAVH | AAV9 | 3.36 | 587/590 |
| Seq ID 603 | AMPDRTMMR | AAV9 | 3.36 | 587/590 |
| Seq ID 604 | VAIGQTKTL | AAV9 | 3.34 | 587/590 |
| Seq ID 605 | PTDYSVKSV | AAV9 | 3.34 | 587/590 |
| Seq ID 606 | IGHHKEMKE | AAV9 | 3.34 | 587/590 |
| Seq ID 607 | ASQAIHYNA | AAV9 | 3.34 | 587/590 |
| Seq ID 608 | QVRMEGAEY | AAV9 | 3.34 | 587/590 |
| Seq ID 609 | MKERHEHFE | AAV9 | 3.34 | 587/590 |
| Seq ID 610 | GQVLMAGTR | AAV9 | 3.34 | 587/590 |
| Seq ID 611 | TVSMPVSHA | AAV9 | 3.33 | 587/590 |
| Seq ID 612 | NHDRVNVQP | AAV9 | 3.33 | 587/590 |
| Seq ID 613 | EELGILNLQ | AAV9 | 3.33 | 587/590 |
| Seq ID 614 | ANGNFQVLK | AAV9 | 3.33 | 587/590 |
| Seq ID 615 | RQYNTESMH | AAV9 | 3.33 | 587/590 |
| Seq ID 616 | MKVSAQVQS | AAV9 | 3.32 | 587/590 |
| Seq ID 617 | NVSQQKMVQ | AAV9 | 3.31 | 587/590 |
| Seq ID 618 | RQQNQDKFS | AAV9 | 3.31 | 587/590 |
| Seq ID 619 | MSSRTMEIH | AAV9 | 3.31 | 587/590 |
| Seq ID 620 | HEMIKQSGP | AAV9 | 3.31 | 587/590 |
| Seq ID 621 | QDTDRAGKF | AAV9 | 3.30 | 587/590 |
| Seq ID 622 | KWEQRGMAH | AAV9 | 3.30 | 587/590 |
| Seq ID 623 | TKQHTEMQQ | AAV9 | 3.29 | 587/590 |
| Seq ID 624 | PMQALQNML | AAV9 | 3.29 | 587/590 |
| Seq ID 625 | KVAFQQQIE | AAV9 | 3.29 | 587/590 |
| Seq ID 626 | QVPRQEVNE | AAV9 | 3.28 | 587/590 |
| Seq ID 627 | HLEREHKWD | AAV9 | 3.28 | 587/590 |
| Seq ID 628 | IQRKIQDLA | AAV9 | 3.28 | 587/590 |
| Seq ID 629 | IKGTDSMQQ | AAV9 | 3.28 | 587/590 |
| Seq ID 630 | TMVSGEARQ | AAV9 | 3.28 | 587/590 |
| Seq ID 631 | TMVASRQPP | AAV9 | 3.28 | 587/590 |
| Seq ID 632 | QQRQHQEIT | AAV9 | 3.27 | 587/590 |
| Seq ID 633 | NPESMEAYI | AAV9 | 3.27 | 587/590 |
| Seq ID 634 | TSQNGKMSV | AAV9 | 3.27 | 587/590 |
| Seq ID 635 | SRTSTQPQE | AAV9 | 3.26 | 587/590 |
| Seq ID 636 | RYAVKGETD | AAV9 | 3.26 | 587/590 |
| Seq ID 637 | VALYPNHAE | AAV9 | 3.26 | 587/590 |
| Seq ID 638 | QQTTQHIDS | AAV9 | 3.26 | 587/590 |
| Seq ID 639 | SSMQEASQI | AAV9 | 3.26 | 587/590 |
| Seq ID 640 | KMNSPNQER | AAV9 | 3.25 | 587/590 |
| Seq ID 641 | MTNRTPASN | AAV9 | 3.25 | 587/590 |
| Seq ID 642 | IGVRPMISP | AAV9 | 3.25 | 587/590 |
| Seq ID 643 | RQADSRTIE | AAV9 | 3.25 | 587/590 |
| Seq ID 644 | EMRNHPAVQ | AAV9 | 3.25 | 587/590 |
| Seq ID 645 | MRTNEPHKE | AAV9 | 3.24 | 587/590 |
| Seq ID 646 | PTIQRQGMN | AAV9 | 3.24 | 587/590 |
| Seq ID 647 | DIDPEDIST | AAV9 | 3.23 | 587/590 |
| Seq ID 648 | EIKMQSTQA | AAV9 | 3.23 | 587/590 |
| Seq ID 649 | QNFPSQNAA | AAV9 | 3.23 | 587/590 |
| Seq ID 650 | YRTGPDEIQ | AAV9 | 3.23 | 587/590 |
| Seq ID 651 | IQHTHSHHE | AAV9 | 3.23 | 587/590 |
| Seq ID 652 | IISTQEGHI | AAV9 | 3.22 | 587/590 |
| Seq ID 653 | PEGDNTFMI | AAV9 | 3.22 | 587/590 |
| Seq ID 654 | RANAEYLSD | AAV9 | 3.22 | 587/590 |
| Seq ID 655 | EVSLPSNVK | AAV9 | 3.22 | 587/590 |
| Seq ID 656 | RMDINRATD | AAV9 | 3.22 | 587/590 |
| Seq ID 657 | HPSPNTAYV | AAV9 | 3.22 | 587/590 |
| Seq ID 658 | SYHSWFPMG | AAV9 | 3.21 | 587/590 |
| Seq ID 659 | AGDRQHMAA | AAV9 | 3.21 | 587/590 |
| Seq ID 660 | QPKKWGADY | AAV9 | 3.21 | 587/590 |
| Seq ID 661 | LKQDSRPTY | AAV9 | 3.21 | 587/590 |
| Seq ID 662 | VSSYQLKSD | AAV9 | 3.21 | 587/590 |
| Seq ID 663 | RGHSVSDNA | AAV9 | 3.21 | 587/590 |
| Seq ID 664 | HQAQNQMFK | AAV9 | 3.20 | 587/590 |
| Seq ID 665 | QASNFITVP | AAV9 | 3.20 | 587/590 |
| Seq ID 666 | VEPHYQQQW | AAV9 | 3.20 | 587/590 |
| Seq ID 667 | TQGRSQEAT | AAV9 | 3.20 | 587/590 |
| Seq ID 668 | ATSSRTTLA | AAV9 | 3.20 | 588/589 |
| Seq ID 669 | PSSIGKHDN | AAV9 | 3.19 | 587/590 |
| Seq ID 670 | RHSGDSIPE | AAV9 | 3.19 | 587/590 |
| Seq ID 671 | ARPATTQSN | AAV9 | 3.19 | 587/590 |
| Seq ID 672 | PAPSGNTMA | AAV9 | 3.19 | 587/590 |
| Seq ID 673 | KSPSMSVVL | AAV9 | 3.18 | 587/590 |
| Seq ID 674 | IPGPNGQIP | AAV9 | 3.18 | 587/590 |
| Seq ID 675 | PQDYHMSEH | AAV9 | 3.18 | 587/590 |
| Seq ID 676 | ATHLRVIYERMNQSLA | AAV9 | 3.18 | 588/589 |
| Seq ID 677 | AHGKREVALHLHPDHA | AAV2 | 3.18 | R588A/589 |
| Seq ID 678 | PHTHAQMNL | AAV9 | 3.18 | 587/590 |
| Seq ID 679 | TVPSQQPGT | AAV9 | 3.18 | 587/590 |
| Seq ID 680 | AEYKDCHLAQVPSHTA | AAV2 | 3.17 | R588A/589 |
| Seq ID 681 | AVGALIQRGFIRPKDA | AAV2 | 3.17 | R588A/589 |
| Seq ID 682 | TETDPMQRM | AAV9 | 3.17 | 587/590 |
| Seq ID 683 | HRQVAGEST | AAV9 | 3.17 | 587/590 |
| Seq ID 684 | SQSPTRIIQ | AAV9 | 3.17 | 587/590 |
| Seq ID 685 | RLAIPDGIT | AAV9 | 3.17 | 587/590 |
| Seq ID 686 | RPPQNPQTI | AAV9 | 3.17 | 587/590 |
| Seq ID 687 | VELMRTHST | AAV9 | 3.17 | 587/590 |
| Seq ID 688 | HQPQYSKSD | AAV9 | 3.16 | 587/590 |
| Seq ID 689 | ATMQCAHTA | AAV2 | 3.16 | R588A/589 |
| Seq ID 690 | KMGGVAAKI | AAV9 | 3.16 | 587/590 |
| Seq ID 691 | TMNKSADRG | AAV9 | 3.16 | 587/590 |
| Seq ID 692 | EVDDFVSYG | AAV9 | 3.16 | 587/590 |
| Seq ID 693 | PMSQKVQSA | AAV9 | 3.15 | 587/590 |
| Seq ID 694 | IHSKANDVL | AAV9 | 3.15 | 587/590 |
| Seq ID 695 | AVVSPANAA | AAV9 | 3.15 | 587/590 |
| Seq ID 696 | RQTQTASEY | AAV9 | 3.14 | 587/590 |
| Seq ID 697 | MESHPQAPQ | AAV9 | 3.14 | 587/590 |
| Seq ID 698 | TFRESEMIM | AAV9 | 3.14 | 587/590 |
| Seq ID 699 | HHTPQNMVN | AAV9 | 3.13 | 587/590 |
| Seq ID 700 | KTQQMTPVT | AAV9 | 3.13 | 587/590 |
| Seq ID 701 | IAPQQTYWK | AAV9 | 3.13 | 587/590 |
| Seq ID 702 | EVHSKVMHP | AAV9 | 3.13 | 587/590 |
| Seq ID 703 | NKSTSTVAW | AAV9 | 3.13 | 587/590 |
| Seq ID 704 | ADSAQAMTK | AAV9 | 3.12 | 587/590 |
| Seq ID 705 | MESRKPNDI | AAV9 | 3.12 | 587/590 |
| Seq ID 706 | NVAQTMIRV | AAV9 | 3.11 | 587/590 |
| Seq ID 707 | HPRDHEHVD | AAV9 | 3.11 | 587/590 |
| Seq ID 708 | RVNGSQKES | AAV9 | 3.11 | 587/590 |
| Seq ID 709 | YSRASSETA | AAV9 | 3.11 | 587/590 |
| Seq ID 710 | NWGQTQAKQ | AAV9 | 3.11 | 587/590 |
| Seq ID 711 | AQEMSKDLEEVKAKVA | AAV2 | 3.11 | R588A/589 |
| Seq ID 712 | REMRASQVE | AAV9 | 3.10 | 587/590 |
| Seq ID 713 | QYIQEHVAK | AAV9 | 3.10 | 587/590 |
| Seq ID 714 | QVAQTTLPI | AAV9 | 3.10 | 587/590 |
| Seq ID 715 | NTVQNMAHQ | AAV9 | 3.10 | 587/590 |
| Seq ID 716 | TQSREMDTQ | AAV9 | 3.10 | 587/590 |
| Seq ID 717 | DDAAHHGFD | AAV9 | 3.10 | 587/590 |
| Seq ID 718 | AATPSPVLPDIVMEAA | AAV2 | 3.10 | R588A/589 |
| Seq ID 719 | QQASGNNLR | AAV9 | 3.10 | 587/590 |
| Seq ID 720 | AKVVTDSMS | AAV9 | 3.09 | 587/590 |
| Seq ID 721 | SQVVSQGSQ | AAV9 | 3.09 | 587/590 |
| Seq ID 722 | FQHRANSME | AAV9 | 3.09 | 587/590 |
| Seq ID 723 | ANRGTEWDA | AAV9 | 3.09 | 588/589 |
| Seq ID 724 | TIDEVDIGY | AAV9 | 3.08 | 587/590 |
| Seq ID 725 | LQYKAMQVS | AAV9 | 3.08 | 587/590 |
| Seq ID 726 | AIPVCEAFQCTALSNA | AAV2 | 3.08 | R588A/589 |
| Seq ID 727 | TEDSPQMTQ | AAV9 | 3.08 | 587/590 |
| Seq ID 728 | SGSMTPTTV | AAV9 | 3.08 | 587/590 |
| Seq ID 729 | LPMPTSQGI | AAV9 | 3.08 | 587/590 |
| Seq ID 730 | SYPTPQSQT | AAV9 | 3.08 | 587/590 |
| Seq ID 731 | SASKSFPTA | AAV9 | 3.08 | 587/590 |
| Seq ID 732 | PSQPMKFIN | AAV9 | 3.08 | 587/590 |
| Seq ID 733 | VEAQHKPAY | AAV9 | 3.07 | 587/590 |
| Seq ID 734 | IPSPVTAYW | AAV9 | 3.07 | 587/590 |
| Seq ID 735 | NYLSTSVPR | AAV9 | 3.07 | 587/590 |
| Seq ID 736 | TNSVKMPPS | AAV9 | 3.07 | 587/590 |
| Seq ID 737 | PQEDVKSVM | AAV9 | 3.07 | 587/590 |
| Seq ID 738 | EPHNMKVEK | AAV9 | 3.06 | 587/590 |
| Seq ID 739 | PQKQSLHTV | AAV9 | 3.06 | 587/590 |
| Seq ID 740 | KPQGVNVTY | AAV9 | 3.06 | 587/590 |
| Seq ID 741 | FQRSEVQTI | AAV9 | 3.05 | 587/590 |
| Seq ID 742 | AAEPAPDFSDYSEMAA | AAV9 | 3.05 | 588/589 |
| Seq ID 743 | QRQGGMEAK | AAV9 | 3.05 | 587/590 |
| Seq ID 744 | TASVKQQNT | AAV9 | 3.05 | 587/590 |
| Seq ID 745 | HRNTESGDQ | AAV9 | 3.05 | 587/590 |
| Seq ID 746 | FPKSYEQVQ | AAV9 | 3.05 | 587/590 |
| Seq ID 747 | QGASQRVAI | AAV9 | 3.05 | 587/590 |
| Seq ID 748 | HPMAKDVVK | AAV9 | 3.04 | 587/590 |
| Seq ID 749 | REQKLYKEP | AAV9 | 3.04 | 587/590 |
| Seq ID 750 | TEQQWYGRM | AAV9 | 3.04 | 587/590 |
| Seq ID 751 | APTNKTVGI | AAV9 | 3.04 | 587/590 |
| Seq ID 752 | QRTAVNEMQ | AAV9 | 3.04 | 587/590 |
| Seq ID 753 | PQNAQRVDT | AAV9 | 3.04 | 587/590 |
| Seq ID 754 | KDDDKVGIV | AAV9 | 3.04 | 587/590 |
| Seq ID 755 | APAPAAPPSTPAAPKA | AAV2 | 3.04 | R588A/589 |
| Seq ID 756 | NHAVMHTVS | AAV9 | 3.04 | 587/590 |
| Seq ID 757 | MRTQAVDGF | AAV9 | 3.04 | 587/590 |
| Seq ID 758 | DIQHATKRE | AAV9 | 3.03 | 587/590 |
| Seq ID 759 | ATQAHTMMY | AAV9 | 3.03 | 587/590 |
| Seq ID 760 | QGTSEASRY | AAV9 | 3.03 | 587/590 |
| Seq ID 761 | RNSHGMSES | AAV9 | 3.03 | 587/590 |
| Seq ID 762 | PNSAHNNSY | AAV9 | 3.02 | 587/590 |
| Seq ID 763 | TTGASKDKV | AAV9 | 3.02 | 587/590 |
| Seq ID 764 | DRTGVWAGE | AAV9 | 3.02 | 587/590 |
| Seq ID 765 | VQTLDRGSQ | AAV9 | 3.02 | 587/590 |
| Seq ID 766 | DATAHSLIQ | AAV9 | 3.02 | 587/590 |
| Seq ID 767 | IGQAPEVND | AAV9 | 3.01 | 587/590 |
| Seq ID 768 | HYAKVTEQA | AAV9 | 3.01 | 587/590 |
| Seq ID 769 | DPSVRSNMI | AAV9 | 3.01 | 587/590 |
| Seq ID 770 | MSKVSTQDR | AAV9 | 3.00 | 587/590 |
| Seq ID 771 | AQDRISQWA | AAV2 | 3.00 | R588A/589 |
| Seq ID 772 | AHLAQPATLSQTIVPA | AAV2 | 3.00 | R588A/589 |
| Seq ID 773 | NEKIRELPK | AAV9 | 2.99 | 587/590 |
| Seq ID 774 | QMTKHETYI | AAV9 | 2.99 | 587/590 |
| Seq ID 775 | GAQQYSQVS | AAV9 | 2.99 | 587/590 |
| Seq ID 776 | IQDDPMTSM | AAV9 | 2.98 | 587/590 |
| Seq ID 777 | VPTVERNSL | AAV9 | 2.98 | 587/590 |
| Seq ID 778 | KLVMTQTEY | AAV9 | 2.98 | 587/590 |
| Seq ID 779 | GGVPMKALI | AAV9 | 2.97 | 587/590 |
| Seq ID 780 | IQSPQQAMR | AAV9 | 2.97 | 587/590 |
| Seq ID 781 | SFHGPIKNY | AAV9 | 2.97 | 587/590 |
| Seq ID 782 | NTKDNAGKD | AAV9 | 2.97 | 587/590 |
| Seq ID 783 | ADFSDLYYHTMNNKHA | AAV2 | 2.97 | R588A/589 |
| Seq ID 784 | QKQIMHPES | AAV9 | 2.97 | 587/590 |
| Seq ID 785 | PQPQMGHSM | AAV9 | 2.96 | 587/590 |
| Seq ID 786 | QLHTGIQNS | AAV9 | 2.96 | 587/590 |
| Seq ID 787 | AFEVMEDHAGTYGLGA | AAV2 | 2.96 | R588A/589 |
| Seq ID 788 | SYVQPTAIM | AAV9 | 2.95 | 587/590 |
| Seq ID 789 | IQATRNVGV | AAV9 | 2.95 | 587/590 |
| Seq ID 790 | MILVVLQSV | AAV9 | 2.95 | 587/590 |
| Seq ID 791 | KMFSSSEQF | AAV9 | 2.95 | 587/590 |
| Seq ID 792 | TTDRDKMAL | AAV9 | 2.95 | 587/590 |
| Seq ID 793 | KAINTMIEA | AAV9 | 2.94 | 587/590 |
| Seq ID 794 | HMTNGTPMM | AAV9 | 2.94 | 587/590 |
| Seq ID 795 | MQNSVKQVW | AAV9 | 2.94 | 587/590 |
| Seq ID 796 | SQPKMQETG | AAV9 | 2.93 | 587/590 |
| Seq ID 797 | TESHSITPM | AAV9 | 2.93 | 587/590 |
| Seq ID 798 | QVSQGVHGQ | AAV9 | 2.92 | 587/590 |
| Seq ID 799 | TTQMQPMVA | AAV9 | 2.92 | 587/590 |
| Seq ID 800 | KLTVTRRMF | AAV9 | 2.92 | 587/590 |
| Seq ID 801 | AGIATSGSA | AAV9 | 2.91 | 588/589 |
| Seq ID 802 | TPNMQSMSY | AAV9 | 2.91 | 587/590 |
| Seq ID 803 | FNDQPQKIF | AAV9 | 2.91 | 587/590 |
| Seq ID 804 | PTSVHGQML | AAV9 | 2.91 | 587/590 |
| Seq ID 805 | SQTHQVGYP | AAV9 | 2.90 | 587/590 |
| Seq ID 806 | QGTTATRPD | AAV9 | 2.90 | 587/590 |
| Seq ID 807 | AGGKVQIINKKLDLSA | AAV2 | 2.89 | R588A/589 |
| Seq ID 808 | ASLSITEDA | AAV2 | 2.89 | R588A/589 |
| Seq ID 809 | TSKPPALQG | AAV9 | 2.89 | 587/590 |
| Seq ID 810 | KVMEKQGMR | AAV9 | 2.88 | 587/590 |
| Seq ID 811 | KTVESPQQQ | AAV9 | 2.88 | 587/590 |
| Seq ID 812 | AGPSVGRAKGQDAPLA | AAV9 | 2.88 | 588/589 |
| Seq ID 813 | RHPSPVNVL | AAV9 | 2.88 | 587/590 |
| Seq ID 814 | YQNDMRKGT | AAV9 | 2.87 | 587/590 |
| Seq ID 815 | LINAHQPQK | AAV9 | 2.87 | 587/590 |
| Seq ID 816 | AGHAIENAVRSSLKGA | AAV2 | 2.87 | R588A/589 |
| Seq ID 817 | GIGTHMTMV | AAV9 | 2.86 | 587/590 |
| Seq ID 818 | RTLHVDNSP | AAV9 | 2.86 | 587/590 |
| Seq ID 819 | ADLENLRDLLHVGGGA | AAV2 | 2.86 | R588A/589 |
| Seq ID 820 | QEQANQRPN | AAV9 | 2.86 | 587/590 |
| Seq ID 821 | VSAQSMRNP | AAV9 | 2.85 | 587/590 |
| Seq ID 822 | TLQQKTYNP | AAV9 | 2.85 | 587/590 |
| Seq ID 823 | FLDSNHSMT | AAV9 | 2.85 | 587/590 |
| Seq ID 824 | TDYYSTDDY | AAV9 | 2.84 | 587/590 |
| Seq ID 825 | AVLTQTMGK | AAV9 | 2.84 | 587/590 |
| Seq ID 826 | PAPHSVVQM | AAV9 | 2.84 | 587/590 |
| Seq ID 827 | RPNASDIKG | AAV9 | 2.84 | 587/590 |
| Seq ID 828 | PQRNTGIVQ | AAV9 | 2.84 | 587/590 |
| Seq ID 829 | IVSQTPVAV | AAV9 | 2.83 | 587/590 |
| Seq ID 830 | PTTRIGEHP | AAV9 | 2.83 | 587/590 |
| Seq ID 831 | RQPTDSYSK | AAV9 | 2.83 | 587/590 |
| Seq ID 832 | TVGGKTYAD | AAV9 | 2.83 | 587/590 |
| Seq ID 833 | LKNPPLGNQ | AAV9 | 2.83 | 587/590 |
| Seq ID 834 | LSQSQSQTY | AAV9 | 2.82 | 587/590 |
| Seq ID 835 | PPTAQAQYD | AAV9 | 2.82 | 587/590 |
| Seq ID 836 | NDQLHISLR | AAV9 | 2.82 | 587/590 |
| Seq ID 837 | AKNQYVEED | AAV9 | 2.82 | 587/590 |
| Seq ID 838 | QARANAFPD | AAV9 | 2.82 | 587/590 |
| Seq ID 839 | NGNFAYDHP | AAV9 | 2.82 | 587/590 |
| Seq ID 840 | ILQSNTRGI | AAV9 | 2.82 | 587/590 |
| Seq ID 841 | TQPQQHNEP | AAV9 | 2.82 | 587/590 |
| Seq ID 842 | QRHNENVQM | AAV9 | 2.82 | 587/590 |
| Seq ID 843 | AQVLRIRKRANSFLEA | AAV2 | 2.82 | R588A/589 |
| Seq ID 844 | QLRDVQLQH | AAV9 | 2.82 | 587/590 |
| Seq ID 845 | YAKQSTQQM | AAV9 | 2.81 | 587/590 |
| Seq ID 846 | TQVLRDKQP | AAV9 | 2.80 | 587/590 |
| Seq ID 847 | QNKIEFRPE | AAV9 | 2.80 | 587/590 |
| Seq ID 848 | SSHASMSST | AAV9 | 2.79 | 587/590 |
| Seq ID 849 | RVVEQGRGI | AAV9 | 2.79 | 587/590 |
| Seq ID 850 | HRNPTVVVD | AAV9 | 2.79 | 587/590 |
| Seq ID 851 | APWNSATVLQQTYHAA | AAV2 | 2.79 | R588A/589 |
| Seq ID 852 | MWNVTSGAE | AAV9 | 2.78 | 587/590 |
| Seq ID 853 | KTQQALVLQ | AAV9 | 2.78 | 587/590 |
| Seq ID 854 | QVHQESMFG | AAV9 | 2.78 | 587/590 |
| Seq ID 855 | QLNTTKPIA | AAV9 | 2.78 | 587/590 |
| Seq ID 856 | EQYAIKQPT | AAV9 | 2.78 | 587/590 |
| Seq ID 857 | ALGKTAETH | AAV9 | 2.77 | 587/590 |
| Seq ID 858 | KVFHTPEMN | AAV9 | 2.77 | 587/590 |
| Seq ID 859 | ASQTAPIVT | AAV9 | 2.77 | 587/590 |
| Seq ID 860 | TLKKTDDVW | AAV9 | 2.77 | 587/590 |
| Seq ID 861 | AHEALSPFYSERLKEA | AAV2 | 2.77 | R588A/589 |
| Seq ID 862 | QINGSHRIV | AAV9 | 2.77 | 587/590 |
| Seq ID 863 | ATAQDLFTA | AAV2 | 2.76 | R588A/589 |
| Seq ID 864 | ATAQDLFTA | AAV9 | 2.76 | 588/589 |
| Seq ID 865 | SQHTSDMES | AAV9 | 2.76 | 587/590 |
| Seq ID 866 | YRTATTTNS | AAV9 | 2.76 | 587/590 |
| Seq ID 867 | PASKQFTPD | AAV9 | 2.76 | 587/590 |
| Seq ID 868 | TATRQEVHH | AAV9 | 2.76 | 587/590 |
| Seq ID 869 | TTTGVMVAK | AAV9 | 2.75 | 587/590 |
| Seq ID 870 | NPVFHQSVA | AAV9 | 2.75 | 587/590 |
| Seq ID 871 | KPVSSQHTP | AAV9 | 2.75 | 587/590 |
| Seq ID 872 | TMATAHQTD | AAV9 | 2.75 | 587/590 |
| Seq ID 873 | VVSGVPMSQ | AAV9 | 2.75 | 587/590 |
| Seq ID 874 | MTFGMQQNK | AAV9 | 2.74 | 587/590 |
| Seq ID 875 | YGQAVVMQN | AAV9 | 2.74 | 587/590 |
| Seq ID 876 | LFHKHPDEM | AAV9 | 2.74 | 587/590 |
| Seq ID 877 | RMRDQDVTS | AAV9 | 2.74 | 587/590 |
| Seq ID 878 | MKQTGGSSF | AAV9 | 2.73 | 587/590 |
| Seq ID 879 | LQASSGAMF | AAV9 | 2.73 | 587/590 |
| Seq ID 880 | NVSTTASQV | AAV9 | 2.73 | 587/590 |
| Seq ID 881 | IMQTSVGRD | AAV9 | 2.73 | 587/590 |
| Seq ID 882 | KLAGVVAIH | AAV9 | 2.73 | 587/590 |
| Seq ID 883 | EIKGAADSH | AAV9 | 2.72 | 587/590 |
| Seq ID 884 | VQQGNPMQR | AAV9 | 2.72 | 587/590 |
| Seq ID 885 | HRWVQLWRV | AAV9 | 2.72 | 587/590 |
| Seq ID 886 | RAETHKYNP | AAV9 | 2.72 | 587/590 |
| Seq ID 887 | RDPAINHDQ | AAV9 | 2.72 | 587/590 |
| Seq ID 888 | PQSQGMQQR | AAV9 | 2.72 | 587/590 |
| Seq ID 889 | GIRQDQEYF | AAV9 | 2.71 | 587/590 |
| Seq ID 890 | ANCRIPA | AAV2 | 2.71 | R588A/589 |
| Seq ID 891 | GTTMKVQMS | AAV9 | 2.71 | 587/590 |
| Seq ID 892 | NTHSSHIMV | AAV9 | 2.71 | 587/590 |
| Seq ID 893 | KQREPGNVG | AAV9 | 2.71 | 587/590 |
| Seq ID 894 | IYFNDAEQI | AAV9 | 2.70 | 587/590 |
| Seq ID 895 | NHTAKAAPL | AAV9 | 2.70 | 587/590 |
| Seq ID 896 | QHVVGQFPQ | AAV9 | 2.69 | 587/590 |
| Seq ID 897 | RIERQHNDQ | AAV9 | 2.69 | 587/590 |
| Seq ID 898 | AEAKRHRPLIALPTQA | AAV2 | 2.69 | R588A/589 |
| Seq ID 899 | EPHTSSQQR | AAV9 | 2.69 | 587/590 |
| Seq ID 900 | AEMYCAPLKPAHSARA | AAV2 | 2.69 | R588A/589 |
| Seq ID 901 | DPHQRMAQQ | AAV9 | 2.69 | 587/590 |
| Seq ID 902 | PSITNRQGA | AAV9 | 2.68 | 587/590 |
| Seq ID 903 | ADEPARKGKVHIPFPA | AAV2 | 2.68 | R588A/589 |
| Seq ID 904 | LLTPQSRSS | AAV9 | 2.67 | 587/590 |
| Seq ID 905 | LTLQGQAKQ | AAV9 | 2.67 | 587/590 |
| Seq ID 906 | QEDDQRSDR | AAV9 | 2.67 | 587/590 |
| Seq ID 907 | PTNPGEVPV | AAV9 | 2.66 | 587/590 |
| Seq ID 908 | MLSNEDGTN | AAV9 | 2.66 | 587/590 |
| Seq ID 909 | PMEMTTELF | AAV9 | 2.65 | 587/590 |
| Seq ID 910 | MPTQSRQYI | AAV9 | 2.65 | 587/590 |
| Seq ID 911 | SKTQHTNPE | AAV9 | 2.65 | 587/590 |
| Seq ID 912 | QDSFFQRWA | AAV9 | 2.64 | 587/590 |
| Seq ID 913 | ITIDVQSAK | AAV9 | 2.64 | 587/590 |
| Seq ID 914 | PQQSHVVSQ | AAV9 | 2.64 | 587/590 |
| Seq ID 915 | VQVGTSVGK | AAV9 | 2.64 | 587/590 |
| Seq ID 916 | SAQTSHPIM | AAV9 | 2.64 | 587/590 |
| Seq ID 917 | PQEMMALVY | AAV9 | 2.63 | 587/590 |
| Seq ID 918 | QHYRMRPHT | AAV9 | 2.63 | 587/590 |
| Seq ID 919 | NMAQHGQLQ | AAV9 | 2.63 | 587/590 |
| Seq ID 920 | RHGLTTPDF | AAV9 | 2.62 | 587/590 |
| Seq ID 921 | TTMQRTMDV | AAV9 | 2.62 | 587/590 |
| Seq ID 922 | PKSHGAHDV | AAV9 | 2.62 | 587/590 |
| Seq ID 923 | AIENAVRSSLKVATSA | AAV9 | 2.62 | 588/589 |
| Seq ID 924 | PMVDAMVSV | AAV9 | 2.62 | 587/590 |
| Seq ID 925 | TEQPEPFVF | AAV9 | 2.62 | 587/590 |
| Seq ID 926 | LDAVPKNIK | AAV9 | 2.61 | 587/590 |
| Seq ID 927 | TGMSQATSR | AAV9 | 2.61 | 587/590 |
| Seq ID 928 | PTRPSSETM | AAV9 | 2.61 | 587/590 |
| Seq ID 929 | RIEGSHPPP | AAV9 | 2.61 | 587/590 |
| Seq ID 930 | VKGHQEFAN | AAV9 | 2.61 | 587/590 |
| Seq ID 931 | MLVGVPQSK | AAV9 | 2.60 | 587/590 |
| Seq ID 932 | QRQQPQPFF | AAV9 | 2.59 | 587/590 |
| Seq ID 933 | ARMQASALA | AAV2 | 2.59 | R588A/589 |
| Seq ID 934 | DWEAKARVA | AAV9 | 2.59 | 587/590 |
| Seq ID 935 | PIASTITNR | AAV9 | 2.58 | 587/590 |
| Seq ID 936 | ENSQPIRPS | AAV9 | 2.58 | 587/590 |
| Seq ID 937 | AHMNVQNGKWDSDPSA | AAV2 | 2.58 | R588A/589 |
| Seq ID 938 | PHQVPRDPV | AAV9 | 2.58 | 587/590 |
| Seq ID 939 | TTSTNREVS | AAV9 | 2.57 | 587/590 |
| Seq ID 940 | PSGVMQDPV | AAV9 | 2.57 | 587/590 |
| Seq ID 941 | AKSHELQVA | AAV2 | 2.57 | R588A/589 |
| Seq ID 942 | AVNQVHHQM | AAV9 | 2.57 | 587/590 |
| Seq ID 943 | SVGRQQQDI | AAV9 | 2.57 | 587/590 |
| Seq ID 944 | VAMIKQSSI | AAV9 | 2.57 | 587/590 |
| Seq ID 945 | DILIWQHSD | AAV9 | 2.56 | 587/590 |
| Seq ID 946 | FTTKHVNEQ | AAV9 | 2.56 | 587/590 |
| Seq ID 947 | AKPYSGRRPDAIRLGA | AAV2 | 2.56 | R588A/589 |
| Seq ID 948 | QSMADPRAM | AAV9 | 2.56 | 587/590 |
| Seq ID 949 | AHLASNLSA | AAV2 | 2.56 | R588A/589 |
| Seq ID 950 | LQHSNQDQE | AAV9 | 2.55 | 587/590 |
| Seq ID 951 | NPWDQTKAM | AAV9 | 2.55 | 587/590 |
| Seq ID 952 | VTDVFDVYD | AAV9 | 2.55 | 587/590 |
| Seq ID 953 | EYDQGVTNF | AAV9 | 2.55 | 587/590 |
| Seq ID 954 | QTYSMPQHL | AAV9 | 2.54 | 587/590 |
| Seq ID 955 | FKTDRDSML | AAV9 | 2.54 | 587/590 |
| Seq ID 956 | AIPVPVDGMEYHWGNA | AAV2 | 2.54 | R588A/589 |
| Seq ID 957 | PASKNVQPM | AAV9 | 2.53 | 587/590 |
| Seq ID 958 | RLEISSQQI | AAV9 | 2.53 | 587/590 |
| Seq ID 959 | IQGLPSMVH | AAV9 | 2.52 | 587/590 |
| Seq ID 960 | NIAQNLLME | AAV9 | 2.52 | 587/590 |
| Seq ID 961 | HPQPSGHFS | AAV9 | 2.52 | 587/590 |
| Seq ID 962 | IPRQGSQTT | AAV9 | 2.52 | 587/590 |
| Seq ID 963 | DVLGQMGKM | AAV9 | 2.52 | 587/590 |
| Seq ID 964 | TYSSPSIGT | AAV9 | 2.52 | 587/590 |
| Seq ID 965 | EHKDFQVSG | AAV9 | 2.52 | 587/590 |
| Seq ID 966 | EVRPKQDFT | AAV9 | 2.51 | 587/590 |
| Seq ID 967 | ARYEAVSFMDVNSTWA | AAV2 | 2.51 | R588A/589 |
| Seq ID 968 | PYTQKTGSV | AAV9 | 2.51 | 587/590 |
| Seq ID 969 | NFVNNQPGY | AAV9 | 2.51 | 587/590 |
| Seq ID 970 | SGLNMKTSV | AAV9 | 2.51 | 587/590 |
| Seq ID 971 | AQHTLKHFEHVRMVDA | AAV2 | 2.50 | R588A/589 |
| Seq ID 972 | AQHTLKHFEHVRMVDA | AAV9 | 2.50 | 588/589 |
| Seq ID 973 | KENPQHGIY | AAV9 | 2.50 | 587/590 |
| Seq ID 974 | KSPTPDINT | AAV9 | 2.49 | 587/590 |
| Seq ID 975 | LTSSKSDPA | AAV9 | 2.49 | 587/590 |
| Seq ID 976 | ARMQASQVA | AAV2 | 2.49 | R588A/589 |
| Seq ID 977 | RYHRDTIDM | AAV9 | 2.49 | 587/590 |
| Seq ID 978 | ASLSITKPA | AAV2 | 2.48 | R588A/589 |
| Seq ID 979 | ASSPGSPGTPGSRSRA | AAV2 | 2.48 | R588A/589 |
| Seq ID 980 | ADRIPA | AAV2 | 2.46 | R588A/589 |
| Seq ID 981 | HETQVKGMQ | AAV9 | 2.45 | 587/590 |
| Seq ID 982 | TPRATPSMP | AAV9 | 2.44 | 587/590 |
| Seq ID 983 | RPTQHQDIN | AAV9 | 2.43 | 587/590 |
| Seq ID 984 | TTNTQGAQV | AAV9 | 2.43 | 587/590 |
| Seq ID 985 | HYGWQQVPD | AAV9 | 2.43 | 587/590 |
| Seq ID 986 | HPGSSMVPT | AAV9 | 2.42 | 587/590 |
| Seq ID 987 | SSVSQVTLR | AAV9 | 2.42 | 587/590 |
| Seq ID 988 | MYAAGNVHA | AAV9 | 2.42 | 587/590 |
| Seq ID 989 | TSGVVQTIH | AAV9 | 2.41 | 587/590 |
| Seq ID 990 | HMHPAQSEY | AAV9 | 2.40 | 587/590 |
| Seq ID 991 | PQKGDMHTS | AAV9 | 2.40 | 587/590 |
| Seq ID 992 | ATYQPLFTA | AAV2 | 2.39 | R588A/589 |
| Seq ID 993 | ATYQPLFTA | AAV9 | 2.39 | 588/589 |
| Seq ID 994 | AQSSDIENA | AAV2 | 2.38 | R588A/589 |
| Seq ID 995 | QVAIQPQEE | AAV9 | 2.38 | 587/590 |
| Seq ID 996 | RAVQNVTTM | AAV9 | 2.37 | 587/590 |
| Seq ID 997 | PSTLVMQSV | AAV9 | 2.37 | 587/590 |
| Seq ID 998 | QQQLLDDIP | AAV9 | 2.35 | 587/590 |
| Seq ID 999 | WEKHIMIDY | AAV9 | 2.34 | 587/590 |
| Seq ID 1000 | NQSTSVNTW | AAV9 | 2.34 | 587/590 |
| Seq ID 1001 | TVQEADSQP | AAV9 | 2.33 | 587/590 |
| Seq ID 1002 | AGDAIENAVRSSLKVA | AAV2 | 2.32 | R588A/589 |
| Seq ID 1003 | RTDKSQYQP | AAV9 | 2.32 | 587/590 |
| Seq ID 1004 | ADGGFIYEAGLAPYKA | AAV2 | 2.31 | R588A/589 |
| Seq ID 1005 | ARMKDNTPP | AAV9 | 2.31 | 587/590 |
| Seq ID 1006 | STITPMRQI | AAV9 | 2.30 | 587/590 |
| Seq ID 1007 | QTPIAPPPI | AAV9 | 2.30 | 587/590 |
| Seq ID 1008 | AQPEHRSER | AAV9 | 2.29 | 587/590 |
| Seq ID 1009 | ASRANISHA | AAV2 | 2.29 | R588A/589 |
| Seq ID 1010 | AAAAGAVVGGLGGYMA | AAV2 | 2.29 | R588A/589 |
| Seq ID 1011 | PQMRQLYHS | AAV9 | 2.29 | 587/590 |
| Seq ID 1012 | LEFHSQAMV | AAV9 | 2.29 | 587/590 |
| Seq ID 1013 | AQKTYQEGD | AAV9 | 2.29 | 587/590 |
| Seq ID 1014 | AMFSDLYYLTMNNKHA | AAV2 | 2.28 | R588A/589 |
| Seq ID 1015 | VLDYMDIVF | AAV9 | 2.27 | 587/590 |
| Seq ID 1016 | ADFSDLCYLTMNNKHA | AAV2 | 2.26 | R588A/589 |
| Seq ID 1017 | ANAVIPA | AAV2 | 2.26 | R588A/589 |
| Seq ID 1018 | KRERGMQDY | AAV9 | 2.25 | 587/590 |
| Seq ID 1019 | GPFTPAGMM | AAV9 | 2.25 | 587/590 |
| Seq ID 1020 | RKMPQAIDV | AAV9 | 2.25 | 587/590 |
| Seq ID 1021 | GSDPAGFKM | AAV9 | 2.25 | 587/590 |
| Seq ID 1022 | DAQSIQVSI | AAV9 | 2.24 | 587/590 |
| Seq ID 1023 | KVNEVMQKD | AAV9 | 2.24 | 587/590 |
| Seq ID 1024 | ATSKVLLLPPDVSALA | AAV2 | 2.24 | R588A/589 |
| Seq ID 1025 | AAAIPA | AAV2 | 2.24 | R588A/589 |
| Seq ID 1026 | ARYADGHAIENAVRSA | AAV2 | 2.23 | R588A/589 |
| Seq ID 1027 | SEHDNHQQT | AAV9 | 2.23 | 587/590 |
| Seq ID 1028 | PRVQGNDQA | AAV9 | 2.22 | 587/590 |
| Seq ID 1029 | AVAVHHTPA | AAV2 | 2.22 | R588A/589 |
| Seq ID 1030 | ADVRDPRGA | AAV2 | 2.21 | R588A/589 |
| Seq ID 1031 | ASKTSVVDLLYWRDIA | AAV2 | 2.21 | R588A/589 |
| Seq ID 1032 | NVNETKGSW | AAV9 | 2.21 | 587/590 |
| Seq ID 1033 | AGHAIENAVKSSLKVA | AAV2 | 2.19 | R588A/589 |
| Seq ID 1034 | ALDWIHGHIRDKEAPA | AAV2 | 2.19 | R588A/589 |
| Seq ID 1035 | SPKYFTPTM | AAV9 | 2.16 | 587/590 |
| Seq ID 1036 | KSQVQPQGT | AAV9 | 2.16 | 587/590 |
| Seq ID 1037 | QMDTRIPQR | AAV9 | 2.16 | 587/590 |
| Seq ID 1038 | AFRALSTGEKGFGYKA | AAV2 | 2.15 | R588A/589 |
| Seq ID 1039 | ATCEAVRCDAVHQPPA | AAV2 | 2.14 | R588A/589 |
| Seq ID 1040 | ERSSQYEKG | AAV9 | 2.14 | 587/590 |
| Seq ID 1041 | IRPGTAMAP | AAV9 | 2.14 | 587/590 |
| Seq ID 1042 | SPHSVQGSG | AAV9 | 2.13 | 587/590 |
| Seq ID 1043 | QQAQKVAGV | AAV9 | 2.13 | 587/590 |
| Seq ID 1044 | PRDISTAMV | AAV9 | 2.13 | 587/590 |
| Seq ID 1045 | PRVTQDLVG | AAV9 | 2.12 | 587/590 |
| Seq ID 1046 | QQVVTRHEM | AAV9 | 2.11 | 587/590 |
| Seq ID 1047 | ARMQAKAVA | AAV2 | 2.10 | R588A/589 |
| Seq ID 1048 | ANPRIPA | AAV2 | 2.10 | R588A/589 |
| Seq ID 1049 | MVEHNHHVM | AAV9 | 2.09 | 587/590 |
| Seq ID 1050 | AQSSDISPA | AAV2 | 2.09 | R588A/589 |
| Seq ID 1051 | ARPQASAVA | AAV2 | 2.09 | R588A/589 |
| Seq ID 1052 | NDFDMPGEI | AAV9 | 2.09 | 587/590 |
| Seq ID 1053 | NRNPTMTGP | AAV9 | 2.08 | 587/590 |
| Seq ID 1054 | ANAGIPA | AAV2 | 2.08 | R588A/589 |
| Seq ID 1055 | ADFSHLYYLTMNNKHA | AAV2 | 2.08 | R588A/589 |
| Seq ID 1056 | ACELSGYGKHEALSPA | AAV2 | 2.07 | R588A/589 |
| Seq ID 1057 | TANKSAPVS | AAV9 | 2.07 | 587/590 |
| Seq ID 1058 | QGHSMPTYG | AAV9 | 2.06 | 587/590 |
| Seq ID 1059 | EYRVTDNTQ | AAV9 | 2.06 | 587/590 |
| Seq ID 1060 | LVAVQQSSG | AAV9 | 2.06 | 587/590 |
| Seq ID 1061 | ASPGGNRYPPQGGGGA | AAV9 | 2.06 | 588/589 |
| Seq ID 1062 | RTDQVMSTK | AAV9 | 2.05 | 587/590 |
| Seq ID 1063 | TSDHLFQYD | AAV9 | 2.05 | 587/590 |
| Seq ID 1064 | ANNRIPA | AAV2 | 2.05 | R588A/589 |
| Seq ID 1065 | MHTEAIMPR | AAV9 | 2.04 | 587/590 |
| Seq ID 1066 | SVVSVTMNQ | AAV9 | 2.04 | 587/590 |
| Seq ID 1067 | AYKSSCAFSCEEGFEA | AAV2 | 2.04 | R588A/589 |
| Seq ID 1068 | VTVQPHVDI | AAV9 | 2.03 | 587/590 |
| Seq ID 1069 | MEDKYAYEM | AAV9 | 2.03 | 587/590 |
| Seq ID 1070 | ASARSPSEA | AAV2 | 2.03 | R588A/589 |
| Seq ID 1071 | QHQDRTPSN | AAV9 | 2.02 | 587/590 |
| Seq ID 1072 | NRQPIHKDD | AAV9 | 2.02 | 587/590 |
| Seq ID 1073 | TTMGSPAPM | AAV9 | 2.02 | 587/590 |
| Seq ID 1074 | KQDPRYSYQ | AAV9 | 2.02 | 587/590 |
| Seq ID 1075 | AQLSITKDA | AAV2 | 2.01 | R588A/589 |
| Seq ID 1076 | PRAGTSIAP | AAV9 | 2.01 | 587/590 |
| Seq ID 1077 | AMPRNSIPA | AAV2 | 2.00 | R588A/589 |
| Seq ID 1078 | ASVTTVVSA | AAV9 | 2.00 | 587/590 |
| Seq ID 1079 | SPQGKVSQV | AAV9 | 2.00 | 587/590 |
| Seq ID 1080 | RVTNVPNAV | AAV9 | 1.99 | 587/590 |
| Seq ID 1081 | MLPQASAHK | AAV9 | 1.97 | 587/590 |
| Seq ID 1082 | ATKEADLPEPSEKQPA | AAV2 | 1.96 | R588A/589 |
| Seq ID 1083 | ARGKRGQDAMYEYMAA | AAV2 | 1.96 | R588A/589 |
| Seq ID 1084 | PNPKSHQWP | AAV9 | 1.96 | 587/590 |
| Seq ID 1085 | ADGAETEVDCNRCVCA | AAV2 | 1.95 | R588A/589 |
| Seq ID 1086 | SDTNTAPQE | AAV9 | 1.95 | 587/590 |
| Seq ID 1087 | ASKSTTDNDIALLHLA | AAV2 | 1.94 | R588A/589 |
| Seq ID 1088 | KYISTGGEW | AAV9 | 1.93 | 587/590 |
| Seq ID 1089 | AGDSCVTIMSKDKPTA | AAV2 | 1.92 | R588A/589 |
| Seq ID 1090 | AVLEGDSCVTIMSKDA | AAV2 | 1.92 | R588A/589 |
| Seq ID 1091 | AELPGFLQSGKDKDAA | AAV2 | 1.91 | R588A/589 |
| Seq ID 1092 | QQMSSHQPM | AAV9 | 1.88 | 587/590 |
| Seq ID 1093 | AYHEREQGQ | AAV9 | 1.88 | 587/590 |
| Seq ID 1094 | PKHERVGDL | AAV9 | 1.88 | 587/590 |
| Seq ID 1095 | MENADVNDG | AAV9 | 1.88 | 587/590 |
| Seq ID 1096 | IIEHVSYQY | AAV9 | 1.87 | 587/590 |
| Seq ID 1097 | NQEKQQSNT | AAV9 | 1.86 | 587/590 |
| Seq ID 1098 | KVIHMDDSY | AAV9 | 1.86 | 587/590 |
| Seq ID 1099 | NITINQREH | AAV9 | 1.85 | 587/590 |
| Seq ID 1100 | AGGCVTITAEGKPSMA | AAV2 | 1.85 | R588A/589 |
| Seq ID 1101 | QVKQSSQLN | AAV9 | 1.85 | 587/590 |
| Seq ID 1102 | AADRSSPGA | AAV2 | 1.84 | R588A/589 |
| Seq ID 1103 | NAPRGYMPD | AAV9 | 1.83 | 587/590 |
| Seq ID 1104 | IATPLQKQM | AAV9 | 1.82 | 587/590 |
| Seq ID 1105 | DVHHQYSQI | AAV9 | 1.82 | 587/590 |
| Seq ID 1106 | HHRDNSQSN | AAV9 | 1.79 | 587/590 |
| Seq ID 1107 | AGHGECVETINNYTCA | AAV2 | 1.79 | R588A/589 |
| Seq ID 1108 | FQWQEETDT | AAV9 | 1.78 | 587/590 |
| Seq ID 1109 | AHAHRSDALQLGLGKA | AAV2 | 1.78 | R588A/589 |
| Seq ID 1110 | MTTPPQSSP | AAV9 | 1.77 | 587/590 |
| Seq ID 1111 | ANSTWRTVDRLSATAA | AAV2 | 1.76 | R588A/589 |
| Seq ID 1112 | TEGRQPVGF | AAV9 | 1.73 | 587/590 |
| Seq ID 1113 | PQEEGPSQF | AAV9 | 1.73 | 587/590 |
| Seq ID 1114 | PMNYSHGAL | AAV9 | 1.72 | 587/590 |
| Seq ID 1115 | ASSSERAHQVLRIRKA | AAV2 | 1.72 | R588A/589 |
| Seq ID 1116 | AILRQQQHLFGSNVTA | AAV2 | 1.72 | R588A/589 |
| Seq ID 1117 | QKPQPQQDP | AAV9 | 1.71 | 587/590 |
| Seq ID 1118 | HTAATSTKD | AAV9 | 1.71 | 587/590 |
| Seq ID 1119 | SVKAVMPFP | AAV9 | 1.71 | 587/590 |
| Seq ID 1120 | ALIFRHHRQ | AAV9 | 1.70 | 587/590 |
| Seq ID 1121 | PMEPEHMWG | AAV9 | 1.70 | 587/590 |
| Seq ID 1122 | EKDVMRVVQ | AAV9 | 1.70 | 587/590 |
| Seq ID 1123 | ARCRAIDGDVTFCRPA | AAV2 | 1.70 | R588A/589 |
| Seq ID 1124 | ASVGGVFNSLGKGIHA | AAV2 | 1.68 | R588A/589 |
| Seq ID 1125 | VASHHFQPQ | AAV9 | 1.67 | 587/590 |
| Seq ID 1126 | TNKQMAYST | AAV9 | 1.67 | 587/590 |
| Seq ID 1127 | ATPSLPTPPTREPKKA | AAV2 | 1.66 | R588A/589 |
| Seq ID 1128 | YMEDLDPES | AAV9 | 1.66 | 587/590 |
| Seq ID 1129 | TMIQAKQLP | AAV9 | 1.66 | 587/590 |
| Seq ID 1130 | PPRVNDHAT | AAV9 | 1.66 | 587/590 |
| Seq ID 1131 | AMFDSSNYHGQDLLFA | AAV2 | 1.65 | R588A/589 |
| Seq ID 1132 | HPTVQTHSQ | AAV9 | 1.65 | 587/590 |
| Seq ID 1133 | LGSSAGVSF | AAV9 | 1.64 | 587/590 |
| Seq ID 1134 | AGLTTRPGSGLTNIKA | AAV2 | 1.62 | R588A/589 |
| Seq ID 1135 | AGHFSGSGA | AAV2 | 1.62 | R588A/589 |
| Seq ID 1136 | RVQVGPVSS | AAV9 | 1.61 | 587/590 |
| Seq ID 1137 | SKINPQTPL | AAV9 | 1.61 | 587/590 |
| Seq ID 1138 | QRDQRVTPQ | AAV9 | 1.61 | 587/590 |
| Seq ID 1139 | SPAGYQANK | AAV9 | 1.60 | 587/590 |
| Seq ID 1140 | MTHAQQLPQ | AAV9 | 1.60 | 587/590 |
| Seq ID 1141 | AFKDSAIGFSRVPPRA | AAV2 | 1.60 | R588A/589 |
| Seq ID 1142 | MTLKREIMS | AAV9 | 1.60 | 587/590 |
| Seq ID 1143 | IPMQGEQMR | AAV9 | 1.60 | 587/590 |
| Seq ID 1144 | AQETHYMTR | AAV9 | 1.59 | 587/590 |
| Seq ID 1145 | AVRHSSDFA | AAV9 | 1.58 | 588/589 |
| Seq ID 1146 | ALSSSMSMA | AAV2 | 1.58 | R588A/589 |
| Seq ID 1147 | ANPRFNENNRRVIVCA | AAV2 | 1.57 | R588A/589 |
| Seq ID 1148 | AAIENAVRSSLKVATA | AAV9 | 1.57 | 588/589 |
| Seq ID 1149 | RQGGNIMEK | AAV9 | 1.56 | 587/590 |
| Seq ID 1150 | PTSQLSAAH | AAV9 | 1.55 | 587/590 |
| Seq ID 1151 | KPSQTHNDM | AAV9 | 1.55 | 587/590 |
| Seq ID 1152 | AKVAVVRTPPKSPSSA | AAV2 | 1.54 | R588A/589 |
| Seq ID 1153 | HDQGHAAQK | AAV9 | 1.53 | 587/590 |
| Seq ID 1154 | AMAAAMGNS | AAV9 | 1.53 | 587/590 |
| Seq ID 1155 | GDTQPRMQY | AAV9 | 1.52 | 587/590 |
| Seq ID 1156 | EYPSEIVYS | AAV9 | 1.50 | 587/590 |
| Seq ID 1157 | TWQHTTETS | AAV9 | 1.49 | 587/590 |
| Seq ID 1158 | TGGAYPVIV | AAV9 | 1.49 | 587/590 |
| Seq ID 1159 | AGYGSSSRRAPQTGIA | AAV2 | 1.48 | R588A/589 |
| Seq ID 1160 | AVADSRSPA | AAV2 | 1.47 | R588A/589 |
| Seq ID 1161 | AGIVSWGRGCALKDKA | AAV2 | 1.45 | R588A/589 |
| Seq ID 1162 | AWESDDFTA | AAV2 | 1.44 | R588A/589 |
| Seq ID 1163 | SVDQSHDPN | AAV9 | 1.43 | 587/590 |
| Seq ID 1164 | ARPNLPLPA | AAV2 | 1.43 | R588A/589 |
| Seq ID 1165 | ARPNLPLPA | AAV9 | 1.43 | 588/589 |
| Seq ID 1166 | TDLADSEWR | AAV9 | 1.41 | 587/590 |
| Seq ID 1167 | AAFTTTLRGAQRLAAA | AAV2 | 1.38 | R588A/589 |
| Seq ID 1168 | VSNPQPPSR | AAV9 | 1.34 | 587/590 |
| Seq ID 1169 | APSLPTPPTREPKKVA | AAV2 | 1.34 | R588A/589 |
| Seq ID 1170 | PTHTQGRES | AAV9 | 1.33 | 587/590 |
| Seq ID 1171 | AQNAKNGNLPEFGDAA | AAV2 | 1.30 | R588A/589 |
| Seq ID 1172 | KNAVMAPGN | AAV9 | 1.29 | 587/590 |
| Seq ID 1173 | ALVPIATQTYEAWLGA | AAV9 | 1.27 | 588/589 |
| Seq ID 1174 | IPMRIETDP | AAV9 | 1.25 | 587/590 |
| Seq ID 1175 | QARDSPKGW | AAV9 | 1.24 | 587/590 |
| Seq ID 1176 | AHRERMSQVMREWEEA | AAV2 | 1.23 | R588A/589 |
| Seq ID 1177 | SRVSQNEHP | AAV9 | 1.20 | 587/590 |
| Seq ID 1178 | QRNEKSEFI | AAV9 | 1.19 | 587/590 |
| Seq ID 1179 | QTLPEVTKW | AAV9 | 1.17 | 587/590 |
| Seq ID 1180 | VKGWQVTER | AAV9 | 1.16 | 587/590 |
| Seq ID 1181 | APTTVESHGNYSTQIA | AAV2 | 1.16 | R588A/589 |
| Seq ID 1182 | PRQGPSTNL | AAV9 | 1.14 | 587/590 |
| Seq ID 1183 | AEATDGMLKIQFSAQA | AAV2 | 1.13 | R588A/589 |
| Seq ID 1184 | ALYRQKVEPLRAELQA | AAV9 | 1.09 | 588/589 |
| Seq ID 1185 | RPLTTEAIV | AAV9 | 1.07 | 587/590 |
| Seq ID 1186 | KPGSQQIMD | AAV9 | 1.06 | 587/590 |
| Seq ID 1187 | AHQLLGDLHQEGPPLA | AAV2 | 1.06 | R588A/589 |
| Seq ID 1188 | MEQLVTFKT | AAV9 | 1.03 | 587/590 |
| Seq ID 1189 | GASMQTALW | AAV9 | 1.01 | 587/590 |
| Seq ID 1190 | AAFRCLAENAGDVAFA | AAV2 | 0.95 | R588A/589 |
| Seq ID 1191 | IQTQLPPGK | AAV9 | 0.93 | 587/590 |
| Seq ID 1192 | NTVHQAQIM | AAV9 | 0.92 | 587/590 |
| Seq ID 1193 | PISNVNQSR | AAV9 | 0.91 | 587/590 |
| Seq ID 1194 | APDLVQEACESELNEA | AAV2 | 0.90 | R588A/589 |
| Seq ID 1195 | AKIQVLVEPDHFKVAA | AAV2 | 0.90 | R588A/589 |
| Seq ID 1196 | AREWFMDLNLPWSSAA | AAV2 | 0.87 | R588A/589 |
| Seq ID 1197 | AEFSSNTVKLTSGHLA | AAV9 | 0.85 | 588/589 |
| Seq ID 1198 | AFTYKSSCAFSCEEGA | AAV2 | 0.84 | R588A/589 |
| Seq ID 1199 | PYPTQASMK | AAV9 | 0.84 | 587/590 |
| Seq ID 1200 | KPTNSGQEH | AAV9 | 0.82 | 587/590 |
| Seq ID 1201 | LSTGPPEKI | AAV9 | 0.76 | 587/590 |
| Seq ID 1202 | APQLHVMSA | AAV9 | 0.74 | 587/590 |
| Seq ID 1203 | TASKMQDSK | AAV9 | 0.68 | 587/590 |
| Seq ID 1204 | AFTSSSGQKAMNGSRA | AAV2 | 0.64 | R588A/589 |
| Seq ID 1205 | AVAEFSEATAELTVSA | AAV2 | 0.64 | R588A/589 |
| Seq ID 1206 | INQNKAHDA | AAV9 | 0.60 | 587/590 |
| Seq ID 1207 | AARLSKELQAAQARLA | AAV2 | 0.56 | R588A/589 |
| Seq ID 1208 | ATKPGYVDSIQKGIQA | AAV2 | 0.55 | R588A/589 |
| Seq ID 1209 | PTTAPSMVL | AAV9 | 0.49 | 587/590 |
| Seq ID 1210 | AGGFVYIAGKCGLVPA | AAV2 | 0.47 | R588A/589 |
| Seq ID 1211 | ANPTAKRQFVQSAKEA | AAV2 | 0.44 | R588A/589 |
| Seq ID 1212 | ALALGRFWDYLRWVQA | AAV2 | 0.42 | R588A/589 |
| Seq ID 1213 | ALRVIYERMNQSLSLA | AAV2 | 0.41 | R588A/589 |
| Seq ID 1214 | VTMTQATNR | AAV9 | 0.41 | 587/590 |
| Seq ID 1215 | ALLQYNHRVKKLNEIA | AAV2 | 0.33 | R588A/589 |
| Seq ID 1216 | ALLGKINLKALAALAA | AAV2 | 0.31 | R588A/589 |
| Seq ID 1217 | RQRVSYMDD | AAV9 | 0.20 | 587/590 |
| Seq ID 1218 | ADMGQQRYD | AAV9 | 0.20 | 587/590 |
| Seq ID 1219 | ATGVLLRALAGVPVAA | AAV2 | 0.13 | R588A/589 |
| Seq ID 1220 | SIKKDPPTV | AAV9 | 0.10 | 587/590 |
| Seq ID 1221 | PGKTMLGNH | AAV9 | 0.10 | 587/590 |
| Seq ID 1222 | HQGDTLQYV | AAV9 | 0.08 | 587/590 |
| Seq ID 1223 | ALGLGKHNYCRNPDGA | AAV2 | −0.05 | R588A/589 |
| Seq ID 1224 | QERQANKSY | AAV9 | −0.07 | 587/590 |
| Seq ID 1225 | QQTRGVELN | AAV9 | −0.10 | 587/590 |
| Seq ID 1226 | ASMRVVPMA | AAV2 | −0.21 | R588A/589 |
| Seq ID 1227 | ASSTDKRDA | AAV2 | −0.22 | R588A/589 |
| Seq ID 1228 | ALIGGSIVA | AAV2 | −0.26 | R588A/589 |
| Seq ID 1229 | LLHTGTSSH | AAV9 | −0.38 | 587/590 |
| Seq ID 1230 | TQHQAQVPR | AAV9 | −0.53 | 587/590 |
| Seq ID 1231 | AAGLFGAAEGQAFHLA | AAV2 | −0.61 | R588A/589 |
| Seq ID 1232 | AENEHAHFQKAKERLA | AAV2 | −0.62 | R588A/589 |

Seq ID 1233: AAV2 R588A
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDTNGVYSEPRPIGTRYLTRNL
Seq ID 1234: AAV9
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
AQAQTGWVQNQGILPGMVWQDRDVYLQGPIWAKIPHTDGNFHPSPLMGG
FGMKHPPPQILIKNTPVPADPPTAFNKDKLNSFITQYSTGQVSVEIEWE
LQKENSKRWNPEIQYTSNYYKSNNVEFAVNTEGVYSEPRPIGTRYLTRN
L

### Example 3. Results from Round 3 Library Evaluation

The 1,236 capsid variants selected for further evaluation from the round 2 library were administered to three cynomolgus macaques in a final selection to nominate lead capsids. The fold change enrichment in brain wide CNS transduction was determined by next-generation sequencing and normalized to the abundance in the administered test article. The average log 2 fold enrichment across all CNS tissue samples and all cynomolgus macaques is shown in Table 2. Enrichment in neuronal mRNA expression was assessed using the neuron specific human synapsin 1 (hSyn1) promoter. Enrichment in mRNA expression in all CNS cells was assessed using the ubiquitous cytomegalovirus (CMV) promoter. #N/A indicates that the capsid was not detected. Many of the capsid variants exhibited substantial improvement over AAV9 in CNS transduction. These data are plotted in FIGS. 7 and 8.

TABLE 2

| | | | | Round 3 | |
|---|---|---|---|---|---|
| | | | Peptide | Log2FC | |
| Sequence | Peptide | Parent | Insertion | Data | |
| ID | Sequence | Capsid | Site | CMV | hSyn1 |
| Seq ID 1 | NFRQSEMLN | AAV9 | 587/590 | 0.58 | −0.30 |
| Seq ID 2 | QRNTDHITP | AAV9 | 587/590 | 1.24 | 0.23 |
| Seq ID 3 | MQFDPQKPD | AAV9 | 587/590 | −1.80 | 0.07 |
| Seq ID 4 | TMSSNDNMR | AAV9 | 587/590 | 0.96 | 1.69 |
| Seq ID 5 | VKESVQKAA | AAV9 | 587/590 | −0.57 | −2.72 |
| Seq ID 6 | ILNVSKPDN | AAV9 | 587/590 | −0.70 | −3.62 |
| Seq ID 7 | SQAAQLPTQ | AAV9 | 587/590 | 1.24 | −0.82 |
| Seq ID 8 | QVMAIDAAI | AAV9 | 587/590 | −2.56 | −0.05 |
| Seq ID 9 | NDTARERAI | AAV9 | 587/590 | −0.38 | −1.74 |
| Seq ID 10 | SMAAKVTAE | AAV9 | 587/590 | 0.46 | −0.06 |
| Seq ID 11 | TLGHQHSPI | AAV9 | 587/590 | 0.64 | −0.22 |
| Seq ID 12 | TVPSSGKQH | AAV9 | 587/590 | −2.24 | −2.87 |
| Seq ID 13 | AMVQISTPL | AAV9 | 587/590 | −1.12 | −1.36 |
| Seq ID 14 | VQVTGTVRF | AAV9 | 587/590 | 1.75 | 1.07 |
| Seq ID 15 | MIAQKTVVH | AAV9 | 587/590 | −0.86 | −0.52 |
| Seq ID 16 | NTHFQGQPP | AAV9 | 587/590 | 0.06 | −0.04 |
| Seq ID 17 | MDMQATGKN | AAV9 | 587/590 | 0.45 | −0.24 |
| Seq ID 18 | NLAHTRQPQ | AAV9 | 587/590 | −1.41 | −2.65 |
| Seq ID 19 | LDIPPMNSS | AAV9 | 587/590 | 1.37 | 0.75 |
| Seq ID 20 | QHSTPSWPS | AAV9 | 587/590 | −1.07 | 0.15 |
| Seq ID 21 | AFTRHNGTG | AAV2 | R588A/589 | #N/A | −2.68 |
| | GKSIYGA | | | | |
| Seq ID 22 | ARGARDEFT | AAV9 | 587/590 | −0.73 | −0.58 |
| Seq ID 23 | RPSNDKAMM | AAV9 | 587/590 | −2.20 | −2.79 |
| Seq ID 24 | AQVHMTMPQ | AAV9 | 587/590 | −1.54 | −2.46 |
| Seq ID 25 | ARQTFQHHL | AAV9 | 587/590 | 0.94 | −0.15 |
| Seq ID 26 | HQTDAPTDW | AAV9 | 587/590 | −1.94 | −0.05 |
| Seq ID 27 | ANMISEPRI | AAV9 | 588/589 | −0.49 | −2.25 |
| | SYGNDAA | | | | |
| Seq ID 28 | IERWQAEII | AAV9 | 587/590 | −1.69 | −3.67 |
| Seq ID 29 | VPHNGLHST | AAV9 | 587/590 | 1.05 | 0.77 |
| Seq ID 30 | RIHDPSLVS | AAV9 | 587/590 | −1.69 | −2.00 |
| Seq ID 31 | IPATHPQLM | AAV9 | 587/590 | 0.56 | 0.47 |
| Seq ID 32 | ITTVKNVTV | AAV9 | 587/590 | −1.52 | −2.47 |
| Seq ID 33 | DKSVKSTAI | AAV9 | 587/590 | −1.59 | −2.09 |
| Seq ID 34 | IRTHEVPLQ | AAV9 | 587/590 | −3.37 | 1.02 |
| Seq ID 35 | SPSSTSNNN | AAV9 | 587/590 | 1.48 | 0.89 |
| Seq ID 36 | LSTTSQVHN | AAV9 | 587/590 | 0.86 | 0.44 |
| Seq ID 37 | NMHNETSMT | AAV9 | 587/590 | 1.19 | 0.73 |
| Seq ID 38 | NDSRKANIG | AAV9 | 587/590 | 0.25 | 0.77 |
| Seq ID 39 | KPPNVQDAK | AAV9 | 587/590 | −0.57 | −0.28 |
| Seq ID 40 | FQHMTPKPS | AAV9 | 587/590 | −0.13 | −1.99 |
| Seq ID 41 | KQPSRLMEW | AAV9 | 587/590 | −4.42 | −4.01 |
| Seq ID 42 | VYDQVKATH | AAV9 | 587/590 | 0.48 | −0.88 |
| Seq ID 43 | SYRNQYDDQ | AAV9 | 587/590 | #N/A | −2.74 |
| Seq ID 44 | VQSTKQSVQ | AAV9 | 587/590 | 0.27 | −1.31 |
| Seq ID 45 | HQGKVDAQN | AAV9 | 587/590 | 0.17 | −0.22 |
| Seq ID 46 | IQGEIRVHT | AAV9 | 587/590 | 0.89 | 0.60 |
| Seq ID 47 | TQTLKVEDR | AAV9 | 587/590 | −1.45 | −0.80 |
| Seq ID 48 | VQSPPGIMP | AAV9 | 587/590 | −0.07 | 1.19 |
| Seq ID 49 | VHYAPNYTT | AAV9 | 587/590 | 0.48 | 0.41 |
| Seq ID 50 | YGGSETNVR | AAV9 | 587/590 | 1.42 | −0.10 |
| Seq ID 51 | IRTQTHPQV | AAV9 | 587/590 | −2.44 | −1.08 |
| Seq ID 52 | ETQVQPIQH | AAV9 | 587/590 | −0.63 | 0.42 |
| Seq ID 53 | FPVSSVNDV | AAV9 | 587/590 | 0.55 | 0.97 |
| Seq ID 54 | IQKISQAMA | AAV9 | 587/590 | −2.97 | −0.97 |
| Seq ID 55 | SQTQIGQPY | AAV9 | 587/590 | −0.48 | −0.04 |
| Seq ID 56 | AIRTNVGPV | AAV9 | 587/590 | −1.74 | −1.93 |
| Seq ID 57 | TQMSGQTPH | AAV9 | 587/590 | −1.88 | −0.71 |
| Seq ID 58 | PAKSVMIPY | AAV9 | 587/590 | #N/A | −0.42 |
| Seq ID 59 | QRVSQTNSF | AAV9 | 587/590 | −2.59 | −1.34 |
| Seq ID 60 | AFAGANYAA | AAV2 | R588A/589 | −0.70 | −0.12 |
| | WAVNVAA | | | | |
| Seq ID 61 | AVVRPQSTM | AAV9 | 587/590 | −0.92 | −1.39 |
| Seq ID 62 | PLQTPPSDN | AAV9 | 587/590 | −0.61 | −0.44 |
| Seq ID 63 | VTVSQQMTK | AAV9 | 587/590 | −0.49 | −0.47 |
| Seq ID 64 | DPFEGRIST | AAV9 | 587/590 | #N/A | −3.86 |
| Seq ID 65 | NNPTGGIAL | AAV9 | 587/590 | 1.05 | 0.27 |
| Seq ID 66 | ARYTHLVAI | AAV2 | R588A/589 | #N/A | −2.52 |
| | QNKEEIA | | | | |
| Seq ID 67 | IVTGGVLKP | AAV9 | 587/590 | −1.45 | −0.25 |
| Seq ID 68 | THMSTQASI | AAV9 | 587/590 | −0.04 | −1.01 |
| Seq ID 69 | AVPGEEEQK | AAV2 | R588A/589 | −2.90 | −4.36 |
| | FEVEKYA | | | | |
| Seq ID 70 | ATDTSHLVV | AAV9 | 587/590 | 0.91 | 0.53 |
| Seq ID 71 | YVNIMDDMD | AAV9 | 587/590 | 8.69 | 9.33 |
| Seq ID 72 | IRPDKQGGV | AAV9 | 587/590 | −1.69 | −1.75 |
| Seq ID 73 | NQTESQWKG | AAV9 | 587/590 | 0.59 | 0.91 |
| Seq ID 74 | TNEGLYKDN | AAV9 | 587/590 | −3.99 | −2.21 |
| Seq ID 75 | IMVGNQTVM | AAV9 | 587/590 | 1.63 | 0.24 |
| Seq ID 76 | IWAEKGLDT | AAV9 | 587/590 | −1.54 | −3.66 |
| Seq ID 77 | MPSQGYQQV | AAV9 | 587/590 | 0.58 | 0.26 |
| Seq ID 78 | NTFGHNVAP | AAV9 | 587/590 | −0.24 | −0.75 |
| Seq ID 79 | ASRTVSDSI | AAV2 | R588A/589 | −2.15 | −1.88 |
| | KKLITSA | | | | |
| Seq ID 80 | NHTSTPPQQ | AAV9 | 587/590 | 0.24 | −0.02 |
| Seq ID 81 | NAHAPIPVE | AAV9 | 587/590 | −1.33 | −0.81 |
| Seq ID 82 | KSTNTHEIG | AAV9 | 587/590 | −5.25 | −3.12 |
| Seq ID 83 | NTRVTNAHD | AAV9 | 587/590 | −1.15 | −1.07 |
| Seq ID 84 | AHRDACLTG | AAV2 | R588A/589 | −1.20 | −1.03 |
| | SKIQVDA | | | | |
| Seq ID 85 | AVAAETGQI | AAV9 | 587/590 | −1.76 | −1.20 |
| Seq ID 86 | NQPSMVNGH | AAV9 | 587/590 | −0.97 | −3.21 |
| Seq ID 87 | PMTQQHHSM | AAV9 | 587/590 | −3.85 | −1.63 |
| Seq ID 88 | YEKVDSLMM | AAV9 | 587/590 | −0.94 | −0.65 |
| Seq ID 89 | MDVGTHGKA | AAV9 | 587/590 | 0.84 | −0.45 |
| Seq ID 90 | THNSQVHPS | AAV9 | 587/590 | 0.24 | −0.48 |
| Seq ID 91 | PTQVRQVQQ | AAV9 | 587/590 | −1.19 | −2.43 |
| Seq ID 92 | ITRTVNTVE | AAV9 | 587/590 | −0.57 | −1.27 |
| Seq ID 93 | AMIMSPFGT | AAV9 | 587/590 | 1.39 | #N/A |
| Seq ID 94 | HAFPSFPAA | AAV9 | 587/590 | −1.65 | −0.66 |
| Seq ID 95 | KMMDNTPFY | AAV9 | 587/590 | 0.59 | −1.73 |
| Seq ID 96 | PQRLSVDPN | AAV9 | 587/590 | −2.16 | −1.57 |
| Seq ID 97 | TWGTYQHSV | AAV9 | 587/590 | −1.65 | −0.29 |
| Seq ID 98 | HYQQRDMSQ | AAV9 | 587/590 | −2.07 | −1.31 |
| Seq ID 99 | SQGHVQERM | AAV9 | 587/590 | 0.95 | 0.21 |
| Seq ID | QPRTTGNDF | AAV9 | 587/590 | −0.54 | −0.86 |
| 100 | | | | | |
| Seq ID | PKGTISSQQ | AAV9 | 587/590 | −2.30 | −2.47 |
| 101 | | | | | |
| Seq ID | REHTAAWFA | AAV9 | 587/590 | −0.96 | −1.00 |
| 102 | | | | | |
| Seq ID | QLTHSNTSQ | AAV9 | 587/590 | 0.68 | −0.28 |
| 103 | | | | | |
| Seq ID | KDMGAGHSM | AAV9 | 587/590 | −1.84 | −2.14 |
| 104 | | | | | |
| Seq ID | ARGAQRLAA | AAV9 | 588/589 | 1.65 | −0.41 |
| 105 | LGDTAWA | | | | |
| Seq ID | KQAHREHEE | AAV9 | 587/590 | −3.26 | −2.73 |
| 106 | | | | | |
| Seq ID | KSSHPYTDE | AAV9 | 587/590 | −0.69 | −1.04 |
| 107 | | | | | |
| Seq ID | MPNHVAYKD | AAV9 | 587/590 | −1.11 | −0.39 |
| 108 | | | | | |
| Seq ID | PPTTQYPQT | AAV9 | 587/590 | −2.73 | −5.78 |
| 109 | | | | | |
| Seq ID | RSHTTGQDS | AAV9 | 587/590 | −1.91 | −1.22 |
| 110 | | | | | |
| Seq ID | SQLKASVTN | AAV9 | 587/590 | 0.37 | −0.72 |
| 111 | | | | | |
| Seq ID | TAPSTRFEV | AAV9 | 587/590 | −0.50 | −0.72 |
| 112 | | | | | |
| Seq ID | PSTLEPEVW | AAV9 | 587/590 | −2.88 | −2.44 |
| 113 | | | | | |
| Seq ID | YTSPPQMMV | AAV9 | 587/590 | 0.66 | 0.18 |
| 114 | | | | | |
| Seq ID | PVPSRQVST | AAV9 | 587/590 | −2.76 | −3.23 |
| 115 | | | | | |
| Seq ID | LEKVQVHQN | AAV9 | 587/590 | −0.75 | 0.68 |
| 116 | | | | | |
| Seq ID | NQIKLQSMS | AAV9 | 587/590 | −0.18 | −1.61 |
| 117 | | | | | |
| Seq ID | GVMSPTPMV | AAV9 | 587/590 | −0.22 | −0.65 |
| 118 | | | | | |
| Seq ID | PTQRAERYF | AAV9 | 587/590 | −2.54 | −3.00 |
| 119 | | | | | |
| Seq ID | PTAKVVDSV | AAV9 | 587/590 | 0.42 | 0.36 |
| 120 | | | | | |
| Seq ID | HRIHEVQGG | AAV9 | 587/590 | −0.44 | −0.99 |
| 121 | | | | | |
| Seq ID | KNTTPVGQQ | AAV9 | 587/590 | −0.75 | −1.11 |
| 122 | | | | | |
| Seq ID | AGPPLKGAG | AAV2 | R588A/589 | −0.72 | −5.99 |
| 123 | GKERPGA | | | | |
| Seq ID | IQPASGQNM | AAV9 | 587/590 | 0.51 | 0.10 |
| 124 | | | | | |
| Seq ID | YQNQGVNVP | AAV9 | 587/590 | 1.37 | 0.67 |
| 125 | | | | | |
| Seq ID | NASSQSNAN | AAV9 | 587/590 | 1.44 | 0.02 |
| 126 | | | | | |
| Seq ID | SNTVRGDAA | AAV9 | 587/590 | −1.06 | −1.09 |
| 127 | | | | | |
| Seq ID | SKKAEFGTE | AAV9 | 587/590 | 1.35 | 0.99 |
| 128 | | | | | |
| Seq ID | KVSSEMMVH | AAV9 | 587/590 | −1.14 | −0.62 |
| 129 | | | | | |
| Seq ID | TMIEQRMDQ | AAV9 | 587/590 | −1.28 | 0.08 |
| 130 | | | | | |
| Seq ID | PPHIYHHMS | AAV9 | 587/590 | 1.30 | −0.02 |
| 131 | | | | | |
| Seq ID | MVNKAPTGA | AAV9 | 587/590 | −3.74 | −2.27 |
| 132 | | | | | |
| Seq ID | KSPVARGDT | AAV9 | 587/590 | −4.07 | −4.08 |
| 133 | | | | | |
| Seq ID | LLKQRQLMD | AAV9 | 587/590 | 0.29 | −2.21 |
| 134 | | | | | |
| Seq ID | KMKQIELVE | AAV9 | 587/590 | 0.17 | −0.69 |
| 135 | | | | | |
| Seq ID | RAPNRVNDE | AAV9 | 587/590 | −1.43 | −2.81 |
| 136 | | | | | |
| Seq ID | TMNQAPPAQ | AAV9 | 587/590 | −1.02 | −1.61 |
| 137 | | | | | |
| Seq ID | LVKEDEHWI | AAV9 | 587/590 | −2.46 | −1.42 |
| 138 | | | | | |
| Seq ID | MIGQRDNNS | AAV9 | 587/590 | −1.65 | −1.69 |
| 139 | | | | | |
| Seq ID | TSHMVSHAV | AAV9 | 587/590 | 0.25 | −0.28 |
| 140 | | | | | |
| Seq ID | IQDEEAKYT | AAV9 | 587/590 | #N/A | −3.36 |
| 141 | | | | | |
| Seq ID | TGGSKFPYM | AAV9 | 587/590 | −1.69 | −2.19 |
| 142 | | | | | |
| Seq ID | VEGSAQMIK | AAV9 | 587/590 | 0.68 | 0.85 |
| 143 | | | | | |
| Seq ID | FTDISKEPS | AAV9 | 587/590 | −0.46 | −2.45 |
| 144 | | | | | |
| Seq ID | TERPMMQQL | AAV9 | 587/590 | −0.07 | −0.07 |
| 145 | | | | | |
| Seq ID | VSLGHDKPV | AAV9 | 587/590 | −1.31 | −0.58 |
| 146 | | | | | |
| Seq ID | TQRTHISDM | AAV9 | 587/590 | −1.17 | −0.47 |
| 147 | | | | | |
| Seq ID | TSYSTHASS | AAV9 | 587/590 | 0.26 | −1.75 |
| 148 | | | | | |
| Seq ID | NTKSADHEY | AAV9 | 587/590 | 0.60 | 0.18 |
| 149 | | | | | |
| Seq ID | RDPLREQRP | AAV9 | 587/590 | −1.28 | −2.81 |
| 150 | | | | | |
| Seq ID | LGQRTVQAQ | AAV9 | 587/590 | −3.80 | −3.60 |
| 151 | | | | | |
| Seq ID | VHDMTHQVN | AAV9 | 587/590 | −0.05 | −0.29 |
| 152 | | | | | |
| Seq ID | DYPEYHDIW | AAV9 | 587/590 | −2.39 | −5.31 |
| 153 | | | | | |
| Seq ID | QTQQGNYPK | AAV9 | 587/590 | −2.71 | −3.99 |
| 154 | | | | | |
| Seq ID | QASVGTVRV | AAV9 | 587/590 | 2.08 | 1.88 |
| 155 | | | | | |
| Seq ID | SPAHQRGVD | AAV9 | 587/590 | 0.48 | −0.10 |
| 156 | | | | | |
| Seq ID | QMYGEKSVS | AAV9 | 587/590 | #N/A | −2.93 |
| 157 | | | | | |
| Seq ID | NTAVQRGSV | AAV9 | 587/590 | −1.82 | −2.58 |
| 158 | | | | | |
| Seq ID | KTIQQTQDV | AAV9 | 587/590 | 0.41 | −1.27 |
| 159 | | | | | |
| Seq ID | QMQTLGDDI | AAV9 | 587/590 | 0.05 | 0.04 |
| 160 | | | | | |
| Seq ID | SHTGQTREL | AAV9 | 587/590 | −0.43 | −1.27 |
| 161 | | | | | |
| Seq ID | KRQNTESAF | AAV9 | 587/590 | −2.34 | 0.51 |
| 162 | | | | | |
| Seq ID | NFDPLSVAE | AAV9 | 587/590 | −0.46 | −1.93 |
| 163 | | | | | |
| Seq ID | KILQQEPLM | AAV9 | 587/590 | 0.42 | −0.13 |
| 164 | | | | | |
| Seq ID | IVHAVIQST | AAV9 | 587/590 | −0.58 | −0.34 |
| 165 | | | | | |
| Seq ID | TMDHNPIQT | AAV9 | 587/590 | −2.00 | 0.30 |
| 166 | | | | | |
| Seq ID | KQPTPTNLW | AAV9 | 587/590 | −3.02 | −2.83 |
| 167 | | | | | |
| Seq ID | EQGNPGVSI | AAV9 | 587/590 | −1.30 | 0.34 |
| 168 | | | | | |
| Seq ID | IHSRVQQVT | AAV9 | 587/590 | −1.00 | 0.15 |
| 169 | | | | | |
| Seq ID | PQSQYRPAN | AAV9 | 587/590 | 0.96 | −4.50 |
| 170 | | | | | |
| Seq ID | YRRDQENAI | AAV9 | 587/590 | −0.03 | −0.40 |
| 171 | | | | | |
| Seq ID | TANDAIVAT | AAV9 | 587/590 | 0.52 | 0.16 |
| 172 | | | | | |
| Seq ID | ALYVGSKTK | AAV9 | 588/589 | −1.32 | −2.61 |
| 173 | EGVVHGA | | | | |
| Seq ID | MASTNVGTS | AAV9 | 587/590 | 0.55 | −0.17 |
| 174 | | | | | |
| Seq ID | AIQASDTYI | AAV9 | 587/590 | −1.14 | 0.59 |
| 175 | | | | | |
| Seq ID | NMQPRIVNA | AAV9 | 587/590 | −0.75 | −2.59 |
| 176 | | | | | |
| Seq ID | QSDKEQFKH | AAV9 | 587/590 | −0.08 | 0.56 |
| 177 | | | | | |
| Seq ID | SVINQQAKA | AAV9 | 587/590 | −0.82 | −0.50 |
| 178 | | | | | |
| Seq ID | NVPGSLKIQ | AAV9 | 587/590 | −2.05 | −2.22 |
| 179 | | | | | |
| Seq ID | RKDNIPMMV | AAV9 | 587/590 | 1.39 | 0.83 |
| 180 | | | | | |
| Seq ID | NKVHGTIQD | AAV9 | 587/590 | 1.31 | −0.04 |
| 181 | | | | | |
| Seq ID | SRKDKSEAE | AAV9 | 587/590 | −0.96 | −2.87 |
| 182 | | | | | |
| Seq ID | HAQASQTQF | AAV9 | 587/590 | 2.24 | 2.05 |
| 183 | | | | | |
| Seq ID | HLGHHDGAA | AAV9 | 587/590 | 0.51 | −0.18 |
| 184 | | | | | |
| Seq ID | QPQMGVMVQ | AAV9 | 587/590 | −1.58 | −1.31 |
| 185 | | | | | |
| Seq ID | ANLVRNIDA | AAV9 | 588/589 | 1.09 | −1.08 |
| 186 | | | | | |
| Seq ID | SDIKKRNAV | AAV9 | 587/590 | 1.13 | 0.96 |
| 187 | | | | | |
| Seq ID | RVMTNSIDV | AAV9 | 587/590 | −0.61 | −0.96 |
| 188 | | | | | |
| Seq ID | KHQQQLAEK | AAV9 | 587/590 | −1.24 | −2.63 |
| 189 | | | | | |
| Seq ID | QQTPQTHPV | AAV9 | 587/590 | −3.45 | −2.26 |
| 190 | | | | | |
| Seq ID | IHNAHVPHQ | AAV9 | 587/590 | 0.01 | 0.36 |
| 191 | | | | | |
| Seq ID | VSRTPHTDF | AAV9 | 587/590 | 0.53 | −0.96 |
| 192 | | | | | |
| Seq ID | MQMKGQQPP | AAV9 | 587/590 | −1.49 | −1.26 |
| 193 | | | | | |
| Seq ID | HQSGQVPNI | AAV9 | 587/590 | 0.30 | 0.28 |
| 194 | | | | | |
| Seq ID | QLTGVASGH | AAV9 | 587/590 | −1.18 | −1.50 |
| 195 | | | | | |
| Seq ID | PEQSPQMMF | AAV9 | 587/590 | −0.70 | −0.42 |
| 196 | | | | | |
| Seq ID | NTTPQASLM | AAV9 | 587/590 | −0.21 | 0.00 |
| 197 | | | | | |
| Seq ID | DEFGAYMPI | AAV9 | 587/590 | −3.39 | −1.18 |
| 198 | | | | | |
| Seq ID | PKLIQQVNT | AAV9 | 587/590 | −2.03 | −2.30 |
| 199 | | | | | |
| Seq ID | KPGPAQVQW | AAV9 | 587/590 | −1.29 | −0.22 |
| 200 | | | | | |
| Seq ID | PLKDVQLSH | AAV9 | 587/590 | −0.69 | −0.83 |
| 201 | | | | | |
| Seq ID | KDPFYRSEN | AAV9 | 587/590 | −3.59 | −3.66 |
| 202 | | | | | |
| Seq ID | SQPGNQLKM | AAV9 | 587/590 | −2.00 | −1.24 |
| 203 | | | | | |
| Seq ID | MQNTMRLQD | AAV9 | 587/590 | −0.58 | −0.10 |
| 204 | | | | | |
| Seq ID | ILVKNKDEI | AAV9 | 587/590 | 0.94 | −0.32 |
| 205 | | | | | |
| Seq ID | PVHGQLQGQ | AAV9 | 587/590 | −0.73 | −0.89 |
| 206 | | | | | |
| Seq ID | PTLPGVGMR | AAV9 | 587/590 | −2.09 | #N/A |
| 207 | | | | | |
| Seq ID | PPQAAQITT | AAV9 | 587/590 | −0.06 | −0.12 |
| 208 | | | | | |
| Seq ID | TKVQQQDSW | AAV9 | 587/590 | 0.44 | −0.27 |
| 209 | | | | | |
| Seq ID | IMTSHTVAV | AAV9 | 587/590 | 0.26 | −1.11 |
| 210 | | | | | |
| Seq ID | HPHMAGQQM | AAV9 | 587/590 | −1.53 | 0.17 |
| 211 | | | | | |
| Seq ID | PVQTQHAVV | AAV9 | 587/590 | 0.31 | 0.31 |
| 212 | | | | | |
| Seq ID | QIGHKQEYN | AAV9 | 587/590 | −0.98 | −1.51 |
| 213 | | | | | |
| Seq ID | HEQQIRRDT | AAV9 | 587/590 | −2.03 | −2.32 |
| 214 | | | | | |
| Seq ID | DRFKTVQNE | AAV9 | 587/590 | −2.23 | −2.77 |
| 215 | | | | | |
| Seq ID | AAFQQTDIT | AAV9 | 587/590 | 1.26 | −0.75 |
| 216 | | | | | |
| Seq ID | ERSAVVMIA | AAV9 | 587/590 | 0.39 | 0.33 |
| 217 | | | | | |
| Seq ID | QYIQGRQPI | AAV9 | 587/590 | −2.24 | −1.77 |
| 218 | | | | | |
| Seq ID | LREHAYPDV | AAV9 | 587/590 | −0.86 | −0.90 |
| 219 | | | | | |
| Seq ID | KLMDQGVTA | AAV9 | 587/590 | −0.61 | −1.28 |
| 220 | | | | | |
| Seq ID | TSQKQATHT | AAV9 | 587/590 | −0.35 | −0.90 |
| 221 | | | | | |
| Seq ID | VPNNKNPQV | AAV9 | 587/590 | −0.44 | −0.79 |
| 222 | | | | | |
| Seq ID | NVVIVMIFM | AAV9 | 587/590 | −0.51 | 0.32 |
| 223 | | | | | |
| Seq ID | AISYRGTWS | AAV2 | R588A/589 | −1.37 | −2.84 |
| 224 | TAESGAA | | | | |
| Seq ID | NTTSQTGNV | AAV9 | 587/590 | 0.51 | −1.35 |
| 225 | | | | | |
| Seq ID | PQESQPENE | AAV9 | 587/590 | #N/A | −2.16 |
| 226 | | | | | |
| Seq ID | TMGSRQTPI | AAV9 | 587/590 | −3.10 | −3.50 |
| 227 | | | | | |
| Seq ID | TQVVSGQEI | AAV9 | 587/590 | 1.39 | 1.03 |
| 228 | | | | | |
| Seq ID | PEELKSKQT | AAV9 | 587/590 | −2.34 | −1.89 |
| 229 | | | | | |
| Seq ID | INKYSQYTT | AAV9 | 587/590 | −0.01 | −1.03 |
| 230 | | | | | |
| Seq ID | QPTQGTLRA | AAV9 | 587/590 | 2.80 | 3.07 |
| 231 | | | | | |
| Seq ID | NQVHTKDDS | AAV9 | 587/590 | −0.56 | −1.57 |
| 232 | | | | | |
| Seq ID | YITQIHEDD | AAV9 | 587/590 | −2.68 | −1.10 |
| 233 | | | | | |
| Seq ID | HIGGSGNTM | AAV9 | 587/590 | 0.84 | 0.27 |
| 234 | | | | | |
| Seq ID | HQNHPEHAV | AAV9 | 587/590 | −1.68 | −1.64 |
| 235 | | | | | |
| Seq ID | LKGSVQQAP | AAV9 | 587/590 | −0.09 | −1.60 |
| 236 | | | | | |
| Seq ID | PLIPRQEVI | AAV9 | 587/590 | −0.80 | −0.66 |
| 237 | | | | | |
| Seq ID | TLVTQSDSS | AAV9 | 587/590 | 0.83 | 0.54 |
| 238 | | | | | |
| Seq ID | LKIDVTKIT | AAV9 | 587/590 | −1.86 | −1.25 |
| 239 | | | | | |
| Seq ID | PSHVGFTSV | AAV9 | 587/590 | −1.26 | −2.27 |
| 240 | | | | | |
| Seq ID | LYRDQGNIT | AAV9 | 587/590 | −2.32 | −1.35 |
| 241 | | | | | |
| Seq ID | SVKQMDATH | AAV9 | 587/590 | 0.70 | 0.97 |
| 242 | | | | | |
| Seq ID | QFDRVAQDK | AAV9 | 587/590 | −1.30 | −1.20 |
| 243 | | | | | |
| Seq ID | YPTGTPPAT | AAV9 | 587/590 | 1.70 | 0.93 |
| 244 | | | | | |
| Seq ID | DRVQSTSPH | AAV9 | 587/590 | 0.30 | −0.51 |
| 245 | | | | | |
| Seq ID | KTQKPYQED | AAV9 | 587/590 | −0.27 | −2.79 |
| 246 | | | | | |
| Seq ID | SQVAEQRGI | AAV9 | 587/590 | −0.89 | −0.68 |
| 247 | | | | | |
| Seq ID | SESMHGSVS | AAV9 | 587/590 | 0.43 | 0.79 |
| 248 | | | | | |
| Seq ID | TITDQQSHG | AAV9 | 587/590 | 0.27 | −0.12 |
| 249 | | | | | |
| Seq ID | TRHVVTGGY | AAV9 | 587/590 | −3.07 | −3.34 |
| 250 | | | | | |
| Seq ID | QQNMKLQVY | AAV9 | 587/590 | −2.87 | −2.20 |
| 251 | | | | | |
| Seq ID | SKFTYYDPW | AAV9 | 587/590 | −0.59 | 0.30 |
| 252 | | | | | |
| Seq ID | MAVQHTTNQ | AAV9 | 587/590 | −1.44 | −0.55 |
| 253 | | | | | |
| Seq ID | SSMQGASGV | AAV9 | 587/590 | 0.70 | −0.14 |
| 254 | | | | | |
| Seq ID | MKYSTQTQS | AAV9 | 587/590 | −1.41 | −1.55 |
| 255 | | | | | |
| Seq ID | IKQPQSFAQ | AAV9 | 587/590 | 1.05 | −2.51 |
| 256 | | | | | |
| Seq ID | TPVAQANKN | AAV9 | 587/590 | −0.25 | −1.05 |
| 257 | | | | | |
| Seq ID | QDVMTSPMQ | AAV9 | 587/590 | −0.15 | −2.30 |
| 258 | | | | | |
| Seq ID | PQVLGQDEA | AAV9 | 587/590 | −2.64 | −2.34 |
| 259 | | | | | |
| Seq ID | TDTVDTGSM | AAV9 | 587/590 | −2.48 | −1.49 |
| 260 | | | | | |
| Seq ID | AQEGARQKL | AAV9 | 588/589 | −0.87 | −4.98 |
| 261 | HELQEKA | | | | |
| Seq ID | ATSEYGRAY | AAV9 | 587/590 | −0.83 | −2.29 |
| 262 | | | | | |
| Seq ID | LKGPQQQAI | AAV9 | 587/590 | −0.09 | −0.98 |
| 263 | | | | | |
| Seq ID | VFREAGNVH | AAV9 | 587/590 | 0.85 | 0.63 |
| 264 | | | | | |
| Seq ID | PKQVSDIMF | AAV9 | 587/590 | −1.39 | −0.95 |
| 265 | | | | | |
| Seq ID | HMMDGGVPY | AAV9 | 587/590 | 0.03 | −0.07 |
| 266 | | | | | |
| Seq ID | IRAASTGHM | AAV9 | 587/590 | −0.89 | −1.74 |
| 267 | | | | | |
| Seq ID | PDGYHVEMF | AAV9 | 587/590 | −0.38 | −0.80 |
| 268 | | | | | |
| Seq ID | QAQSNNIYF | AAV9 | 587/590 | −4.37 | −1.17 |
| 269 | | | | | |
| Seq ID | TMNGQSKNF | AAV9 | 587/590 | 0.38 | 0.14 |
| 270 | | | | | |
| Seq ID | HQARREVEY | AAV9 | 587/590 | −0.71 | −1.18 |
| 271 | | | | | |
| Seq ID | GDQGRVVSH | AAV9 | 587/590 | −2.75 | −0.57 |
| 272 | | | | | |
| Seq ID | ACCPLAEES | AAV2 | R588A/589 | −1.11 | −1.39 |
| 273 | DNVDSAA | | | | |
| Seq ID | QQNQSIFNI | AAV9 | 587/590 | −0.60 | −0.67 |
| 274 | | | | | |
| Seq ID | TSDAVKTDQ | AAV9 | 587/590 | −3.22 | −2.36 |
| 275 | | | | | |
| Seq ID | TQAPDVLPV | AAV9 | 587/590 | −0.46 | −0.69 |
| 276 | | | | | |
| Seq ID | TTDTAKATY | AAV9 | 587/590 | −2.59 | −0.81 |
| 277 | | | | | |
| Seq ID | IDEHSEHYV | AAV9 | 587/590 | −2.39 | −1.23 |
| 278 | | | | | |
| Seq ID | PINSRIVDP | AAV9 | 587/590 | −0.60 | −1.81 |
| 279 | | | | | |
| Seq ID | PDQRGHMYA | AAV9 | 587/590 | −2.38 | −1.05 |
| 280 | | | | | |
| Seq ID | FLMSHSEVQ | AAV9 | 587/590 | −1.71 | −2.37 |
| 281 | | | | | |
| Seq ID | FIQQSNERN | AAV9 | 587/590 | −0.83 | −0.95 |
| 282 | | | | | |
| Seq ID | NPTRDAKPP | AAV9 | 587/590 | 0.40 | 0.87 |
| 283 | | | | | |
| Seq ID | RWGRAQEDE | AAV9 | 587/590 | −1.26 | −2.32 |
| 284 | | | | | |
| Seq ID | PRNMQPMTD | AAV9 | 587/590 | 0.75 | 0.56 |
| 285 | | | | | |
| Seq ID | MVSQREINQ | AAV9 | 587/590 | 0.15 | −0.67 |
| 286 | | | | | |
| Seq ID | MSSSTRMAV | AAV9 | 587/590 | −1.23 | −2.62 |
| 287 | | | | | |
| Seq ID | PLNQHQKIS | AAV9 | 587/590 | −2.00 | −1.27 |
| 288 | | | | | |
| Seq ID | DNASHVHMQ | AAV9 | 587/590 | −0.62 | −0.73 |
| 289 | | | | | |
| Seq ID | DMKRVDYMQ | AAV9 | 587/590 | −4.04 | −2.77 |
| 290 | | | | | |
| Seq ID | NHLVDRTTQ | AAV9 | 587/590 | 2.23 | 0.06 |
| 291 | | | | | |
| Seq ID | VIQASSITA | AAV9 | 587/590 | 0.24 | −0.06 |
| 292 | | | | | |
| Seq ID | AQAMTNKNK | AAV9 | 587/590 | −1.44 | 0.00 |
| 293 | | | | | |
| Seq ID | INFKQEVHQ | AAV9 | 587/590 | 1.07 | −0.11 |
| 294 | | | | | |
| Seq ID | AYAQLPHEK | AAV9 | 587/590 | −0.47 | −1.31 |
| 295 | | | | | |
| Seq ID | AVTIQNWCK | AAV2 | R588A/589 | −0.91 | 0.46 |
| 296 | RGRKQCA | | | | |
| Seq ID | NPQNSGMAM | AAV9 | 587/590 | 0.46 | −0.23 |
| 297 | | | | | |
| Seq ID | SRDRDTAYA | AAV9 | 587/590 | 0.39 | 0.51 |
| 298 | | | | | |
| Seq ID | PHNSPGMTR | AAV9 | 587/590 | 0.27 | −1.22 |
| 299 | | | | | |
| Seq ID | KMMPSQMQD | AAV9 | 587/590 | −0.35 | −0.82 |
| 300 | | | | | |
| Seq ID | NHAPVHERM | AAV9 | 587/590 | #N/A | −1.17 |
| 301 | | | | | |
| Seq ID | RALKQIDAD | AAV9 | 587/590 | −0.36 | −0.43 |
| 302 | | | | | |
| Seq ID | QRVEGFIQD | AAV9 | 587/590 | 1.00 | 1.09 |
| 303 | | | | | |
| Seq ID | AKTDSCQGD | AAV2 | R588A/589 | −2.21 | −2.40 |
| 304 | SGGPLVA | | | | |
| Seq ID | TSASVQSAM | AAV9 | 587/590 | 1.43 | 1.13 |
| 305 | | | | | |
| Seq ID | NVTTHAPTI | AAV9 | 587/590 | 1.45 | 0.02 |
| 306 | | | | | |
| Seq ID | RVQTPDHDH | AAV9 | 587/590 | −1.30 | −1.34 |
| 307 | | | | | |
| Seq ID | QVTKEFASF | AAV9 | 587/590 | −0.98 | −2.05 |
| 308 | | | | | |
| Seq ID | ASVTPQYLY | AAV9 | 587/590 | −1.59 | 1.04 |
| 309 | | | | | |
| Seq ID | VTQKTDNTL | AAV9 | 587/590 | 1.52 | 2.31 |
| 310 | | | | | |
| Seq ID | VKMGIQAIA | AAV9 | 587/590 | −0.85 | −0.33 |
| 311 | | | | | |
| Seq ID | PLDNYLASM | AAV9 | 587/590 | −1.42 | −2.13 |
| 312 | | | | | |
| Seq ID | DENGGVPRI | AAV9 | 587/590 | −3.69 | −0.10 |
| 313 | | | | | |
| Seq ID | HWQQPSIDG | AAV9 | 587/590 | −2.26 | −1.22 |
| 314 | | | | | |
| Seq ID | TPSQGSHGS | AAV9 | 587/590 | 0.41 | −1.70 |
| 315 | | | | | |
| Seq ID | HNGMNQVDK | AAV9 | 587/590 | 0.85 | 0.32 |
| 316 | | | | | |
| Seq ID | PHMHNMATP | AAV9 | 587/590 | −2.92 | −1.50 |
| 317 | | | | | |
| Seq ID | IQVRDRTPE | AAV9 | 587/590 | 0.26 | 0.21 |
| 318 | | | | | |
| Seq ID | APDAHTRMP | AAV9 | 587/590 | 1.69 | 0.37 |
| 319 | | | | | |
| Seq ID | GAVSHHQMI | AAV9 | 587/590 | 0.67 | −1.27 |
| 320 | | | | | |
| Seq ID | NVQRTGVGN | AAV9 | 587/590 | −1.52 | −2.72 |
| 321 | | | | | |
| Seq ID | PDMLQGKMY | AAV9 | 587/590 | −1.67 | −2.66 |
| 322 | | | | | |
| Seq ID | NYFASSDQM | AAV9 | 587/590 | 0.13 | −0.10 |
| 323 | | | | | |
| Seq ID | PRQGSLAHM | AAV9 | 587/590 | −2.61 | −3.67 |
| 324 | | | | | |
| Seq ID | SQQVSKQMQ | AAV9 | 587/590 | −3.78 | −3.15 |
| 325 | | | | | |
| Seq ID | KRNQEGNGV | AAV9 | 587/590 | −1.64 | −2.15 |
| 326 | | | | | |
| Seq ID | PMHSSHNDK | AAV9 | 587/590 | −3.60 | −1.68 |
| 327 | | | | | |
| Seq ID | TLQPQHIAK | AAV9 | 587/590 | −1.22 | −2.78 |
| 328 | | | | | |
| Seq ID | SAGVVISVV | AAV9 | 587/590 | −0.69 | −0.28 |
| 329 | | | | | |
| Seq ID | PLVPRNTPQ | AAV9 | 587/590 | −5.22 | −2.68 |
| 330 | | | | | |
| Seq ID | PNQTQMLNK | AAV9 | 587/590 | −1.71 | −2.61 |
| 331 | | | | | |
| Seq ID | PMSSEYRPS | AAV9 | 587/590 | −0.88 | −1.60 |
| 332 | | | | | |
| Seq ID | ESAHSEFKV | AAV9 | 587/590 | 0.04 | −1.84 |
| 333 | | | | | |
| Seq ID | PQRVQEQGT | AAV9 | 587/590 | −2.73 | −1.59 |
| 334 | | | | | |
| Seq ID | MVQQSLKPA | AAV9 | 587/590 | −1.68 | −1.97 |
| 335 | | | | | |
| Seq ID | EQQPAQYHG | AAV9 | 587/590 | 0.07 | 0.05 |
| 336 | | | | | |
| Seq ID | TRNAQTTPL | AAV9 | 587/590 | −1.15 | −1.97 |
| 337 | | | | | |
| Seq ID | PARSGVQIV | AAV9 | 587/590 | −2.79 | −1.01 |
| 338 | | | | | |
| Seq ID | DVFTRQSEG | AAV9 | 587/590 | −1.02 | −0.19 |
| 339 | | | | | |
| Seq ID | SMHTHQGGQ | AAV9 | 587/590 | 0.03 | −0.76 |
| 340 | | | | | |
| Seq ID | VNQNVRMED | AAV9 | 587/590 | −1.54 | −0.88 |
| 341 | | | | | |
| Seq ID | LPQAKMADM | AAV9 | 587/590 | 0.52 | 0.16 |
| 342 | | | | | |
| Seq ID | RMMDNSIHT | AAV9 | 587/590 | −0.76 | −0.90 |
| 343 | | | | | |
| Seq ID | MEATAEHRI | AAV9 | 587/590 | −2.26 | 0.10 |
| 344 | | | | | |
| Seq ID | DARAHTQQV | AAV9 | 587/590 | 0.33 | 0.35 |
| 345 | | | | | |
| Seq ID | IKDMEPQLH | AAV9 | 587/590 | 0.43 | 0.13 |
| 346 | | | | | |
| Seq ID | ALITILGTV | AAV9 | 588/589 | −1.18 | −2.19 |
| 347 | KPNANRA | | | | |
| Seq ID | NHQARTGEF | AAV9 | 587/590 | −0.44 | −1.31 |
| 348 | | | | | |
| Seq ID | PKNHPVSAV | AAV9 | 587/590 | −5.32 | −3.54 |
| 349 | | | | | |
| Seq ID | KFPTEDKYI | AAV9 | 587/590 | −0.67 | −2.91 |
| 350 | | | | | |
| Seq ID | SPVATQKMV | AAV9 | 587/590 | −0.25 | −0.54 |
| 351 | | | | | |
| Seq ID | DLAEHTRFK | AAV9 | 587/590 | 0.28 | 0.21 |
| 352 | | | | | |
| Seq ID | SNTQPRPQQ | AAV9 | 587/590 | 0.33 | −1.71 |
| 353 | | | | | |
| Seq ID | TIDITPRKL | AAV9 | 587/590 | −2.36 | −1.33 |
| 354 | | | | | |
| Seq ID | PAFVDENPP | AAV9 | 587/590 | −0.59 | −0.58 |
| 355 | | | | | |
| Seq ID | VSQETRTTI | AAV9 | 587/590 | 0.60 | 0.72 |
| 356 | | | | | |
| Seq ID | EHTGGQFRL | AAV9 | 587/590 | −0.83 | −0.73 |
| 357 | | | | | |
| Seq ID | MLGKSDQVW | AAV9 | 587/590 | −1.27 | −0.56 |
| 358 | | | | | |
| Seq ID | PQRDMVNMK | AAV9 | 587/590 | −0.83 | −3.03 |
| 359 | | | | | |
| Seq ID | NSRPGAAMA | AAV9 | 587/590 | −1.20 | −2.59 |
| 360 | | | | | |
| Seq ID | TQQAPHNAY | AAV9 | 587/590 | 0.03 | −1.36 |
| 361 | | | | | |
| Seq ID | PNITGGAIM | AAV9 | 587/590 | 0.59 | 0.22 |
| 362 | | | | | |
| Seq ID | PDQKMMFKL | AAV9 | 587/590 | −4.98 | −2.35 |
| 363 | | | | | |
| Seq ID | LKHATVEST | AAV9 | 587/590 | −1.22 | −1.06 |
| 364 | | | | | |
| Seq ID | QLSRDTRQD | AAV9 | 587/590 | 1.05 | 1.37 |
| 365 | | | | | |
| Seq ID | PDSKPHAYH | AAV9 | 587/590 | −1.34 | −1.47 |
| 366 | | | | | |
| Seq ID | MQPPAQQRT | AAV9 | 587/590 | 0.47 | −0.11 |
| 367 | | | | | |
| Seq ID | VTIFDMFDQ | AAV9 | 587/590 | #N/A | −0.80 |
| 368 | | | | | |
| Seq ID | IMAYTGQPV | AAV9 | 587/590 | −0.90 | −1.41 |
| 369 | | | | | |
| Seq ID | RTEYEMSTR | AAV9 | 587/590 | −0.38 | −1.46 |
| 370 | | | | | |
| Seq ID | QAQRKTEFI | AAV9 | 587/590 | −1.34 | −1.11 |
| 371 | | | | | |
| Seq ID | QGNETKPNS | AAV9 | 587/590 | −2.22 | −1.50 |
| 372 | | | | | |
| Seq ID | RMASQGQDM | AAV9 | 587/590 | −5.99 | −1.90 |
| 373 | | | | | |
| Seq ID | PKDGVPQMQ | AAV9 | 587/590 | −3.70 | −1.15 |
| 374 | | | | | |
| Seq ID | TIAPAIANL | AAV9 | 587/590 | 1.08 | −0.35 |
| 375 | | | | | |
| Seq ID | RTHQSSPDS | AAV9 | 587/590 | −1.95 | −2.10 |
| 376 | | | | | |
| Seq ID | PTTDMQSNI | AAV9 | 587/590 | −0.95 | −0.26 |
| 377 | | | | | |
| Seq ID | HATSQKYET | AAV9 | 587/590 | −0.96 | −1.84 |
| 378 | | | | | |
| Seq ID | ALGQARSDA | AAV9 | 588/589 | 0.19 | −0.56 |
| 379 | | | | | |
| Seq ID | PIATAYKTG | AAV9 | 587/590 | 0.21 | −0.23 |
| 380 | | | | | |
| Seq ID | HFKPTQDTS | AAV9 | 587/590 | −1.41 | −2.91 |
| 381 | | | | | |
| Seq ID | RPNTKAMQE | AAV9 | 587/590 | −2.12 | 0.96 |
| 382 | | | | | |
| Seq ID | NMPQGRVAM | AAV9 | 587/590 | −1.17 | −3.97 |
| 383 | | | | | |
| Seq ID | KTINTPNIL | AAV9 | 587/590 | −1.59 | −3.06 |
| 384 | | | | | |
| Seq ID | SRIQHDGAQ | AAV9 | 587/590 | 0.50 | 0.06 |
| 385 | | | | | |
| Seq ID | HPPMPNNTK | AAV9 | 587/590 | −0.56 | −0.59 |
| 386 | | | | | |
| Seq ID | RIMPQMQHD | AAV9 | 587/590 | −0.07 | −0.27 |
| 387 | | | | | |
| Seq ID | AVILELDKT | AAV2 | R588A/589 | −3.67 | −3.26 |
| 388 | VEHLPTA | | | | |
| Seq ID | HSTARSGEF | AAV9 | 587/590 | −1.15 | −1.04 |
| 389 | | | | | |
| Seq ID | SQPQHMNVS | AAV9 | 587/590 | 0.80 | 0.31 |
| 390 | | | | | |
| Seq ID | PGGSYSHVV | AAV9 | 587/590 | 2.13 | −0.54 |
| 391 | | | | | |
| Seq ID | TVTHAHPTT | AAV9 | 587/590 | 1.43 | 0.52 |
| 392 | | | | | |
| Seq ID | IVATAPRVA | AAV9 | 587/590 | −2.06 | −2.67 |
| 393 | | | | | |
| Seq ID | VTRTSVAIE | AAV9 | 587/590 | −2.22 | −1.79 |
| 394 | | | | | |
| Seq ID | MIVPTPMNQ | AAV9 | 587/590 | −1.10 | −1.37 |
| 395 | | | | | |
| Seq ID | IQVMHSTTH | AAV9 | 587/590 | 0.81 | 0.33 |
| 396 | | | | | |
| Seq ID | QEPTYKNEV | AAV9 | 587/590 | −2.47 | −2.48 |
| 397 | | | | | |
| Seq ID | VVPTMNNTR | AAV9 | 587/590 | −1.63 | −2.01 |
| 398 | | | | | |
| Seq ID | RETTVYPLQ | AAV9 | 587/590 | 0.22 | 0.29 |
| 399 | | | | | |
| Seq ID | RITTESTIH | AAV9 | 587/590 | −0.28 | −0.65 |
| 400 | | | | | |
| Seq ID | AEEVAARRA | AAV2 | R588A/589 | −1.12 | −1.93 |
| 401 | RVVWCAA | | | | |
| Seq ID | DPVRRKMQV | AAV9 | 587/590 | −0.63 | −0.45 |
| 402 | | | | | |
| Seq ID | PYSSKTQDP | AAV9 | 587/590 | −0.96 | −0.57 |
| 403 | | | | | |
| Seq ID | WPETKQEKS | AAV9 | 587/590 | −1.24 | −1.61 |
| 404 | | | | | |
| Seq ID | PNTQHLKAE | AAV9 | 587/590 | −2.49 | −3.80 |
| 405 | | | | | |
| Seq ID | WQSTGKQSI | AAV9 | 587/590 | −2.14 | −1.88 |
| 406 | | | | | |
| Seq ID | KASTDPHSR | AAV9 | 587/590 | 1.38 | −1.38 |
| 407 | | | | | |
| Seq ID | KPTGTIQGY | AAV9 | 587/590 | −0.37 | −0.14 |
| 408 | | | | | |
| Seq ID | HLGTAIVQP | AAV9 | 587/590 | 0.34 | −0.23 |
| 409 | | | | | |
| Seq ID | PKFSHATSE | AAV9 | 587/590 | −1.22 | −2.68 |
| 410 | | | | | |
| Seq ID | IRDGIQMHV | AAV9 | 587/590 | 3.45 | 3.59 |
| 411 | | | | | |
| Seq ID | TTNQERLQS | AAV9 | 587/590 | 0.18 | −0.42 |
| 412 | | | | | |
| Seq ID | QIKVPPLDQ | AAV9 | 587/590 | −2.18 | −2.89 |
| 413 | | | | | |
| Seq ID | LHHERGPSA | AAV9 | 587/590 | −0.76 | −0.93 |
| 414 | | | | | |
| Seq ID | KPLGQAQVQ | AAV9 | 587/590 | −0.82 | −1.08 |
| 415 | | | | | |
| Seq ID | TPASATRDS | AAV9 | 587/590 | −1.63 | −1.17 |
| 416 | | | | | |
| Seq ID | PQQTQVSGQ | AAV9 | 587/590 | −5.19 | −1.77 |
| 417 | | | | | |
| Seq ID | ERSSQSIIQ | AAV9 | 587/590 | −2.96 | −1.78 |
| 418 | | | | | |
| Seq ID | YHEWRHIPT | AAV9 | 587/590 | −1.07 | −1.80 |
| 419 | | | | | |
| Seq ID | PLQASGTAY | AAV9 | 587/590 | 0.01 | 0.03 |
| 420 | | | | | |
| Seq ID | TNNGVRSII | AAV9 | 587/590 | −0.46 | −1.04 |
| 421 | | | | | |
| Seq ID | VQPEITPGQ | AAV9 | 587/590 | #N/A | −4.83 |
| 422 | | | | | |
| Seq ID | QDDHNKVQQ | AAV9 | 587/590 | −1.22 | −1.17 |
| 423 | | | | | |
| Seq ID | PIVKQNTDT | AAV9 | 587/590 | 0.81 | −0.44 |
| 424 | | | | | |
| Seq ID | HTLPRLQEE | AAV9 | 587/590 | −1.63 | −2.36 |
| 425 | | | | | |
| Seq ID | QPTQGTVRA | AAV9 | 587/590 | 3.48 | 3.38 |
| 426 | | | | | |
| Seq ID | TRPQGPLSP | AAV9 | 587/590 | 0.05 | −1.34 |
| 427 | | | | | |
| Seq ID | TYAQPHQNQ | AAV9 | 587/590 | −2.42 | −1.68 |
| 428 | | | | | |
| Seq ID | IQMEQKQQV | AAV9 | 587/590 | 0.03 | −0.69 |
| 429 | | | | | |
| Seq ID | TQTPPQDAS | AAV9 | 587/590 | 0.02 | 0.59 |
| 430 | | | | | |
| Seq ID | SGAQEKWEM | AAV9 | 587/590 | −0.10 | −0.11 |
| 431 | | | | | |
| Seq ID | ASDTEKEDR | AAV9 | 588/589 | −2.66 | −2.02 |
| 432 | SPSAIFA | | | | |
| Seq ID | PNYQTKMSF | AAV9 | 587/590 | −3.32 | −3.07 |
| 433 | | | | | |
| Seq ID | RPGAAVAPA | AAV9 | 587/590 | −2.06 | −3.65 |
| 434 | | | | | |
| Seq ID | LAGSHVSTQ | AAV9 | 587/590 | 1.71 | 0.92 |
| 435 | | | | | |
| Seq ID | LPPGNQTNN | AAV9 | 587/590 | 0.39 | −1.21 |
| 436 | | | | | |
| Seq ID | NHQHDIEWF | AAV9 | 587/590 | −4.07 | −1.21 |
| 437 | | | | | |
| Seq ID | ISEDGSNRK | AAV9 | 587/590 | −2.91 | −0.11 |
| 438 | | | | | |
| Seq ID | SMGIHPTNA | AAV9 | 587/590 | 1.25 | 0.46 |
| 439 | | | | | |
| Seq ID | TPLAMASQK | AAV9 | 587/590 | 0.91 | 1.07 |
| 440 | | | | | |
| Seq ID | NVTSNTIQF | AAV9 | 587/590 | 0.05 | 0.97 |
| 441 | | | | | |
| Seq ID | NYNTVPQPK | AAV9 | 587/590 | −0.29 | −1.65 |
| 442 | | | | | |
| Seq ID | PRVLQGVQV | AAV9 | 587/590 | −2.14 | −2.14 |
| 443 | | | | | |
| Seq ID | TGTRTSAQQ | AAV9 | 587/590 | 0.17 | 0.05 |
| 444 | | | | | |
| Seq ID | NHHRPDVFV | AAV9 | 587/590 | −2.04 | −1.43 |
| 445 | | | | | |
| Seq ID | PGPNERQIM | AAV9 | 587/590 | −1.53 | −2.08 |
| 446 | | | | | |
| Seq ID | PHDRLPQEV | AAV9 | 587/590 | 0.58 | −1.37 |
| 447 | | | | | |
| Seq ID | PNAGVMDEE | AAV9 | 587/590 | −1.40 | −1.81 |
| 448 | | | | | |
| Seq ID | TAHYRTAET | AAV9 | 587/590 | 0.17 | −0.84 |
| 449 | | | | | |
| Seq ID | RPQSSATTH | AAV9 | 587/590 | −2.72 | −2.67 |
| 450 | | | | | |
| Seq ID | IAHFQPKDT | AAV9 | 587/590 | −1.46 | −1.49 |
| 451 | | | | | |
| Seq ID | FAHPEKGFF | AAV9 | 587/590 | −0.52 | −1.00 |
| 452 | | | | | |
| Seq ID | QAYAKTAQL | AAV9 | 587/590 | −0.42 | −1.69 |
| 453 | | | | | |
| Seq ID | TTSVSVMPK | AAV9 | 587/590 | −1.54 | −1.30 |
| 454 | | | | | |
| Seq ID | ASVDTEQRR | AAV9 | 587/590 | 1.29 | 0.74 |
| 455 | | | | | |
| Seq ID | WHEAIIVTG | AAV9 | 587/590 | 0.11 | −0.93 |
| 456 | | | | | |
| Seq ID | VDNDIEWKF | AAV9 | 587/590 | #N/A | −4.65 |
| 457 | | | | | |
| Seq ID | NRVGSQAPT | AAV9 | 587/590 | −3.57 | −2.84 |
| 458 | | | | | |
| Seq ID | AAVIVANPR | AAV2 | R588A/589 | −1.36 | −2.73 |
| 459 | EEIIVKA | | | | |
| Seq ID | PTTPQMVSL | AAV9 | 587/590 | −1.27 | −1.58 |
| 460 | | | | | |
| Seq ID | ALKNFAKYF | AAV2 | R588A/589 | −2.60 | −2.06 |
| 461 | LHQSHEA | | | | |
| Seq ID | KYVNVEVQT | AAV9 | 587/590 | −1.37 | 0.72 |
| 462 | | | | | |
| Seq ID | LLDAGTSGK | AAV9 | 587/590 | 1.40 | 0.45 |
| 463 | | | | | |
| Seq ID | NAPAQQKMM | AAV9 | 587/590 | −1.02 | −1.77 |
| 464 | | | | | |
| Seq ID | VDDHAHRQQ | AAV9 | 587/590 | 0.60 | −2.37 |
| 465 | | | | | |
| Seq ID | PVKLANPTN | AAV9 | 587/590 | 0.27 | −2.00 |
| 466 | | | | | |
| Seq ID | HALVHQGAV | AAV9 | 587/590 | 0.55 | 0.25 |
| 467 | | | | | |
| Seq ID | VFQKSHSTE | AAV9 | 587/590 | −0.43 | −0.59 |
| 468 | | | | | |
| Seq ID | FKQRDSNET | AAV9 | 587/590 | 1.24 | −0.66 |
| 469 | | | | | |
| Seq ID | EQMSQRVGF | AAV9 | 587/590 | #N/A | −3.13 |
| 470 | | | | | |
| Seq ID | RQAGQFPDS | AAV9 | 587/590 | −3.22 | −2.62 |
| 471 | | | | | |
| Seq ID | ESQANAHNA | AAV9 | 587/590 | −1.28 | −4.83 |
| 472 | | | | | |
| Seq ID | PPDSKSLNQ | AAV9 | 587/590 | −0.51 | −0.19 |
| 473 | | | | | |
| Seq ID | PEQPSQSQY | AAV9 | 587/590 | −0.21 | −1.54 |
| 474 | | | | | |
| Seq ID | DGIHTSVQM | AAV9 | 587/590 | 0.82 | 1.20 |
| 475 | | | | | |
| Seq ID | RFDLQEGQS | AAV9 | 587/590 | 0.88 | 0.07 |
| 476 | | | | | |
| Seq ID | DRDRAMMNI | AAV9 | 587/590 | −0.43 | −1.53 |
| 477 | | | | | |
| Seq ID | YPPEAQMEM | AAV9 | 587/590 | −2.42 | −2.77 |
| 478 | | | | | |
| Seq ID | TAVTTHNGS | AAV9 | 587/590 | −0.73 | −2.53 |
| 479 | | | | | |
| Seq ID | EWALDQYGA | AAV9 | 587/590 | #N/A | −2.16 |
| 480 | | | | | |
| Seq ID | ITQSNERMT | AAV9 | 587/590 | −0.64 | −2.82 |
| 481 | | | | | |
| Seq ID | NITHQTMLQ | AAV9 | 587/590 | 0.68 | 1.49 |
| 482 | | | | | |
| Seq ID | MMNGSQQFQ | AAV9 | 587/590 | −0.65 | −0.37 |
| 483 | | | | | |
| Seq ID | QQTTGRVQQ | AAV9 | 587/590 | −1.50 | −3.52 |
| 484 | | | | | |
| Seq ID | LVNPDPMQY | AAV9 | 587/590 | 0.18 | −0.64 |
| 485 | | | | | |
| Seq ID | HTHDMDMTP | AAV9 | 587/590 | −2.20 | −0.24 |
| 486 | | | | | |
| Seq ID | PQTKNTMDD | AAV9 | 587/590 | −0.07 | −0.32 |
| 487 | | | | | |
| Seq ID | DPERQTRPY | AAV9 | 587/590 | −3.11 | −3.81 |
| 488 | | | | | |
| Seq ID | TKARSDPLE | AAV9 | 587/590 | −2.29 | −1.89 |
| 489 | | | | | |
| Seq ID | RVNDQPYQM | AAV9 | 587/590 | −0.21 | −0.36 |
| 490 | | | | | |
| Seq ID | RPSNVDSAT | AAV9 | 587/590 | 0.90 | 0.64 |
| 491 | | | | | |
| Seq ID | RMSNDQKSF | AAV9 | 587/590 | −1.26 | −1.91 |
| 492 | | | | | |
| Seq ID | SMTSAFDRI | AAV9 | 587/590 | −0.80 | −1.65 |
| 493 | | | | | |
| Seq ID | IHDKQMNNQ | AAV9 | 587/590 | −0.93 | −0.10 |
| 494 | | | | | |
| Seq ID | NEYFPYEDL | AAV9 | 587/590 | −4.76 | −2.77 |
| 495 | | | | | |
| Seq ID | SIVPTANQD | AAV9 | 587/590 | −2.01 | −1.82 |
| 496 | | | | | |
| Seq ID | IFEQAHTNK | AAV9 | 587/590 | −3.74 | −1.91 |
| 497 | | | | | |
| Seq ID | AGDTRSLQV | AAV9 | 587/590 | 0.86 | 0.07 |
| 498 | | | | | |
| Seq ID | LQNQTKPVI | AAV9 | 587/590 | −0.73 | −1.53 |
| 499 | | | | | |
| Seq ID | QGDIVQHHF | AAV9 | 587/590 | −0.09 | −0.90 |
| 500 | | | | | |
| Seq ID | EIQTYKSIV | AAV9 | 587/590 | −0.58 | −2.00 |
| 501 | | | | | |
| Seq ID | KAPSIVQGT | AAV9 | 587/590 | −3.59 | −2.57 |
| 502 | | | | | |
| Seq ID | QTPQHGTVL | AAV9 | 587/590 | −0.22 | −1.00 |
| 503 | | | | | |
| Seq ID | KLMTMYDGQ | AAV9 | 587/590 | −3.23 | −1.30 |
| 504 | | | | | |
| Seq ID | LWTNQPSAW | AAV9 | 587/590 | −0.95 | −1.59 |
| 505 | | | | | |
| Seq ID | HSDQMRQMF | AAV9 | 587/590 | 0.32 | −0.14 |
| 506 | | | | | |
| Seq ID | EWQNEATPA | AAV9 | 587/590 | −2.42 | −2.88 |
| 507 | | | | | |
| Seq ID | RPERDPMHI | AAV9 | 587/590 | −0.21 | 0.31 |
| 508 | | | | | |
| Seq ID | AVWQAANTH | AAV9 | 587/590 | −0.35 | −2.32 |
| 509 | | | | | |
| Seq ID | RTATTQGGF | AAV9 | 587/590 | −2.09 | −2.27 |
| 510 | | | | | |
| Seq ID | PTNLVEAGE | AAV9 | 587/590 | −0.19 | −2.10 |
| 511 | | | | | |
| Seq ID | TRMNQTPLQ | AAV9 | 587/590 | −5.17 | −2.79 |
| 512 | | | | | |
| Seq ID | PSFTQDRDR | AAV9 | 587/590 | −1.63 | −2.28 |
| 513 | | | | | |
| Seq ID | SSAAHTVTY | AAV9 | 587/590 | 1.41 | 1.22 |
| 514 | | | | | |
| Seq ID | SHPQPFMKW | AAV9 | 587/590 | −1.14 | −1.38 |
| 515 | | | | | |
| Seq ID | QQGVREAFI | AAV9 | 587/590 | 0.60 | −0.51 |
| 516 | | | | | |
| Seq ID | AQSPLVSSS | AAV2 | R588A/589 | −0.95 | −1.67 |
| 517 | DSPPRPA | | | | |
| Seq ID | DSAVRQNPF | AAV9 | 587/590 | −0.67 | −0.85 |
| 518 | | | | | |
| Seq ID | KSNSMQEAH | AAV9 | 587/590 | −1.01 | −1.15 |
| 519 | | | | | |
| Seq ID | PRQLSSIPS | AAV9 | 587/590 | −2.70 | −3.58 |
| 520 | | | | | |
| Seq ID | DPSAQKQNY | AAV9 | 587/590 | 1.79 | 1.41 |
| 521 | | | | | |
| Seq ID | TGAFDDHDI | AAV9 | 587/590 | −1.29 | −0.96 |
| 522 | | | | | |
| Seq ID | NGTHLVTRA | AAV9 | 587/590 | −0.28 | 0.16 |
| 523 | | | | | |
| Seq ID | TGGKVQLGS | AAV9 | 587/590 | −0.65 | −2.50 |
| 524 | | | | | |
| Seq ID | KQLKTMNDF | AAV9 | 587/590 | −5.41 | −1.97 |
| 525 | | | | | |
| Seq ID | PHDTRIAVD | AAV9 | 587/590 | −0.32 | −0.33 |
| 526 | | | | | |
| Seq ID | ERHIAHTWT | AAV9 | 587/590 | 1.55 | 0.16 |
| 527 | | | | | |
| Seq ID | YRPHPQQHP | AAV9 | 587/590 | #N/A | −2.38 |
| 528 | | | | | |
| Seq ID | YNLPQPKEI | AAV9 | 587/590 | −0.58 | −1.41 |
| 529 | | | | | |
| Seq ID | DTQQQPPYK | AAV9 | 587/590 | 0.49 | 0.34 |
| 530 | | | | | |
| Seq ID | QSNQVKQMD | AAV9 | 587/590 | 0.99 | −1.67 |
| 531 | | | | | |
| Seq ID | IEVKQQQNG | AAV9 | 587/590 | 0.05 | 0.15 |
| 532 | | | | | |
| Seq ID | DQTHNIMQK | AAV9 | 587/590 | 0.76 | 0.55 |
| 533 | | | | | |
| Seq ID | VQEEGWLYA | AAV9 | 587/590 | −5.17 | −1.59 |
| 534 | | | | | |
| Seq ID | KQMVAQMSM | AAV9 | 587/590 | −0.21 | −2.49 |
| 535 | | | | | |
| Seq ID | SINGQQHDY | AAV9 | 587/590 | −2.97 | −3.33 |
| 536 | | | | | |
| Seq ID | TPDMFKSPN | AAV9 | 587/590 | #N/A | −2.47 |
| 537 | | | | | |
| Seq ID | GNHSVHHQQ | AAV9 | 587/590 | −2.13 | −3.51 |
| 538 | | | | | |
| Seq ID | ENSRTTTVG | AAV9 | 587/590 | −0.03 | −0.78 |
| 539 | | | | | |
| Seq ID | TFVVGQSGK | AAV9 | 587/590 | −1.98 | −2.43 |
| 540 | | | | | |
| Seq ID | APLRPQTGE | AAV9 | 587/590 | −2.28 | −1.25 |
| 541 | | | | | |
| Seq ID | QTSSSTHHA | AAV9 | 587/590 | −2.32 | −2.06 |
| 542 | | | | | |
| Seq ID | KIAATALSS | AAV9 | 587/590 | −0.60 | −2.60 |
| 543 | | | | | |
| Seq ID | PESGQSKMH | AAV9 | 587/590 | 0.32 | −0.04 |
| 544 | | | | | |
| Seq ID | VISVGQMAK | AAV9 | 587/590 | −0.72 | −1.58 |
| 545 | | | | | |
| Seq ID | AERPQQGMH | AAV9 | 587/590 | −0.08 | −0.11 |
| 546 | | | | | |
| Seq ID | TQQNQIIQR | AAV9 | 587/590 | −1.31 | −1.13 |
| 547 | | | | | |
| Seq ID | ARQAQEKFG | AAV2 | R588A/589 | #N/A | −1.50 |
| 548 | KDKSPKA | | | | |
| Seq ID | VTAVTTPSH | AAV9 | 587/590 | 0.03 | −0.97 |
| 549 | | | | | |
| Seq ID | SQVRGPETT | AAV9 | 587/590 | −1.48 | −2.54 |
| 550 | | | | | |
| Seq ID | PLNHQQIQR | AAV9 | 587/590 | −2.15 | −2.20 |
| 551 | | | | | |
| Seq ID | AVVPRPHEI | AAV9 | 587/590 | −0.23 | −1.45 |
| 552 | | | | | |
| Seq ID | TPNMEDNRQ | AAV9 | 587/590 | −2.08 | −0.21 |
| 553 | | | | | |
| Seq ID | HRVTGVNDV | AAV9 | 587/590 | −1.63 | −0.74 |
| 554 | | | | | |
| Seq ID | FTGNQGAQN | AAV9 | 587/590 | 1.46 | −0.34 |
| 555 | | | | | |
| Seq ID | PRLAETSSQ | AAV9 | 587/590 | −0.20 | −1.30 |
| 556 | | | | | |
| Seq ID | SPRQSDPAH | AAV9 | 587/590 | −1.33 | −0.63 |
| 557 | | | | | |
| Seq ID | MNDDQQPPP | AAV9 | 587/590 | −0.11 | −1.64 |
| 558 | | | | | |
| Seq ID | QMKPLPQAM | AAV9 | 587/590 | −4.08 | −2.65 |
| 559 | | | | | |
| Seq ID | YSRHENISY | AAV9 | 587/590 | −2.91 | −3.89 |
| 560 | | | | | |
| Seq ID | TEGERVGML | AAV9 | 587/590 | 0.27 | 0.45 |
| 561 | | | | | |
| Seq ID | NHVPVLWAD | AAV9 | 587/590 | −0.16 | −2.45 |
| 562 | | | | | |
| Seq ID | TLAPLATTS | AAV9 | 587/590 | 0.21 | 0.65 |
| 563 | | | | | |
| Seq ID | NQRDVTNLT | AAV9 | 587/590 | 1.75 | 2.12 |
| 564 | | | | | |
| Seq ID | QQNNTVPNQ | AAV9 | 587/590 | 0.05 | −0.98 |
| 565 | | | | | |
| Seq ID | EKNMHTGVH | AAV9 | 587/590 | 0.31 | 0.28 |
| 566 | | | | | |
| Seq ID | VTLQGATPN | AAV9 | 587/590 | 1.23 | 0.24 |
| 567 | | | | | |
| Seq ID | PTKTSTGQM | AAV9 | 587/590 | −0.94 | −3.07 |
| 568 | | | | | |
| Seq ID | FNVDQHRNS | AAV9 | 587/590 | −1.07 | −0.74 |
| 569 | | | | | |
| Seq ID | PMTLLKDIQ | AAV9 | 587/590 | −1.29 | −1.55 |
| 570 | | | | | |
| Seq ID | DPTRPIETV | AAV9 | 587/590 | 0.13 | −1.83 |
| 571 | | | | | |
| Seq ID | ERKQQLSEM | AAV9 | 587/590 | −0.42 | −1.96 |
| 572 | | | | | |
| Seq ID | NQEQFGMIV | AAV9 | 587/590 | −0.61 | −0.51 |
| 573 | | | | | |
| Seq ID | TTQIMVGTK | AAV9 | 587/590 | −0.15 | −0.81 |
| 574 | | | | | |
| Seq ID | MYAKTEVQW | AAV9 | 587/590 | 0.27 | 0.26 |
| 575 | | | | | |
| Seq ID | PVGHQMDVY | AAV9 | 587/590 | −0.46 | −0.17 |
| 576 | | | | | |
| Seq ID | QVFQDRTSL | AAV9 | 587/590 | −0.01 | −0.75 |
| 577 | | | | | |
| Seq ID | LKDNQQATF | AAV9 | 587/590 | −2.46 | −1.38 |
| 578 | | | | | |
| Seq ID | AGNTHTLIN | AAV9 | 587/590 | −0.73 | −0.37 |
| 579 | | | | | |
| Seq ID | KVPPQHLTA | AAV9 | 587/590 | −0.97 | −2.15 |
| 580 | | | | | |
| Seq ID | GIVTATRVQ | AAV9 | 587/590 | −2.67 | −2.21 |
| 581 | | | | | |
| Seq ID | NIAHHQGSN | AAV9 | 587/590 | −1.45 | −1.73 |
| 582 | | | | | |
| Seq ID | PAMPGAAAR | AAV9 | 587/590 | −2.38 | −2.00 |
| 583 | | | | | |
| Seq ID | VQSITPPQS | AAV9 | 587/590 | 0.48 | 0.39 |
| 584 | | | | | |
| Seq ID | QATHQGQNH | AAV9 | 587/590 | 0.51 | −1.57 |
| 585 | | | | | |
| Seq ID | PTNAPGVTE | AAV9 | 587/590 | −0.60 | 0.66 |
| 586 | | | | | |
| Seq ID | MHRAQAIAE | AAV9 | 587/590 | −3.45 | −2.20 |
| 587 | | | | | |
| Seq ID | AQASEKMMK | AAV9 | 587/590 | −0.48 | −0.82 |
| 588 | | | | | |
| Seq ID | RMNIPPNGT | AAV9 | 587/590 | −0.83 | −1.98 |
| 589 | | | | | |
| Seq ID | RPSDINSGD | AAV9 | 587/590 | 0.34 | −0.67 |
| 590 | | | | | |
| Seq ID | MQPKSAQNV | AAV9 | 587/590 | −2.22 | −2.85 |
| 591 | | | | | |
| Seq ID | LAASMQGAH | AAV9 | 587/590 | −1.42 | −0.94 |
| 592 | | | | | |
| Seq ID | TSIITQAAN | AAV9 | 587/590 | 0.26 | −1.38 |
| 593 | | | | | |
| Seq ID | SDREETFNM | AAV9 | 587/590 | 0.09 | −0.48 |
| 594 | | | | | |
| Seq ID | NQYNMQPSK | AAV9 | 587/590 | −2.98 | −2.90 |
| 595 | | | | | |
| Seq ID | WVSEEHFLT | AAV9 | 587/590 | 1.95 | −0.85 |
| 596 | | | | | |
| Seq ID | QSATSVTSQ | AAV9 | 587/590 | 0.46 | −0.77 |
| 597 | | | | | |
| Seq ID | HARQSYNDP | AAV9 | 587/590 | −0.97 | −2.18 |
| 598 | | | | | |
| Seq ID | AMNVISHDG | AAV2 | R588A/589 | −2.24 | −1.85 |
| 599 | TPA | | | | |
| Seq ID | STIMSEAEF | AAV9 | 587/590 | −2.01 | −1.75 |
| 600 | | | | | |
| Seq ID | HANTNVLEK | AAV9 | 587/590 | −0.44 | −0.32 |
| 601 | | | | | |
| Seq ID | KPQVTPAVH | AAV9 | 587/590 | −1.22 | −1.90 |
| 602 | | | | | |
| Seq ID | AMPDRTMMR | AAV9 | 587/590 | −2.88 | −2.71 |
| 603 | | | | | |
| Seq ID | VAIGQTKTL | AAV9 | 587/590 | 0.53 | −1.51 |
| 604 | | | | | |
| Seq ID | PTDYSVKSV | AAV9 | 587/590 | −1.83 | −1.14 |
| 605 | | | | | |
| Seq ID | IGHHKEMKE | AAV9 | 587/590 | −1.36 | −0.07 |
| 606 | | | | | |
| Seq ID | ASQAIHYNA | AAV9 | 587/590 | −0.14 | −0.28 |
| 607 | | | | | |
| Seq ID | QVRMEGAEY | AAV9 | 587/590 | 0.66 | 1.09 |
| 608 | | | | | |
| Seq ID | MKERHEHFE | AAV9 | 587/590 | 0.49 | 0.44 |
| 609 | | | | | |
| Seq ID | GQVLMAGTR | AAV9 | 587/590 | −0.59 | −1.10 |
| 610 | | | | | |
| Seq ID | TVSMPVSHA | AAV9 | 587/590 | 1.29 | 0.71 |
| 611 | | | | | |
| Seq ID | NHDRVNVQP | AAV9 | 587/590 | −1.67 | −1.31 |
| 612 | | | | | |
| Seq ID | EELGILNLQ | AAV9 | 587/590 | −0.70 | −1.82 |
| 613 | | | | | |
| Seq ID | ANGNFQVLK | AAV9 | 587/590 | 0.86 | 0.18 |
| 614 | | | | | |
| Seq ID | RQYNTESMH | AAV9 | 587/590 | 0.03 | −0.06 |
| 615 | | | | | |
| Seq ID | MKVSAQVQS | AAV9 | 587/590 | −3.03 | −2.79 |
| 616 | | | | | |
| Seq ID | NVSQQKMVQ | AAV9 | 587/590 | −0.30 | −2.02 |
| 617 | | | | | |
| Seq ID | RQQNQDKFS | AAV9 | 587/590 | −1.18 | −3.69 |
| 618 | | | | | |
| Seq ID | MSSRTMEIH | AAV9 | 587/590 | −0.76 | −0.62 |
| 619 | | | | | |
| Seq ID | HEMIKQSGP | AAV9 | 587/590 | −1.55 | −1.21 |
| 620 | | | | | |
| Seq ID | QDTDRAGKF | AAV9 | 587/590 | −0.02 | 0.98 |
| 621 | | | | | |
| Seq ID | KWEQRGMAH | AAV9 | 587/590 | −1.85 | 0.14 |
| 622 | | | | | |
| Seq ID | TKQHTEMQQ | AAV9 | 587/590 | −2.48 | −0.97 |
| 623 | | | | | |
| Seq ID | PMQALQNML | AAV9 | 587/590 | −0.42 | −1.64 |
| 624 | | | | | |
| Seq ID | KVAFQQQIE | AAV9 | 587/590 | −0.07 | −1.08 |
| 625 | | | | | |
| Seq ID | QVPRQEVNE | AAV9 | 587/590 | −0.58 | −0.33 |
| 626 | | | | | |
| Seq ID | HLEREHKWD | AAV9 | 587/590 | −0.92 | −1.58 |
| 627 | | | | | |
| Seq ID | IQRKIQDLA | AAV9 | 587/590 | −3.99 | −0.34 |
| 628 | | | | | |
| Seq ID | IKGTDSMQQ | AAV9 | 587/590 | 1.26 | 1.22 |
| 629 | | | | | |
| Seq ID | TMVSGEARQ | AAV9 | 587/590 | 0.93 | 0.87 |
| 630 | | | | | |
| Seq ID | TMVASRQPP | AAV9 | 587/590 | −1.76 | −3.61 |
| 631 | | | | | |
| Seq ID | QQRQHQEIT | AAV9 | 587/590 | −0.23 | 0.07 |
| 632 | | | | | |
| Seq ID | NPESMEAYI | AAV9 | 587/590 | −1.39 | −0.86 |
| 633 | | | | | |
| Seq ID | TSQNGKMSV | AAV9 | 587/590 | −1.71 | −2.28 |
| 634 | | | | | |
| Seq ID | SRTSTQPQE | AAV9 | 587/590 | −1.03 | −1.44 |
| 635 | | | | | |
| Seq ID | RYAVKGETD | AAV9 | 587/590 | −1.26 | −1.18 |
| 636 | | | | | |
| Seq ID | VALYPNHAE | AAV9 | 587/590 | 0.53 | 0.78 |
| 637 | | | | | |
| Seq ID | QQTTQHIDS | AAV9 | 587/590 | 1.33 | 0.19 |
| 638 | | | | | |
| Seq ID | SSMQEASQI | AAV9 | 587/590 | 0.00 | −0.55 |
| 639 | | | | | |
| Seq ID | KMNSPNQER | AAV9 | 587/590 | −3.27 | −4.41 |
| 640 | | | | | |
| Seq ID | MTNRTPASN | AAV9 | 587/590 | −2.99 | −2.57 |
| 641 | | | | | |
| Seq ID | IGVRPMISP | AAV9 | 587/590 | −2.77 | −2.74 |
| 642 | | | | | |
| Seq ID | RQADSRTIE | AAV9 | 587/590 | −1.81 | −1.98 |
| 643 | | | | | |
| Seq ID | EMRNHPAVQ | AAV9 | 587/590 | −3.41 | −1.01 |
| 644 | | | | | |
| Seq ID | MRTNEPHKE | AAV9 | 587/590 | 1.15 | 0.22 |
| 645 | | | | | |
| Seq ID | PTIQRQGMN | AAV9 | 587/590 | −2.11 | −3.15 |
| 646 | | | | | |
| Seq ID | DIDPEDIST | AAV9 | 587/590 | −3.71 | −1.11 |
| 647 | | | | | |
| Seq ID | EIKMQSTQA | AAV9 | 587/590 | −1.94 | −1.00 |
| 648 | | | | | |
| Seq ID | QNFPSQNAA | AAV9 | 587/590 | −1.44 | −1.06 |
| 649 | | | | | |
| Seq ID | YRTGPDEIQ | AAV9 | 587/590 | 0.93 | −0.12 |
| 650 | | | | | |
| Seq ID | IQHTHSHHE | AAV9 | 587/590 | 0.58 | −0.48 |
| 651 | | | | | |
| Seq ID | IISTQEGHI | AAV9 | 587/590 | 0.17 | 0.44 |
| 652 | | | | | |
| Seq ID | PEGDNTFMI | AAV9 | 587/590 | −0.91 | −0.91 |
| 653 | | | | | |
| Seq ID | RANAEYLSD | AAV9 | 587/590 | −0.10 | −0.02 |
| 654 | | | | | |
| Seq ID | EVSLPSNVK | AAV9 | 587/590 | 0.71 | 0.16 |
| 655 | | | | | |
| Seq ID | RMDINRATD | AAV9 | 587/590 | 0.31 | −0.38 |
| 656 | | | | | |
| Seq ID | HPSPNTAYV | AAV9 | 587/590 | 0.37 | 0.24 |
| 657 | | | | | |
| Seq ID | SYHSWFPMG | AAV9 | 587/590 | 0.78 | −0.93 |
| 658 | | | | | |
| Seq ID | AGDRQHMAA | AAV9 | 587/590 | −0.73 | −1.44 |
| 659 | | | | | |
| Seq ID | QPKKWGADY | AAV9 | 587/590 | −1.74 | −0.07 |
| 660 | | | | | |
| Seq ID | LKQDSRPTY | AAV9 | 587/590 | −0.58 | −1.90 |
| 661 | | | | | |
| Seq ID | VSSYQLKSD | AAV9 | 587/590 | −1.94 | −1.35 |
| 662 | | | | | |
| Seq ID | RGHSVSDNA | AAV9 | 587/590 | −0.65 | −0.54 |
| 663 | | | | | |
| Seq ID | HQAQNQMFK | AAV9 | 587/590 | −0.56 | 0.01 |
| 664 | | | | | |
| Seq ID | QASNFITVP | AAV9 | 587/590 | −1.50 | −0.76 |
| 665 | | | | | |
| Seq ID | VEPHYQQQW | AAV9 | 587/590 | −2.24 | −1.38 |
| 666 | | | | | |
| Seq ID | TQGRSQEAT | AAV9 | 587/590 | 0.02 | 0.28 |
| 667 | | | | | |
| Seq ID | ATSSRTTLA | AAV9 | 588/589 | −1.10 | −2.47 |
| 668 | | | | | |
| Seq ID | PSSIGKHDN | AAV9 | 587/590 | −0.93 | −1.03 |
| 669 | | | | | |
| Seq ID | RHSGDSIPE | AAV9 | 587/590 | −0.04 | −0.97 |
| 670 | | | | | |
| Seq ID | ARPATTQSN | AAV9 | 587/590 | −1.54 | −3.12 |
| 671 | | | | | |
| Seq ID | PAPSGNTMA | AAV9 | 587/590 | −0.38 | −0.66 |
| 672 | | | | | |
| Seq ID | KSPSMSVVL | AAV9 | 587/590 | −3.55 | −2.92 |
| 673 | | | | | |
| Seq ID | IPGPNGQIP | AAV9 | 587/590 | −3.16 | −2.75 |
| 674 | | | | | |
| Seq ID | PQDYHMSEH | AAV9 | 587/590 | −1.56 | −2.23 |
| 675 | | | | | |
| Seq ID | ATHLRVIYE | AAV9 | 588/589 | −1.82 | −0.28 |
| 676 | RMNQSLA | | | | |
| Seq ID | AHGKREVAL | AAV2 | R588A/589 | −3.05 | −3.03 |
| 677 | HLHPDHA | | | | |
| Seq ID | PHTHAQMNL | AAV9 | 587/590 | −0.67 | −1.21 |
| 678 | | | | | |
| Seq ID | TVPSQQPGT | AAV9 | 587/590 | −0.51 | −1.16 |
| 679 | | | | | |
| Seq ID | AEYKDCHLA | AAV2 | R588A/589 | #N/A | −2.75 |
| 680 | QVPSHTA | | | | |
| Seq ID | AVGALIQRG | AAV2 | R588A/589 | −1.61 | −2.44 |
| 681 | FIRPKDA | | | | |
| Seq ID | TETDPMQRM | AAV9 | 587/590 | −1.63 | −0.97 |
| 682 | | | | | |
| Seq ID | HRQVAGEST | AAV9 | 587/590 | −0.05 | −0.56 |
| 683 | | | | | |
| Seq ID | SQSPTRIIQ | AAV9 | 587/590 | −1.11 | −0.92 |
| 684 | | | | | |
| Seq ID | RLAIPDGIT | AAV9 | 587/590 | 0.46 | −0.95 |
| 685 | | | | | |
| Seq ID | RPPQNPQTI | AAV9 | 587/590 | −0.16 | 0.19 |
| 686 | | | | | |
| Seq ID | VELMRTHST | AAV9 | 587/590 | #N/A | −4.21 |
| 687 | | | | | |
| Seq ID | HQPQYSKSD | AAV9 | 587/590 | −3.16 | −2.97 |
| 688 | | | | | |
| Seq ID | ATMQCAHTA | AAV2 | R588A/589 | −2.05 | −2.28 |
| 689 | | | | | |
| Seq ID | KMGGVAAKI | AAV9 | 587/590 | 0.73 | −1.08 |
| 690 | | | | | |
| Seq ID | TMNKSADRG | AAV9 | 587/590 | −0.34 | −1.99 |
| 691 | | | | | |
| Seq ID | EVDDFVSYG | AAV9 | 587/590 | −2.48 | −2.81 |
| 692 | | | | | |
| Seq ID | PMSQKVQSA | AAV9 | 587/590 | −1.85 | −1.62 |
| 693 | | | | | |
| Seq ID | IHSKANDVL | AAV9 | 587/590 | 0.59 | 0.30 |
| 694 | | | | | |
| Seq ID | AVVSPANAA | AAV9 | 587/590 | 0.10 | −0.37 |
| 695 | | | | | |
| Seq ID | RQTQTASEY | AAV9 | 587/590 | −2.90 | −2.82 |
| 696 | | | | | |
| Seq ID | MESHPQAPQ | AAV9 | 587/590 | −2.83 | −1.63 |
| 697 | | | | | |
| Seq ID | TFRESEMIM | AAV9 | 587/590 | 0.63 | 0.83 |
| 698 | | | | | |
| Seq ID | HHTPQNMVN | AAV9 | 587/590 | −2.31 | −2.10 |
| 699 | | | | | |
| Seq ID | KTQQMTPVT | AAV9 | 587/590 | −0.96 | −3.19 |
| 700 | | | | | |
| Seq ID | IAPQQTYWK | AAV9 | 587/590 | 1.06 | −0.36 |
| 701 | | | | | |
| Seq ID | EVHSKVMHP | AAV9 | 587/590 | −0.04 | 0.08 |
| 702 | | | | | |
| Seq ID | NKSTSTVAW | AAV9 | 587/590 | −0.51 | −1.52 |
| 703 | | | | | |
| Seq ID | ADSAQAMTK | AAV9 | 587/590 | 0.52 | 0.44 |
| 704 | | | | | |
| Seq ID | MESRKPNDI | AAV9 | 587/590 | −2.78 | −2.43 |
| 705 | | | | | |
| Seq ID | NVAQTMIRV | AAV9 | 587/590 | −1.72 | −0.45 |
| 706 | | | | | |
| Seq ID | HPRDHEHVD | AAV9 | 587/590 | −2.94 | −0.54 |
| 707 | | | | | |
| Seq ID | RVNGSQKES | AAV9 | 587/590 | #N/A | −6.87 |
| 708 | | | | | |
| Seq ID | YSRASSETA | AAV9 | 587/590 | −2.49 | −2.11 |
| 709 | | | | | |
| Seq ID | NWGQTQAKQ | AAV9 | 587/590 | 0.28 | −0.72 |
| 710 | | | | | |
| Seq ID | AQEMSKDLE | AAV2 | R588A/589 | −1.12 | −1.00 |
| 711 | EVKAKVA | | | | |
| Seq ID | REMRASQVE | AAV9 | 587/590 | −3.73 | −2.67 |
| 712 | | | | | |
| Seq ID | QYIQEHVAK | AAV9 | 587/590 | 1.04 | 0.43 |
| 713 | | | | | |
| Seq ID | QVAQTTLPI | AAV9 | 587/590 | −0.50 | −0.55 |
| 714 | | | | | |
| Seq ID | NTVQNMAHQ | AAV9 | 587/590 | 0.14 | −0.22 |
| 715 | | | | | |
| Seq ID | TQSREMDTQ | AAV9 | 587/590 | 1.48 | 0.17 |
| 716 | | | | | |
| Seq ID | DDAAHHGFD | AAV9 | 587/590 | −1.91 | −0.69 |
| 717 | | | | | |
| Seq ID | AATPSPVLP | AAV2 | R588A/589 | −4.48 | −2.69 |
| 718 | DIVMEAA | | | | |
| Seq ID | QQASGNNLR | AAV9 | 587/590 | −1.34 | −1.83 |
| 719 | | | | | |
| Seq ID | AKVVTDSMS | AAV9 | 587/590 | −1.04 | −0.38 |
| 720 | | | | | |
| Seq ID | SQVVSQGSQ | AAV9 | 587/590 | 0.12 | −0.67 |
| 721 | | | | | |
| Seq ID | FQHRANSME | AAV9 | 587/590 | −2.53 | −2.21 |
| 722 | | | | | |
| Seq ID | ANRGTEWDA | AAV9 | 588/589 | 0.28 | −0.70 |
| 723 | | | | | |
| Seq ID | TIDEVDIGY | AAV9 | 587/590 | −0.94 | −1.57 |
| 724 | | | | | |
| Seq ID | LQYKAMQVS | AAV9 | 587/590 | −3.20 | −0.27 |
| 725 | | | | | |
| Seq ID | AIPVCEAFQ | AAV2 | R588A/589 | −0.41 | −1.60 |
| 726 | CTALSNA | | | | |
| Seq ID | TEDSPQMTQ | AAV9 | 587/590 | −0.55 | −1.84 |
| 727 | | | | | |
| Seq ID | SGSMTPTTV | AAV9 | 587/590 | 1.07 | 0.86 |
| 728 | | | | | |
| Seq ID | LPMPTSQGI | AAV9 | 587/590 | −2.70 | −1.29 |
| 729 | | | | | |
| Seq ID | SYPTPQSQT | AAV9 | 587/590 | −0.23 | −1.84 |
| 730 | | | | | |
| Seq ID | SASKSFPTA | AAV9 | 587/590 | −0.47 | −0.38 |
| 731 | | | | | |
| Seq ID | PSQPMKFIN | AAV9 | 587/590 | −1.85 | −0.78 |
| 732 | | | | | |
| Seq ID | VEAQHKPAY | AAV9 | 587/590 | 0.36 | −1.25 |
| 733 | | | | | |
| Seq ID | IPSPVTAYW | AAV9 | 587/590 | −3.11 | 1.06 |
| 734 | | | | | |
| Seq ID | NYLSTSVPR | AAV9 | 587/590 | −2.13 | −2.35 |
| 735 | | | | | |
| Seq ID | TNSVKMPPS | AAV9 | 587/590 | −0.57 | −3.55 |
| 736 | | | | | |
| Seq ID | PQEDVKSVM | AAV9 | 587/590 | −2.96 | −1.49 |
| 737 | | | | | |
| Seq ID | EPHNMKVEK | AAV9 | 587/590 | −6.08 | −2.50 |
| 738 | | | | | |
| Seq ID | PQKQSLHTV | AAV9 | 587/590 | −1.39 | −2.04 |
| 739 | | | | | |
| Seq ID | KPQGVNVTY | AAV9 | 587/590 | −1.57 | −2.75 |
| 740 | | | | | |
| Seq ID | FQRSEVQTI | AAV9 | 587/590 | 0.22 | 0.83 |
| 741 | | | | | |
| Seq ID | AAEPAPDFS | AAV9 | 588/589 | −1.62 | −2.81 |
| 742 | DYSEMAA | | | | |
| Seq ID | QRQGGMEAK | AAV9 | 587/590 | −3.57 | −2.81 |
| 743 | | | | | |
| Seq ID | TASVKQQNT | AAV9 | 587/590 | −1.28 | −2.36 |
| 744 | | | | | |
| Seq ID | HRNTESGDQ | AAV9 | 587/590 | −1.52 | −0.48 |
| 745 | | | | | |
| Seq ID | FPKSYEQVQ | AAV9 | 587/590 | 0.17 | −0.47 |
| 746 | | | | | |
| Seq ID | QGASQRVAI | AAV9 | 587/590 | −3.45 | −3.63 |
| 747 | | | | | |
| Seq ID | HPMAKDVVK | AAV9 | 587/590 | −0.79 | −2.58 |
| 748 | | | | | |
| Seq ID | REQKLYKEP | AAV9 | 587/590 | −3.51 | −2.05 |
| 749 | | | | | |
| Seq ID | TEQQWYGRM | AAV9 | 587/590 | −0.83 | −2.29 |
| 750 | | | | | |
| Seq ID | APTNKTVGI | AAV9 | 587/590 | −1.80 | −2.50 |
| 751 | | | | | |
| Seq ID | QRTAVNEMQ | AAV9 | 587/590 | −0.35 | −1.05 |
| 752 | | | | | |
| Seq ID | PQNAQRVDT | AAV9 | 587/590 | −2.00 | −2.19 |
| 753 | | | | | |
| Seq ID | KDDDKVGIV | AAV9 | 587/590 | −2.23 | −1.72 |
| 754 | | | | | |
| Seq ID | APAPAAPPS | AAV2 | R588A/589 | −4.79 | −3.03 |
| 755 | TPAAPKA | | | | |
| Seq ID | NHAVMHTVS | AAV9 | 587/590 | 0.69 | 1.08 |
| 756 | | | | | |
| Seq ID | MRTQAVDGF | AAV9 | 587/590 | −1.48 | −2.99 |
| 757 | | | | | |
| Seq ID | DIQHATKRE | AAV9 | 587/590 | −0.83 | −0.76 |
| 758 | | | | | |
| Seq ID | ATQAHTMMY | AAV9 | 587/590 | 0.49 | 0.13 |
| 759 | | | | | |
| Seq ID | QGTSEASRY | AAV9 | 587/590 | 1.13 | 0.69 |
| 760 | | | | | |
| Seq ID | RNSHGMSES | AAV9 | 587/590 | −0.60 | −1.23 |
| 761 | | | | | |
| Seq ID | PNSAHNNSY | AAV9 | 587/590 | 0.60 | −0.40 |
| 762 | | | | | |
| Seq ID | TTGASKDKV | AAV9 | 587/590 | −2.21 | −0.75 |
| 763 | | | | | |
| Seq ID | DRTGVWAGE | AAV9 | 587/590 | −1.02 | −3.32 |
| 764 | | | | | |
| Seq ID | VQTLDRGSQ | AAV9 | 587/590 | −2.35 | −4.04 |
| 765 | | | | | |
| Seq ID | DATAHSLIQ | AAV9 | 587/590 | −0.29 | −0.42 |
| 766 | | | | | |
| Seq ID | IGQAPEVND | AAV9 | 587/590 | −4.80 | −1.98 |
| 767 | | | | | |
| Seq ID | HYAKVTEQA | AAV9 | 587/590 | −2.00 | −0.16 |
| 768 | | | | | |
| Seq ID | DPSVRSNMI | AAV9 | 587/590 | −0.74 | −1.20 |
| 769 | | | | | |
| Seq ID | MSKVSTQDR | AAV9 | 587/590 | −3.35 | −2.46 |
| 770 | | | | | |
| Seq ID | AQDRISQWA | AAV2 | R588A/589 | −2.45 | −1.84 |
| 771 | | | | | |
| Seq ID | AHLAQPATL | AAV2 | R588A/589 | −4.63 | −2.87 |
| 772 | SQTIVPA | | | | |
| Seq ID | NEKIRELPK | AAV9 | 587/590 | −1.86 | −1.76 |
| 773 | | | | | |
| Seq ID | QMTKHETYI | AAV9 | 587/590 | −3.83 | −0.94 |
| 774 | | | | | |
| Seq ID | GAQQYSQVS | AAV9 | 587/590 | 1.68 | −0.64 |
| 775 | | | | | |
| Seq ID | IQDDPMTSM | AAV9 | 587/590 | −3.25 | −0.66 |
| 776 | | | | | |
| Seq ID | VPTVERNSL | AAV9 | 587/590 | 1.35 | 0.71 |
| 777 | | | | | |
| Seq ID | KLVMTQTEY | AAV9 | 587/590 | −1.71 | 0.16 |
| 778 | | | | | |
| Seq ID | GGVPMKALI | AAV9 | 587/590 | −0.47 | −1.29 |
| 779 | | | | | |
| Seq ID | IQSPQQAMR | AAV9 | 587/590 | −1.29 | −2.31 |
| 780 | | | | | |
| Seq ID | SFHGPIKNY | AAV9 | 587/590 | −1.20 | −0.90 |
| 781 | | | | | |
| Seq ID | NTKDNAGKD | AAV9 | 587/590 | −0.68 | −0.38 |
| 782 | | | | | |
| Seq ID | ADFSDLYYH | AAV2 | R588A/589 | −2.15 | −3.02 |
| 783 | TMNNKHA | | | | |
| Seq ID | QKQIMHPES | AAV9 | 587/590 | −4.18 | −3.18 |
| 784 | | | | | |
| Seq ID | PQPQMGHSM | AAV9 | 587/590 | −1.91 | −1.45 |
| 785 | | | | | |
| Seq ID | QLHTGIQNS | AAV9 | 587/590 | −0.40 | −0.92 |
| 786 | | | | | |
| Seq ID | AFEVMEDHA | AAV2 | R588A/589 | −1.99 | −3.31 |
| 787 | GTYGLGA | | | | |
| Seq ID | SYVQPTAIM | AAV9 | 587/590 | −0.56 | −1.26 |
| 788 | | | | | |
| Seq ID | IQATRNVGV | AAV9 | 587/590 | −2.66 | −2.34 |
| 789 | | | | | |
| Seq ID | MILVVLQSV | AAV9 | 587/590 | −0.95 | 0.38 |
| 790 | | | | | |
| Seq ID | KMFSSSEQF | AAV9 | 587/590 | −2.04 | −1.95 |
| 791 | | | | | |
| Seq ID | TTDRDKMAL | AAV9 | 587/590 | −1.48 | −1.56 |
| 792 | | | | | |
| Seq ID | KAINTMIEA | AAV9 | 587/590 | #N/A | −1.90 |
| 793 | | | | | |
| Seq ID | HMTNGTPMM | AAV9 | 587/590 | −0.89 | −1.83 |
| 794 | | | | | |
| Seq ID | MQNSVKQVW | AAV9 | 587/590 | −1.74 | −1.11 |
| 795 | | | | | |
| Seq ID | SQPKMQETG | AAV9 | 587/590 | 0.52 | 0.68 |
| 796 | | | | | |
| Seq ID | TESHSITPM | AAV9 | 587/590 | 1.40 | 0.88 |
| 797 | | | | | |
| Seq ID | QVSQGVHGQ | AAV9 | 587/590 | −0.80 | −1.39 |
| 798 | | | | | |
| Seq ID | TTQMQPMVA | AAV9 | 587/590 | 0.20 | 0.43 |
| 799 | | | | | |
| Seq ID | KLTVTRRMF | AAV9 | 587/590 | −0.34 | 0.91 |
| 800 | | | | | |
| Seq ID | AGIATSGSA | AAV9 | 588/589 | 0.42 | −1.31 |
| 801 | | | | | |
| Seq ID | TPNMQSMSY | AAV9 | 587/590 | 0.85 | 0.29 |
| 802 | | | | | |
| Seq ID | FNDQPQKIF | AAV9 | 587/590 | 0.45 | −0.41 |
| 803 | | | | | |
| Seq ID | PTSVHGQML | AAV9 | 587/590 | −0.44 | −0.72 |
| 804 | | | | | |
| Seq ID | SQTHQVGYP | AAV9 | 587/590 | 2.23 | 1.64 |
| 805 | | | | | |
| Seq ID | QGTTATRPD | AAV9 | 587/590 | −4.84 | −1.37 |
| 806 | | | | | |
| Seq ID | AGGKVQIIN | AAV2 | R588A/589 | −3.91 | −1.47 |
| 807 | KKLDLSA | | | | |
| Seq ID | ASLSITEDA | AAV2 | R588A/589 | −2.30 | −0.72 |
| 808 | | | | | |
| Seq ID | TSKPPALQG | AAV9 | 587/590 | −0.14 | −0.21 |
| 809 | | | | | |
| Seq ID | KVMEKQGMR | AAV9 | 587/590 | 0.43 | 0.29 |
| 810 | | | | | |
| Seq ID | KTVESPQQQ | AAV9 | 587/590 | 0.78 | 0.19 |
| 811 | | | | | |
| Seq ID | AGPSVGRAK | AAV9 | 588/589 | −1.52 | −3.98 |
| 812 | GQDAPLA | | | | |
| Seq ID | RHPSPVNVL | AAV9 | 587/590 | −1.67 | −2.13 |
| 813 | | | | | |
| Seq ID | YQNDMRKGT | AAV9 | 587/590 | −5.98 | −4.30 |
| 814 | | | | | |
| Seq ID | LINAHQPQK | AAV9 | 587/590 | 0.30 | 1.15 |
| 815 | | | | | |
| Seq ID | AGHAIENAV | AAV2 | R588A/589 | −3.17 | −2.76 |
| 816 | RSSLKGA | | | | |
| Seq ID | GIGTHMTMV | AAV9 | 587/590 | 0.51 | −0.14 |
| 817 | | | | | |
| Seq ID | RTLHVDNSP | AAV9 | 587/590 | 0.44 | 0.48 |
| 818 | | | | | |
| Seq ID | ADLENLRDL | AAV2 | R588A/589 | −4.21 | −3.67 |
| 819 | LHVGGGA | | | | |
| Seq ID | QEQANQRPN | AAV9 | 587/590 | −4.82 | −2.55 |
| 820 | | | | | |
| Seq ID | VSAQSMRNP | AAV9 | 587/590 | −1.44 | −4.21 |
| 821 | | | | | |
| Seq ID | TLQQKTYNP | AAV9 | 587/590 | −1.46 | −2.17 |
| 822 | | | | | |
| Seq ID | FLDSNHSMT | AAV9 | 587/590 | 0.66 | 0.67 |
| 823 | | | | | |
| Seq ID | TDYYSTDDY | AAV9 | 587/590 | −0.57 | −2.84 |
| 824 | | | | | |
| Seq ID | AVLTQTMGK | AAV9 | 587/590 | −0.81 | −1.67 |
| 825 | | | | | |
| Seq ID | PAPHSVVQM | AAV9 | 587/590 | −0.50 | −1.70 |
| 826 | | | | | |
| Seq ID | RPNASDIKG | AAV9 | 587/590 | −2.73 | −2.50 |
| 827 | | | | | |
| Seq ID | PQRNTGIVQ | AAV9 | 587/590 | −1.85 | −4.56 |
| 828 | | | | | |
| Seq ID | IVSQTPVAV | AAV9 | 587/590 | −0.90 | −2.03 |
| 829 | | | | | |
| Seq ID | PTTRIGEHP | AAV9 | 587/590 | 0.09 | −0.51 |
| 830 | | | | | |
| Seq ID | RQPTDSYSK | AAV9 | 587/590 | −1.07 | −1.87 |
| 831 | | | | | |
| Seq ID | TVGGKTYAD | AAV9 | 587/590 | 0.45 | −0.22 |
| 832 | | | | | |
| Seq ID | LKNPPLGNQ | AAV9 | 587/590 | 0.21 | 0.06 |
| 833 | | | | | |
| Seq ID | LSQSQSQTY | AAV9 | 587/590 | 0.89 | 0.37 |
| 834 | | | | | |
| Seq ID | PPTAQAQYD | AAV9 | 587/590 | −0.98 | 0.19 |
| 835 | | | | | |
| Seq ID | NDQLHISLR | AAV9 | 587/590 | −0.54 | 0.37 |
| 836 | | | | | |
| Seq ID | AKNQYVEED | AAV9 | 587/590 | −0.31 | −0.17 |
| 837 | | | | | |
| Seq ID | QARANAFPD | AAV9 | 587/590 | −1.83 | −2.14 |
| 838 | | | | | |
| Seq ID | NGNFAYDHP | AAV9 | 587/590 | −2.59 | −1.81 |
| 839 | | | | | |
| Seq ID | ILQSNTRGI | AAV9 | 587/590 | −0.70 | −1.27 |
| 840 | | | | | |
| Seq ID | TQPQQHNEP | AAV9 | 587/590 | 0.22 | −0.80 |
| 841 | | | | | |
| Seq ID | QRHNENVQM | AAV9 | 587/590 | −0.16 | −1.73 |
| 842 | | | | | |
| Seq ID | AQVLRIRKR | AAV2 | R588A/589 | −1.95 | −3.41 |
| 843 | ANSFLEA | | | | |
| Seq ID | QLRDVQLQH | AAV9 | 587/590 | −0.49 | −0.34 |
| 844 | | | | | |
| Seq ID | YAKQSTQQM | AAV9 | 587/590 | −1.03 | −2.49 |
| 845 | | | | | |
| Seq ID | TQVLRDKQP | AAV9 | 587/590 | −2.24 | 0.00 |
| 846 | | | | | |
| Seq ID | QNKIEFRPE | AAV9 | 587/590 | 0.15 | −0.06 |
| 847 | | | | | |
| Seq ID | SSHASMSST | AAV9 | 587/590 | 1.24 | 0.76 |
| 848 | | | | | |
| Seq ID | RVVEQGRGI | AAV9 | 587/590 | −1.90 | −0.91 |
| 849 | | | | | |
| Seq ID | HRNPTVVVD | AAV9 | 587/590 | 0.65 | 0.09 |
| 850 | | | | | |
| Seq ID | APWNSATVL | AAV2 | R588A/589 | −2.24 | −1.32 |
| 851 | QQTYHAA | | | | |
| Seq ID | MWNVTSGAE | AAV9 | 587/590 | 0.21 | −0.65 |
| 852 | | | | | |
| Seq ID | KTQQALVLQ | AAV9 | 587/590 | −2.09 | −0.49 |
| 853 | | | | | |
| Seq ID | QVHQESMFG | AAV9 | 587/590 | −1.08 | −1.91 |
| 854 | | | | | |
| Seq ID | QLNTTKPIA | AAV9 | 587/590 | −2.38 | −1.39 |
| 855 | | | | | |
| Seq ID | EQYAIKQPT | AAV9 | 587/590 | −0.12 | −2.37 |
| 856 | | | | | |
| Seq ID | ALGKTAETH | AAV9 | 587/590 | 0.24 | −0.58 |
| 857 | | | | | |
| Seq ID | KVFHTPEMN | AAV9 | 587/590 | −0.40 | −0.04 |
| 858 | | | | | |
| Seq ID | ASQTAPIVT | AAV9 | 587/590 | −0.08 | 0.12 |
| 859 | | | | | |
| Seq ID | TLKKTDDVW | AAV9 | 587/590 | −2.76 | −0.94 |
| 860 | | | | | |
| Seq ID | AHEALSPFY | AAV2 | R588A/589 | −0.40 | −4.32 |
| 861 | SERLKEA | | | | |
| Seq ID | QINGSHRIV | AAV9 | 587/590 | −1.70 | −1.08 |
| 862 | | | | | |
| Seq ID | ATAQDLFTA | AAV2 | R588A/589 | −0.98 | −1.73 |
| 863 | | | | | |
| Seq ID | ATAQDLFTA | AAV9 | 588/589 | −0.98 | −1.73 |
| 864 | | | | | |
| Seq ID | SQHTSDMES | AAV9 | 587/590 | −0.71 | 0.30 |
| 865 | | | | | |
| Seq ID | YRTATTTNS | AAV9 | 587/590 | −1.33 | −3.58 |
| 866 | | | | | |
| Seq ID | PASKQFTPD | AAV9 | 587/590 | −2.30 | −1.14 |
| 867 | | | | | |
| Seq ID | TATRQEVHH | AAV9 | 587/590 | −0.23 | −0.48 |
| 868 | | | | | |
| Seq ID | TTTGVMVAK | AAV9 | 587/590 | −0.94 | −1.85 |
| 869 | | | | | |
| Seq ID | NPVFHQSVA | AAV9 | 587/590 | −2.16 | −2.10 |
| 870 | | | | | |
| Seq ID | KPVSSQHTP | AAV9 | 587/590 | 0.07 | −3.14 |
| 871 | | | | | |
| Seq ID | TMATAHQTD | AAV9 | 587/590 | −1.87 | −0.93 |
| 872 | | | | | |
| Seq ID | VVSGVPMSQ | AAV9 | 587/590 | 0.79 | 0.42 |
| 873 | | | | | |
| Seq ID | MTFGMQQNK | AAV9 | 587/590 | −1.21 | −0.97 |
| 874 | | | | | |
| Seq ID | YGQAVVMQN | AAV9 | 587/590 | −0.26 | −0.32 |
| 875 | | | | | |
| Seq ID | LFHKHPDEM | AAV9 | 587/590 | −2.73 | −4.19 |
| 876 | | | | | |
| Seq ID | RMRDQDVTS | AAV9 | 587/590 | 0.78 | −0.32 |
| 877 | | | | | |
| Seq ID | MKQTGGSSF | AAV9 | 587/590 | −1.46 | −2.78 |
| 878 | | | | | |
| Seq ID | LQASSGAMF | AAV9 | 587/590 | 0.13 | −0.80 |
| 879 | | | | | |
| Seq ID | NVSTTASQV | AAV9 | 587/590 | 1.45 | 0.12 |
| 880 | | | | | |
| Seq ID | IMQTSVGRD | AAV9 | 587/590 | −2.67 | −1.43 |
| 881 | | | | | |
| Seq ID | KLAGVVAIH | AAV9 | 587/590 | 0.89 | −0.82 |
| 882 | | | | | |
| Seq ID | EIKGAADSH | AAV9 | 587/590 | −0.59 | −0.68 |
| 883 | | | | | |
| Seq ID | VQQGNPMQR | AAV9 | 587/590 | −0.76 | −0.80 |
| 884 | | | | | |
| Seq ID | HRWVQLWRV | AAV9 | 587/590 | 0.65 | −0.34 |
| 885 | | | | | |
| Seq ID | RAETHKYNP | AAV9 | 587/590 | 0.93 | 2.99 |
| 886 | | | | | |
| Seq ID | RDPAINHDQ | AAV9 | 587/590 | 0.24 | −0.84 |
| 887 | | | | | |
| Seq ID | PQSQGMQQR | AAV9 | 587/590 | −2.78 | −3.07 |
| 888 | | | | | |
| Seq ID | GIRQDQEYF | AAV9 | 587/590 | −0.68 | −1.24 |
| 889 | | | | | |
| Seq ID | ANCRIPA | AAV2 | R588A/589 | −6.26 | −2.51 |
| 890 | | | | | |
| Seq ID | GTTMKVQMS | AAV9 | 587/590 | −2.51 | −2.68 |
| 891 | | | | | |
| Seq ID | NTHSSHIMV | AAV9 | 587/590 | −0.32 | −1.41 |
| 892 | | | | | |
| Seq ID | KQREPGNVG | AAV9 | 587/590 | −4.49 | −2.95 |
| 893 | | | | | |
| Seq ID | IYFNDAEQI | AAV9 | 587/590 | −0.86 | −1.43 |
| 894 | | | | | |
| Seq ID | NHTAKAAPL | AAV9 | 587/590 | −0.92 | −1.89 |
| 895 | | | | | |
| Seq ID | QHVVGQFPQ | AAV9 | 587/590 | −2.18 | −2.06 |
| 896 | | | | | |
| Seq ID | RIERQHNDQ | AAV9 | 587/590 | −0.09 | −2.18 |
| 897 | | | | | |
| Seq ID | AEAKRHRPL | AAV2 | R588A/589 | −1.98 | −0.91 |
| 898 | IALPTQA | | | | |
| Seq ID | EPHTSSQQR | AAV9 | 587/590 | −0.83 | −0.17 |
| 899 | | | | | |
| Seq ID | AEMYCAPLK | AAV2 | R588A/589 | −2.07 | −1.14 |
| 900 | PAHSARA | | | | |
| Seq ID | DPHQRMAQQ | AAV9 | 587/590 | 1.42 | 0.00 |
| 901 | | | | | |
| Seq ID | PSITNRQGA | AAV9 | 587/590 | −2.45 | −3.19 |
| 902 | | | | | |
| Seq ID | ADEPARKGK | AAV2 | R588A/589 | 0.20 | −0.88 |
| 903 | VHIPFPA | | | | |
| Seq ID | LLTPQSRSS | AAV9 | 587/590 | −2.95 | −2.57 |
| 904 | | | | | |
| Seq ID | LTLQGQAKQ | AAV9 | 587/590 | −0.39 | −0.08 |
| 905 | | | | | |
| Seq ID | QEDDQRSDR | AAV9 | 587/590 | −2.83 | −2.94 |
| 906 | | | | | |
| Seq ID | PTNPGEVPV | AAV9 | 587/590 | −0.87 | −1.95 |
| 907 | | | | | |
| Seq ID | MLSNEDGTN | AAV9 | 587/590 | −4.58 | −1.32 |
| 908 | | | | | |
| Seq ID | PMEMTTELF | AAV9 | 587/590 | −3.10 | −2.71 |
| 909 | | | | | |
| Seq ID | MPTQSRQYI | AAV9 | 587/590 | 0.29 | −2.17 |
| 910 | | | | | |
| Seq ID | SKTQHTNPE | AAV9 | 587/590 | 0.26 | −0.72 |
| 911 | | | | | |
| Seq ID | QDSFFQRWA | AAV9 | 587/590 | 1.52 | 0.00 |
| 912 | | | | | |
| Seq ID | ITIDVQSAK | AAV9 | 587/590 | −1.28 | −1.52 |
| 913 | | | | | |
| Seq ID | PQQSHVVSQ | AAV9 | 587/590 | −1.07 | −3.34 |
| 914 | | | | | |
| Seq ID | VQVGTSVGK | AAV9 | 587/590 | −2.16 | −2.65 |
| 915 | | | | | |
| Seq ID | SAQTSHPIM | AAV9 | 587/590 | 1.95 | 1.07 |
| 916 | | | | | |
| Seq ID | PQEMMALVY | AAV9 | 587/590 | −2.21 | −0.89 |
| 917 | | | | | |
| Seq ID | QHYRMRPHT | AAV9 | 587/590 | −0.77 | −1.36 |
| 918 | | | | | |
| Seq ID | NMAQHGQLQ | AAV9 | 587/590 | 1.17 | 0.20 |
| 919 | | | | | |
| Seq ID | RHGLTTPDF | AAV9 | 587/590 | −3.37 | −2.39 |
| 920 | | | | | |
| Seq ID | TTMQRTMDV | AAV9 | 587/590 | −0.25 | −2.15 |
| 921 | | | | | |
| Seq ID | PKSHGAHDV | AAV9 | 587/590 | −1.19 | −1.54 |
| 922 | | | | | |
| Seq ID | AIENAVRSS | AAV9 | 588/589 | −2.39 | −2.56 |
| 923 | LKVATSA | | | | |
| Seq ID | PMVDAMVSV | AAV9 | 587/590 | −0.79 | −0.56 |
| 924 | | | | | |
| Seq ID | TEQPEPFVF | AAV9 | 587/590 | −1.15 | −1.59 |
| 925 | | | | | |
| Seq ID | LDAVPKNIK | AAV9 | 587/590 | −0.62 | −0.82 |
| 926 | | | | | |
| Seq ID | TGMSQATSR | AAV9 | 587/590 | −1.02 | −1.39 |
| 927 | | | | | |
| Seq ID | PTRPSSETM | AAV9 | 587/590 | −1.27 | −2.83 |
| 928 | | | | | |
| Seq ID | RIEGSHPPP | AAV9 | 587/590 | −1.76 | 0.04 |
| 929 | | | | | |
| Seq ID | VKGHQEFAN | AAV9 | 587/590 | −0.13 | −0.14 |
| 930 | | | | | |
| Seq ID | MLVGVPQSK | AAV9 | 587/590 | −0.17 | −0.55 |
| 931 | | | | | |
| Seq ID | QRQQPQPFF | AAV9 | 587/590 | −3.54 | −1.88 |
| 932 | | | | | |
| Seq ID | ARMQASALA | AAV2 | R588A/589 | −3.59 | −5.23 |
| 933 | | | | | |
| Seq ID | DWEAKARVA | AAV9 | 587/590 | −1.43 | −0.20 |
| 934 | | | | | |
| Seq ID | PIASTITNR | AAV9 | 587/590 | −1.72 | −2.26 |
| 935 | | | | | |
| Seq ID | ENSQPIRPS | AAV9 | 587/590 | −0.51 | −2.39 |
| 936 | | | | | |
| Seq ID | AHMNVQNGK | AAV2 | R588A/589 | −2.97 | −2.93 |
| 937 | WDSDPSA | | | | |
| Seq ID | PHQVPRDPV | AAV9 | 587/590 | −2.04 | −2.06 |
| 938 | | | | | |
| Seq ID | TTSTNREVS | AAV9 | 587/590 | 1.42 | −0.03 |
| 939 | | | | | |
| Seq ID | PSGVMQDPV | AAV9 | 587/590 | −0.24 | −0.45 |
| 940 | | | | | |
| Seq ID | AKSHELQVA | AAV2 | R588A/589 | −0.08 | −1.18 |
| 941 | | | | | |
| Seq ID | AVNQVHHQM | AAV9 | 587/590 | −1.02 | −1.19 |
| 942 | | | | | |
| Seq ID | SVGRQQQDI | AAV9 | 587/590 | −1.74 | −2.01 |
| 943 | | | | | |
| Seq ID | VAMIKQSSI | AAV9 | 587/590 | 0.01 | −0.79 |
| 944 | | | | | |
| Seq ID | DILIWQHSD | AAV9 | 587/590 | 1.39 | −1.35 |
| 945 | | | | | |
| Seq ID | FTTKHVNEQ | AAV9 | 587/590 | −2.90 | −1.47 |
| 946 | | | | | |
| Seq ID | AKPYSGRRP | AAV2 | R588A/589 | 0.17 | −0.78 |
| 947 | DAIRLGA | | | | |
| Seq ID | QSMADPRAM | AAV9 | 587/590 | 0.18 | −1.70 |
| 948 | | | | | |
| Seq ID | AHLASNLSA | AAV2 | R588A/589 | −2.35 | −3.13 |
| 949 | | | | | |
| Seq ID | LQHSNQDQE | AAV9 | 587/590 | 0.25 | 0.44 |
| 950 | | | | | |
| Seq ID | NPWDQTKAM | AAV9 | 587/590 | −4.40 | −2.87 |
| 951 | | | | | |
| Seq ID | VTDVFDVYD | AAV9 | 587/590 | −1.82 | −2.56 |
| 952 | | | | | |
| Seq ID | EYDQGVTNF | AAV9 | 587/590 | −2.12 | −1.90 |
| 953 | | | | | |
| Seq ID | QTYSMPQHL | AAV9 | 587/590 | −2.97 | −1.07 |
| 954 | | | | | |
| Seq ID | FKTDRDSML | AAV9 | 587/590 | 0.43 | 0.21 |
| 955 | | | | | |
| Seq ID | AIPVPVDGM | AAV2 | R588A/589 | −0.63 | −2.96 |
| 956 | EYHWGNA | | | | |
| Seq ID | PASKNVQPM | AAV9 | 587/590 | −1.04 | −1.85 |
| 957 | | | | | |
| Seq ID | RLEISSQQI | AAV9 | 587/590 | −1.26 | −2.91 |
| 958 | | | | | |
| Seq ID | IQGLPSMVH | AAV9 | 587/590 | −1.63 | −1.07 |
| 959 | | | | | |
| Seq ID | NIAQNLLME | AAV9 | 587/590 | −1.11 | −0.96 |
| 960 | | | | | |
| Seq ID | HPQPSGHFS | AAV9 | 587/590 | 0.30 | −0.43 |
| 961 | | | | | |
| Seq ID | IPRQGSQTT | AAV9 | 587/590 | −3.31 | −2.57 |
| 962 | | | | | |
| Seq ID | DVLGQMGKM | AAV9 | 587/590 | 1.16 | 0.50 |
| 963 | | | | | |
| Seq ID | TYSSPSIGT | AAV9 | 587/590 | −2.09 | −2.97 |
| 964 | | | | | |
| Seq ID | EHKDFQVSG | AAV9 | 587/590 | −0.60 | −0.60 |
| 965 | | | | | |
| Seq ID | EVRPKQDFT | AAV9 | 587/590 | −1.45 | −0.30 |
| 966 | | | | | |
| Seq ID | ARYEAVSFM | AAV2 | R588A/589 | 0.01 | −0.74 |
| 967 | DVNSTWA | | | | |
| Seq ID | PYTQKTGSV | AAV9 | 587/590 | −1.35 | −1.58 |
| 968 | | | | | |
| Seq ID | NFVNNQPGY | AAV9 | 587/590 | −3.09 | −3.40 |
| 969 | | | | | |
| Seq ID | SGLNMKTSV | AAV9 | 587/590 | −2.85 | −1.77 |
| 970 | | | | | |
| Seq ID | AQHTLKHFE | AAV2 | R588A/589 | −5.46 | −3.11 |
| 971 | HVRMVDA | | | | |
| Seq ID | AQHTLKHFE | AAV9 | 588/589 | −5.46 | −3.11 |
| 972 | HVRMVDA | | | | |
| Seq ID | KENPQHGIY | AAV9 | 587/590 | 0.23 | −0.47 |
| 973 | | | | | |
| Seq ID | KSPTPDINT | AAV9 | 587/590 | −3.67 | −3.36 |
| 974 | | | | | |
| Seq ID | LTSSKSDPA | AAV9 | 587/590 | −0.70 | −0.50 |
| 975 | | | | | |
| Seq ID | ARMQASQVA | AAV2 | R588A/589 | −3.14 | −3.17 |
| 976 | | | | | |
| Seq ID | RYHRDTIDM | AAV9 | 587/590 | −3.07 | −1.55 |
| 977 | | | | | |
| Seq ID | ASLSITKPA | AAV2 | R588A/589 | −3.34 | −2.90 |
| 978 | | | | | |
| Seq ID | ASSPGSPGT | AAV2 | R588A/589 | −0.44 | −2.64 |
| 979 | PGSRSRA | | | | |
| Seq ID | ADRIPA | AAV2 | R588A/589 | −3.23 | −4.11 |
| 980 | | | | | |
| Seq ID | HETQVKGMQ | AAV9 | 587/590 | −0.50 | −1.86 |
| 981 | | | | | |
| Seq ID | TPRATPSMP | AAV9 | 587/590 | 0.31 | −0.61 |
| 982 | | | | | |
| Seq ID | RPTQHQDIN | AAV9 | 587/590 | −1.90 | −0.35 |
| 983 | | | | | |
| Seq ID | TTNTQGAQV | AAV9 | 587/590 | 1.00 | 0.20 |
| 984 | | | | | |
| Seq ID | HYGWQQVPD | AAV9 | 587/590 | −4.03 | −2.49 |
| 985 | | | | | |
| Seq ID | HPGSSMVPT | AAV9 | 587/590 | −0.54 | −1.73 |
| 986 | | | | | |
| Seq ID | SSVSQVTLR | AAV9 | 587/590 | 0.00 | −0.38 |
| 987 | | | | | |
| Seq ID | MYAAGNVHA | AAV9 | 587/590 | 0.17 | 0.30 |
| 988 | | | | | |
| Seq ID | TSGVVQTIH | AAV9 | 587/590 | 0.69 | −0.41 |
| 989 | | | | | |
| Seq ID | HMHPAQSEY | AAV9 | 587/590 | −2.96 | −3.00 |
| 990 | | | | | |
| Seq ID | PQKGDMHTS | AAV9 | 587/590 | −0.37 | −0.69 |
| 991 | | | | | |
| Seq ID | ATYQPLFTA | AAV2 | R588A/589 | −2.47 | −1.22 |
| 992 | | | | | |
| Seq ID | ATYQPLFTA | AAV9 | 588/589 | −2.47 | −1.22 |
| 993 | | | | | |
| Seq ID | AQSSDIENA | AAV2 | R588A/589 | −1.52 | −1.55 |
| 994 | | | | | |
| Seq ID | QVAIQPQEE | AAV9 | 587/590 | −1.36 | −1.15 |
| 995 | | | | | |
| Seq ID | RAVQNVTTM | AAV9 | 587/590 | −4.05 | −1.35 |
| 996 | | | | | |
| Seq ID | PSTLVMQSV | AAV9 | 587/590 | −0.05 | −0.89 |
| 997 | | | | | |
| Seq ID | QQQLLDDIP | AAV9 | 587/590 | −1.12 | −2.09 |
| 998 | | | | | |
| Seq ID | WEKHIMIDY | AAV9 | 587/590 | −2.33 | −1.12 |
| 999 | | | | | |
| Seq ID | NQSTSVNTW | AAV9 | 587/590 | −2.74 | −1.91 |
| 1000 | | | | | |
| Seq ID | TVQEADSQP | AAV9 | 587/590 | −0.79 | −1.52 |
| 1001 | | | | | |
| Seq ID | AGDAIENAV | AAV2 | R588A/589 | −2.70 | −2.21 |
| 1002 | RSSLKVA | | | | |
| Seq ID | RTDKSQYQP | AAV9 | 587/590 | #N/A | −1.45 |
| 1003 | | | | | |
| Seq ID | ADGGFIYEA | AAV2 | R588A/589 | −1.18 | −1.70 |
| 1004 | GLAPYKA | | | | |
| Seq ID | ARMKDNTPP | AAV9 | 587/590 | −0.98 | −1.02 |
| 1005 | | | | | |
| Seq ID | STITPMRQI | AAV9 | 587/590 | −1.49 | −0.61 |
| 1006 | | | | | |
| Seq ID | QTPIAPPPI | AAV9 | 587/590 | −0.22 | −0.85 |
| 1007 | | | | | |
| Seq ID | AQPEHRSER | AAV9 | 587/590 | −3.61 | −3.99 |
| 1008 | | | | | |
| Seq ID | ASRANISHA | AAV2 | R588A/589 | −2.98 | −3.72 |
| 1009 | | | | | |
| Seq ID | AAAAGAVVG | AAV2 | R588A/589 | #N/A | −1.92 |
| 1010 | GLGGYMA | | | | |
| Seq ID | PQMRQLYHS | AAV9 | 587/590 | −2.08 | −0.04 |
| 1011 | | | | | |
| Seq ID | LEFHSQAMV | AAV9 | 587/590 | −0.04 | −1.80 |
| 1012 | | | | | |
| Seq ID | AQKTYQEGD | AAV9 | 587/590 | −0.40 | −0.62 |
| 1013 | | | | | |
| Seq ID | AMFSDLYYL | AAV2 | R588A/589 | 0.09 | −0.61 |
| 1014 | TMNNKHA | | | | |
| Seq ID | VLDYMDIVF | AAV9 | 587/590 | −1.15 | −1.06 |
| 1015 | | | | | |
| Seq ID | ADFSDLCYL | AAV2 | R588A/589 | −0.80 | 0.64 |
| 1016 | TMNNKHA | | | | |
| Seq ID | ANAVIPA | AAV2 | R588A/589 | −0.49 | −1.40 |
| 1017 | | | | | |
| Seq ID | KRERGMQDY | AAV9 | 587/590 | 0.31 | −1.29 |
| 1018 | | | | | |
| Seq ID | GPFTPAGMM | AAV9 | 587/590 | 0.22 | −0.53 |
| 1019 | | | | | |
| Seq ID | RKMPQAIDV | AAV9 | 587/590 | −1.52 | −3.58 |
| 1020 | | | | | |
| Seq ID | GSDPAGFKM | AAV9 | 587/590 | −4.72 | −2.86 |
| 1021 | | | | | |
| Seq ID | DAQSIQVSI | AAV9 | 587/590 | −0.70 | −0.66 |
| 1022 | | | | | |
| Seq ID | KVNEVMQKD | AAV9 | 587/590 | 1.22 | 0.08 |
| 1023 | | | | | |
| Seq ID | ATSKVLLLP | AAV2 | R588A/589 | −0.71 | −2.25 |
| 1024 | PDVSALA | | | | |
| Seq ID | AAAIPA | AAV2 | R588A/589 | −2.53 | −3.02 |
| 1025 | | | | | |
| Seq ID | ARYADGHAI | AAV2 | R588A/589 | −1.15 | −2.09 |
| 1026 | ENAVRSA | | | | |
| Seq ID | SEHDNHQQT | AAV9 | 587/590 | −4.27 | −1.18 |
| 1027 | | | | | |
| Seq ID | PRVQGNDQA | AAV9 | 587/590 | 0.06 | −0.72 |
| 1028 | | | | | |
| Seq ID | AVAVHHTPA | AAV2 | R588A/589 | −3.34 | −5.68 |
| 1029 | | | | | |
| Seq ID | ADVRDPRGA | AAV2 | R588A/589 | −4.58 | −2.35 |
| 1030 | | | | | |
| Seq ID | ASKTSVVDL | AAV2 | R588A/589 | #N/A | −1.24 |
| 1031 | LYWRDIA | | | | |
| Seq ID | NVNETKGSW | AAV9 | 587/590 | −3.04 | −0.93 |
| 1032 | | | | | |
| Seq ID | AGHAIENAV | AAV2 | R588A/589 | −3.42 | −4.46 |
| 1033 | KSSLKVA | | | | |
| Seq ID | ALDWIHGHI | AAV2 | R588A/589 | #N/A | −0.21 |
| 1034 | RDKEAPA | | | | |
| Seq ID | SPKYFTPTM | AAV9 | 587/590 | 0.47 | 1.22 |
| 1035 | | | | | |
| Seq ID | KSQVQPQGT | AAV9 | 587/590 | #N/A | −2.75 |
| 1036 | | | | | |
| Seq ID | QMDTRIPQR | AAV9 | 587/590 | −1.40 | −2.23 |
| 1037 | | | | | |
| Seq ID | AFRALSTGE | AAV2 | R588A/589 | #N/A | −1.23 |
| 1038 | KGFGYKA | | | | |
| Seq ID | ATCEAVRCD | AAV2 | R588A/589 | −1.99 | −3.92 |
| 1039 | AVHQPPA | | | | |
| Seq ID | ERSSQYEKG | AAV9 | 587/590 | 1.01 | 1.72 |
| 1040 | | | | | |
| Seq ID | IRPGTAMAP | AAV9 | 587/590 | −2.50 | −2.36 |
| 1041 | | | | | |
| Seq ID | SPHSVQGSG | AAV9 | 587/590 | 0.49 | 0.50 |
| 1042 | | | | | |
| Seq ID | QQAQKVAGV | AAV9 | 587/590 | −2.40 | −4.47 |
| 1043 | | | | | |
| Seq ID | PRDISTAMV | AAV9 | 587/590 | −2.00 | −0.62 |
| 1044 | | | | | |
| Seq ID | PRVTQDLVG | AAV9 | 587/590 | 0.21 | 0.35 |
| 1045 | | | | | |
| Seq ID | QQVVTRHEM | AAV9 | 587/590 | −5.91 | −1.41 |
| 1046 | | | | | |
| Seq ID | ARMQAKAVA | AAV2 | R588A/589 | −0.76 | −0.88 |
| 1047 | | | | | |
| Seq ID | ANPRIPA | AAV2 | R588A/589 | −3.24 | −3.72 |
| 1048 | | | | | |
| Seq ID | MVEHNHHVM | AAV9 | 587/590 | 0.61 | 0.63 |
| 1049 | | | | | |
| Seq ID | AQSSDISPA | AAV2 | R588A/589 | −1.62 | −1.60 |
| 1050 | | | | | |
| Seq ID | ARPQASAVA | AAV2 | R588A/589 | −2.30 | −3.17 |
| 1051 | | | | | |
| Seq ID | NDFDMPGEI | AAV9 | 587/590 | −1.07 | −2.98 |
| 1052 | | | | | |
| Seq ID | NRNPTMTGP | AAV9 | 587/590 | −2.18 | −3.21 |
| 1053 | | | | | |
| Seq ID | ANAGIPA | AAV2 | R588A/589 | 0.21 | −0.86 |
| 1054 | | | | | |
| Seq ID | ADFSHLYYL | AAV2 | R588A/589 | −4.66 | −0.27 |
| 1055 | TMNNKHA | | | | |
| Seq ID | ACELSGYGK | AAV2 | R588A/589 | −2.66 | −1.39 |
| 1056 | HEALSPA | | | | |
| Seq ID | TANKSAPVS | AAV9 | 587/590 | −0.93 | −1.36 |
| 1057 | | | | | |
| Seq ID | QGHSMPTYG | AAV9 | 587/590 | −0.52 | −0.61 |
| 1058 | | | | | |
| Seq ID | EYRVTDNTQ | AAV9 | 587/590 | −1.77 | −0.08 |
| 1059 | | | | | |
| Seq ID | LVAVQQSSG | AAV9 | 587/590 | 0.65 | −0.57 |
| 1060 | | | | | |
| Seq ID | ASPGGNRYP | AAV9 | 588/589 | −1.79 | −3.22 |
| 1061 | PQGGGGA | | | | |
| Seq ID | RTDQVMSTK | AAV9 | 587/590 | −0.40 | −1.67 |
| 1062 | | | | | |
| Seq ID | TSDHLFQYD | AAV9 | 587/590 | −1.09 | −1.07 |
| 1063 | | | | | |
| Seq ID | ANNRIPA | AAV2 | R588A/589 | −2.76 | −4.27 |
| 1064 | | | | | |
| Seq ID | MHTEAIMPR | AAV9 | 587/590 | 0.20 | −0.72 |
| 1065 | | | | | |
| Seq ID | SVVSVTMNQ | AAV9 | 587/590 | −0.77 | −1.23 |
| 1066 | | | | | |
| Seq ID | AYKSSCAFS | AAV2 | R588A/589 | −2.46 | −4.38 |
| 1067 | CEEGFEA | | | | |
| Seq ID | VTVQPHVDI | AAV9 | 587/590 | −2.21 | −4.21 |
| 1068 | | | | | |
| Seq ID | MEDKYAYEM | AAV9 | 587/590 | −1.32 | −2.48 |
| 1069 | | | | | |
| Seq ID | ASARSPSEA | AAV2 | R588A/589 | −0.66 | −2.26 |
| 1070 | | | | | |
| Seq ID | QHQDRTPSN | AAV9 | 587/590 | −3.46 | −0.84 |
| 1071 | | | | | |
| Seq ID | NRQPIHKDD | AAV9 | 587/590 | −0.50 | −0.36 |
| 1072 | | | | | |
| Seq ID | TTMGSPAPM | AAV9 | 587/590 | 1.14 | 0.08 |
| 1073 | | | | | |
| Seq ID | KQDPRYSYQ | AAV9 | 587/590 | −1.01 | #N/A |
| 1074 | | | | | |
| Seq ID | AQLSITKDA | AAV2 | R588A/589 | −1.80 | −2.12 |
| 1075 | | | | | |
| Seq ID | PRAGTSIAP | AAV9 | 587/590 | −3.40 | −3.73 |
| 1076 | | | | | |
| Seq ID | AMPRNSIPA | AAV2 | R588A/589 | −3.80 | −4.41 |
| 1077 | | | | | |
| Seq ID | ASVTTVVSA | AAV9 | 587/590 | 1.47 | 0.42 |
| 1078 | | | | | |
| Seq ID | SPQGKVSQV | AAV9 | 587/590 | −2.03 | −0.58 |
| 1079 | | | | | |
| Seq ID | RVTNVPNAV | AAV9 | 587/590 | −0.84 | −2.42 |
| 1080 | | | | | |
| Seq ID | MLPQASAHK | AAV9 | 587/590 | −1.64 | −1.94 |
| 1081 | | | | | |
| Seq ID | ATKEADLPE | AAV2 | R588A/589 | −0.01 | −1.98 |
| 1082 | PSEKQPA | | | | |
| Seq ID | ARGKRGQDA | AAV2 | R588A/589 | #N/A | −1.85 |
| 1083 | MYEYMAA | | | | |
| Seq ID | PNPKSHQWP | AAV9 | 587/590 | −4.20 | −3.00 |
| 1084 | | | | | |
| Seq ID | ADGAETEVD | AAV2 | R588A/589 | −1.09 | −3.72 |
| 1085 | CNRCVCA | | | | |
| Seq ID | SDTNTAPQE | AAV9 | 587/590 | −2.17 | −1.73 |
| 1086 | | | | | |
| Seq ID | ASKSTTDND | AAV2 | R588A/589 | −3.19 | −2.15 |
| 1087 | IALLHLA | | | | |
| Seq ID | KYISTGGEW | AAV9 | 587/590 | −1.10 | −3.20 |
| 1088 | | | | | |
| Seq ID | AGDSCVTIM | AAV2 | R588A/589 | #N/A | −0.88 |
| 1089 | SKDKPTA | | | | |
| Seq ID | AVLEGDSCV | AAV2 | R588A/589 | −1.77 | −2.17 |
| 1090 | TIMSKDA | | | | |
| Seq ID | AELPGFLQS | AAV2 | R588A/589 | −2.49 | −1.82 |
| 1091 | GKDKDAA | | | | |
| Seq ID | QQMSSHQPM | AAV9 | 587/590 | 0.03 | −0.65 |
| 1092 | | | | | |
| Seq ID | AYHEREQGQ | AAV9 | 587/590 | −0.84 | −1.13 |
| 1093 | | | | | |
| Seq ID | PKHERVGDL | AAV9 | 587/590 | −0.97 | −1.25 |
| 1094 | | | | | |
| Seq ID | MENADVNDG | AAV9 | 587/590 | −2.27 | −3.15 |
| 1095 | | | | | |
| Seq ID | IIEHVSYQY | AAV9 | 587/590 | #N/A | 0.29 |
| 1096 | | | | | |
| Seq ID | NQEKQQSNT | AAV9 | 587/590 | −0.94 | −1.34 |
| 1097 | | | | | |
| Seq ID | KVIHMDDSY | AAV9 | 587/590 | −2.05 | −0.86 |
| 1098 | | | | | |
| Seq ID | NITINQREH | AAV9 | 587/590 | −3.24 | −3.22 |
| 1099 | | | | | |
| Seq ID | AGGCVTITA | AAV2 | R588A/589 | −2.89 | −6.35 |
| 1100 | EGKPSMA | | | | |
| Seq ID | QVKQSSQLN | AAV9 | 587/590 | −0.06 | −1.46 |
| 1101 | | | | | |
| Seq ID | AADRSSPGA | AAV2 | R588A/589 | −0.51 | −2.29 |
| 1102 | | | | | |
| Seq ID | NAPRGYMPD | AAV9 | 587/590 | −1.14 | −3.35 |
| 1103 | | | | | |
| Seq ID | IATPLQKQM | AAV9 | 587/590 | −1.26 | −1.27 |
| 1104 | | | | | |
| Seq ID | DVHHQYSQI | AAV9 | 587/590 | −1.98 | −0.11 |
| 1105 | | | | | |
| Seq ID | HHRDNSQSN | AAV9 | 587/590 | −0.04 | 0.04 |
| 1106 | | | | | |
| Seq ID | AGHGECVET | AAV2 | R588A/589 | −2.48 | −2.57 |
| 1107 | INNYTCA | | | | |
| Seq ID | FQWQEETDT | AAV9 | 587/590 | −4.98 | −2.40 |
| 1108 | | | | | |
| Seq ID | AHAHRSDAL | AAV2 | R588A/589 | −0.86 | −4.57 |
| 1109 | QLGLGKA | | | | |
| Seq ID | MTTPPQSSP | AAV9 | 587/590 | 1.26 | 0.57 |
| 1110 | | | | | |
| Seq ID | ANSTWRTVD | AAV2 | R588A/589 | #N/A | −2.23 |
| 1111 | RLSATAA | | | | |
| Seq ID | TEGRQPVGF | AAV9 | 587/590 | −1.11 | −1.58 |
| 1112 | | | | | |
| Seq ID | PQEEGPSQF | AAV9 | 587/590 | −1.44 | −2.17 |
| 1113 | | | | | |
| Seq ID | PMNYSHGAL | AAV9 | 587/590 | −1.42 | −2.71 |
| 1114 | | | | | |
| Seq ID | ASSSERAHQ | AAV2 | R588A/589 | −2.88 | −1.02 |
| 1115 | VLRIRKA | | | | |
| Seq ID | AILRQQQHL | AAV2 | R588A/589 | −1.18 | 0.01 |
| 1116 | FGSNVTA | | | | |
| Seq ID | QKPQPQQDP | AAV9 | 587/590 | −0.53 | −1.53 |
| 1117 | | | | | |
| Seq ID | HTAATSTKD | AAV9 | 587/590 | −0.50 | 1.45 |
| 1118 | | | | | |
| Seq ID | SVKAVMPFP | AAV9 | 587/590 | −2.71 | −1.35 |
| 1119 | | | | | |
| Seq ID | ALIFRHHRQ | AAV9 | 587/590 | −2.45 | −0.79 |
| 1120 | | | | | |
| Seq ID | PMEPEHMWG | AAV9 | 587/590 | −3.82 | −3.70 |
| 1121 | | | | | |
| Seq ID | EKDVMRVVQ | AAV9 | 587/590 | 0.13 | −0.69 |
| 1122 | | | | | |
| Seq ID | ARCRAIDGD | AAV2 | R588A/589 | −0.07 | −1.07 |
| 1123 | VTFCRPA | | | | |
| Seq ID | ASVGGVFNS | AAV2 | R588A/589 | #N/A | −2.55 |
| 1124 | LGKGIHA | | | | |
| Seq ID | VASHHFQPQ | AAV9 | 587/590 | −0.23 | 0.20 |
| 1125 | | | | | |
| Seq ID | TNKQMAYST | AAV9 | 587/590 | −1.58 | −2.56 |
| 1126 | | | | | |
| Seq ID | ATPSLPTPP | AAV2 | R588A/589 | −2.68 | −3.14 |
| 1127 | TREPKKA | | | | |
| Seq ID | YMEDLDPES | AAV9 | 587/590 | −1.24 | −3.44 |
| 1128 | | | | | |
| Seq ID | TMIQAKQLP | AAV9 | 587/590 | 0.91 | −1.27 |
| 1129 | | | | | |
| Seq ID | PPRVNDHAT | AAV9 | 587/590 | −0.49 | −1.67 |
| 1130 | | | | | |
| Seq ID | AMFDSSNYH | AAV2 | R588A/589 | −4.27 | −0.73 |
| 1131 | GQDLLFA | | | | |
| Seq ID | HPTVQTHSQ | AAV9 | 587/590 | −2.65 | −2.03 |
| 1132 | | | | | |
| Seq ID | LGSSAGVSF | AAV9 | 587/590 | −1.16 | −1.00 |
| 1133 | | | | | |
| Seq ID | AGLTTRPGS | AAV2 | R588A/589 | #N/A | 0.01 |
| 1134 | GLTNIKA | | | | |
| Seq ID | AGHFSGSGA | AAV2 | R588A/589 | −2.45 | −3.02 |
| 1135 | | | | | |
| Seq ID | RVQVGPVSS | AAV9 | 587/590 | −3.86 | −4.32 |
| 1136 | | | | | |
| Seq ID | SKINPQTPL | AAV9 | 587/590 | −3.56 | −3.29 |
| 1137 | | | | | |
| Seq ID | QRDQRVTPQ | AAV9 | 587/590 | −2.20 | −3.08 |
| 1138 | | | | | |
| Seq ID | SPAGYQANK | AAV9 | 587/590 | 0.49 | 0.45 |
| 1139 | | | | | |
| Seq ID | MTHAQQLPQ | AAV9 | 587/590 | −1.09 | −1.79 |
| 1140 | | | | | |
| Seq ID | AFKDSAIGF | AAV2 | R588A/589 | −0.12 | −0.79 |
| 1141 | SRVPPRA | | | | |
| Seq ID | MTLKREIMS | AAV9 | 587/590 | −4.91 | −2.67 |
| 1142 | | | | | |
| Seq ID | IPMQGEQMR | AAV9 | 587/590 | −0.93 | −0.75 |
| 1143 | | | | | |
| Seq ID | AQETHYMTR | AAV9 | 587/590 | −0.70 | −1.69 |
| 1144 | | | | | |
| Seq ID | AVRHSSDFA | AAV9 | 588/589 | 0.04 | −0.81 |
| 1145 | | | | | |
| Seq ID | ALSSSMSMA | AAV2 | R588A/589 | −3.38 | −3.29 |
| 1146 | | | | | |
| Seq ID | ANPRFNENN | AAV2 | R588A/589 | −0.42 | −0.83 |
| 1147 | RRVIVCA | | | | |
| Seq ID | AAIENAVRS | AAV9 | 588/589 | −2.19 | −2.33 |
| 1148 | SLKVATA | | | | |
| Seq ID | RQGGNIMEK | AAV9 | 587/590 | −2.06 | −2.37 |
| 1149 | | | | | |
| Seq ID | PTSQLSAAH | AAV9 | 587/590 | −0.98 | −1.53 |
| 1150 | | | | | |
| Seq ID | KPSQTHNDM | AAV9 | 587/590 | 0.27 | −1.63 |
| 1151 | | | | | |
| Seq ID | AKVAVVRTP | AAV2 | R588A/589 | −1.08 | −1.84 |
| 1152 | PKSPSSA | | | | |
| Seq ID | HDQGHAAQK | AAV9 | 587/590 | −0.93 | −0.76 |
| 1153 | | | | | |
| Seq ID | AMAAAMGNS | AAV9 | 587/590 | 1.66 | 1.16 |
| 1154 | | | | | |
| Seq ID | GDTQPRMQY | AAV9 | 587/590 | 0.88 | 0.73 |
| 1155 | | | | | |
| Seq ID | EYPSEIVYS | AAV9 | 587/590 | −0.76 | −2.39 |
| 1156 | | | | | |
| Seq ID | TWQHTTETS | AAV9 | 587/590 | −0.05 | −0.10 |
| 1157 | | | | | |
| Seq ID | TGGAYPVIV | AAV9 | 587/590 | −0.90 | −0.88 |
| 1158 | | | | | |
| Seq ID | AGYGSSSRR | AAV2 | R588A/589 | −0.01 | −2.72 |
| 1159 | APQTGIA | | | | |
| Seq ID | AVADSRSPA | AAV2 | R588A/589 | −0.95 | −2.52 |
| 1160 | | | | | |
| Seq ID | AGIVSWGRG | AAV2 | R588A/589 | −2.71 | −5.17 |
| 1161 | CALKDKA | | | | |
| Seq ID | AWESDDFTA | AAV2 | R588A/589 | −3.56 | −3.32 |
| 1162 | | | | | |
| Seq ID | SVDQSHDPN | AAV9 | 587/590 | −1.01 | 0.13 |
| 1163 | | | | | |
| Seq ID | ARPNLPLPA | AAV2 | R588A/589 | −3.34 | −3.84 |
| 1164 | | | | | |
| Seq ID | ARPNLPLPA | AAV9 | 588/589 | −3.34 | −3.84 |
| 1165 | | | | | |
| Seq ID | TDLADSEWR | AAV9 | 587/590 | −1.34 | −0.78 |
| 1166 | | | | | |
| Seq ID | AAFTTTLRG | AAV2 | R588A/589 | #N/A | −0.87 |
| 1167 | AQRLAAA | | | | |
| Seq ID | VSNPQPPSR | AAV9 | 587/590 | 0.17 | 0.18 |
| 1168 | | | | | |
| Seq ID | APSLPTPPT | AAV2 | R588A/589 | −1.44 | −1.82 |
| 1169 | REPKKVA | | | | |
| Seq ID | PTHTQGRES | AAV9 | 587/590 | −2.01 | −4.18 |
| 1170 | | | | | |
| Seq ID | AQNAKNGNL | AAV2 | R588A/589 | −4.96 | −3.25 |
| 1171 | PEFGDAA | | | | |
| Seq ID | KNAVMAPGN | AAV9 | 587/590 | −2.32 | −3.51 |
| 1172 | | | | | |
| Seq ID | QALVPIATQ | AAV9 | 588/589 | 1.01 | #N/A |
| 1173 | TYEAWLGA | | | | |
| Seq ID | IPMRIETDP | AAV9 | 587/590 | 0.23 | −1.03 |
| 1174 | | | | | |
| Seq ID | QARDSPKGW | AAV9 | 587/590 | 3.78 | 3.16 |
| 1175 | | | | | |
| Seq ID | AHRERMSQV | AAV2 | R588A/589 | #N/A | −3.33 |
| 1176 | MREWEEA | | | | |
| Seq ID | SRVSQNEHP | AAV9 | 587/590 | −0.83 | −0.91 |
| 1177 | | | | | |
| Seq ID | QRNEKSEFI | AAV9 | 587/590 | −4.31 | −3.65 |
| 1178 | | | | | |
| Seq ID | QTLPEVTKW | AAV9 | 587/590 | −0.26 | −0.38 |
| 1179 | | | | | |
| Seq ID | VKGWQVTER | AAV9 | 587/590 | #N/A | −0.06 |
| 1180 | | | | | |
| Seq ID | APTTVESHG | AAV2 | R588A/589 | #N/A | −3.22 |
| 1181 | NYSTQIA | | | | |
| Seq ID | PRQGPSTNL | AAV9 | 587/590 | −3.69 | −2.37 |
| 1182 | | | | | |
| Seq ID | AEATDGMLK | AAV2 | R588A/589 | #N/A | −4.75 |
| 1183 | IQFSAQA | | | | |
| Seq ID | ALYRQKVEP | AAV9 | 588/589 | −1.99 | −0.92 |
| 1184 | LRAELQA | | | | |
| Seq ID | RPLTTEAIV | AAV9 | 587/590 | 0.68 | 0.08 |
| 1185 | | | | | |
| Seq ID | KPGSQQIMD | AAV9 | 587/590 | 0.03 | 1.63 |
| 1186 | | | | | |
| Seq ID | AHQLLGDLH | AAV2 | R588A/589 | −1.47 | −2.42 |
| 1187 | QEGPPLA | | | | |
| Seq ID | MEQLVTFKT | AAV9 | 587/590 | −0.05 | 0.04 |
| 1188 | | | | | |
| Seq ID | GASMQTALW | AAV9 | 587/590 | 1.04 | −1.58 |
| 1189 | | | | | |
| Seq ID | AAFRCLAEN | AAV2 | R588A/589 | −2.71 | 0.65 |
| 1190 | AGDVAFA | | | | |
| Seq ID | IQTQLPPGK | AAV9 | 587/590 | −1.02 | −1.17 |
| 1191 | | | | | |
| Seq ID | NTVHQAQIM | AAV9 | 587/590 | 0.28 | 0.83 |
| 1192 | | | | | |
| Seq ID | PISNVNQSR | AAV9 | 587/590 | −6.49 | −3.67 |
| 1193 | | | | | |
| Seq ID | APDLVQEAC | AAV2 | R588A/589 | −0.43 | −1.60 |
| 1194 | ESELNEA | | | | |
| Seq ID | AKIQVLVEP | AAV2 | R588A/589 | #N/A | −3.69 |
| 1195 | DHFKVAA | | | | |
| Seq ID | AREWFMDLN | AAV2 | R588A/589 | −1.74 | −0.40 |
| 1196 | LPWSSAA | | | | |
| Seq ID | AEFSSNTVK | AAV9 | 588/589 | −3.08 | −2.61 |
| 1197 | LTSGHLA | | | | |
| Seq ID | AFTYKSSCA | AAV2 | R588A/589 | −2.11 | #N/A |
| 1198 | FSCEEGA | | | | |
| Seq ID | PYPTQASMK | AAV9 | 587/590 | −3.09 | −2.63 |
| 1199 | | | | | |
| Seq ID | KPTNSGQEH | AAV9 | 587/590 | −1.70 | −3.29 |
| 1200 | | | | | |
| Seq ID | LSTGPPEKI | AAV9 | 587/590 | 0.06 | 0.18 |
| 1201 | | | | | |
| Seq ID | APQLHVMSA | AAV9 | 587/590 | −0.29 | −1.68 |
| 1202 | | | | | |
| Seq ID | TASKMQDSK | AAV9 | 587/590 | −1.42 | −2.93 |
| 1203 | | | | | |
| Seq ID | AFTSSSGQK | AAV2 | R588A/589 | #N/A | −0.59 |
| 1204 | AMNGSRA | | | | |
| Seq ID | AVAEFSEAT | AAV2 | R588A/589 | −1.81 | −4.01 |
| 1205 | AELTVSA | | | | |
| Seq ID | INQNKAHDA | AAV9 | 587/590 | −0.12 | −1.93 |
| 1206 | | | | | |
| Seq ID | AARLSKELQ | AAV2 | R588A/589 | −2.27 | −0.87 |
| 1207 | AAQARLA | | | | |
| Seq ID | ATKPGYVDS | AAV2 | R588A/589 | −5.83 | −3.06 |
| 1208 | IQKGIQA | | | | |
| Seq ID | PTTAPSMVL | AAV9 | 587/590 | −1.51 | −2.21 |
| 1209 | | | | | |
| Seq ID | AGGFVYIAG | AAV2 | R588A/589 | #N/A | −3.97 |
| 1210 | KCGLVPA | | | | |
| Seq ID | ANPTAKRQF | AAV2 | R588A/589 | #N/A | −1.74 |
| 1211 | VQSAKEA | | | | |
| Seq ID | ALALGRFWD | AAV2 | R588A/589 | −0.38 | −1.08 |
| 1212 | YLRWVQA | | | | |
| Seq ID | ALRVIYERM | AAV2 | R588A/589 | 1.38 | −2.89 |
| 1213 | NQSLSLA | | | | |
| Seq ID | VTMTQATNR | AAV9 | 587/590 | −0.96 | −1.34 |
| 1214 | | | | | |
| Seq ID | ALLQYNHRV | AAV2 | R588A/589 | −1.27 | −1.96 |
| 1215 | KKLNEIA | | | | |
| Seq ID | ALLGKINLK | AAV2 | R588A/589 | −1.28 | −0.93 |
| 1216 | ALAALAA | | | | |
| Seq ID | RQRVSYMDD | AAV9 | 587/590 | −2.48 | −1.42 |
| 1217 | | | | | |
| Seq ID | ADMGQQRYD | AAV9 | 587/590 | −0.40 | −1.50 |
| 1218 | | | | | |
| Seq ID | ATGVLLRAL | AAV2 | R588A/589 | −3.18 | −1.12 |
| 1219 | AGVPVAA | | | | |
| Seq ID | SIKKDPPTV | AAV9 | 587/590 | −3.77 | −3.06 |
| 1220 | | | | | |
| Seq ID | PGKTMLGNH | AAV9 | 587/590 | −1.71 | −2.75 |
| 1221 | | | | | |
| Seq ID | HQGDTLQYV | AAV9 | 587/590 | 0.01 | −0.17 |
| 1222 | | | | | |
| Seq ID | ALGLGKHNY | AAV2 | R588A/589 | 0.15 | −0.87 |
| 1223 | CRNPDGA | | | | |
| Seq ID | QERQANKSY | AAV9 | 587/590 | −2.45 | −4.07 |
| 1224 | | | | | |
| Seq ID | QQTRGVELN | AAV9 | 587/590 | −0.13 | −0.82 |
| 1225 | | | | | |
| Seq ID | ASMRVVPMA | AAV2 | R588A/589 | −2.48 | −2.59 |
| 1226 | | | | | |
| Seq ID | ASSTDKRDA | AAV2 | R588A/589 | −0.85 | −1.84 |
| 1227 | | | | | |
| Seq ID | ALIGGSIVA | AAV2 | R588A/589 | −2.49 | −1.72 |
| 1228 | | | | | |
| Seq ID | LLHTGTSSH | AAV9 | 587/590 | −0.03 | −0.56 |
| 1229 | | | | | |
| Seq ID | TQHQAQVPR | AAV9 | 587/590 | −1.83 | −3.05 |
| 1230 | | | | | |
| Seq ID | AAGLFGAAE | AAV2 | R588A/589 | −1.50 | −3.15 |
| 1231 | GQAFHLA | | | | |
| Seq ID | AENEHAHFQ | AAV2 | R588A/589 | −4.22 | −3.36 |
| 1232 | KAKERLA | | | | |

### Example 4. Immunohistochemistry Data Showing the Ability of CNSRCV300 to Cross the Blood Brain Barrier

### Background

Round 2 and 3 library evaluation results highlighted the exceptional ability of the capsid CNSRCV300 to cross the blood-brain barrier and mediate transgene expression throughout the central nervous system. Based on these results, next an individual evaluation of capsid CNSRCV300 in cynomolgus macaques was conducted. CNSRCV300 was manufactured in HEK293 cells with a vector encoding the ubiquitous CAG promoter and a nuclear localized GFP reporter. CNSRCV300 was administered intravenously in three cynomolgus macaques at a dose of 2E13 vector genomes per kilogram.

### Methods

Brain tissue was collected at necropsy on Day 19 after intravenous administration of CNSRCV300. Brain slices of 4 mm thickness were designated for immunohistochemistry evaluation and placed in labelled cassettes, immersion fixed in 10% paraformaldehyde, refrigerated at 4° C. for 16-24 hrs, transferred to phosphate buffered saline (1×PBS) with 0.01% sodium azide, and refrigerated at 4° C. until processing.

To prepare brain slices for processing, brains were treated overnight with 20% glycerol and 2% dimethylsulfoxide to avoid freeze artifacts. Slices were embedded in a gelatin matrix. Blocks were rapidly frozen, after curing by immersion in 2-methylbuteane chilled with crushed dry ice and mounted on a freezing stage of a AO 860 sliding microtome.

Blocks were sectioned coronally at 40 μM thickness on the microtome. All sections were cut through the entire length of the specimen segment and collected sequentially into a series of 24 containers, pre-filled with Antigen Preserve solution (50% PBS, pH 7.0, 50% ethylene glycol, 1% polyvinyl pyrrolidone).

For IHC revealing GFP, free floating sections were stained. All incubation solution from the blocking serum onward used TRIS-buffered saline (TBS) with Triton-X-100 as the vehicle; all rinses were with TBS. After a hydrogen peroxide treatment and blocking serum, the sections were immune-stained with the primary antibody (Rabbit monoclonal anti-GFP antibody; 1:50; Cat #G10362) overnight at room temperature. Vehicle solutions contained Triton-X-100 for permeabilization. Following rinses, a biotinylated secondary antibody (anti-rabbit IgG) was applied. After further rinses, ABC solution (avidin-biotin-HRP complex; VECTASTAIN® Elite ABC, Vector Labs, Burlingame, CA) was applied. The sections were again rinsed, then treated with diaminobenzidine tetrahydrochloride (DAB) and Nickel sulphate to create a visible (black) reaction product. Following further rinses, the sections were mounted on gelatin-coated glass-slides and air dried.

FIGS. 15-24 show that capsid CNSRCV300 is able to cross the blood-brain barrier and mediate transgene expression throughout the central nervous system.

Seq ID 1235: CNSRCV300
MAADGYLPDWLEDNLSEGIREWWALKPGAPQPKANQQHQDNARGL
VLPGYKYLGPGNGLDKGEPVNAADAAALEHDKAYDQQLKAGDNPY
LKYNHADAEFQERLKEDTSFGGNLGRAVFQAKKRLLEPLGLVEEA
AKTAPGKKRPVEQSPQEPDSSAGIGKSGAQPAKKRLNFGQTGDTE
SVPDPQPIGEPPAAPSGVGSLTMASGGGAPVADNNEGADGVGSSS
GNWHCDSQWLGDRVITTSTRTWALPTYNNHLYKQISNSTSGGSSN
DNAYFGYSTPWGYFDFNRFHCHFSPRDWQRLINNNWGFRPKRLNF
KLFNIQVKEVTDNNGVKTIANNLTSTVQVFTDSDYQLPYVLGSAH
EGCLPPFPADVFMIPQYGYLTLNDGSQAVGRSSFYCLEYFPSQML
RTGNNFQFSYEFENVPFHSSYAHSQSLDRLMNPLIDQYLYYLSKT
INGSGQNQQTLKFSVAGPSNMAVQGRNYIPGPSYRQQRVSTTVTQ
NNNSEFAWPGASSWALNGRNSLMNPGPAMASHKEGEDRFFPLSGS
LIFGKQGTGRDNVDADKVMITNEEEIKTTNPVATESYGQVATNHQ
SAYVNIMDDMDQAQTGWVQNQGILPGMVWQDRDVYLQGPIWAKIP
HTDGNFHPSPLMGGFGMKHPPPQILIKNTPVPADPPTAFNKDKLN
SFITQYSTGQVSVEIEWELQKENSKRWNPEIQYTSNYYKSNNVEF
AVNTEGVYSEPRPIGTRYLTRNL

What is claimed is:

1. An adeno-associated virus (AAV) capsid protein comprising at least 3, 4, 5, 6, 7, 8, or 9 contiguous amino acids of an amino acid sequence set forth in any one of SEQ ID NO: 1-1232.

2. The AAV capsid protein of claim 1, wherein the amino acid sequence comprises at least 3, 4, 5, 6, 7, 8, or 9 contiguous amino acids of the amino acid sequence set forth in SEQ ID NO: 71 (YVNIMDDMD).

3. The AAV capsid protein of claim 1, wherein the AAV capsid protein comprises serotype AAV2 or serotype AAV9.

4. The AAV capsid protein of claim 3, wherein the serotype AAV2 comprises SEQ ID NO: 1233.

5. The AAV capsid protein of claim 4, wherein the amino acid sequence is inserted into the AAV2 capsid protein between amino acids 588 and 589.

6. (canceled)

7. The AAV capsid protein of claim 3, wherein the serotype AAV9 comprises a sequence having at least 80% sequence identity to the sequence set forth in SEQ ID NO: 1234.

8. The AAV capsid protein of claim 7, wherein the amino acid sequence is inserted into the AAV9 capsid protein between amino acids 587 and 590.

9. The AAV capsid protein of claim 3, wherein the AAV capsid protein comprises a sequence having at least 80% sequence identity to the sequence set forth in SEQ ID NO: 1235.

10. The AAV capsid protein of claim 9, wherein the AAV capsid protein comprises the sequence set forth in SEQ ID NO: 1235.

11. The AAV capsid protein of claim 1, wherein the AAV capsid protein comprises an amino acid sequence set forth in any one of SEQ ID NO: 1-1232.

12. The AAV capsid protein of claim 2, wherein the AAV capsid protein comprises an amino acid sequence set forth in SEQ ID NO: 71 (YVNIMDDMD).

13. The AAV capsid protein of claim 1, wherein the amino acid sequence is inserted into any of the parental capsids AAV1, AAV2, AAV3B, AAV5, AAV6, AAV8, AAV9, AAV3, AAV4, AAV7, AAV11, AAVrh10, AAVrh39, or AAVrh74.

14. (canceled)

15. The AAV capsid protein of claim 1, wherein the amino acid sequence is inserted into a surface exposed region of the AAV capsid.

16. An AAV capsid protein, comprising at least 3, 4, 5, 6, 7, 8, 9 or all contiguous amino acids of an amino acid sequence set forth in any of SEQ ID NO: 1-1232, wherein the amino acid sequence is inserted between amino acid positions 450 and 600 of the AAV capsid protein.

17. The AAV capsid protein of claim 16, wherein the amino acid sequence is inserted between amino acid positions 588 and 589 of the AAV capsid protein or is inserted between amino acid positions 587 and 590 of the AAV capsid protein.

18. The AAV capsid protein of claim 17, wherein the AAV capsid protein comprises a sequence having at least 80% sequence identity to the sequence forth in SEQ ID NO: 1233 or at least 80% sequence identity to the sequence set forth in SEQ ID NO: 1234.

19. The AAV capsid protein of claim 16, wherein the amino acid sequence comprises the sequence set forth in SEQ ID NO: 71 (YVNIMDDMD).

20. (canceled)

21. (canceled)

22. The AAV capsid protein of claim 17, wherein the amino acid sequence comprises SEQ ID NO: 71 (YVNIMDDMD).

23. The AAV capsid protein of claim 19, wherein the AAV capsid protein comprises a sequence having at least 80% sequence identity to the sequence set forth in SEQ ID NO: 1235.

24. (canceled)

25. A CNS-targeting molecule, comprising at least 3, 4, 5, 6, 7, 8, 9, or all contiguous amino acids of an amino acid sequence set forth in any of SEQ ID NO: 1-1232.

26. (canceled)

27. (canceled)


SEQUENCE LISTING (1236 sequences)

SEQ ID NO: 1 - protein, 9 aa, synthetic construct
  source 1..9: note = AAV9 587/590
NFRQSEMLN

SEQ ID NO: 2 - protein, 9 aa, synthetic construct
  source 1..9: note = AAV9 587/590
QRNTDHITP

SEQ ID NO: 3 - protein, 9 aa, synthetic construct
  source 1..9: note = AAV9 587/590
MQFDPQKPD

SEQ ID NO: 4 - protein, 9 aa, synthetic construct
  source 1..9: note = AAV9 587/590
TMSSNDNMR

SEQ ID NO: 5 - protein, 9 aa, synthetic construct
  source 1..9: note = AAV9 587/590
VKESVQKAA

SEQ ID NO: 6 - protein, 9 aa, synthetic construct
  source 1..9: note = AAV9 587/590
ILNVSKPDN

SEQ ID NO: 7 - protein, 9 aa, synthetic construct
  source 1..9: note = AAV9 587/590
SQAAQLPTQ

SEQ ID NO: 8 - protein, 9 aa, synthetic construct
  source 1..9: note = AAV9 587/590
QVMAIDAAI

SEQ ID NO: 9 - protein, 9 aa, synthetic construct
  source 1..9: note = AAV9 587/590
NDTARERAI

SEQ ID NO: 10 - protein, 9 aa, synthetic construct
  source 1..9: note = AAV9 587/590
SMAAKVTAE

SEQ ID NO: 11 - protein, 9 aa, synthetic construct
  source 1..9: note = AAV9 587/590
TLGHQHSPI

SEQ ID NO: 12 - protein, 9 aa, synthetic construct
  source 1..9: note = AAV9 587/590
TVPSSGKQH

SEQ ID NO: 13 - protein, 9 aa, synthetic construct
  source 1..9: note = AAV9 587/590
AMVQISTPL

SEQ ID NO: 14 - protein, 9 aa, synthetic construct
  source 1..9: note = AAV9 587/590
VQVTGTVRF

SEQ ID NO: 15 - protein, 9 aa, synthetic construct
  source 1..9: note = AAV9 587/590
MIAQKTVVH

SEQ ID NO: 16 - protein, 9 aa, synthetic construct
  source 1..9: note = AAV9 587/590
NTHFQGQPP

SEQ ID NO: 17 - protein, 9 aa, synthetic construct
  source 1..9: note = AAV9 587/590
MDMQATGKN

SEQ ID NO: 18 - protein, 9 aa, synthetic construct
  source 1..9: note = AAV9 587/590
NLAHTRQPQ

SEQ ID NO: 19 - protein, 9 aa, synthetic construct
  source 1..9: note = AAV9 587/590
LDIPPMNSS

SEQ ID NO: 20 - protein, 9 aa, synthetic construct
  source 1..9: note = AAV9 587/590
QHSTPSWPS

SEQ ID NO: 21 - protein, 16 aa, synthetic construct
  source 1..16: note = AAV2 R588A/589
AFTRHNGTGGKSIYGA

SEQ ID NO: 22 - protein, 9 aa, synthetic construct
  source 1..9: note = AAV9 587/590
ARGARDEFT

SEQ ID NO: 23 - protein, 9 aa, synthetic construct
  source 1..9: note = AAV9 587/590
RPSNDKAMM

SEQ ID NO: 24 - protein, 9 aa, synthetic construct
  source 1..9: note = AAV9 587/590
AQVHMTMPQ

SEQ ID NO: 25 - protein, 9 aa, synthetic construct
  source 1..9: note = AAV9 587/590
ARQTFQHHL

SEQ ID NO: 26 - protein, 9 aa, synthetic construct
  source 1..9: note = AAV9 587/590
HQTDAPTDW

SEQ ID NO: 27 - protein, 16 aa, synthetic construct
  source 1..16: note = AAV9 588/589
ANMISEPRISYGNDAA

SEQ ID NO: 28 - protein, 9 aa, synthetic construct
  source 1..9: note = AAV9 587/590
IERWQAEII

SEQ ID NO: 29 - protein, 9 aa, synthetic construct
  source 1..9: note = AAV9 587/590
VPHNGLHST

SEQ ID NO: 30 - protein, 9 aa, synthetic construct
  source 1..9: note = AAV9 587/590
RIHDPSLVS

SEQ ID NO: 31 - protein, 9 aa, synthetic construct
  source 1..9: note = AAV9 587/590
IPATHPQLM

SEQ ID NO: 32 - protein, 9 aa, synthetic construct
  source 1..9: note = AAV9 587/590
ITTVKNVTV

SEQ ID NO: 33 - protein, 9 aa, synthetic construct
  source 1..9: note = AAV9 587/590
DKSVKSTAI

SEQ ID NO: 34 - protein, 9 aa, synthetic construct
  source 1..9: note = AAV9 587/590
IRTHEVPLQ

SEQ ID NO: 35 - protein, 9 aa, synthetic construct
  source 1..9: note = AAV9 587/590
SPSSTSNNN

SEQ ID NO: 36 - protein, 9 aa, synthetic construct
  source 1..9: note = AAV9 587/590
LSTTSQVHN

SEQ ID NO: 37 - protein, 9 aa, synthetic construct
  source 1..9: note = AAV9 587/590
NMHNETSMT

SEQ ID NO: 38 - protein, 9 aa, synthetic construct
  source 1..9: note = AAV9 587/590
NDSRKANIG

SEQ ID NO: 39 - protein, 9 aa, synthetic construct
  source 1..9: note = AAV9 587/590
KPPNVQDAK

SEQ ID NO: 40 - protein, 9 aa, synthetic construct
  source 1..9: note = AAV9 587/590
FQHMTPKPS

SEQ ID NO: 41 - protein, 9 aa, synthetic construct
  source 1..9: note = AAV9 587/590
KQPSRLMEW

SEQ ID NO: 42 - protein, 9 aa, synthetic construct
  source 1..9: note = AAV9 587/590
VYDQVKATH

SEQ ID NO: 43 - protein, 9 aa, synthetic construct
  source 1..9: note = AAV9 587/590
SYRNQYDDQ

SEQ ID NO: 44 - protein, 9 aa, synthetic construct
  source 1..9: note = AAV9 587/590
VQSTKQSVQ

SEQ ID NO: 45 - protein, 9 aa, synthetic construct
  source 1..9: note = AAV9 587/590
HQGKVDAQN

SEQ ID NO: 46 - protein, 9 aa, synthetic construct
  source 1..9: note = AAV9 587/590
IQGEIRVHT

SEQ ID NO: 47 - protein, 9 aa, synthetic construct
  source 1..9: note = AAV9 587/590
TQTLKVEDR

SEQ ID NO: 48 - protein, 9 aa, synthetic construct
  source 1..9: note = AAV9 587/590
VQSPPGIMP

SEQ ID NO: 49 - protein, 9 aa, synthetic construct
  source 1..9: note = AAV9 587/590
VHYAPNYTT

SEQ ID NO: 50 - protein, 9 aa, synthetic construct
  source 1..9: note = AAV9 587/590
YGGSETNVR

SEQ ID NO: 51 - protein, 9 aa, synthetic construct
  source 1..9: note = AAV9 587/590
IRTQTHPQV

SEQ ID NO: 52 - protein, 9 aa, synthetic construct
  source 1..9: note = AAV9 587/590
ETQVQPIQH

SEQ ID NO: 53 - protein, 9 aa, synthetic construct
  source 1..9: note = AAV9 587/590
FPVSSVNDV

SEQ ID NO: 54 - protein, 9 aa, synthetic construct
  source 1..9: note = AAV9 587/590
IQKISQAMA

SEQ ID NO: 55 - protein, 9 aa, synthetic construct
  source 1..9: note = AAV9 587/590
SQTQIGQPY

SEQ ID NO: 56 - protein, 9 aa, synthetic construct
  source 1..9: note = AAV9 587/590
AIRTNVGPV

SEQ ID NO: 57 - protein, 9 aa, synthetic construct
  source 1..9: note = AAV9 587/590
TQMSGQTPH

SEQ ID NO: 58 - protein, 9 aa, synthetic construct
  source 1..9: note = AAV9 587/590
PAKSVMIPY

SEQ ID NO: 59 - protein, 9 aa, synthetic construct
  source 1..9: note = AAV9 587/590
QRVSQTNSF

SEQ ID NO: 60 - protein, 16 aa, synthetic construct
  source 1..16: note = AAV2 R588A/589
AFAGANYAAWAVNVAA

SEQ ID NO: 61 - protein, 9 aa, synthetic construct
  source 1..9: note = AAV9 587/590
AVVRPQSTM

SEQ ID NO: 62 - protein, 9 aa, synthetic construct
  source 1..9: note = AAV9 587/590
PLQTPPSDN

SEQ ID NO: 63 - protein, 9 aa, synthetic construct
  source 1..9: note = AAV9 587/590
VTVSQQMTK

SEQ ID NO: 64 - protein, 9 aa, synthetic construct
  source 1..9: note = AAV9 587/590
DPFEGRIST

SEQ ID NO: 65 - protein, 9 aa, synthetic construct
  source 1..9: note = AAV9 587/590
NNPTGGIAL

SEQ ID NO: 66 - protein, 16 aa, synthetic construct
  source 1..16: note = AAV2 R588A/589
ARYTHLVAIQNKEEIA

SEQ ID NO: 67 - protein, 9 aa, synthetic construct
  source 1..9: note = AAV9 587/590
IVTGGVLKP

SEQ ID NO: 68 - protein, 9 aa, synthetic construct
  source 1..9: note = AAV9 587/590
THMSTQASI

SEQ ID NO: 69 - protein, 16 aa, synthetic construct
  source 1..16: note = AAV2 R588A/589
AVPGEEEQKFEVEKYA

SEQ ID NO: 70 - protein, 9 aa, synthetic construct
  source 1..9: note = AAV9 587/590
ATDTSHLVV

SEQ ID NO: 71 - protein, 9 aa, synthetic construct
  source 1..9: note = AAV9 587/590
YVNIMDDMD

SEQ ID NO: 72 - protein, 9 aa, synthetic construct
  source 1..9: note = AAV9 587/590
IRPDKQGGV

SEQ ID NO: 73 - protein, 9 aa, synthetic construct
  source 1..9: note = AAV9 587/590
NQTESQWKG

SEQ ID NO: 74 - protein, 9 aa, synthetic construct
  source 1..9: note = AAV9 587/590
TNEGLYKDN

SEQ ID NO: 75 - protein, 9 aa, synthetic construct
  source 1..9: note = AAV9 587/590
IMVGNQTVM

SEQ ID NO: 76 - protein, 9 aa, synthetic construct
  source 1..9: note = AAV9 587/590
IWAEKGLDT

SEQ ID NO: 77 - protein, 9 aa, synthetic construct
  source 1..9: note = AAV9 587/590
MPSQGYQQV

SEQ ID NO: 78 - protein, 9 aa, synthetic construct
  source 1..9: note = AAV9 587/590
NTFGHNVAP

SEQ ID NO: 79 - protein, 16 aa, synthetic construct
  source 1..16: note = AAV2 R588A/589
ASRTVSDSIKKLITSA

SEQ ID NO: 80 - protein, 9 aa, synthetic construct
  source 1..9: note = AAV9 587/590
NHTSTPPQQ

SEQ ID NO: 81 - protein, 9 aa, synthetic construct
  source 1..9: note = AAV9 587/590
NAHAPIPVE

SEQ ID NO: 82 - protein, 9 aa, synthetic construct
  source 1..9: note = AAV9 587/590
KSTNTHEIG

SEQ ID NO: 83 - protein, 9 aa, synthetic construct
  source 1..9: note = AAV9 587/590
NTRVTNAHD

SEQ ID NO: 84 - protein, 16 aa, synthetic construct
  source 1..16: note = AAV2 R588A/589
AHRDACLTGSKIQVDA

SEQ ID NO: 85 - protein, 9 aa, synthetic construct
  source 1..9: note = AAV9 587/590
AVAAETGQI

SEQ ID NO: 86 - protein, 9 aa, synthetic construct
  source 1..9: note = AAV9 587/590
NQPSMVNGH

SEQ ID NO: 87 - protein, 9 aa, synthetic construct
  source 1..9: note = AAV9 587/590
PMTQQHHSM

SEQ ID NO: 88 - protein, 9 aa, synthetic construct
  source 1..9: note = AAV9 587/590
YEKVDSLMM

SEQ ID NO: 89 - protein, 9 aa, synthetic construct
  source 1..9: note = AAV9 587/590
MDVGTHGKA

SEQ ID NO: 90 - protein, 9 aa, synthetic construct
  source 1..9: note = AAV9 587/590
THNSQVHPS

SEQ ID NO: 91 - protein, 9 aa, synthetic construct
  source 1..9: note = AAV9 587/590
PTQVRQVQQ

SEQ ID NO: 92 - protein, 9 aa, synthetic construct
  source 1..9: note = AAV9 587/590
ITRTVNTVE

SEQ ID NO: 93 - protein, 9 aa, synthetic construct
  source 1..9: note = AAV9 587/590
AMIMSPFGT

SEQ ID NO: 94 - protein, 9 aa, synthetic construct
  source 1..9: note = AAV9 587/590
HAFPSFPAA

SEQ ID NO: 95 - protein, 9 aa, synthetic construct
  source 1..9: note = AAV9 587/590
KMMDNTPFY

SEQ ID NO: 96 - protein, 9 aa, synthetic construct
  source 1..9: note = AAV9 587/590
PQRLSVDPN

SEQ ID NO: 97 - protein, 9 aa, synthetic construct
  source 1..9: note = AAV9 587/590
TWGTYQHSV

SEQ ID NO: 98 - protein, 9 aa, synthetic construct
  source 1..9: note = AAV9 587/590
HYQQRDMSQ

SEQ ID NO: 99 - protein, 9 aa, synthetic construct
  source 1..9: note = AAV9 587/590
SQGHVQERM

SEQ ID NO: 100 - protein, 9 aa, synthetic construct
  source 1..9: note = AAV9 587/590
QPRTTGNDF

SEQ ID NO: 101 - protein, 9 aa, synthetic construct
  source 1..9: note = AAV9 587/590
PKGTISSQQ

SEQ ID NO: 102 - protein, 9 aa, synthetic construct
  source 1..9: note = AAV9 587/590
REHTAAWFA

SEQ ID NO: 103 - protein, 9 aa, synthetic construct
  source 1..9: note = AAV9 587/590
QLTHSNTSQ

SEQ ID NO: 104 - protein, 9 aa, synthetic construct
  source 1..9: note = AAV9 587/590
KDMGAGHSM

SEQ ID NO: 105 - protein, 16 aa, synthetic construct
  source 1..16: note = AAV9 588/589
ARGAQRLAALGDTAWA

SEQ ID NO: 106 - protein, 9 aa, synthetic construct
  source 1..9: note = AAV9 587/590
KQAHREHEE

SEQ ID NO: 107 - protein, 9 aa, synthetic construct
  source 1..9: note = AAV9 587/590
KSSHPYTDE

SEQ ID NO: 108 - protein, 9 aa, synthetic construct
  source 1..9: note = AAV9 587/590
MPNHVAYKD

SEQ ID NO: 109 - protein, 9 aa, synthetic construct
  source 1..9: note = AAV9 587/590
PPTTQYPQT

SEQ ID NO: 110 - protein, 9 aa, synthetic construct
  source 1..9: note = AAV9 587/590
RSHTTGQDS

SEQ ID NO: 111 - protein, 9 aa, synthetic construct
  source 1..9: note = AAV9 587/590
SQLKASVTN

SEQ ID NO: 112 - protein, 9 aa, synthetic construct
  source 1..9: note = AAV9 587/590
TAPSTRFEV

SEQ ID NO: 113 - protein, 9 aa, synthetic construct
  source 1..9: note = AAV9 587/590
PSTLEPEVW

SEQ ID NO: 114 - protein, 9 aa, synthetic construct
  source 1..9: note = AAV9 587/590
YTSPPQMMV

SEQ ID NO: 115 - protein, 9 aa, synthetic construct
  source 1..9: note = AAV9 587/590
PVPSRQVST

SEQ ID NO: 116 - protein, 9 aa, synthetic construct
  source 1..9: note = AAV9 587/590
LEKVQVHQN

SEQ ID NO: 117 - protein, 9 aa, synthetic construct
  source 1..9: note = AAV9 587/590
NQIKLQSMS

SEQ ID NO: 118 - protein, 9 aa, synthetic construct
  source 1..9: note = AAV9 587/590
GVMSPTPMV

SEQ ID NO: 119 - protein, 9 aa, synthetic construct
  source 1..9: note = AAV9 587/590
PTQRAERYF

SEQ ID NO: 120 - protein, 9 aa, synthetic construct
  source 1..9: note = AAV9 587/590
PTAKVVDSV

SEQ ID NO: 121 - protein, 9 aa, synthetic construct
  source 1..9: note = AAV9 587/590
HRIHEVQGG

SEQ ID NO: 122 - protein, 9 aa, synthetic construct
  source 1..9: note = AAV9 587/590
KNTTPVGQQ

SEQ ID NO: 123 - protein, 16 aa, synthetic construct
  source 1..16: note = AAV2 R588A/589
AGPPLKGAGGKERPGA

SEQ ID NO: 124 - protein, 9 aa, synthetic construct
  source 1..9: note = AAV9 587/590
IQPASGQNM

SEQ ID NO: 125 - protein, 9 aa, synthetic construct
  source 1..9: note = AAV9 587/590
YQNQGVNVP

SEQ ID NO: 126 - protein, 9 aa, synthetic construct
  source 1..9: note = AAV9 587/590
NASSQSNAN

SEQ ID NO: 127 - protein, 9 aa, synthetic construct
  source 1..9: note = AAV9 587/590
SNTVRGDAA

SEQ ID NO: 128 - protein, 9 aa, synthetic construct
  source 1..9: note = AAV9 587/590
SKKAEFGTE

SEQ ID NO: 129 - protein, 9 aa, synthetic construct
  source 1..9: note = AAV9 587/590
KVSSEMMVH

SEQ ID NO: 130 - protein, 9 aa, synthetic construct
  source 1..9: note = AAV9 587/590
TMIEQRMDQ

SEQ ID NO: 131 - protein, 9 aa, synthetic construct
  source 1..9: note = AAV9 587/590
PPHIYHHMS

SEQ ID NO: 132 - protein, 9 aa, synthetic construct
  source 1..9: note = AAV9 587/590
MVNKAPTGA

SEQ ID NO: 133 - protein, 9 aa, synthetic construct
  source 1..9: note = AAV9 587/590
KSPVARGDT

SEQ ID NO: 134 - protein, 9 aa, synthetic construct
  source 1..9: note = AAV9 587/590
LLKQRQLMD

SEQ ID NO: 135 - protein, 9 aa, synthetic construct
  source 1..9: note = AAV9 587/590
KMKQIELVE

SEQ ID NO: 136 - protein, 9 aa, synthetic construct
  source 1..9: note = AAV9 587/590
RAPNRVNDE

SEQ ID NO: 137 - protein, 9 aa, synthetic construct
  source 1..9: note = AAV9 587/590
TMNQAPPAQ

SEQ ID NO: 138 - protein, 9 aa, synthetic construct
  source 1..9: note = AAV9 587/590
LVKEDEHWI

SEQ ID NO: 139 - protein, 9 aa, synthetic construct
  source 1..9: note = AAV9 587/590
MIGQRDNNS

SEQ ID NO: 140 - protein, 9 aa, synthetic construct
  source 1..9: note = AAV9 587/590
TSHMVSHAV

SEQ ID NO: 141 - protein, 9 aa, synthetic construct
  source 1..9: note = AAV9 587/590
IQDEEAKYT

SEQ ID NO: 142 - protein, 9 aa, synthetic construct
  source 1..9: note = AAV9 587/590
TGGSKFPYM

SEQ ID NO: 143 - protein, 9 aa, synthetic construct
  source 1..9: note = AAV9 587/590
VEGSAQMIK

SEQ ID NO: 144 - protein, 9 aa, synthetic construct
  source 1..9: note = AAV9 587/590
FTDISKEPS

SEQ ID NO: 145 - protein, 9 aa, synthetic construct
  source 1..9: note = AAV9 587/590
TERPMMQQL

SEQ ID NO: 146 - protein, 9 aa, synthetic construct
  source 1..9: note = AAV9 587/590
VSLGHDKPV

SEQ ID NO: 147 - protein, 9 aa, synthetic construct
  source 1..9: note = AAV9 587/590
TQRTHISDM

SEQ ID NO: 148 - protein, 9 aa, synthetic construct
  source 1..9: note = AAV9 587/590
TSYSTHASS

SEQ ID NO: 149 - protein, 9 aa, synthetic construct
  source 1..9: note = AAV9 587/590
NTKSADHEY

SEQ ID NO: 150 - protein, 9 aa, synthetic construct
  source 1..9: note = AAV9 587/590
RDPLREQRP

SEQ ID NO: 151 - protein, 9 aa, synthetic construct
  source 1..9: note = AAV9 587/590
LGQRTVQAQ

SEQ ID NO: 152 - protein, 9 aa, synthetic construct
  source 1..9: note = AAV9 587/590
VHDMTHQVN

SEQ ID NO: 153 - protein, 9 aa, synthetic construct
  source 1..9: note = AAV9 587/590
DYPEYHDIW

SEQ ID NO: 154 - protein, 9 aa, synthetic construct
  source 1..9: note = AAV9 587/590
QTQQGNYPK

SEQ ID NO: 155 - protein, 9 aa, synthetic construct
  source 1..9: note = AAV9 587/590
QASVGTVRV

SEQ ID NO: 156 - protein, 9 aa, synthetic construct
  source 1..9: note = AAV9 587/590
SPAHQRGVD

SEQ ID NO: 157 - protein, 9 aa, synthetic construct
  source 1..9: note = AAV9 587/590
QMYGEKSVS

SEQ ID NO: 158 - protein, 9 aa, synthetic construct
  source 1..9: note = AAV9 587/590
NTAVQRGSV

SEQ ID NO: 159 - protein, 9 aa, synthetic construct
  source 1..9: note = AAV9 587/590
KTIQQTQDV

SEQ ID NO: 160 - protein, 9 aa, synthetic construct
  source 1..9: note = AAV9 587/590
QMQTLGDDI

SEQ ID NO: 161 - protein, 9 aa, synthetic construct
  source 1..9: note = AAV9 587/590
SHTGQTREL

SEQ ID NO: 162 - protein, 9 aa, synthetic construct
  source 1..9: note = AAV9 587/590
KRQNTESAF

SEQ ID NO: 163 - protein, 9 aa, synthetic construct
  source 1..9: note = AAV9 587/590
NFDPLSVAE

SEQ ID NO: 164 - protein, 9 aa, synthetic construct
  source 1..9: note = AAV9 587/590
KILQQEPLM

SEQ ID NO: 165 - protein, 9 aa, synthetic construct
  source 1..9: note = AAV9 587/590
IVHAVIQST

SEQ ID NO: 166 - protein, 9 aa, synthetic construct
  source 1..9: note = AAV9 587/590
TMDHNPIQT

SEQ ID NO: 167 - protein, 9 aa, synthetic construct
  source 1..9: note = AAV9 587/590
KQPTPTNLW

SEQ ID NO: 168 - protein, 9 aa, synthetic construct
  source 1..9: note = AAV9 587/590
EQGNPGVSI

SEQ ID NO: 169 - protein, 9 aa, synthetic construct
  source 1..9: note = AAV9 587/590
IHSRVQQVT

SEQ ID NO: 170 - protein, 9 aa, synthetic construct
  source 1..9: note = AAV9 587/590
PQSQYRPAN

SEQ ID NO: 171 - protein, 9 aa, synthetic construct
  source 1..9: note = AAV9 587/590
YRRDQENAI

SEQ ID NO: 172 - protein, 9 aa, synthetic construct
  source 1..9: note = AAV9 587/590
TANDAIVAT

SEQ ID NO: 173 - protein, 16 aa, synthetic construct
  source 1..16: note = AAV9 588/589
ALYVGSKTKEGVVHGA

SEQ ID NO: 174 - protein, 9 aa, synthetic construct
  source 1..9: note = AAV9 587/590
MASTNVGTS

SEQ ID NO: 175 - protein, 9 aa, synthetic construct
  source 1..9: note = AAV9 587/590
AIQASDTYI

SEQ ID NO: 176 - protein, 9 aa, synthetic construct
  source 1..9: note = AAV9 587/590
NMQPRIVNA

SEQ ID NO: 177 - protein, 9 aa, synthetic construct
  source 1..9: note = AAV9 587/590
QSDKEQFKH

SEQ ID NO: 178 - protein, 9 aa, synthetic construct
  source 1..9: note = AAV9 587/590
SVINQQAKA

SEQ ID NO: 179 - protein, 9 aa, synthetic construct
  source 1..9: note = AAV9 587/590
NVPGSLKIQ

SEQ ID NO: 180 - protein, 9 aa, synthetic construct
  source 1..9: note = AAV9 587/590
RKDNIPMMV

SEQ ID NO: 181 - protein, 9 aa, synthetic construct
  source 1..9: note = AAV9 587/590
NKVHGTIQD

SEQ ID NO: 182 - protein, 9 aa, synthetic construct
  source 1..9: note = AAV9 587/590
SRKDKSEAE

SEQ ID NO: 183 - protein, 9 aa, synthetic construct
  source 1..9: note = AAV9 587/590
HAQASQTQF

SEQ ID NO: 184 - protein, 9 aa, synthetic construct
  source 1..9: note = AAV9 587/590
HLGHHDGAA

SEQ ID NO: 185 - protein, 9 aa, synthetic construct
  source 1..9: note = AAV9 587/590
QPQMGVMVQ

SEQ ID NO: 186 - protein, 9 aa, synthetic construct
  source 1..9: note = AAV9 588/589
ANLVRNIDA

SEQ ID NO: 187 - protein, 9 aa, synthetic construct
  source 1..9: note = AAV9 587/590
SDIKKRNAV

SEQ ID NO: 188 - protein, 9 aa, synthetic construct
  source 1..9: note = AAV9 587/590
RVMTNSIDV

SEQ ID NO: 189 - protein, 9 aa, synthetic construct
  source 1..9: note = AAV9 587/590
KHQQQLAEK

SEQ ID NO: 190 - protein, 9 aa, synthetic construct
  source 1..9: note = AAV9 587/590
QQTPQTHPV

SEQ ID NO: 191 - protein, 9 aa, synthetic construct
  source 1..9: note = AAV9 587/590
IHNAHVPHQ

SEQ ID NO: 192 - protein, 9 aa, synthetic construct
  source 1..9: note = AAV9 587/590
VSRTPHTDF

SEQ ID NO: 193 - protein, 9 aa, synthetic construct
  source 1..9: note = AAV9 587/590
MQMKGQQPP

SEQ ID NO: 194 - protein, 9 aa, synthetic construct
  source 1..9: note = AAV9 587/590
HQSGQVPNI

SEQ ID NO: 195 - protein, 9 aa, synthetic construct
  source 1..9: note = AAV9 587/590
QLTGVASGH

SEQ ID NO: 196 - protein, 9 aa, synthetic construct
  source 1..9: note = AAV9 587/590
PEQSPQMMF

SEQ ID NO: 197 - protein, 9 aa, synthetic construct
  source 1..9: note = AAV9 587/590
NTTPQASLM

SEQ ID NO: 198 - protein, 9 aa, synthetic construct
  source 1..9: note = AAV9 587/590
DEFGAYMPI

SEQ ID NO: 199 - protein, 9 aa, synthetic construct
  source 1..9: note = AAV9 587/590
PKLIQQVNT

SEQ ID NO: 200 - protein, 9 aa, synthetic construct
  source 1..9: note = AAV9 587/590
KPGPAQVQW

SEQ ID NO: 201 - protein, 9 aa, synthetic construct
  source 1..9: note = AAV9 587/590
PLKDVQLSH

SEQ ID NO: 202 - protein, 9 aa, synthetic construct
  source 1..9: note = AAV9 587/590
KDPFYRSEN

SEQ ID NO: 203 - protein, 9 aa, synthetic construct
  source 1..9: note = AAV9 587/590
SQPGNQLKM

SEQ ID NO: 204 - protein, 9 aa, synthetic construct
  source 1..9: note = AAV9 587/590
MQNTMRLQD

SEQ ID NO: 205 - protein, 9 aa, synthetic construct
  source 1..9: note = AAV9 587/590
ILVKNKDEI

SEQ ID NO: 206 - protein, 9 aa, synthetic construct
  source 1..9: note = AAV9 587/590
PVHGQLQGQ

SEQ ID NO: 207 - protein, 9 aa, synthetic construct
  source 1..9: note = AAV9 587/590
PTLPGVGMR

SEQ ID NO: 208 - protein, 9 aa, synthetic construct
  source 1..9: note = AAV9 587/590
PPQAAQITT

SEQ ID NO: 209 - protein, 9 aa, synthetic construct
  source 1..9: note = AAV9 587/590
TKVQQQDSW

SEQ ID NO: 210 - protein, 9 aa, synthetic construct
  source 1..9: note = AAV9 587/590
IMTSHTVAV

SEQ ID NO: 211 - protein, 9 aa, synthetic construct
  source 1..9: note = AAV9 587/590
HPHMAGQQM

SEQ ID NO: 212 - protein, 9 aa, synthetic construct
  source 1..9: note = AAV9 587/590
PVQTQHAVV

SEQ ID NO: 213 - protein, 9 aa, synthetic construct
  source 1..9: note = AAV9 587/590
QIGHKQEYN

SEQ ID NO: 214 - protein, 9 aa, synthetic construct
  source 1..9: note = AAV9 587/590
HEQQIRRDT

SEQ ID NO: 215 - protein, 9 aa, synthetic construct
  source 1..9: note = AAV9 587/590
DRFKTVQNE

SEQ ID NO: 216 - protein, 9 aa, synthetic construct
  source 1..9: note = AAV9 587/590
AAFQQTDIT

SEQ ID NO: 217 - protein, 9 aa, synthetic construct
  source 1..9: note = AAV9 587/590
ERSAVVMIA

SEQ ID NO: 218 - protein, 9 aa, synthetic construct
  source 1..9: note = AAV9 587/590
QYIQGRQPI

SEQ ID NO: 219 - protein, 9 aa, synthetic construct
  source 1..9: note = AAV9 587/590
LREHAYPDV

SEQ ID NO: 220 - protein, 9 aa, synthetic construct
  source 1..9: note = AAV9 587/590
KLMDQGVTA

SEQ ID NO: 221 - protein, 9 aa, synthetic construct
  source 1..9: note = AAV9 587/590
TSQKQATHT

SEQ ID NO: 222 - protein, 9 aa, synthetic construct
  source 1..9: note = AAV9 587/590
VPNNKNPQV

SEQ ID NO: 223 - protein, 9 aa, synthetic construct
  source 1..9: note = AAV9 587/590
NVVIVMIFM

SEQ ID NO: 224 - protein, 16 aa, synthetic construct
  source 1..16: note = AAV2 R588A/589
AISYRGTWSTAESGAA

SEQ ID NO: 225 - protein, 9 aa, synthetic construct
  source 1..9: note = AAV9 587/590
NTTSQTGNV

SEQ ID NO: 226 - protein, 9 aa, synthetic construct
  source 1..9: note = AAV9 587/590
PQESQPENE

SEQ ID NO: 227 - protein, 9 aa, synthetic construct
  source 1..9: note = AAV9 587/590
TMGSRQTPI

SEQ ID NO: 228 - protein, 9 aa, synthetic construct
  source 1..9: note = AAV9 587/590
TQVVSGQEI

SEQ ID NO: 229 - protein, 9 aa, synthetic construct
  source 1..9: note = AAV9 587/590
PEELKSKQT

SEQ ID NO: 230 - protein, 9 aa, synthetic construct
  source 1..9: note = AAV9 587/590
INKYSQYTT

SEQ ID NO: 231 - protein, 9 aa, synthetic construct
  source 1..9: note = AAV9 587/590
QPTQGTLRA

SEQ ID NO: 232 - protein, 9 aa, synthetic construct
  source 1..9: note = AAV9 587/590
NQVHTKDDS

SEQ ID NO: 233 - protein, 9 aa, synthetic construct
  source 1..9: note = AAV9 587/590
YITQIHEDD

SEQ ID NO: 234 - protein, 9 aa, synthetic construct
  source 1..9: note = AAV9 587/590
HIGGSGNTM

SEQ ID NO: 235 - protein, 9 aa, synthetic construct
  source 1..9: note = AAV9 587/590
HQNHPEHAV

SEQ ID NO: 236 - protein, 9 aa, synthetic construct
  source 1..9: note = AAV9 587/590
LKGSVQQAP

SEQ ID NO: 237 - protein, 9 aa, synthetic construct
  source 1..9: note = AAV9 587/590
PLIPRQEVI

SEQ ID NO: 238 - protein, 9 aa, synthetic construct
  source 1..9: note = AAV9 587/590
TLVTQSDSS

SEQ ID NO: 239 - protein, 9 aa, synthetic construct
  source 1..9: note = AAV9 587/590
LKIDVTKIT

SEQ ID NO: 240 - protein, 9 aa, synthetic construct
  source 1..9: note = AAV9 587/590
PSHVGFTSV

SEQ ID NO: 241 - protein, 9 aa, synthetic construct
  source 1..9: note = AAV9 587/590
LYRDQGNIT

SEQ ID NO: 242 - protein, 9 aa, synthetic construct
  source 1..9: note = AAV9 587/590
SVKQMDATH

SEQ ID NO: 243 - protein, 9 aa, synthetic construct
  source 1..9: note = AAV9 587/590
QFDRVAQDK

SEQ ID NO: 244 - protein, 9 aa, synthetic construct
  source 1..9: note = AAV9 587/590
YPTGTPPAT

SEQ ID NO: 245 - protein, 9 aa, synthetic construct
  source 1..9: note = AAV9 587/590
DRVQSTSPH

SEQ ID NO: 246 - protein, 9 aa, synthetic construct
  source 1..9: note = AAV9 587/590
KTQKPYQED

SEQ ID NO: 247 - protein, 9 aa, synthetic construct
  source 1..9: note = AAV9 587/590
SQVAEQRGI

SEQ ID NO: 248 - protein, 9 aa, synthetic construct
  source 1..9: note = AAV9 587/590
SESMHGSVS

SEQ ID NO: 249 - protein, 9 aa, synthetic construct
  source 1..9: note = AAV9 587/590
TITDQQSHG

SEQ ID NO: 250 - protein, 9 aa, synthetic construct
  source 1..9: note = AAV9 587/590
TRHVVTGGY

SEQ ID NO: 251 - protein, 9 aa, synthetic construct
  source 1..9: note = AAV9 587/590
QQNMKLQVY

SEQ ID NO: 252 - protein, 9 aa, synthetic construct
  source 1..9: note = AAV9 587/590
SKFTYYDPW

SEQ ID NO: 253 - protein, 9 aa, synthetic construct
  source 1..9: note = AAV9 587/590
MAVQHTTNQ

SEQ ID NO: 254 - protein, 9 aa, synthetic construct
  source 1..9: note = AAV9 587/590
SSMQGASGV

SEQ ID NO: 255 - protein, 9 aa, synthetic construct
  source 1..9: note = AAV9 587/590
MKYSTQTQS

SEQ ID NO: 256 - protein, 9 aa, synthetic construct
  source 1..9: note = AAV9 587/590
IKQPQSFAQ

SEQ ID NO: 257 - protein, 9 aa, synthetic construct
  source 1..9: note = AAV9 587/590
TPVAQANKN

SEQ ID NO: 258 - protein, 9 aa, synthetic construct
  source 1..9: note = AAV9 587/590
QDVMTSPMQ

SEQ ID NO: 259 - protein, 9 aa, synthetic construct
  source 1..9: note = AAV9 587/590
PQVLGQDEA

SEQ ID NO: 260 - protein, 9 aa, synthetic construct
  source 1..9: note = AAV9 587/590
TDTVDTGSM

SEQ ID NO: 261 - protein, 16 aa, synthetic construct
  source 1..16: note = AAV9 588/589
AQEGARQKLHELQEKA

SEQ ID NO: 262 - protein, 9 aa, synthetic construct
  source 1..9: note = AAV9 587/590
ATSEYGRAY

SEQ ID NO: 263 - protein, 9 aa, synthetic construct
  source 1..9: note = AAV9 587/590
LKGPQQQAI

SEQ ID NO: 264 - protein, 9 aa, synthetic construct
  source 1..9: note = AAV9 587/590
VFREAGNVH

SEQ ID NO: 265 - protein, 9 aa, synthetic construct
  source 1..9: note = AAV9 587/590
PKQVSDIMF

SEQ ID NO: 266 - protein, 9 aa, synthetic construct
  source 1..9: note = AAV9 587/590
HMMDGGVPY

SEQ ID NO: 267 - protein, 9 aa, synthetic construct
  source 1..9: note = AAV9 587/590
IRAASTGHM

SEQ ID NO: 268 - protein, 9 aa, synthetic construct
  source 1..9: note = AAV9 587/590
PDGYHVEMF

SEQ ID NO: 269 - protein, 9 aa, synthetic construct
  source 1..9: note = AAV9 587/590
QAQSNNIYF

SEQ ID NO: 270 - protein, 9 aa, synthetic construct
  source 1..9: note = AAV9 587/590
TMNGQSKNF

SEQ ID NO: 271 - protein, 9 aa, synthetic construct
  source 1..9: note = AAV9 587/590
HQARREVEY

SEQ ID NO: 272 - protein, 9 aa, synthetic construct
  source 1..9: note = AAV9 587/590
GDQGRVVSH

SEQ ID NO: 273 - protein, 16 aa, synthetic construct
  source 1..16: note = AAV2 R588A/589
ACCPLAEESDNVDSAA

SEQ ID NO: 274 - protein, 9 aa, synthetic construct
  source 1..9: note = AAV9 587/590
QQNQSIFNI

SEQ ID NO: 275 - protein, 9 aa, synthetic construct
  source 1..9: note = AAV9 587/590
TSDAVKTDQ

SEQ ID NO: 276 - protein, 9 aa, synthetic construct
  source 1..9: note = AAV9 587/590
TQAPDVLPV

SEQ ID NO: 277 - protein, 9 aa, synthetic construct
  source 1..9: note = AAV9 587/590
TTDTAKATY

SEQ ID NO: 278 - protein, 9 aa, synthetic construct
  source 1..9: note = AAV9 587/590
IDEHSEHYV

SEQ ID NO: 279 - protein, 9 aa, synthetic construct
  source 1..9: note = AAV9 587/590
PINSRIVDP

SEQ ID NO: 280 - protein, 9 aa, synthetic construct
  source 1..9: note = AAV9 587/590
PDQRGHMYA

SEQ ID NO: 281 - protein, 9 aa, synthetic construct
  source 1..9: note = AAV9 587/590
FLMSHSEVQ

SEQ ID NO: 282 - protein, 9 aa, synthetic construct
  source 1..9: note = AAV9 587/590
FIQQSNERN

SEQ ID NO: 283 - protein, 9 aa, synthetic construct
  source 1..9: note = AAV9 587/590
NPTRDAKPP

SEQ ID NO: 284 - protein, 9 aa, synthetic construct
  source 1..9: note = AAV9 587/590
RWGRAQEDE

SEQ ID NO: 285 - protein, 9 aa, synthetic construct
  source 1..9: note = AAV9 587/590
PRNMQPMTD

SEQ ID NO: 286 - protein, 9 aa, synthetic construct
  source 1..9: note = AAV9 587/590
MVSQREINQ

SEQ ID NO: 287 - protein, 9 aa, synthetic construct
  source 1..9: note = AAV9 587/590
MSSSTRMAV

SEQ ID NO: 288 - protein, 9 aa, synthetic construct
  source 1..9: note = AAV9 587/590
PLNQHQKIS

SEQ ID NO: 289 - protein, 9 aa, synthetic construct
  source 1..9: note = AAV9 587/590
DNASHVHMQ

SEQ ID NO: 290 - protein, 9 aa, synthetic construct
  source 1..9: note = AAV9 587/590
DMKRVDYMQ

SEQ ID NO: 291 - protein, 9 aa, synthetic construct
  source 1..9: note = AAV9 587/590
NHLVDRTTQ

SEQ ID NO: 292 - protein, 9 aa, synthetic construct
  source 1..9: note = AAV9 587/590
VIQASSITA

SEQ ID NO: 293 - protein, 9 aa, synthetic construct
  source 1..9: note = AAV9 587/590
AQAMTNKNK

SEQ ID NO: 294 - protein, 9 aa, synthetic construct
  source 1..9: note = AAV9 587/590
INFKQEVHQ

SEQ ID NO: 295 - protein, 9 aa, synthetic construct
  source 1..9: note = AAV9 587/590
AYAQLPHEK

SEQ ID NO: 296 - protein, 16 aa, synthetic construct
  source 1..16: note = AAV2 R588A/589
AVTIQNWCKRGRKQCA

SEQ ID NO: 297 - protein, 9 aa, synthetic construct
  source 1..9: note = AAV9 587/590
NPQNSGMAM

SEQ ID NO: 298 - protein, 9 aa, synthetic construct
  source 1..9: note = AAV9 587/590
SRDRDTAYA

SEQ ID NO: 299 - protein, 9 aa, synthetic construct
  source 1..9: note = AAV9 587/590
PHNSPGMTR

SEQ ID NO: 300 - protein, 9 aa, synthetic construct
  source 1..9: note = AAV9 587/590
KMMPSQMQD

SEQ ID NO: 301 - protein, 9 aa, synthetic construct
  source 1..9: note = AAV9 587/590
NHAPVHERM

SEQ ID NO: 302 - protein, 9 aa, synthetic construct
  source 1..9: note = AAV9 587/590
RALKQIDAD

SEQ ID NO: 303 - protein, 9 aa, synthetic construct
  source 1..9: note = AAV9 587/590
QRVEGFIQD

SEQ ID NO: 304 - protein, 16 aa, synthetic construct
  source 1..16: note = AAV2 R588A/589
AKTDSCQGDSGGPLVA

SEQ ID NO: 305 - protein, 9 aa, synthetic construct
  source 1..9: note = AAV9 587/590
TSASVQSAM

SEQ ID NO: 306 - protein, 9 aa, synthetic construct
  source 1..9: note = AAV9 587/590
NVTTHAPTI

SEQ ID NO: 307 - protein, 9 aa, synthetic construct
  source 1..9: note = AAV9 587/590
RVQTPDHDH

SEQ ID NO: 308 - protein, 9 aa, synthetic construct
  source 1..9: note = AAV9 587/590
QVTKEFASF

SEQ ID NO: 309 - protein, 9 aa, synthetic construct
  source 1..9: note = AAV9 587/590
ASVTPQYLY

SEQ ID NO: 310 - protein, 9 aa, synthetic construct
  source 1..9: note = AAV9 587/590
VTQKTDNTL

SEQ ID NO: 311 - protein, 9 aa, synthetic construct
  source 1..9: note = AAV9 587/590
VKMGIQAIA

SEQ ID NO: 312 - protein, 9 aa, synthetic construct
  source 1..9: note = AAV9 587/590
PLDNYLASM

SEQ ID NO: 313 - protein, 9 aa, synthetic construct
  source 1..9: note = AAV9 587/590
DENGGVPRI

SEQ ID NO: 314 - protein, 9 aa, synthetic construct
  source 1..9: note = AAV9 587/590
HWQQPSIDG

SEQ ID NO: 315 - protein, 9 aa, synthetic construct
  source 1..9: note = AAV9 587/590
TPSQGSHGS

SEQ ID NO: 316 - protein, 9 aa, synthetic construct
  source 1..9: note = AAV9 587/590
HNGMNQVDK

SEQ ID NO: 317 - protein, 9 aa, synthetic construct
  source 1..9: note = AAV9 587/590
PHMHNMATP

SEQ ID NO: 318 - protein, 9 aa, synthetic construct
  source 1..9: note = AAV9 587/590
IQVRDRTPE

SEQ ID NO: 319 - protein, 9 aa, synthetic construct
  source 1..9: note = AAV9 587/590
APDAHTRMP

SEQ ID NO: 320 - protein, 9 aa, synthetic construct
  source 1..9: note = AAV9 587/590
GAVSHHQMI

SEQ ID NO: 321 - protein, 9 aa, synthetic construct
  source 1..9: note = AAV9 587/590
NVQRTGVGN

SEQ ID NO: 322 - protein, 9 aa, synthetic construct
  source 1..9: note = AAV9 587/590
PDMLQGKMY

SEQ ID NO: 323 - protein, 9 aa, synthetic construct
  source 1..9: note = AAV9 587/590
NYFASSDQM

SEQ ID NO: 324 - protein, 9 aa, synthetic construct
  source 1..9: note = AAV9 587/590
PRQGSLAHM

SEQ ID NO: 325 - protein, 9 aa, synthetic construct
  source 1..9: note = AAV9 587/590
SQQVSKQMQ

SEQ ID NO: 326 - protein, 9 aa, synthetic construct
  source 1..9: note = AAV9 587/590
KRNQEGNGV

SEQ ID NO: 327 - protein, 9 aa, synthetic construct
  source 1..9: note = AAV9 587/590
PMHSSHNDK

SEQ ID NO: 328 - protein, 9 aa, synthetic construct
  source 1..9: note = AAV9 587/590
TLQPQHIAK

SEQ ID NO: 329 - protein, 9 aa, synthetic construct
  source 1..9: note = AAV9 587/590
SAGVVISVV

SEQ ID NO: 330 - protein, 9 aa, synthetic construct
  source 1..9: note = AAV9 587/590
PLVPRNTPQ

SEQ ID NO: 331 - protein, 9 aa, synthetic construct
  source 1..9: note = AAV9 587/590
PNQTQMLNK

SEQ ID NO: 332 - protein, 9 aa, synthetic construct
  source 1..9: note = AAV9 587/590
PMSSEYRPS

SEQ ID NO: 333 - protein, 9 aa, synthetic construct
  source 1..9: note = AAV9 587/590
ESAHSEFKV

SEQ ID NO: 334 - protein, 9 aa, synthetic construct
  source 1..9: note = AAV9 587/590
PQRVQEQGT

SEQ ID NO: 335 - protein, 9 aa, synthetic construct
  source 1..9: note = AAV9 587/590
MVQQSLKPA

SEQ ID NO: 336 - protein, 9 aa, synthetic construct
  source 1..9: note = AAV9 587/590
EQQPAQYHG

SEQ ID NO: 337 - protein, 9 aa, synthetic construct
  source 1..9: note = AAV9 587/590
TRNAQTTPL

SEQ ID NO: 338 - protein, 9 aa, synthetic construct
  source 1..9: note = AAV9 587/590
PARSGVQIV

SEQ ID NO: 339 - protein, 9 aa, synthetic construct
  source 1..9: note = AAV9 587/590
DVFTRQSEG

SEQ ID NO: 340 - protein, 9 aa, synthetic construct
  source 1..9: note = AAV9 587/590
SMHTHQGGQ

SEQ ID NO: 341 - protein, 9 aa, synthetic construct
  source 1..9: note = AAV9 587/590
VNQNVRMED

SEQ ID NO: 342 - protein, 9 aa, synthetic construct
  source 1..9: note = AAV9 587/590
LPQAKMADM

SEQ ID NO: 343 - protein, 9 aa, synthetic construct
  source 1..9: note = AAV9 587/590
RMMDNSIHT

SEQ ID NO: 344 - protein, 9 aa, synthetic construct
  source 1..9: note = AAV9 587/590
MEATAEHRI

SEQ ID NO: 345 - protein, 9 aa, synthetic construct
  source 1..9: note = AAV9 587/590
DARAHTQQV

SEQ ID NO: 346 - protein, 9 aa, synthetic construct
  source 1..9: note = AAV9 587/590
IKDMEPQLH

SEQ ID NO: 347 - protein, 16 aa, synthetic construct
  source 1..16: note = AAV9 588/589
ALITILGTVKPNANRA

SEQ ID NO: 348 - protein, 9 aa, synthetic construct
  source 1..9: note = AAV9 587/590
NHQARTGEF

SEQ ID NO: 349 - protein, 9 aa, synthetic construct
  source 1..9: note = AAV9 587/590
PKNHPVSAV

SEQ ID NO: 350 - protein, 9 aa, synthetic construct
  source 1..9: note = AAV9 587/590
KFPTEDKYI

SEQ ID NO: 351 - protein, 9 aa, synthetic construct
  source 1..9: note = AAV9 587/590
SPVATQKMV

SEQ ID NO: 352 - protein, 9 aa, synthetic construct
  source 1..9: note = AAV9 587/590
DLAEHTRFK

SEQ ID NO: 353 - protein, 9 aa, synthetic construct
  source 1..9: note = AAV9 587/590
SNTQPRPQQ

SEQ ID NO: 354 - protein, 9 aa, synthetic construct
  source 1..9: note = AAV9 587/590
TIDITPRKL

SEQ ID NO: 355 - protein, 9 aa, synthetic construct
  source 1..9: note = AAV9 587/590
PAFVDENPP

SEQ ID NO: 356 - protein, 9 aa, synthetic construct
  source 1..9: note = AAV9 587/590
VSQETRTTI

SEQ ID NO: 357 - protein, 9 aa, synthetic construct
  source 1..9: note = AAV9 587/590
EHTGGQFRL

SEQ ID NO: 358 - protein, 9 aa, synthetic construct
  source 1..9: note = AAV9 587/590
MLGKSDQVW

SEQ ID NO: 359 - protein, 9 aa, synthetic construct
  source 1..9: note = AAV9 587/590
PQRDMVNMK

SEQ ID NO: 360 - protein, 9 aa, synthetic construct
  source 1..9: note = AAV9 587/590
NSRPGAAMA

SEQ ID NO: 361 - protein, 9 aa, synthetic construct
  source 1..9: note = AAV9 587/590
TQQAPHNAY

SEQ ID NO: 362 - protein, 9 aa, synthetic construct
  source 1..9: note = AAV9 587/590
PNITGGAIM

SEQ ID NO: 363 - protein, 9 aa, synthetic construct
  source 1..9: note = AAV9 587/590
PDQKMMFKL

SEQ ID NO: 364 - protein, 9 aa, synthetic construct
  source 1..9: note = AAV9 587/590
LKHATVEST

SEQ ID NO: 365 - protein, 9 aa, synthetic construct
  source 1..9: note = AAV9 587/590
QLSRDTRQD

SEQ ID NO: 366 - protein, 9 aa, synthetic construct
  source 1..9: note = AAV9 587/590
PDSKPHAYH

SEQ ID NO: 367 - protein, 9 aa, synthetic construct
  source 1..9: note = AAV9 587/590
MQPPAQQRT

SEQ ID NO: 368 - protein, 9 aa, synthetic construct
  source 1..9: note = AAV9 587/590
VTIFDMFDQ

SEQ ID NO: 369 - protein, 9 aa, synthetic construct
  source 1..9: note = AAV9 587/590
IMAYTGQPV

SEQ ID NO: 370 - protein, 9 aa, synthetic construct
  source 1..9: note = AAV9 587/590
RTEYEMSTR

SEQ ID NO: 371 - protein, 9 aa, synthetic construct
  source 1..9: note = AAV9 587/590
QAQRKTEFI

SEQ ID NO: 372 - protein, 9 aa, synthetic construct
  source 1..9: note = AAV9 587/590
QGNETKPNS

SEQ ID NO: 373 - protein, 9 aa, synthetic construct
  source 1..9: note = AAV9 587/590
RMASQGQDM

SEQ ID NO: 374 - protein, 9 aa, synthetic construct
  source 1..9: note = AAV9 587/590
PKDGVPQMQ

SEQ ID NO: 375 - protein, 9 aa, synthetic construct
  source 1..9: note = AAV9 587/590
TIAPAIANL

SEQ ID NO: 376 - protein, 9 aa, synthetic construct
  source 1..9: note = AAV9 587/590
RTHQSSPDS

SEQ ID NO: 377 - protein, 9 aa, synthetic construct
  source 1..9: note = AAV9 587/590
PTTDMQSNI

SEQ ID NO: 378 - protein, 9 aa, synthetic construct
  source 1..9: note = AAV9 587/590
HATSQKYET

SEQ ID NO: 379 - protein, 9 aa, synthetic construct
  source 1..9: note = AAV9 588/589
ALGQARSDA

SEQ ID NO: 380 - protein, 9 aa, synthetic construct
  source 1..9: note = AAV9 587/590
PIATAYKTG

SEQ ID NO: 381 - protein, 9 aa, synthetic construct
  source 1..9: note = AAV9 587/590
HFKPTQDTS

SEQ ID NO: 382 - protein, 9 aa, synthetic construct
  source 1..9: note = AAV9 587/590
RPNTKAMQE

SEQ ID NO: 383 - protein, 9 aa, synthetic construct
  source 1..9: note = AAV9 587/590
NMPQGRVAM

SEQ ID NO: 384 - protein, 9 aa, synthetic construct
  source 1..9: note = AAV9 587/590
KTINTPNIL

SEQ ID NO: 385 - protein, 9 aa, synthetic construct
  source 1..9: note = AAV9 587/590
SRIQHDGAQ

SEQ ID NO: 386 - protein, 9 aa, synthetic construct
  source 1..9: note = AAV9 587/590
HPPMPNNTK

SEQ ID NO: 387 - protein, 9 aa, synthetic construct
  source 1..9: note = AAV9 587/590
RIMPQMQHD

SEQ ID NO: 388 - protein, 16 aa, synthetic construct
  source 1..16: note = AAV2 R588A/589
AVILELDKTVEHLPTA

SEQ ID NO: 389 - protein, 9 aa, synthetic construct
  source 1..9: note = AAV9 587/590
HSTARSGEF

SEQ ID NO: 390 - protein, 9 aa, synthetic construct
  source 1..9: note = AAV9 587/590
SQPQHMNVS

SEQ ID NO: 391 - protein, 9 aa, synthetic construct
  source 1..9: note = AAV9 587/590
PGGSYSHVV

SEQ ID NO: 392 - protein, 9 aa, synthetic construct
  source 1..9: note = AAV9 587/590
TVTHAHPTT

SEQ ID NO: 393 - protein, 9 aa, synthetic construct
  source 1..9: note = AAV9 587/590
IVATAPRVA

SEQ ID NO: 394 - protein, 9 aa, synthetic construct
  source 1..9: note = AAV9 587/590
VTRTSVAIE

SEQ ID NO: 395 - protein, 9 aa, synthetic construct
  source 1..9: note = AAV9 587/590
MIVPTPMNQ

SEQ ID NO: 396 - protein, 9 aa, synthetic construct
  source 1..9: note = AAV9 587/590
IQVMHSTTH

SEQ ID NO: 397 - protein, 9 aa, synthetic construct
  source 1..9: note = AAV9 587/590
QEPTYKNEV

SEQ ID NO: 398 - protein, 9 aa, synthetic construct
  source 1..9: note = AAV9 587/590
VVPTMNNTR

SEQ ID NO: 399 - protein, 9 aa, synthetic construct
  source 1..9: note = AAV9 587/590
RETTVYPLQ

SEQ ID NO: 400 - protein, 9 aa, synthetic construct
  source 1..9: note = AAV9 587/590
RITTESTIH

SEQ ID NO: 401 - protein, 16 aa, synthetic construct
  source 1..16: note = AAV2 R588A/589
AEEVAARRARVVWCAA

SEQ ID NO: 402 - protein, 9 aa, synthetic construct
  source 1..9: note = AAV9 587/590
DPVRRKMQV

SEQ ID NO: 403 - protein, 9 aa, synthetic construct
  source 1..9: note = AAV9 587/590
PYSSKTQDP

SEQ ID NO: 404 - protein, 9 aa, synthetic construct
  source 1..9: note = AAV9 587/590
WPETKQEKS

SEQ ID NO: 405 - protein, 9 aa, synthetic construct
  source 1..9: note = AAV9 587/590
PNTQHLKAE

SEQ ID NO: 406 - protein, 9 aa, synthetic construct
  source 1..9: note = AAV9 587/590
WQSTGKQSI

SEQ ID NO: 407 - protein, 9 aa, synthetic construct
  source 1..9: note = AAV9 587/590
KASTDPHSR

SEQ ID NO: 408 - protein, 9 aa, synthetic construct
  source 1..9: note = AAV9 587/590
KPTGTIQGY

SEQ ID NO: 409 - protein, 9 aa, synthetic construct
  source 1..9: note = AAV9 587/590
HLGTAIVQP

SEQ ID NO: 410 - protein, 9 aa, synthetic construct
  source 1..9: note = AAV9 587/590
PKFSHATSE

SEQ ID NO: 411 - protein, 9 aa, synthetic construct
  source 1..9: note = AAV9 587/590
IRDGIQMHV

SEQ ID NO: 412 - protein, 9 aa, synthetic construct
  source 1..9: note = AAV9 587/590
TTNQERLQS

SEQ ID NO: 413 - protein, 9 aa, synthetic construct
  source 1..9: note = AAV9 587/590
QIKVPPLDQ

SEQ ID NO: 414 - protein, 9 aa, synthetic construct
  source 1..9: note = AAV9 587/590
LHHERGPSA

SEQ ID NO: 415 - protein, 9 aa, synthetic construct
  source 1..9: note = AAV9 587/590
KPLGQAQVQ

SEQ ID NO: 416 - protein, 9 aa, synthetic construct
  source 1..9: note = AAV9 587/590
TPASATRDS

SEQ ID NO: 417 - protein, 9 aa, synthetic construct
  source 1..9: note = AAV9 587/590
PQQTQVSGQ

SEQ ID NO: 418 - protein, 9 aa, synthetic construct
  source 1..9: note = AAV9 587/590
ERSSQSIIQ

SEQ ID NO: 419 - protein, 9 aa, synthetic construct
  source 1..9: note = AAV9 587/590
YHEWRHIPT

SEQ ID NO: 420 - protein, 9 aa, synthetic construct
  source 1..9: note = AAV9 587/590
PLQASGTAY

SEQ ID NO: 421 - protein, 9 aa, synthetic construct
  source 1..9: note = AAV9 587/590
TNNGVRSII

SEQ ID NO: 422 - protein, 9 aa, synthetic construct
  source 1..9: note = AAV9 587/590
VQPEITPGQ

SEQ ID NO: 423 - protein, 9 aa, synthetic construct
  source 1..9: note = AAV9 587/590
QDDHNKVQQ

SEQ ID NO: 424 - protein, 9 aa, synthetic construct
  source 1..9: note = AAV9 587/590
PIVKQNTDT

SEQ ID NO: 425 - protein, 9 aa, synthetic construct
  source 1..9: note = AAV9 587/590
HTLPRLQEE

SEQ ID NO: 426 - protein, 9 aa, synthetic construct
  source 1..9: note = AAV9 587/590
QPTQGTVRA

SEQ ID NO: 427 - protein, 9 aa, synthetic construct
  source 1..9: note = AAV9 587/590
TRPQGPLSP

SEQ ID NO: 428 - protein, 9 aa, synthetic construct
  source 1..9: note = AAV9 587/590
TYAQPHQNQ

SEQ ID NO: 429 - protein, 9 aa, synthetic construct
  source 1..9: note = AAV9 587/590
IQMEQKQQV

SEQ ID NO: 430 - protein, 9 aa, synthetic construct
  source 1..9: note = AAV9 587/590
TQTPPQDAS

SEQ ID NO: 431 - protein, 9 aa, synthetic construct
  source 1..9: note = AAV9 587/590
SGAQEKWEM

SEQ ID NO: 432 - protein, 16 aa, synthetic construct
  source 1..16: note = AAV9 588/589
ASDTEKEDRSPSAIFA

SEQ ID NO: 433 - protein, 9 aa, synthetic construct
  source 1..9: note = AAV9 587/590
PNYQTKMSF

SEQ ID NO: 434 - protein, 9 aa, synthetic construct
  source 1..9: note = AAV9 587/590
RPGAAVAPA

SEQ ID NO: 435 - protein, 9 aa, synthetic construct
  source 1..9: note = AAV9 587/590
LAGSHVSTQ

SEQ ID NO: 436 - protein, 9 aa, synthetic construct
  source 1..9: note = AAV9 587/590
LPPGNQTNN

SEQ ID NO: 437 - protein, 9 aa, synthetic construct
  source 1..9: note = AAV9 587/590
NHQHDIEWF

SEQ ID NO: 438 - protein, 9 aa, synthetic construct
  source 1..9: note = AAV9 587/590
ISEDGSNRK

SEQ ID NO: 439 - protein, 9 aa, synthetic construct
  source 1..9: note = AAV9 587/590
SMGIHPTNA

SEQ ID NO: 440 - protein, 9 aa, synthetic construct
  source 1..9: note = AAV9 587/590
TPLAMASQK

SEQ ID NO: 441 - protein, 9 aa, synthetic construct
  source 1..9: note = AAV9 587/590
NVTSNTIQF

SEQ ID NO: 442 - protein, 9 aa, synthetic construct
  source 1..9: note = AAV9 587/590
NYNTVPQPK

SEQ ID NO: 443 - protein, 9 aa, synthetic construct
  source 1..9: note = AAV9 587/590
PRVLQGVQV

SEQ ID NO: 444 - protein, 9 aa, synthetic construct
  source 1..9: note = AAV9 587/590
TGTRTSAQQ

SEQ ID NO: 445 - protein, 9 aa, synthetic construct
  source 1..9: note = AAV9 587/590
NHHRPDVFV

SEQ ID NO: 446 - protein, 9 aa, synthetic construct
  source 1..9: note = AAV9 587/590
PGPNERQIM

SEQ ID NO: 447 - protein, 9 aa, synthetic construct
  source 1..9: note = AAV9 587/590
PHDRLPQEV

SEQ ID NO: 448 - protein, 9 aa, synthetic construct
  source 1..9: note = AAV9 587/590
PNAGVMDEE

SEQ ID NO: 449 - protein, 9 aa, synthetic construct
  source 1..9: note = AAV9 587/590
TAHYRTAET

SEQ ID NO: 450 - protein, 9 aa, synthetic construct
  source 1..9: note = AAV9 587/590
RPQSSATTH

SEQ ID NO: 451 - protein, 9 aa, synthetic construct
  source 1..9: note = AAV9 587/590
IAHFQPKDT

SEQ ID NO: 452 - protein, 9 aa, synthetic construct
  source 1..9: note = AAV9 587/590
FAHPEKGFF

SEQ ID NO: 453 - protein, 9 aa, synthetic construct
  source 1..9: note = AAV9 587/590
QAYAKTAQL

SEQ ID NO: 454 - protein, 9 aa, synthetic construct
  source 1..9: note = AAV9 587/590
TTSVSVMPK

SEQ ID NO: 455 - protein, 9 aa, synthetic construct
  source 1..9: note = AAV9 587/590
ASVDTEQRR

SEQ ID NO: 456 - protein, 9 aa, synthetic construct
  source 1..9: note = AAV9 587/590
WHEAIIVTG

SEQ ID NO: 457 - protein, 9 aa, synthetic construct
  source 1..9: note = AAV9 587/590
VDNDIEWKF

SEQ ID NO: 458 - protein, 9 aa, synthetic construct
  source 1..9: note = AAV9 587/590
NRVGSQAPT

SEQ ID NO: 459 - protein, 16 aa, synthetic construct
  source 1..16: note = AAV2 R588A/589
AAVIVANPREEIIVKA

SEQ ID NO: 460 - protein, 9 aa, synthetic construct
  source 1..9: note = AAV9 587/590
PTTPQMVSL

SEQ ID NO: 461 - protein, 16 aa, synthetic construct
  source 1..16: note = AAV2 R588A/589
ALKNFAKYFLHQSHEA

SEQ ID NO: 462 - protein, 9 aa, synthetic construct
  source 1..9: note = AAV9 587/590
KYVNVEVQT

SEQ ID NO: 463 - protein, 9 aa, synthetic construct
  source 1..9: note = AAV9 587/590
LLDAGTSGK

SEQ ID NO: 464 - protein, 9 aa, synthetic construct
  source 1..9: note = AAV9 587/590
NAPAQQKMM

SEQ ID NO: 465 - protein, 9 aa, synthetic construct
  source 1..9: note = AAV9 587/590
VDDHAHRQQ

SEQ ID NO: 466 - protein, 9 aa, synthetic construct
  source 1..9: note = AAV9 587/590
PVKLANPTN

SEQ ID NO: 467 - protein, 9 aa, synthetic construct
  source 1..9: note = AAV9 587/590
HALVHQGAV

SEQ ID NO: 468 - protein, 9 aa, synthetic construct
  source 1..9: note = AAV9 587/590
VFQKSHSTE

SEQ ID NO: 469 - protein, 9 aa, synthetic construct
  source 1..9: note = AAV9 587/590
FKQRDSNET

SEQ ID NO: 470 - protein, 9 aa, synthetic construct
  source 1..9: note = AAV9 587/590
EQMSQRVGF

SEQ ID NO: 471 - protein, 9 aa, synthetic construct
  source 1..9: note = AAV9 587/590
RQAGQFPDS

SEQ ID NO: 472 - protein, 9 aa, synthetic construct
  source 1..9: note = AAV9 587/590
ESQANAHNA

SEQ ID NO: 473 - protein, 9 aa, synthetic construct
  source 1..9: note = AAV9 587/590
PPDSKSLNQ

SEQ ID NO: 474 - protein, 9 aa, synthetic construct
  source 1..9: note = AAV9 587/590
PEQPSQSQY

SEQ ID NO: 475 - protein, 9 aa, synthetic construct
  source 1..9: note = AAV9 587/590
DGIHTSVQM

SEQ ID NO: 476 - protein, 9 aa, synthetic construct
  source 1..9: note = AAV9 587/590
RFDLQEGQS

SEQ ID NO: 477 - protein, 9 aa, synthetic construct
  source 1..9: note = AAV9 587/590
DRDRAMMNI

SEQ ID NO: 478 - protein, 9 aa, synthetic construct
  source 1..9: note = AAV9 587/590
YPPEAQMEM

SEQ ID NO: 479 - protein, 9 aa, synthetic construct
  source 1..9: note = AAV9 587/590
TAVTTHNGS

SEQ ID NO: 480 - protein, 9 aa, synthetic construct
  source 1..9: note = AAV9 587/590
EWALDQYGA

SEQ ID NO: 481 - protein, 9 aa, synthetic construct
  source 1..9: note = AAV9 587/590
ITQSNERMT

SEQ ID NO: 482 - protein, 9 aa, synthetic construct
  source 1..9: note = AAV9 587/590
NITHQTMLQ

SEQ ID NO: 483 - protein, 9 aa, synthetic construct
  source 1..9: note = AAV9 587/590
MMNGSQQFQ

SEQ ID NO: 484 - protein, 9 aa, synthetic construct
  source 1..9: note = AAV9 587/590
QQTTGRVQQ

SEQ ID NO: 485 - protein, 9 aa, synthetic construct
  source 1..9: note = AAV9 587/590
LVNPDPMQY

SEQ ID NO: 486 - protein, 9 aa, synthetic construct
  source 1..9: note = AAV9 587/590
HTHDMDMTP

SEQ ID NO: 487 - protein, 9 aa, synthetic construct
  source 1..9: note = AAV9 587/590
PQTKNTMDD

SEQ ID NO: 488 - protein, 9 aa, synthetic construct
  source 1..9: note = AAV9 587/590
DPERQTRPY

SEQ ID NO: 489 - protein, 9 aa, synthetic construct
  source 1..9: note = AAV9 587/590
TKARSDPLE

SEQ ID NO: 490 - protein, 9 aa, synthetic construct
  source 1..9: note = AAV9 587/590
RVNDQPYQM

SEQ ID NO: 491 - protein, 9 aa, synthetic construct
  source 1..9: note = AAV9 587/590
RPSNVDSAT

SEQ ID NO: 492 - protein, 9 aa, synthetic construct
  source 1..9: note = AAV9 587/590
RMSNDQKSF

SEQ ID NO: 493 - protein, 9 aa, synthetic construct
  source 1..9: note = AAV9 587/590
SMTSAFDRI

SEQ ID NO: 494 - protein, 9 aa, synthetic construct
  source 1..9: note = AAV9 587/590
IHDKQMNNQ

SEQ ID NO: 495 - protein, 9 aa, synthetic construct
  source 1..9: note = AAV9 587/590
NEYFPYEDL

SEQ ID NO: 496 - protein, 9 aa, synthetic construct
  source 1..9: note = AAV9 587/590
SIVPTANQD

SEQ ID NO: 497 - protein, 9 aa, synthetic construct
  source 1..9: note = AAV9 587/590
IFEQAHTNK

SEQ ID NO: 498 - protein, 9 aa, synthetic construct
  source 1..9: note = AAV9 587/590
AGDTRSLQV

SEQ ID NO: 499 - protein, 9 aa, synthetic construct
  source 1..9: note = AAV9 587/590
LQNQTKPVI

SEQ ID NO: 500 - protein, 9 aa, synthetic construct
  source 1..9: note = AAV9 587/590
QGDIVQHHF

SEQ ID NO: 501 - protein, 9 aa, synthetic construct
  source 1..9: note = AAV9 587/590
EIQTYKSIV

SEQ ID NO: 502 - protein, 9 aa, synthetic construct
  source 1..9: note = AAV9 587/590
KAPSIVQGT

SEQ ID NO: 503 - protein, 9 aa, synthetic construct
  source 1..9: note = AAV9 587/590
QTPQHGTVL

SEQ ID NO: 504 - protein, 9 aa, synthetic construct
  source 1..9: note = AAV9 587/590
KLMTMYDGQ

SEQ ID NO: 505 - protein, 9 aa, synthetic construct
  source 1..9: note = AAV9 587/590
LWTNQPSAW

SEQ ID NO: 506 - protein, 9 aa, synthetic construct
  source 1..9: note = AAV9 587/590
HSDQMRQMF

SEQ ID NO: 507 - protein, 9 aa, synthetic construct
  source 1..9: note = AAV9 587/590
EWQNEATPA

SEQ ID NO: 508 - protein, 9 aa, synthetic construct
  source 1..9: note = AAV9 587/590
RPERDPMHI

SEQ ID NO: 509 - protein, 9 aa, synthetic construct
  source 1..9: note = AAV9 587/590
AVWQAANTH

SEQ ID NO: 510 - protein, 9 aa, synthetic construct
  source 1..9: note = AAV9 587/590
RTATTQGGF

SEQ ID NO: 511 - protein, 9 aa, synthetic construct
  source 1..9: note = AAV9 587/590
PTNLVEAGE

SEQ ID NO: 512 - protein, 9 aa, synthetic construct
  source 1..9: note = AAV9 587/590
TRMNQTPLQ

SEQ ID NO: 513 - protein, 9 aa, synthetic construct
  source 1..9: note = AAV9 587/590
PSFTQDRDR

SEQ ID NO: 514 - protein, 9 aa, synthetic construct
  source 1..9: note = AAV9 587/590
SSAAHTVTY

SEQ ID NO: 515 - protein, 9 aa, synthetic construct
  source 1..9: note = AAV9 587/590
SHPQPFMKW

SEQ ID NO: 516 - protein, 9 aa, synthetic construct
  source 1..9: note = AAV9 587/590
QQGVREAFI

SEQ ID NO: 517 - protein, 16 aa, synthetic construct
  source 1..16: note = AAV2 R588A/589
AQSPLVSSSDSPPRPA

SEQ ID NO: 518 - protein, 9 aa, synthetic construct
  source 1..9: note = AAV9 587/590
DSAVRQNPF

SEQ ID NO: 519 - protein, 9 aa, synthetic construct
  source 1..9: note = AAV9 587/590
KSNSMQEAH

SEQ ID NO: 520 - protein, 9 aa, synthetic construct
  source 1..9: note = AAV9 587/590
PRQLSSIPS

SEQ ID NO: 521 - protein, 9 aa, synthetic construct
  source 1..9: note = AAV9 587/590
DPSAQKQNY

SEQ ID NO: 522 - protein, 9 aa, synthetic construct
  source 1..9: note = AAV9 587/590
TGAFDDHDI

SEQ ID NO: 523 - protein, 9 aa, synthetic construct
  source 1..9: note = AAV9 587/590
NGTHLVTRA

SEQ ID NO: 524 - protein, 9 aa, synthetic construct
  source 1..9: note = AAV9 587/590
TGGKVQLGS

SEQ ID NO: 525 - protein, 9 aa, synthetic construct
  source 1..9: note = AAV9 587/590
KQLKTMNDF

SEQ ID NO: 526 - protein, 9 aa, synthetic construct
  source 1..9: note = AAV9 587/590
PHDTRIAVD

SEQ ID NO: 527 - protein, 9 aa, synthetic construct
  source 1..9: note = AAV9 587/590
ERHIAHTWT

SEQ ID NO: 528 - protein, 9 aa, synthetic construct
  source 1..9: note = AAV9 587/590
YRPHPQQHP

SEQ ID NO: 529 - protein, 9 aa, synthetic construct
  source 1..9: note = AAV9 587/590
YNLPQPKEI

SEQ ID NO: 530 - protein, 9 aa, synthetic construct
  source 1..9: note = AAV9 587/590
DTQQQPPYK

SEQ ID NO: 531 - protein, 9 aa, synthetic construct
  source 1..9: note = AAV9 587/590
QSNQVKQMD

SEQ ID NO: 532 - protein, 9 aa, synthetic construct
  source 1..9: note = AAV9 587/590
IEVKQQQNG

SEQ ID NO: 533 - protein, 9 aa, synthetic construct
  source 1..9: note = AAV9 587/590
DQTHNIMQK

SEQ ID NO: 534 - protein, 9 aa, synthetic construct
  source 1..9: note = AAV9 587/590
VQEEGWLYA

SEQ ID NO: 535 - protein, 9 aa, synthetic construct
  source 1..9: note = AAV9 587/590
KQMVAQMSM

SEQ ID NO: 536 - protein, 9 aa, synthetic construct
  source 1..9: note = AAV9 587/590
SINGQQHDY

SEQ ID NO: 537 - protein, 9 aa, synthetic construct
  source 1..9: note = AAV9 587/590
TPDMFKSPN

SEQ ID NO: 538 - protein, 9 aa, synthetic construct
  source 1..9: note = AAV9 587/590
GNHSVHHQQ

SEQ ID NO: 539 - protein, 9 aa, synthetic construct
  source 1..9: note = AAV9 587/590
ENSRTTTVG

SEQ ID NO: 540 - protein, 9 aa, synthetic construct
  source 1..9: note = AAV9 587/590
TFVVGQSGK

SEQ ID NO: 541 - protein, 9 aa, synthetic construct
  source 1..9: note = AAV9 587/590
APLRPQTGE

SEQ ID NO: 542 - protein, 9 aa, synthetic construct
  source 1..9: note = AAV9 587/590
QTSSSTHHA

SEQ ID NO: 543 - protein, 9 aa, synthetic construct
  source 1..9: note = AAV9 587/590
KIAATALSS

SEQ ID NO: 544 - protein, 9 aa, synthetic construct
  source 1..9: note = AAV9 587/590
PESGQSKMH

SEQ ID NO: 545 - protein, 9 aa, synthetic construct
  source 1..9: note = AAV9 587/590
VISVGQMAK

SEQ ID NO: 546 - protein, 9 aa, synthetic construct
  source 1..9: note = AAV9 587/590
AERPQQGMH

SEQ ID NO: 547 - protein, 9 aa, synthetic construct
  source 1..9: note = AAV9 587/590
TQQNQIIQR

SEQ ID NO: 548 - protein, 16 aa, synthetic construct
  source 1..16: note = AAV2 R588A/589
ARQAQEKFGKDKSPKA

SEQ ID NO: 549 - protein, 9 aa, synthetic construct
  source 1..9: note = AAV9 587/590
VTAVTTPSH

SEQ ID NO: 550 - protein, 9 aa, synthetic construct
  source 1..9: note = AAV9 587/590
SQVRGPETT

SEQ ID NO: 551 - protein, 9 aa, synthetic construct
  source 1..9: note = AAV9 587/590
PLNHQQIQR

SEQ ID NO: 552 - protein, 9 aa, synthetic construct
  source 1..9: note = AAV9 587/590
AVVPRPHEI

SEQ ID NO: 553 - protein, 9 aa, synthetic construct
  source 1..9: note = AAV9 587/590
TPNMEDNRQ

SEQ ID NO: 554 - protein, 9 aa, synthetic construct
  source 1..9: note = AAV9 587/590
HRVTGVNDV

SEQ ID NO: 555 - protein, 9 aa, synthetic construct
  source 1..9: note = AAV9 587/590
FTGNQGAQN

SEQ ID NO: 556 - protein, 9 aa, synthetic construct
  source 1..9: note = AAV9 587/590
PRLAETSSQ

SEQ ID NO: 557 - protein, 9 aa, synthetic construct
  source 1..9: note = AAV9 587/590
SPRQSDPAH

SEQ ID NO: 558 - protein, 9 aa, synthetic construct
  source 1..9: note = AAV9 587/590
MNDDQQPPP

SEQ ID NO: 559 - protein, 9 aa, synthetic construct
  source 1..9: note = AAV9 587/590
QMKPLPQAM

SEQ ID NO: 560 - protein, 9 aa, synthetic construct
  source 1..9: note = AAV9 587/590
YSRHENISY

SEQ ID NO: 561 - protein, 9 aa, synthetic construct
  source 1..9: note = AAV9 587/590
TEGERVGML

SEQ ID NO: 562 - protein, 9 aa, synthetic construct
  source 1..9: note = AAV9 587/590
NHVPVLWAD

SEQ ID NO: 563 - protein, 9 aa, synthetic construct
  source 1..9: note = AAV9 587/590
TLAPLATTS

SEQ ID NO: 564 - protein, 9 aa, synthetic construct
  source 1..9: note = AAV9 587/590
NQRDVTNLT

SEQ ID NO: 565 - protein, 9 aa, synthetic construct
  source 1..9: note = AAV9 587/590
QQNNTVPNQ

SEQ ID NO: 566 - protein, 9 aa, synthetic construct
  source 1..9: note = AAV9 587/590
EKNMHTGVH

SEQ ID NO: 567 - protein, 9 aa, synthetic construct
  source 1..9: note = AAV9 587/590
VTLQGATPN

SEQ ID NO: 568 - protein, 9 aa, synthetic construct
  source 1..9: note = AAV9 587/590
PTKTSTGQM

SEQ ID NO: 569 - protein, 9 aa, synthetic construct
  source 1..9: note = AAV9 587/590
FNVDQHRNS

SEQ ID NO: 570 - protein, 9 aa, synthetic construct
  source 1..9: note = AAV9 587/590
PMTLLKDIQ

SEQ ID NO: 571 - protein, 9 aa, synthetic construct
  source 1..9: note = AAV9 587/590
DPTRPIETV

SEQ ID NO: 572 - protein, 9 aa, synthetic construct
  source 1..9: note = AAV9 587/590
ERKQQLSEM

SEQ ID NO: 573 - protein, 9 aa, synthetic construct
  source 1..9: note = AAV9 587/590
NQEQFGMIV

SEQ ID NO: 574 - protein, 9 aa, synthetic construct
  source 1..9: note = AAV9 587/590
TTQIMVGTK

SEQ ID NO: 575 - protein, 9 aa, synthetic construct
  source 1..9: note = AAV9 587/590
MYAKTEVQW

SEQ ID NO: 576 - protein, 9 aa, synthetic construct
  source 1..9: note = AAV9 587/590
PVGHQMDVY

SEQ ID NO: 577 - protein, 9 aa, synthetic construct
  source 1..9: note = AAV9 587/590
QVFQDRTSL

SEQ ID NO: 578 - protein, 9 aa, synthetic construct
  source 1..9: note = AAV9 587/590
LKDNQQATF

SEQ ID NO: 579 - protein, 9 aa, synthetic construct
  source 1..9: note = AAV9 587/590
AGNTHTLIN

SEQ ID NO: 580 - protein, 9 aa, synthetic construct
  source 1..9: note = AAV9 587/590
KVPPQHLTA

SEQ ID NO: 581 - protein, 9 aa, synthetic construct
  source 1..9: note = AAV9 587/590
GIVTATRVQ

SEQ ID NO: 582 - protein, 9 aa, synthetic construct
  source 1..9: note = AAV9 587/590
NIAHHQGSN

SEQ ID NO: 583 - protein, 9 aa, synthetic construct
  source 1..9: note = AAV9 587/590
PAMPGAAAR

SEQ ID NO: 584 - protein, 9 aa, synthetic construct
  source 1..9: note = AAV9 587/590
VQSITPPQS

SEQ ID NO: 585 - protein, 9 aa, synthetic construct
  source 1..9: note = AAV9 587/590
QATHQGQNH

SEQ ID NO: 586 - protein, 9 aa, synthetic construct
  source 1..9: note = AAV9 587/590
PTNAPGVTE

SEQ ID NO: 587 - protein, 9 aa, synthetic construct
  source 1..9: note = AAV9 587/590
MHRAQAIAE

SEQ ID NO: 588 - protein, 9 aa, synthetic construct
  source 1..9: note = AAV9 587/590
AQASEKMMK

SEQ ID NO: 589 - protein, 9 aa, synthetic construct
  source 1..9: note = AAV9 587/590
RMNIPPNGT

SEQ ID NO: 590 - protein, 9 aa, synthetic construct
  source 1..9: note = AAV9 587/590
RPSDINSGD

SEQ ID NO: 591 - protein, 9 aa, synthetic construct
  source 1..9: note = AAV9 587/590
MQPKSAQNV

SEQ ID NO: 592 - protein, 9 aa, synthetic construct
  source 1..9: note = AAV9 587/590
LAASMQGAH

SEQ ID NO: 593 - protein, 9 aa, synthetic construct
  source 1..9: note = AAV9 587/590
TSIITQAAN

SEQ ID NO: 594 - protein, 9 aa, synthetic construct
  source 1..9: note = AAV9 587/590
SDREETFNM

SEQ ID NO: 595 - protein, 9 aa, synthetic construct
  source 1..9: note = AAV9 587/590
NQYNMQPSK

SEQ ID NO: 596 - protein, 9 aa, synthetic construct
  source 1..9: note = AAV9 587/590
WVSEEHFLT

SEQ ID NO: 597 - protein, 9 aa, synthetic construct
  source 1..9: note = AAV9 587/590
QSATSVTSQ

SEQ ID NO: 598 - protein, 9 aa, synthetic construct
  source 1..9: note = AAV9 587/590
HARQSYNDP

SEQ ID NO: 599 - protein, 12 aa, synthetic construct
  source 1..12: note = AAV2 R588A/589
AMNVISHDGTPA

SEQ ID NO: 600 - protein, 9 aa, synthetic construct
  source 1..9: note = AAV9 587/590
STIMSEAEF

SEQ ID NO: 601 - protein, 9 aa, synthetic construct
  source 1..9: note = AAV9 587/590
HANTNVLEK

SEQ ID NO: 602 - protein, 9 aa, synthetic construct
  source 1..9: note = AAV9 587/590
KPQVTPAVH

SEQ ID NO: 603 - protein, 9 aa, synthetic construct
  source 1..9: note = AAV9 587/590
AMPDRTMMR

SEQ ID NO: 604 - protein, 9 aa, synthetic construct
  source 1..9: note = AAV9 587/590
VAIGQTKTL

SEQ ID NO: 605 - protein, 9 aa, synthetic construct
  source 1..9: note = AAV9 587/590
PTDYSVKSV

SEQ ID NO: 606 - protein, 9 aa, synthetic construct
  source 1..9: note = AAV9 587/590
IGHHKEMKE

SEQ ID NO: 607 - protein, 9 aa, synthetic construct
  source 1..9: note = AAV9 587/590
ASQAIHYNA

SEQ ID NO: 608 - protein, 9 aa, synthetic construct
  source 1..9: note = AAV9 587/590
QVRMEGAEY

SEQ ID NO: 609 - protein, 9 aa, synthetic construct
  source 1..9: note = AAV9 587/590
MKERHEHFE

SEQ ID NO: 610 - protein, 9 aa, synthetic construct
  source 1..9: note = AAV9 587/590
GQVLMAGTR

SEQ ID NO: 611 - protein, 9 aa, synthetic construct
  source 1..9: note = AAV9 587/590
TVSMPVSHA

SEQ ID NO: 612 - protein, 9 aa, synthetic construct
  source 1..9: note = AAV9 587/590
NHDRVNVQP

SEQ ID NO: 613 - protein, 9 aa, synthetic construct
  source 1..9: note = AAV9 587/590
EELGILNLQ

SEQ ID NO: 614 - protein, 9 aa, synthetic construct
  source 1..9: note = AAV9 587/590
ANGNFQVLK

SEQ ID NO: 615 - protein, 9 aa, synthetic construct
  source 1..9: note = AAV9 587/590
RQYNTESMH

SEQ ID NO: 616 - protein, 9 aa, synthetic construct
  source 1..9: note = AAV9 587/590
MKVSAQVQS

SEQ ID NO: 617 - protein, 9 aa, synthetic construct
  source 1..9: note = AAV9 587/590
NVSQQKMVQ

SEQ ID NO: 618 - protein, 9 aa, synthetic construct
  source 1..9: note = AAV9 587/590
RQQNQDKFS

SEQ ID NO: 619 - protein, 9 aa, synthetic construct
  source 1..9: note = AAV9 587/590
MSSRTMEIH

SEQ ID NO: 620 - protein, 9 aa, synthetic construct
  source 1..9: note = AAV9 587/590
HEMIKQSGP

SEQ ID NO: 621 - protein, 9 aa, synthetic construct
  source 1..9: note = AAV9 587/590
QDTDRAGKF

SEQ ID NO: 622 - protein, 9 aa, synthetic construct
  source 1..9: note = AAV9 587/590
KWEQRGMAH

SEQ ID NO: 623 - protein, 9 aa, synthetic construct
  source 1..9: note = AAV9 587/590
TKQHTEMQQ

SEQ ID NO: 624 - protein, 9 aa, synthetic construct
  source 1..9: note = AAV9 587/590
PMQALQNML

SEQ ID NO: 625 - protein, 9 aa, synthetic construct
  source 1..9: note = AAV9 587/590
KVAFQQQIE

SEQ ID NO: 626 - protein, 9 aa, synthetic construct
  source 1..9: note = AAV9 587/590
QVPRQEVNE

SEQ ID NO: 627 - protein, 9 aa, synthetic construct
  source 1..9: note = AAV9 587/590
HLEREHKWD

SEQ ID NO: 628 - protein, 9 aa, synthetic construct
  source 1..9: note = AAV9 587/590
IQRKIQDLA

SEQ ID NO: 629 - protein, 9 aa, synthetic construct
  source 1..9: note = AAV9 587/590
IKGTDSMQQ

SEQ ID NO: 630 - protein, 9 aa, synthetic construct
  source 1..9: note = AAV9 587/590
TMVSGEARQ

SEQ ID NO: 631 - protein, 9 aa, synthetic construct
  source 1..9: note = AAV9 587/590
TMVASRQPP

SEQ ID NO: 632 - protein, 9 aa, synthetic construct
  source 1..9: note = AAV9 587/590
QQRQHQEIT

SEQ ID NO: 633 - protein, 9 aa, synthetic construct
  source 1..9: note = AAV9 587/590
NPESMEAYI

SEQ ID NO: 634 - protein, 9 aa, synthetic construct
  source 1..9: note = AAV9 587/590
TSQNGKMSV

SEQ ID NO: 635 - protein, 9 aa, synthetic construct
  source 1..9: note = AAV9 587/590
SRTSTQPQE

SEQ ID NO: 636 - protein, 9 aa, synthetic construct
  source 1..9: note = AAV9 587/590
RYAVKGETD

SEQ ID NO: 637 - protein, 9 aa, synthetic construct
  source 1..9: note = AAV9 587/590
VALYPNHAE

SEQ ID NO: 638 - protein, 9 aa, synthetic construct
  source 1..9: note = AAV9 587/590
QQTTQHIDS

SEQ ID NO: 639 - protein, 9 aa, synthetic construct
  source 1..9: note = AAV9 587/590
SSMQEASQI

SEQ ID NO: 640 - protein, 9 aa, synthetic construct
  source 1..9: note = AAV9 587/590
KMNSPNQER

SEQ ID NO: 641 - protein, 9 aa, synthetic construct
  source 1..9: note = AAV9 587/590
MTNRTPASN

SEQ ID NO: 642 - protein, 9 aa, synthetic construct
  source 1..9: note = AAV9 587/590
IGVRPMISP

SEQ ID NO: 643 - protein, 9 aa, synthetic construct
  source 1..9: note = AAV9 587/590
RQADSRTIE

SEQ ID NO: 644 - protein, 9 aa, synthetic construct
  source 1..9: note = AAV9 587/590
EMRNHPAVQ

SEQ ID NO: 645 - protein, 9 aa, synthetic construct
  source 1..9: note = AAV9 587/590
MRTNEPHKE

SEQ ID NO: 646 - protein, 9 aa, synthetic construct
  source 1..9: note = AAV9 587/590
PTIQRQGMN

SEQ ID NO: 647 - protein, 9 aa, synthetic construct
  source 1..9: note = AAV9 587/590
DIDPEDIST

SEQ ID NO: 648 - protein, 9 aa, synthetic construct
  source 1..9: note = AAV9 587/590
EIKMQSTQA

SEQ ID NO: 649 - protein, 9 aa, synthetic construct
  source 1..9: note = AAV9 587/590
QNFPSQNAA

SEQ ID NO: 650 - protein, 9 aa, synthetic construct
  source 1..9: note = AAV9 587/590
YRTGPDEIQ

SEQ ID NO: 651 - protein, 9 aa, synthetic construct
  source 1..9: note = AAV9 587/590
IQHTHSHHE

SEQ ID NO: 652 - protein, 9 aa, synthetic construct
  source 1..9: note = AAV9 587/590
IISTQEGHI

SEQ ID NO: 653 - protein, 9 aa, synthetic construct
  source 1..9: note = AAV9 587/590
PEGDNTFMI

SEQ ID NO: 654 - protein, 9 aa, synthetic construct
  source 1..9: note = AAV9 587/590
RANAEYLSD

SEQ ID NO: 655 - protein, 9 aa, synthetic construct
  source 1..9: note = AAV9 587/590
EVSLPSNVK

SEQ ID NO: 656 - protein, 9 aa, synthetic construct
  source 1..9: note = AAV9 587/590
RMDINRATD

SEQ ID NO: 657 - protein, 9 aa, synthetic construct
  source 1..9: note = AAV9 587/590
HPSPNTAYV

SEQ ID NO: 658 - protein, 9 aa, synthetic construct
  source 1..9: note = AAV9 587/590
SYHSWFPMG

SEQ ID NO: 659 - protein, 9 aa, synthetic construct
  source 1..9: note = AAV9 587/590
AGDRQHMAA

SEQ ID NO: 660 - protein, 9 aa, synthetic construct
  source 1..9: note = AAV9 587/590
QPKKWGADY

SEQ ID NO: 661 - protein, 9 aa, synthetic construct
  source 1..9: note = AAV9 587/590
LKQDSRPTY

SEQ ID NO: 662 - protein, 9 aa, synthetic construct
  source 1..9: note = AAV9 587/590
VSSYQLKSD

SEQ ID NO: 663 - protein, 9 aa, synthetic construct
  source 1..9: note = AAV9 587/590
RGHSVSDNA

SEQ ID NO: 664 - protein, 9 aa, synthetic construct
  source 1..9: note = AAV9 587/590
HQAQNQMFK

SEQ ID NO: 665 - protein, 9 aa, synthetic construct
  source 1..9: note = AAV9 587/590
QASNFITVP

SEQ ID NO: 666 - protein, 9 aa, synthetic construct
  source 1..9: note = AAV9 587/590
VEPHYQQQW

SEQ ID NO: 667 - protein, 9 aa, synthetic construct
  source 1..9: note = AAV9 587/590
TQGRSQEAT

SEQ ID NO: 668 - protein, 9 aa, synthetic construct
  source 1..9: note = AAV9 588/589
ATSSRTTLA

SEQ ID NO: 669 - protein, 9 aa, synthetic construct
  source 1..9: note = AAV9 587/590
PSSIGKHDN

SEQ ID NO: 670 - protein, 9 aa, synthetic construct
  source 1..9: note = AAV9 587/590
RHSGDSIPE

SEQ ID NO: 671 - protein, 9 aa, synthetic construct
  source 1..9: note = AAV9 587/590
ARPATTQSN

SEQ ID NO: 672 - protein, 9 aa, synthetic construct
  source 1..9: note = AAV9 587/590
PAPSGNTMA

SEQ ID NO: 673 - protein, 9 aa, synthetic construct
  source 1..9: note = AAV9 587/590
KSPSMSVVL

SEQ ID NO: 674 - protein, 9 aa, synthetic construct
  source 1..9: note = AAV9 587/590
IPGPNGQIP

SEQ ID NO: 675 - protein, 9 aa, synthetic construct
  source 1..9: note = AAV9 587/590
PQDYHMSEH

SEQ ID NO: 676 - protein, 16 aa, synthetic construct
  source 1..16: note = AAV9 588/589
ATHLRVIYERMNQSLA

SEQ ID NO: 677 - protein, 16 aa, synthetic construct
  source 1..16: note = AAV2 R588A/589
AHGKREVALHLHPDHA

SEQ ID NO: 678 - protein, 9 aa, synthetic construct
  source 1..9: note = AAV9 587/590
PHTHAQMNL

SEQ ID NO: 679 - protein, 9 aa, synthetic construct
  source 1..9: note = AAV9 587/590
TVPSQQPGT

SEQ ID NO: 680 - protein, 16 aa, synthetic construct
  source 1..16: note = AAV2 R588A/589
AEYKDCHLAQVPSHTA

SEQ ID NO: 681 - protein, 16 aa, synthetic construct
  source 1..16: note = AAV2 R588A/589
AVGALIQRGFIRPKDA

SEQ ID NO: 682 - protein, 9 aa, synthetic construct
  source 1..9: note = AAV9 587/590
TETDPMQRM

SEQ ID NO: 683 - protein, 9 aa, synthetic construct
  source 1..9: note = AAV9 587/590
HRQVAGEST

SEQ ID NO: 684 - protein, 9 aa, synthetic construct
  source 1..9: note = AAV9 587/590
SQSPTRIIQ

SEQ ID NO: 685 - protein, 9 aa, synthetic construct
  source 1..9: note = AAV9 587/590
RLAIPDGIT

SEQ ID NO: 686 - protein, 9 aa, synthetic construct
  source 1..9: note = AAV9 587/590
RPPQNPQTI

SEQ ID NO: 687 - protein, 9 aa, synthetic construct
  source 1..9: note = AAV9 587/590
VELMRTHST

SEQ ID NO: 688 - protein, 9 aa, synthetic construct
  source 1..9: note = AAV9 587/590
HQPQYSKSD

SEQ ID NO: 689 - protein, 9 aa, synthetic construct
  source 1..9: note = AAV2 R588A/589
ATMQCAHTA

SEQ ID NO: 690 - protein, 9 aa, synthetic construct
  source 1..9: note = AAV9 587/590
KMGGVAAKI

SEQ ID NO: 691 - protein, 9 aa, synthetic construct
  source 1..9: note = AAV9 587/590
TMNKSADRG

SEQ ID NO: 692 - protein, 9 aa, synthetic construct
  source 1..9: note = AAV9 587/590
EVDDFVSYG

SEQ ID NO: 693 - protein, 9 aa, synthetic construct
  source 1..9: note = AAV9 587/590
PMSQKVQSA

SEQ ID NO: 694 - protein, 9 aa, synthetic construct
  source 1..9: note = AAV9 587/590
IHSKANDVL

SEQ ID NO: 695 - protein, 9 aa, synthetic construct
  source 1..9: note = AAV9 587/590
AVVSPANAA

SEQ ID NO: 696 - protein, 9 aa, synthetic construct
  source 1..9: note = AAV9 587/590
RQTQTASEY

SEQ ID NO: 697 - protein, 9 aa, synthetic construct
  source 1..9: note = AAV9 587/590
MESHPQAPQ

SEQ ID NO: 698 - protein, 9 aa, synthetic construct
  source 1..9: note = AAV9 587/590
TFRESEMIM

SEQ ID NO: 699 - protein, 9 aa, synthetic construct
  source 1..9: note = AAV9 587/590
HHTPQNMVN

SEQ ID NO: 700 - protein, 9 aa, synthetic construct
  source 1..9: note = AAV9 587/590
KTQQMTPVT

SEQ ID NO: 701 - protein, 9 aa, synthetic construct
  source 1..9: note = AAV9 587/590
IAPQQTYWK

SEQ ID NO: 702 - protein, 9 aa, synthetic construct
  source 1..9: note = AAV9 587/590
EVHSKVMHP

SEQ ID NO: 703 - protein, 9 aa, synthetic construct
  source 1..9: note = AAV9 587/590
NKSTSTVAW

SEQ ID NO: 704 - protein, 9 aa, synthetic construct
  source 1..9: note = AAV9 587/590
ADSAQAMTK

SEQ ID NO: 705 - protein, 9 aa, synthetic construct
  source 1..9: note = AAV9 587/590
MESRKPNDI

SEQ ID NO: 706 - protein, 9 aa, synthetic construct
  source 1..9: note = AAV9 587/590
NVAQTMIRV

SEQ ID NO: 707 - protein, 9 aa, synthetic construct
  source 1..9: note = AAV9 587/590
HPRDHEHVD

SEQ ID NO: 708 - protein, 9 aa, synthetic construct
  source 1..9: note = AAV9 587/590
RVNGSQKES

SEQ ID NO: 709 - protein, 9 aa, synthetic construct
  source 1..9: note = AAV9 587/590
YSRASSETA

SEQ ID NO: 710 - protein, 9 aa, synthetic construct
  source 1..9: note = AAV9 587/590
NWGQTQAKQ

SEQ ID NO: 711 - protein, 16 aa, synthetic construct
  source 1..16: note = AAV2 R588A/589
AQEMSKDLEEVKAKVA

SEQ ID NO: 712 - protein, 9 aa, synthetic construct
  source 1..9: note = AAV9 587/590
REMRASQVE

SEQ ID NO: 713 - protein, 9 aa, synthetic construct
  source 1..9: note = AAV9 587/590
QYIQEHVAK

SEQ ID NO: 714 - protein, 9 aa, synthetic construct
  source 1..9: note = AAV9 587/590
QVAQTTLPI

SEQ ID NO: 715 - protein, 9 aa, synthetic construct
  source 1..9: note = AAV9 587/590
NTVQNMAHQ

SEQ ID NO: 716 - protein, 9 aa, synthetic construct
  source 1..9: note = AAV9 587/590
TQSREMDTQ

SEQ ID NO: 717 - protein, 9 aa, synthetic construct
  source 1..9: note = AAV9 587/590
DDAAHHGFD

SEQ ID NO: 718 - protein, 16 aa, synthetic construct
  source 1..16: note = AAV2 R588A/589
AATPSPVLPDIVMEAA

SEQ ID NO: 719 - protein, 9 aa, synthetic construct
  source 1..9: note = AAV9 587/590
QQASGNNLR

SEQ ID NO: 720 - protein, 9 aa, synthetic construct
  source 1..9: note = AAV9 587/590
AKVVTDSMS

SEQ ID NO: 721 - protein, 9 aa, synthetic construct
  source 1..9: note = AAV9 587/590
SQVVSQGSQ

SEQ ID NO: 722 - protein, 9 aa, synthetic construct
  source 1..9: note = AAV9 587/590
FQHRANSME

SEQ ID NO: 723 - protein, 9 aa, synthetic construct
  source 1..9: note = AAV9 588/589
ANRGTEWDA

SEQ ID NO: 724 - protein, 9 aa, synthetic construct
  source 1..9: note = AAV9 587/590
TIDEVDIGY

SEQ ID NO: 725 - protein, 9 aa, synthetic construct
  source 1..9: note = AAV9 587/590
LQYKAMQVS

SEQ ID NO: 726 - protein, 16 aa, synthetic construct
  source 1..16: note = AAV2 R588A/589
AIPVCEAFQCTALSNA

SEQ ID NO: 727 - protein, 9 aa, synthetic construct
  source 1..9: note = AAV9 587/590
TEDSPQMTQ

SEQ ID NO: 728 - protein, 9 aa, synthetic construct
  source 1..9: note = AAV9 587/590
SGSMTPTTV

SEQ ID NO: 729 - protein, 9 aa, synthetic construct
  source 1..9: note = AAV9 587/590
LPMPTSQGI

SEQ ID NO: 730 - protein, 9 aa, synthetic construct
  source 1..9: note = AAV9 587/590
SYPTPQSQT

SEQ ID NO: 731 - protein, 9 aa, synthetic construct
  source 1..9: note = AAV9 587/590
SASKSFPTA

SEQ ID NO: 732 - protein, 9 aa, synthetic construct
  source 1..9: note = AAV9 587/590
PSQPMKFIN

SEQ ID NO: 733 - protein, 9 aa, synthetic construct
  source 1..9: note = AAV9 587/590
VEAQHKPAY

SEQ ID NO: 734 - protein, 9 aa, synthetic construct
  source 1..9: note = AAV9 587/590
IPSPVTAYW

SEQ ID NO: 735 - protein, 9 aa, synthetic construct
  source 1..9: note = AAV9 587/590
NYLSTSVPR

SEQ ID NO: 736 - protein, 9 aa, synthetic construct
  source 1..9: note = AAV9 587/590
TNSVKMPPS

SEQ ID NO: 737 - protein, 9 aa, synthetic construct
  source 1..9: note = AAV9 587/590
PQEDVKSVM

SEQ ID NO: 738 - protein, 9 aa, synthetic construct
  source 1..9: note = AAV9 587/590
EPHNMKVEK

SEQ ID NO: 739 - protein, 9 aa, synthetic construct
  source 1..9: note = AAV9 587/590
PQKQSLHTV

SEQ ID NO: 740 - protein, 9 aa, synthetic construct
  source 1..9: note = AAV9 587/590
KPQGVNVTY

SEQ ID NO: 741 - protein, 9 aa, synthetic construct
  source 1..9: note = AAV9 587/590
FQRSEVQTI

SEQ ID NO: 742 - protein, 16 aa, synthetic construct
  source 1..16: note = AAV9 588/589
AAEPAPDFSDYSEMAA

SEQ ID NO: 743 - protein, 9 aa, synthetic construct
  source 1..9: note = AAV9 587/590
QRQGGMEAK

SEQ ID NO: 744 - protein, 9 aa, synthetic construct
  source 1..9: note = AAV9 587/590
TASVKQQNT

SEQ ID NO: 745 - protein, 9 aa, synthetic construct
  source 1..9: note = AAV9 587/590
HRNTESGDQ

SEQ ID NO: 746 - protein, 9 aa, synthetic construct
  source 1..9: note = AAV9 587/590
FPKSYEQVQ

SEQ ID NO: 747 - protein, 9 aa, synthetic construct
  source 1..9: note = AAV9 587/590
QGASQRVAI

SEQ ID NO: 748 - protein, 9 aa, synthetic construct
  source 1..9: note = AAV9 587/590
HPMAKDVVK

SEQ ID NO: 749 - protein, 9 aa, synthetic construct
  source 1..9: note = AAV9 587/590
REQKLYKEP

SEQ ID NO: 750 - protein, 9 aa, synthetic construct
  source 1..9: note = AAV9 587/590
TEQQWYGRM

SEQ ID NO: 751 - protein, 9 aa, synthetic construct
  source 1..9: note = AAV9 587/590
APTNKTVGI

SEQ ID NO: 752 - protein, 9 aa, synthetic construct
  source 1..9: note = AAV9 587/590
QRTAVNEMQ

SEQ ID NO: 753 - protein, 9 aa, synthetic construct
  source 1..9: note = AAV9 587/590
PQNAQRVDT

SEQ ID NO: 754 - protein, 9 aa, synthetic construct
  source 1..9: note = AAV9 587/590
KDDDKVGIV

SEQ ID NO: 755 - protein, 16 aa, synthetic construct
  source 1..16: note = AAV2 R588A/589
APAPAAPPSTPAAPKA

SEQ ID NO: 756 - protein, 9 aa, synthetic construct
  source 1..9: note = AAV9 587/590
NHAVMHTVS

SEQ ID NO: 757 - protein, 9 aa, synthetic construct
  source 1..9: note = AAV9 587/590
MRTQAVDGF

SEQ ID NO: 758 - protein, 9 aa, synthetic construct
  source 1..9: note = AAV9 587/590
DIQHATKRE

SEQ ID NO: 759 - protein, 9 aa, synthetic construct
  source 1..9: note = AAV9 587/590
ATQAHTMMY

SEQ ID NO: 760 - protein, 9 aa, synthetic construct
  source 1..9: note = AAV9 587/590
QGTSEASRY

SEQ ID NO: 761 - protein, 9 aa, synthetic construct
  source 1..9: note = AAV9 587/590
RNSHGMSES

SEQ ID NO: 762 - protein, 9 aa, synthetic construct
  source 1..9: note = AAV9 587/590
PNSAHNNSY

SEQ ID NO: 763 - protein, 9 aa, synthetic construct
  source 1..9: note = AAV9 587/590
TTGASKDKV

SEQ ID NO: 764 - protein, 9 aa, synthetic construct
  source 1..9: note = AAV9 587/590
DRTGVWAGE

SEQ ID NO: 765 - protein, 9 aa, synthetic construct
  source 1..9: note = AAV9 587/590
VQTLDRGSQ

SEQ ID NO: 766 - protein, 9 aa, synthetic construct
  source 1..9: note = AAV9 587/590
DATAHSLIQ

SEQ ID NO: 767 - protein, 9 aa, synthetic construct
  source 1..9: note = AAV9 587/590
IGQAPEVND

SEQ ID NO: 768 - protein, 9 aa, synthetic construct
  source 1..9: note = AAV9 587/590
HYAKVTEQA

SEQ ID NO: 769 - protein, 9 aa, synthetic construct
  source 1..9: note = AAV9 587/590
DPSVRSNMI

SEQ ID NO: 770 - protein, 9 aa, synthetic construct
  source 1..9: note = AAV9 587/590
MSKVSTQDR

SEQ ID NO: 771 - protein, 9 aa, synthetic construct
  source 1..9: note = AAV2 R588A/589
AQDRISQWA

SEQ ID NO: 772 - protein, 16 aa, synthetic construct
  source 1..16: note = AAV2 R588A/589
AHLAQPATLSQTIVPA

SEQ ID NO: 773 - protein, 9 aa, synthetic construct
  source 1..9: note = AAV9 587/590
NEKIRELPK

SEQ ID NO: 774 - protein, 9 aa, synthetic construct
  source 1..9: note = AAV9 587/590
QMTKHETYI

SEQ ID NO: 775 - protein, 9 aa, synthetic construct
  source 1..9: note = AAV9 587/590
GAQQYSQVS

SEQ ID NO: 776 - protein, 9 aa, synthetic construct
  source 1..9: note = AAV9 587/590
IQDDPMTSM

SEQ ID NO: 777 - protein, 9 aa, synthetic construct
  source 1..9: note = AAV9 587/590
VPTVERNSL

SEQ ID NO: 778 - protein, 9 aa, synthetic construct
  source 1..9: note = AAV9 587/590
KLVMTQTEY

SEQ ID NO: 779 - protein, 9 aa, synthetic construct
  source 1..9: note = AAV9 587/590
GGVPMKALI

SEQ ID NO: 780 - protein, 9 aa, synthetic construct
  source 1..9: note = AAV9 587/590
IQSPQQAMR

SEQ ID NO: 781 - protein, 9 aa, synthetic construct
  source 1..9: note = AAV9 587/590
SFHGPIKNY

SEQ ID NO: 782 - protein, 9 aa, synthetic construct
  source 1..9: note = AAV9 587/590
NTKDNAGKD

SEQ ID NO: 783 - protein, 16 aa, synthetic construct
  source 1..16: note = AAV2 R588A/589
ADFSDLYYHTMNNKHA

SEQ ID NO: 784 - protein, 9 aa, synthetic construct
  source 1..9: note = AAV9 587/590
QKQIMHPES

SEQ ID NO: 785 - protein, 9 aa, synthetic construct
  source 1..9: note = AAV9 587/590
PQPQMGHSM

SEQ ID NO: 786 - protein, 9 aa, synthetic construct
  source 1..9: note = AAV9 587/590
QLHTGIQNS

SEQ ID NO: 787 - protein, 16 aa, synthetic construct
  source 1..16: note = AAV2 R588A/589
AFEVMEDHAGTYGLGA

SEQ ID NO: 788 - protein, 9 aa, synthetic construct
  source 1..9: note = AAV9 587/590
SYVQPTAIM

SEQ ID NO: 789 - protein, 9 aa, synthetic construct
  source 1..9: note = AAV9 587/590
IQATRNVGV

SEQ ID NO: 790 - protein, 9 aa, synthetic construct
  source 1..9: note = AAV9 587/590
MILVVLQSV

SEQ ID NO: 791 - protein, 9 aa, synthetic construct
  source 1..9: note = AAV9 587/590
KMFSSSEQF

SEQ ID NO: 792 - protein, 9 aa, synthetic construct
  source 1..9: note = AAV9 587/590
TTDRDKMAL

SEQ ID NO: 793 - protein, 9 aa, synthetic construct
  source 1..9: note = AAV9 587/590
KAINTMIEA

SEQ ID NO: 794 - protein, 9 aa, synthetic construct
  source 1..9: note = AAV9 587/590
HMTNGTPMM

SEQ ID NO: 795 - protein, 9 aa, synthetic construct
  source 1..9: note = AAV9 587/590
MQNSVKQVW

SEQ ID NO: 796 - protein, 9 aa, synthetic construct
  source 1..9: note = AAV9 587/590
SQPKMQETG

SEQ ID NO: 797 - protein, 9 aa, synthetic construct
  source 1..9: note = AAV9 587/590
TESHSITPM

SEQ ID NO: 798 - protein, 9 aa, synthetic construct
  source 1..9: note = AAV9 587/590
QVSQGVHGQ

SEQ ID NO: 799 - protein, 9 aa, synthetic construct
  source 1..9: note = AAV9 587/590
TTQMQPMVA

SEQ ID NO: 800 - protein, 9 aa, synthetic construct
  source 1..9: note = AAV9 587/590
KLTVTRRMF

SEQ ID NO: 801 - protein, 9 aa, synthetic construct
  source 1..9: note = AAV9 588/589
AGIATSGSA

SEQ ID NO: 802 - protein, 9 aa, synthetic construct
  source 1..9: note = AAV9 587/590
TPNMQSMSY

SEQ ID NO: 803 - protein, 9 aa, synthetic construct
  source 1..9: note = AAV9 587/590
FNDQPQKIF

SEQ ID NO: 804 - protein, 9 aa, synthetic construct
  source 1..9: note = AAV9 587/590
PTSVHGQML

SEQ ID NO: 805 - protein, 9 aa, synthetic construct
  source 1..9: note = AAV9 587/590
SQTHQVGYP

SEQ ID NO: 806 - protein, 9 aa, synthetic construct
  source 1..9: note = AAV9 587/590
QGTTATRPD

SEQ ID NO: 807 - protein, 16 aa, synthetic construct
  source 1..16: note = AAV2 R588A/589
AGGKVQIINKKLDLSA

SEQ ID NO: 808 - protein, 9 aa, synthetic construct
  source 1..9: note = AAV2 R588A/589
ASLSITEDA

SEQ ID NO: 809 - protein, 9 aa, synthetic construct
  source 1..9: note = AAV9 587/590
TSKPPALQG

SEQ ID NO: 810 - protein, 9 aa, synthetic construct
  source 1..9: note = AAV9 587/590
KVMEKQGMR

SEQ ID NO: 811 - protein, 9 aa, synthetic construct
  source 1..9: note = AAV9 587/590
KTVESPQQQ

SEQ ID NO: 812 - protein, 16 aa, synthetic construct
  source 1..16: note = AAV9 588/589
AGPSVGRAKGQDAPLA

SEQ ID NO: 813 - protein, 9 aa, synthetic construct
  source 1..9: note = AAV9 587/590
RHPSPVNVL

SEQ ID NO: 814 - protein, 9 aa, synthetic construct
  source 1..9: note = AAV9 587/590
YQNDMRKGT

SEQ ID NO: 815 - protein, 9 aa, synthetic construct
  source 1..9: note = AAV9 587/590
LINAHQPQK

SEQ ID NO: 816 - protein, 16 aa, synthetic construct
  source 1..16: note = AAV2 R588A/589
AGHAIENAVRSSLKGA

SEQ ID NO: 817 - protein, 9 aa, synthetic construct
  source 1..9: note = AAV9 587/590
GIGTHMTMV

SEQ ID NO: 818 - protein, 9 aa, synthetic construct
  source 1..9: note = AAV9 587/590
RTLHVDNSP

SEQ ID NO: 819 - protein, 16 aa, synthetic construct
  source 1..16: note = AAV2 R588A/589
ADLENLRDLLHVGGGA

SEQ ID NO: 820 - protein, 9 aa, synthetic construct
  source 1..9: note = AAV9 587/590
QEQANQRPN

SEQ ID NO: 821 - protein, 9 aa, synthetic construct
  source 1..9: note = AAV9 587/590
VSAQSMRNP

SEQ ID NO: 822 - protein, 9 aa, synthetic construct
  source 1..9: note = AAV9 587/590
TLQQKTYNP

SEQ ID NO: 823 - protein, 9 aa, synthetic construct
  source 1..9: note = AAV9 587/590
FLDSNHSMT

SEQ ID NO: 824 - protein, 9 aa, synthetic construct
  source 1..9: note = AAV9 587/590
TDYYSTDDY

SEQ ID NO: 825 - protein, 9 aa, synthetic construct
  source 1..9: note = AAV9 587/590
AVLTQTMGK

SEQ ID NO: 826 - protein, 9 aa, synthetic construct
  source 1..9: note = AAV9 587/590
PAPHSVVQM

SEQ ID NO: 827 - protein, 9 aa, synthetic construct
  source 1..9: note = AAV9 587/590
RPNASDIKG

SEQ ID NO: 828 - protein, 9 aa, synthetic construct
  source 1..9: note = AAV9 587/590
PQRNTGIVQ

SEQ ID NO: 829 - protein, 9 aa, synthetic construct
  source 1..9: note = AAV9 587/590
IVSQTPVAV

SEQ ID NO: 830 - protein, 9 aa, synthetic construct
  source 1..9: note = AAV9 587/590
PTTRIGEHP

SEQ ID NO: 831 - protein, 9 aa, synthetic construct
  source 1..9: note = AAV9 587/590
RQPTDSYSK

SEQ ID NO: 832 - protein, 9 aa, synthetic construct
  source 1..9: note = AAV9 587/590
TVGGKTYAD

SEQ ID NO: 833 - protein, 9 aa, synthetic construct
  source 1..9: note = AAV9 587/590
LKNPPLGNQ

SEQ ID NO: 834 - protein, 9 aa, synthetic construct
  source 1..9: note = AAV9 587/590
LSQSQSQTY

SEQ ID NO: 835 - protein, 9 aa, synthetic construct
  source 1..9: note = AAV9 587/590
PPTAQAQYD

SEQ ID NO: 836 - protein, 9 aa, synthetic construct
  source 1..9: note = AAV9 587/590
NDQLHISLR

SEQ ID NO: 837 - protein, 9 aa, synthetic construct
  source 1..9: note = AAV9 587/590
AKNQYVEED

SEQ ID NO: 838 - protein, 9 aa, synthetic construct
  source 1..9: note = AAV9 587/590
QARANAFPD

SEQ ID NO: 839 - protein, 9 aa, synthetic construct
  source 1..9: note = AAV9 587/590
NGNFAYDHP

SEQ ID NO: 840 - protein, 9 aa, synthetic construct
  source 1..9: note = AAV9 587/590
ILQSNTRGI

SEQ ID NO: 841 - protein, 9 aa, synthetic construct
  source 1..9: note = AAV9 587/590
TQPQQHNEP

SEQ ID NO: 842 - protein, 9 aa, synthetic construct
  source 1..9: note = AAV9 587/590
QRHNENVQM

SEQ ID NO: 843 - protein, 16 aa, synthetic construct
  source 1..16: note = AAV2 R588A/589
AQVLRIRKRANSFLEA

SEQ ID NO: 844 - protein, 9 aa, synthetic construct
  source 1..9: note = AAV9 587/590
QLRDVQLQH

SEQ ID NO: 845 - protein, 9 aa, synthetic construct
  source 1..9: note = AAV9 587/590
YAKQSTQQM

SEQ ID NO: 846 - protein, 9 aa, synthetic construct
  source 1..9: note = AAV9 587/590
TQVLRDKQP

SEQ ID NO: 847 - protein, 9 aa, synthetic construct
  source 1..9: note = AAV9 587/590
QNKIEFRPE

SEQ ID NO: 848 - protein, 9 aa, synthetic construct
  source 1..9: note = AAV9 587/590
SSHASMSST

SEQ ID NO: 849 - protein, 9 aa, synthetic construct
  source 1..9: note = AAV9 587/590
RVVEQGRGI

SEQ ID NO: 850 - protein, 9 aa, synthetic construct
  source 1..9: note = AAV9 587/590
HRNPTVVVD

SEQ ID NO: 851 - protein, 16 aa, synthetic construct
  source 1..16: note = AAV2 R588A/589
APWNSATVLQQTYHAA

SEQ ID NO: 852 - protein, 9 aa, synthetic construct
  source 1..9: note = AAV9 587/590
MWNVTSGAE

SEQ ID NO: 853 - protein, 9 aa, synthetic construct
  source 1..9: note = AAV9 587/590
KTQQALVLQ

SEQ ID NO: 854 - protein, 9 aa, synthetic construct
  source 1..9: note = AAV9 587/590
QVHQESMFG

SEQ ID NO: 855 - protein, 9 aa, synthetic construct
  source 1..9: note = AAV9 587/590
QLNTTKPIA

SEQ ID NO: 856 - protein, 9 aa, synthetic construct
  source 1..9: note = AAV9 587/590
EQYAIKQPT

SEQ ID NO: 857 - protein, 9 aa, synthetic construct
  source 1..9: note = AAV9 587/590
ALGKTAETH

SEQ ID NO: 858 - protein, 9 aa, synthetic construct
  source 1..9: note = AAV9 587/590
KVFHTPEMN

SEQ ID NO: 859 - protein, 9 aa, synthetic construct
  source 1..9: note = AAV9 587/590
ASQTAPIVT

SEQ ID NO: 860 - protein, 9 aa, synthetic construct
  source 1..9: note = AAV9 587/590
TLKKTDDVW

SEQ ID NO: 861 - protein, 16 aa, synthetic construct
  source 1..16: note = AAV2 R588A/589
AHEALSPFYSERLKEA

SEQ ID NO: 862 - protein, 9 aa, synthetic construct
  source 1..9: note = AAV9 587/590
QINGSHRIV

SEQ ID NO: 863 - protein, 9 aa, synthetic construct
  source 1..9: note = AAV2 R588A/589
ATAQDLFTA

SEQ ID NO: 864 - protein, 9 aa, synthetic construct
  source 1..9: note = AAV9 588/589
ATAQDLFTA

SEQ ID NO: 865 - protein, 9 aa, synthetic construct
  source 1..9: note = AAV9 587/590
SQHTSDMES

SEQ ID NO: 866 - protein, 9 aa, synthetic construct
  source 1..9: note = AAV9 587/590
YRTATTTNS

SEQ ID NO: 867 - protein, 9 aa, synthetic construct
  source 1..9: note = AAV9 587/590
PASKQFTPD

SEQ ID NO: 868 - protein, 9 aa, synthetic construct
  source 1..9: note = AAV9 587/590
TATRQEVHH

SEQ ID NO: 869 - protein, 9 aa, synthetic construct
  source 1..9: note = AAV9 587/590
TTTGVMVAK

SEQ ID NO: 870 - protein, 9 aa, synthetic construct
  source 1..9: note = AAV9 587/590
NPVFHQSVA

SEQ ID NO: 871 - protein, 9 aa, synthetic construct
  source 1..9: note = AAV9 587/590
KPVSSQHTP

SEQ ID NO: 872 - protein, 9 aa, synthetic construct
  source 1..9: note = AAV9 587/590
TMATAHQTD

SEQ ID NO: 873 - protein, 9 aa, synthetic construct
  source 1..9: note = AAV9 587/590
VVSGVPMSQ

SEQ ID NO: 874 - protein, 9 aa, synthetic construct
  source 1..9: note = AAV9 587/590
MTFGMQQNK

SEQ ID NO: 875 - protein, 9 aa, synthetic construct
  source 1..9: note = AAV9 587/590
YGQAVVMQN

SEQ ID NO: 876 - protein, 9 aa, synthetic construct
  source 1..9: note = AAV9 587/590
LFHKHPDEM

SEQ ID NO: 877 - protein, 9 aa, synthetic construct
  source 1..9: note = AAV9 587/590
RMRDQDVTS

SEQ ID NO: 878 - protein, 9 aa, synthetic construct
  source 1..9: note = AAV9 587/590
MKQTGGSSF

SEQ ID NO: 879 - protein, 9 aa, synthetic construct
  source 1..9: note = AAV9 587/590
LQASSGAMF

SEQ ID NO: 880 - protein, 9 aa, synthetic construct
  source 1..9: note = AAV9 587/590
NVSTTASQV

SEQ ID NO: 881 - protein, 9 aa, synthetic construct
  source 1..9: note = AAV9 587/590
IMQTSVGRD

SEQ ID NO: 882 - protein, 9 aa, synthetic construct
  source 1..9: note = AAV9 587/590
KLAGVVAIH

SEQ ID NO: 883 - protein, 9 aa, synthetic construct
  source 1..9: note = AAV9 587/590
EIKGAADSH

SEQ ID NO: 884 - protein, 9 aa, synthetic construct
  source 1..9: note = AAV9 587/590
VQQGNPMQR

SEQ ID NO: 885 - protein, 9 aa, synthetic construct
  source 1..9: note = AAV9 587/590
HRWVQLWRV

SEQ ID NO: 886 - protein, 9 aa, synthetic construct
  source 1..9: note = AAV9 587/590
RAETHKYNP

SEQ ID NO: 887 - protein, 9 aa, synthetic construct
  source 1..9: note = AAV9 587/590
RDPAINHDQ

SEQ ID NO: 888 - protein, 9 aa, synthetic construct
  source 1..9: note = AAV9 587/590
PQSQGMQQR

SEQ ID NO: 889 - protein, 9 aa, synthetic construct
  source 1..9: note = AAV9 587/590
GIRQDQEYF

SEQ ID NO: 890 - protein, 7 aa, synthetic construct
  source 1..7: note = AAV2 R588A/589
ANCRIPA

SEQ ID NO: 891 - protein, 9 aa, synthetic construct
  source 1..9: note = AAV9 587/590
GTTMKVQMS

SEQ ID NO: 892 - protein, 9 aa, synthetic construct
  source 1..9: note = AAV9 587/590
NTHSSHIMV

SEQ ID NO: 893 - protein, 9 aa, synthetic construct
  source 1..9: note = AAV9 587/590
KQREPGNVG

SEQ ID NO: 894 - protein, 9 aa, synthetic construct
  source 1..9: note = AAV9 587/590
IYFNDAEQI

SEQ ID NO: 895 - protein, 9 aa, synthetic construct
  source 1..9: note = AAV9 587/590
NHTAKAAPL

SEQ ID NO: 896 - protein, 9 aa, synthetic construct
  source 1..9: note = AAV9 587/590
QHVVGQFPQ

SEQ ID NO: 897 - protein, 9 aa, synthetic construct
  source 1..9: note = AAV9 587/590
RIERQHNDQ

SEQ ID NO: 898 - protein, 16 aa, synthetic construct
  source 1..16: note = AAV2 R588A/589
AEAKRHRPLIALPTQA

SEQ ID NO: 899 - protein, 9 aa, synthetic construct
  source 1..9: note = AAV9 587/590
EPHTSSQQR

SEQ ID NO: 900 - protein, 16 aa, synthetic construct
  source 1..16: note = AAV2 R588A/589
AEMYCAPLKPAHSARA

SEQ ID NO: 901 - protein, 9 aa, synthetic construct
  source 1..9: note = AAV9 587/590
DPHQRMAQQ

SEQ ID NO: 902 - protein, 9 aa, synthetic construct
  source 1..9: note = AAV9 587/590
PSITNRQGA

SEQ ID NO: 903 - protein, 16 aa, synthetic construct
  source 1..16: note = AAV2 R588A/589
ADEPARKGKVHIPFPA

SEQ ID NO: 904 - protein, 9 aa, synthetic construct
  source 1..9: note = AAV9 587/590
LLTPQSRSS

SEQ ID NO: 905 - protein, 9 aa, synthetic construct
  source 1..9: note = AAV9 587/590
LTLQGQAKQ

SEQ ID NO: 906 - protein, 9 aa, synthetic construct
  source 1..9: note = AAV9 587/590
QEDDQRSDR

SEQ ID NO: 907 - protein, 9 aa, synthetic construct
  source 1..9: note = AAV9 587/590
PTNPGEVPV

SEQ ID NO: 908 - protein, 9 aa, synthetic construct
  source 1..9: note = AAV9 587/590
MLSNEDGTN

SEQ ID NO: 909 - protein, 9 aa, synthetic construct
  source 1..9: note = AAV9 587/590
PMEMTTELF

SEQ ID NO: 910 - protein, 9 aa, synthetic construct
  source 1..9: note = AAV9 587/590
MPTQSRQYI

SEQ ID NO: 911 - protein, 9 aa, synthetic construct
  source 1..9: note = AAV9 587/590
SKTQHTNPE

SEQ ID NO: 912 - protein, 9 aa, synthetic construct
  source 1..9: note = AAV9 587/590
QDSFFQRWA

SEQ ID NO: 913 - protein, 9 aa, synthetic construct
  source 1..9: note = AAV9 587/590
ITIDVQSAK

SEQ ID NO: 914 - protein, 9 aa, synthetic construct
  source 1..9: note = AAV9 587/590
PQQSHVVSQ

SEQ ID NO: 915 - protein, 9 aa, synthetic construct
  source 1..9: note = AAV9 587/590
VQVGTSVGK

SEQ ID NO: 916 - protein, 9 aa, synthetic construct
  source 1..9: note = AAV9 587/590
SAQTSHPIM

SEQ ID NO: 917 - protein, 9 aa, synthetic construct
  source 1..9: note = AAV9 587/590
PQEMMALVY

SEQ ID NO: 918 - protein, 9 aa, synthetic construct
  source 1..9: note = AAV9 587/590
QHYRMRPHT

SEQ ID NO: 919 - protein, 9 aa, synthetic construct
  source 1..9: note = AAV9 587/590
NMAQHGQLQ

SEQ ID NO: 920 - protein, 9 aa, synthetic construct
  source 1..9: note = AAV9 587/590
RHGLTTPDF

SEQ ID NO: 921 - protein, 9 aa, synthetic construct
  source 1..9: note = AAV9 587/590
TTMQRTMDV

SEQ ID NO: 922 - protein, 9 aa, synthetic construct
  source 1..9: note = AAV9 587/590
PKSHGAHDV

SEQ ID NO: 923 - protein, 16 aa, synthetic construct
  source 1..16: note = AAV9 588/589
AIENAVRSSLKVATSA

SEQ ID NO: 924 - protein, 9 aa, synthetic construct
  source 1..9: note = AAV9 587/590
PMVDAMVSV

SEQ ID NO: 925 - protein, 9 aa, synthetic construct
  source 1..9: note = AAV9 587/590
TEQPEPFVF

SEQ ID NO: 926 - protein, 9 aa, synthetic construct
  source 1..9: note = AAV9 587/590
LDAVPKNIK

SEQ ID NO: 927 - protein, 9 aa, synthetic construct
  source 1..9: note = AAV9 587/590
TGMSQATSR

SEQ ID NO: 928 - protein, 9 aa, synthetic construct
  source 1..9: note = AAV9 587/590
PTRPSSETM

SEQ ID NO: 929 - protein, 9 aa, synthetic construct
  source 1..9: note = AAV9 587/590
RIEGSHPPP

SEQ ID NO: 930 - protein, 9 aa, synthetic construct
  source 1..9: note = AAV9 587/590
VKGHQEFAN

SEQ ID NO: 931 - protein, 9 aa, synthetic construct
  source 1..9: note = AAV9 587/590
MLVGVPQSK

SEQ ID NO: 932 - protein, 9 aa, synthetic construct
  source 1..9: note = AAV9 587/590
QRQQPQPFF

SEQ ID NO: 933 - protein, 9 aa, synthetic construct
  source 1..9: note = AAV2 R588A/589
ARMQASALA

SEQ ID NO: 934 - protein, 9 aa, synthetic construct
  source 1..9: note = AAV9 587/590
DWEAKARVA

SEQ ID NO: 935 - protein, 9 aa, synthetic construct
  source 1..9: note = AAV9 587/590
PIASTITNR

SEQ ID NO: 936 - protein, 9 aa, synthetic construct
  source 1..9: note = AAV9 587/590
ENSQPIRPS

SEQ ID NO: 937 - protein, 16 aa, synthetic construct
  source 1..16: note = AAV2 R588A/589
AHMNVQNGKWDSDPSA

SEQ ID NO: 938 - protein, 9 aa, synthetic construct
  source 1..9: note = AAV9 587/590
PHQVPRDPV

SEQ ID NO: 939 - protein, 9 aa, synthetic construct
  source 1..9: note = AAV9 587/590
TTSTNREVS

SEQ ID NO: 940 - protein, 9 aa, synthetic construct
  source 1..9: note = AAV9 587/590
PSGVMQDPV

SEQ ID NO: 941 - protein, 9 aa, synthetic construct
  source 1..9: note = AAV2 R588A/589
AKSHELQVA

SEQ ID NO: 942 - protein, 9 aa, synthetic construct
  source 1..9: note = AAV9 587/590
AVNQVHHQM

SEQ ID NO: 943 - protein, 9 aa, synthetic construct
  source 1..9: note = AAV9 587/590
SVGRQQQDI

SEQ ID NO: 944 - protein, 9 aa, synthetic construct
  source 1..9: note = AAV9 587/590
VAMIKQSSI

SEQ ID NO: 945 - protein, 9 aa, synthetic construct
  source 1..9: note = AAV9 587/590
DILIWQHSD

SEQ ID NO: 946 - protein, 9 aa, synthetic construct
  source 1..9: note = AAV9 587/590
FTTKHVNEQ

SEQ ID NO: 947 - protein, 16 aa, synthetic construct
  source 1..16: note = AAV2 R588A/589
AKPYSGRRPDAIRLGA

SEQ ID NO: 948 - protein, 9 aa, synthetic construct
  source 1..9: note = AAV9 587/590
QSMADPRAM

SEQ ID NO: 949 - protein, 9 aa, synthetic construct
  source 1..9: note = AAV2 R588A/589
AHLASNLSA

SEQ ID NO: 950 - protein, 9 aa, synthetic construct
  source 1..9: note = AAV9 587/590
LQHSNQDQE

SEQ ID NO: 951 - protein, 9 aa, synthetic construct
  source 1..9: note = AAV9 587/590
NPWDQTKAM

SEQ ID NO: 952 - protein, 9 aa, synthetic construct
  source 1..9: note = AAV9 587/590
VTDVFDVYD

SEQ ID NO: 953 - protein, 9 aa, synthetic construct
  source 1..9: note = AAV9 587/590
EYDQGVTNF

SEQ ID NO: 954 - protein, 9 aa, synthetic construct
  source 1..9: note = AAV9 587/590
QTYSMPQHL

SEQ ID NO: 955 - protein, 9 aa, synthetic construct
  source 1..9: note = AAV9 587/590
FKTDRDSML

SEQ ID NO: 956 - protein, 16 aa, synthetic construct
  source 1..16: note = AAV2 R588A/589
AIPVPVDGMEYHWGNA

SEQ ID NO: 957 - protein, 9 aa, synthetic construct
  source 1..9: note = AAV9 587/590
PASKNVQPM

SEQ ID NO: 958 - protein, 9 aa, synthetic construct
  source 1..9: note = AAV9 587/590
RLEISSQQI

SEQ ID NO: 959 - protein, 9 aa, synthetic construct
  source 1..9: note = AAV9 587/590
IQGLPSMVH

SEQ ID NO: 960 - protein, 9 aa, synthetic construct
  source 1..9: note = AAV9 587/590
NIAQNLLME

SEQ ID NO: 961 - protein, 9 aa, synthetic construct
  source 1..9: note = AAV9 587/590
HPQPSGHFS

SEQ ID NO: 962 - protein, 9 aa, synthetic construct
  source 1..9: note = AAV9 587/590
IPRQGSQTT

SEQ ID NO: 963 - protein, 9 aa, synthetic construct
  source 1..9: note = AAV9 587/590
DVLGQMGKM

SEQ ID NO: 964 - protein, 9 aa, synthetic construct
  source 1..9: note = AAV9 587/590
TYSSPSIGT

SEQ ID NO: 965 - protein, 9 aa, synthetic construct
  source 1..9: note = AAV9 587/590
EHKDFQVSG

SEQ ID NO: 966 - protein, 9 aa, synthetic construct
  source 1..9: note = AAV9 587/590
EVRPKQDFT

SEQ ID NO: 967 - protein, 16 aa, synthetic construct
  source 1..16: note = AAV2 R588A/589
ARYEAVSFMDVNSTWA

SEQ ID NO: 968 - protein, 9 aa, synthetic construct
  source 1..9: note = AAV9 587/590
PYTQKTGSV

SEQ ID NO: 969 - protein, 9 aa, synthetic construct
  source 1..9: note = AAV9 587/590
NFVNNQPGY

SEQ ID NO: 970 - protein, 9 aa, synthetic construct
  source 1..9: note = AAV9 587/590
SGLNMKTSV

SEQ ID NO: 971 - protein, 16 aa, synthetic construct
  source 1..16: note = AAV2 R588A/589
AQHTLKHFEHVRMVDA

SEQ ID NO: 972 - protein, 16 aa, synthetic construct
  source 1..16: note = AAV9 588/589
AQHTLKHFEHVRMVDA

SEQ ID NO: 973 - protein, 9 aa, synthetic construct
  source 1..9: note = AAV9 587/590
KENPQHGIY

SEQ ID NO: 974 - protein, 9 aa, synthetic construct
  source 1..9: note = AAV9 587/590
KSPTPDINT

SEQ ID NO: 975 - protein, 9 aa, synthetic construct
  source 1..9: note = AAV9 587/590
LTSSKSDPA

SEQ ID NO: 976 - protein, 9 aa, synthetic construct
  source 1..9: note = AAV2 R588A/589
ARMQASQVA

SEQ ID NO: 977 - protein, 9 aa, synthetic construct
  source 1..9: note = AAV9 587/590
RYHRDTIDM

SEQ ID NO: 978 - protein, 9 aa, synthetic construct
  source 1..9: note = AAV2 R588A/589
ASLSITKPA

SEQ ID NO: 979 - protein, 16 aa, synthetic construct
  source 1..16: note = AAV2 R588A/589
ASSPGSPGTPGSRSRA

SEQ ID NO: 980 - protein, 6 aa, synthetic construct
  source 1..6: note = AAV2 R588A/589
ADRIPA

SEQ ID NO: 981 - protein, 9 aa, synthetic construct
  source 1..9: note = AAV9 587/590
HETQVKGMQ

SEQ ID NO: 982 - protein, 9 aa, synthetic construct
  source 1..9: note = AAV9 587/590
TPRATPSMP

SEQ ID NO: 983 - protein, 9 aa, synthetic construct
  source 1..9: note = AAV9 587/590
RPTQHQDIN

SEQ ID NO: 984 - protein, 9 aa, synthetic construct
  source 1..9: note = AAV9 587/590
TTNTQGAQV

SEQ ID NO: 985 - protein, 9 aa, synthetic construct
  source 1..9: note = AAV9 587/590
HYGWQQVPD

SEQ ID NO: 986 - protein, 9 aa, synthetic construct
  source 1..9: note = AAV9 587/590
HPGSSMVPT

SEQ ID NO: 987 - protein, 9 aa, synthetic construct
  source 1..9: note = AAV9 587/590
SSVSQVTLR

SEQ ID NO: 988 - protein, 9 aa, synthetic construct
  source 1..9: note = AAV9 587/590
MYAAGNVHA

SEQ ID NO: 989 - protein, 9 aa, synthetic construct
  source 1..9: note = AAV9 587/590
TSGVVQTIH

SEQ ID NO: 990 - protein, 9 aa, synthetic construct
  source 1..9: note = AAV9 587/590
HMHPAQSEY

SEQ ID NO: 991 - protein, 9 aa, synthetic construct
  source 1..9: note = AAV9 587/590
PQKGDMHTS

SEQ ID NO: 992 - protein, 9 aa, synthetic construct
  source 1..9: note = AAV2 R588A/589
ATYQPLFTA

SEQ ID NO: 993 - protein, 9 aa, synthetic construct
  source 1..9: note = AAV9 588/589
ATYQPLFTA

SEQ ID NO: 994 - protein, 9 aa, synthetic construct
  source 1..9: note = AAV2 R588A/589
AQSSDIENA

SEQ ID NO: 995 - protein, 9 aa, synthetic construct
  source 1..9: note = AAV9 587/590
QVAIQPQEE

SEQ ID NO: 996 - protein, 9 aa, synthetic construct
  source 1..9: note = AAV9 587/590
RAVQNVTTM

SEQ ID NO: 997 - protein, 9 aa, synthetic construct
  source 1..9: note = AAV9 587/590
PSTLVMQSV

SEQ ID NO: 998 - protein, 9 aa, synthetic construct
  source 1..9: note = AAV9 587/590
QQQLLDDIP

SEQ ID NO: 999 - protein, 9 aa, synthetic construct
  source 1..9: note = AAV9 587/590
WEKHIMIDY

SEQ ID NO: 1000 - protein, 9 aa, synthetic construct
  source 1..9: note = AAV9 587/590
NQSTSVNTW

SEQ ID NO: 1001 - protein, 9 aa, synthetic construct
  source 1..9: note = AAV9 587/590
TVQEADSQP

SEQ ID NO: 1002 - protein, 16 aa, synthetic construct
  source 1..16: note = AAV2 R588A/589
AGDAIENAVRSSLKVA

SEQ ID NO: 1003 - protein, 9 aa, synthetic construct
  source 1..9: note = AAV9 587/590
RTDKSQYQP

SEQ ID NO: 1004 - protein, 16 aa, synthetic construct
  source 1..16: note = AAV2 R588A/589
ADGGFIYEAGLAPYKA

SEQ ID NO: 1005 - protein, 9 aa, synthetic construct
  source 1..9: note = AAV9 587/590
ARMKDNTPP

SEQ ID NO: 1006 - protein, 9 aa, synthetic construct
  source 1..9: note = AAV9 587/590
STITPMRQI

SEQ ID NO: 1007 - protein, 9 aa, synthetic construct
  source 1..9: note = AAV9 587/590
QTPIAPPPI

SEQ ID NO: 1008 - protein, 9 aa, synthetic construct
  source 1..9: note = AAV9 587/590
AQPEHRSER

SEQ ID NO: 1009 - protein, 9 aa, synthetic construct
  source 1..9: note = AAV2 R588A/589
ASRANISHA

SEQ ID NO: 1010 - protein, 16 aa, synthetic construct
  source 1..16: note = AAV2 R588A/589
AAAAGAVVGGLGGYMA

SEQ ID NO: 1011 - protein, 9 aa, synthetic construct
  source 1..9: note = AAV9 587/590
PQMRQLYHS

SEQ ID NO: 1012 - protein, 9 aa, synthetic construct
  source 1..9: note = AAV9 587/590
LEFHSQAMV

SEQ ID NO: 1013 - protein, 9 aa, synthetic construct
  source 1..9: note = AAV9 587/590
AQKTYQEGD

SEQ ID NO: 1014 - protein, 16 aa, synthetic construct
  source 1..16: note = AAV2 R588A/589
AMFSDLYYLTMNNKHA

SEQ ID NO: 1015 - protein, 9 aa, synthetic construct
  source 1..9: note = AAV9 587/590
VLDYMDIVF

SEQ ID NO: 1016 - protein, 16 aa, synthetic construct
  source 1..16: note = AAV2 R588A/589
ADFSDLCYLTMNNKHA

SEQ ID NO: 1017 - protein, 7 aa, synthetic construct
  source 1..7: note = AAV2 R588A/589
ANAVIPA

SEQ ID NO: 1018 - protein, 9 aa, synthetic construct
  source 1..9: note = AAV9 587/590
KRERGMQDY

SEQ ID NO: 1019 - protein, 9 aa, synthetic construct
  source 1..9: note = AAV9 587/590
GPFTPAGMM

SEQ ID NO: 1020 - protein, 9 aa, synthetic construct
  source 1..9: note = AAV9 587/590
RKMPQAIDV

SEQ ID NO: 1021 - protein, 9 aa, synthetic construct
  source 1..9: note = AAV9 587/590
GSDPAGFKM

SEQ ID NO: 1022 - protein, 9 aa, synthetic construct
  source 1..9: note = AAV9 587/590
DAQSIQVSI

SEQ ID NO: 1023 - protein, 9 aa, synthetic construct
  source 1..9: note = AAV9 587/590
KVNEVMQKD

SEQ ID NO: 1024 - protein, 16 aa, synthetic construct
  source 1..16: note = AAV2 R588A/589
ATSKVLLLPPDVSALA

SEQ ID NO: 1025 - protein, 6 aa, synthetic construct
  source 1..6: note = AAV2 R588A/589
AAAIPA

SEQ ID NO: 1026 - protein, 16 aa, synthetic construct
  source 1..16: note = AAV2 R588A/589
ARYADGHAIENAVRSA

SEQ ID NO: 1027 - protein, 9 aa, synthetic construct
  source 1..9: note = AAV9 587/590
SEHDNHQQT

SEQ ID NO: 1028 - protein, 9 aa, synthetic construct
  source 1..9: note = AAV9 587/590
PRVQGNDQA

SEQ ID NO: 1029 - protein, 9 aa, synthetic construct
  source 1..9: note = AAV2 R588A/589
AVAVHHTPA

SEQ ID NO: 1030 - protein, 9 aa, synthetic construct
  source 1..9: note = AAV2 R588A/589
ADVRDPRGA

SEQ ID NO: 1031 - protein, 16 aa, synthetic construct
  source 1..16: note = AAV2 R588A/589
ASKTSVVDLLYWRDIA

SEQ ID NO: 1032 - protein, 9 aa, synthetic construct
  source 1..9: note = AAV9 587/590
NVNETKGSW

SEQ ID NO: 1033 - protein, 16 aa, synthetic construct
  source 1..16: note = AAV2 R588A/589
AGHAIENAVKSSLKVA

SEQ ID NO: 1034 - protein, 16 aa, synthetic construct
  source 1..16: note = AAV2 R588A/589
ALDWIHGHIRDKEAPA

SEQ ID NO: 1035 - protein, 9 aa, synthetic construct
  source 1..9: note = AAV9 587/590
SPKYFTPTM

SEQ ID NO: 1036 - protein, 9 aa, synthetic construct
  source 1..9: note = AAV9 587/590
KSQVQPQGT

SEQ ID NO: 1037 - protein, 9 aa, synthetic construct
  source 1..9: note = AAV9 587/590
QMDTRIPQR

SEQ ID NO: 1038 - protein, 16 aa, synthetic construct
  source 1..16: note = AAV2 R588A/589
AFRALSTGEKGFGYKA

SEQ ID NO: 1039 - protein, 16 aa, synthetic construct
  source 1..16: note = AAV2 R588A/589
ATCEAVRCDAVHQPPA

SEQ ID NO: 1040 - protein, 9 aa, synthetic construct
  source 1..9: note = AAV9 587/590
ERSSQYEKG

SEQ ID NO: 1041 - protein, 9 aa, synthetic construct
  source 1..9: note = AAV9 587/590
IRPGTAMAP

SEQ ID NO: 1042 - protein, 9 aa, synthetic construct
  source 1..9: note = AAV9 587/590
SPHSVQGSG

SEQ ID NO: 1043 - protein, 9 aa, synthetic construct
  source 1..9: note = AAV9 587/590
QQAQKVAGV

SEQ ID NO: 1044 - protein, 9 aa, synthetic construct
  source 1..9: note = AAV9 587/590
PRDISTAMV

SEQ ID NO: 1045 - protein, 9 aa, synthetic construct
  source 1..9: note = AAV9 587/590
PRVTQDLVG

SEQ ID NO: 1046 - protein, 9 aa, synthetic construct
  source 1..9: note = AAV9 587/590
QQVVTRHEM

SEQ ID NO: 1047 - protein, 9 aa, synthetic construct
  source 1..9: note = AAV2 R588A/589
ARMQAKAVA

SEQ ID NO: 1048 - protein, 7 aa, synthetic construct
  source 1..7: note = AAV2 R588A/589
ANPRIPA

SEQ ID NO: 1049 - protein, 9 aa, synthetic construct
  source 1..9: note = AAV9 587/590
MVEHNHHVM

SEQ ID NO: 1050 - protein, 9 aa, synthetic construct
  source 1..9: note = AAV2 R588A/589
AQSSDISPA

SEQ ID NO: 1051 - protein, 9 aa, synthetic construct
  source 1..9: note = AAV2 R588A/589
ARPQASAVA

SEQ ID NO: 1052 - protein, 9 aa, synthetic construct
  source 1..9: note = AAV9 587/590
NDFDMPGEI

SEQ ID NO: 1053 - protein, 9 aa, synthetic construct
  source 1..9: note = AAV9 587/590
NRNPTMTGP

SEQ ID NO: 1054 - protein, 7 aa, synthetic construct
  source 1..7: note = AAV2 R588A/589
ANAGIPA

SEQ ID NO: 1055 - protein, 16 aa, synthetic construct
  source 1..16: note = AAV2 R588A/589
ADFSHLYYLTMNNKHA

SEQ ID NO: 1056 - protein, 16 aa, synthetic construct
  source 1..16: note = AAV2 R588A/589
ACELSGYGKHEALSPA

SEQ ID NO: 1057 - protein, 9 aa, synthetic construct
  source 1..9: note = AAV9 587/590
TANKSAPVS

SEQ ID NO: 1058 - protein, 9 aa, synthetic construct
  source 1..9: note = AAV9 587/590
QGHSMPTYG

SEQ ID NO: 1059 - protein, 9 aa, synthetic construct
  source 1..9: note = AAV9 587/590
EYRVTDNTQ

SEQ ID NO: 1060 - protein, 9 aa, synthetic construct
  source 1..9: note = AAV9 587/590
LVAVQQSSG

SEQ ID NO: 1061 - protein, 16 aa, synthetic construct
  source 1..16: note = AAV9 588/589
ASPGGNRYPPQGGGGA

SEQ ID NO: 1062 - protein, 9 aa, synthetic construct
  source 1..9: note = AAV9 587/590
RTDQVMSTK

SEQ ID NO: 1063 - protein, 9 aa, synthetic construct
  source 1..9: note = AAV9 587/590
TSDHLFQYD

SEQ ID NO: 1064 - protein, 7 aa, synthetic construct
  source 1..7: note = AAV2 R588A/589
ANNRIPA

SEQ ID NO: 1065 - protein, 9 aa, synthetic construct
  source 1..9: note = AAV9 587/590
MHTEAIMPR

SEQ ID NO: 1066 - protein, 9 aa, synthetic construct
  source 1..9: note = AAV9 587/590
SVVSVTMNQ

SEQ ID NO: 1067 - protein, 16 aa, synthetic construct
  source 1..16: note = AAV2 R588A/589
AYKSSCAFSCEEGFEA

SEQ ID NO: 1068 - protein, 9 aa, synthetic construct
  source 1..9: note = AAV9 587/590
VTVQPHVDI

SEQ ID NO: 1069 - protein, 9 aa, synthetic construct
  source 1..9: note = AAV9 587/590
MEDKYAYEM

SEQ ID NO: 1070 - protein, 9 aa, synthetic construct
  source 1..9: note = AAV2 R588A/589
ASARSPSEA

SEQ ID NO: 1071 - protein, 9 aa, synthetic construct
  source 1..9: note = AAV9 587/590
QHQDRTPSN

SEQ ID NO: 1072 - protein, 9 aa, synthetic construct
  source 1..9: note = AAV9 587/590
NRQPIHKDD

SEQ ID NO: 1073 - protein, 9 aa, synthetic construct
  source 1..9: note = AAV9 587/590
TTMGSPAPM

SEQ ID NO: 1074 - protein, 9 aa, synthetic construct
  source 1..9: note = AAV9 587/590
KQDPRYSYQ

SEQ ID NO: 1075 - protein, 9 aa, synthetic construct
  source 1..9: note = AAV2 R588A/589
AQLSITKDA

SEQ ID NO: 1076 - protein, 9 aa, synthetic construct
  source 1..9: note = AAV9 587/590
PRAGTSIAP

SEQ ID NO: 1077 - protein, 9 aa, synthetic construct
  source 1..9: note = AAV2 R588A/589
AMPRNSIPA

SEQ ID NO: 1078 - protein, 9 aa, synthetic construct
  source 1..9: note = AAV9 587/590
ASVTTVVSA

SEQ ID NO: 1079 - protein, 9 aa, synthetic construct
  source 1..9: note = AAV9 587/590
SPQGKVSQV

SEQ ID NO: 1080 - protein, 9 aa, synthetic construct
  source 1..9: note = AAV9 587/590
RVTNVPNAV

SEQ ID NO: 1081 - protein, 9 aa, synthetic construct
  source 1..9: note = AAV9 587/590
MLPQASAHK

SEQ ID NO: 1082 - protein, 16 aa, synthetic construct
  source 1..16: note = AAV2 R588A/589
ATKEADLPEPSEKQPA

SEQ ID NO: 1083 - protein, 16 aa, synthetic construct
  source 1..16: note = AAV2 R588A/589
ARGKRGQDAMYEYMAA

SEQ ID NO: 1084 - protein, 9 aa, synthetic construct
  source 1..9: note = AAV9 587/590
PNPKSHQWP

SEQ ID NO: 1085 - protein, 16 aa, synthetic construct
  source 1..16: note = AAV2 R588A/589
ADGAETEVDCNRCVCA

SEQ ID NO: 1086 - protein, 9 aa, synthetic construct
  source 1..9: note = AAV9 587/590
SDTNTAPQE

SEQ ID NO: 1087 - protein, 16 aa, synthetic construct
  source 1..16: note = AAV2 R588A/589
ASKSTTDNDIALLHLA

SEQ ID NO: 1088 - protein, 9 aa, synthetic construct
  source 1..9: note = AAV9 587/590
KYISTGGEW

SEQ ID NO: 1089 - protein, 16 aa, synthetic construct
  source 1..16: note = AAV2 R588A/589
AGDSCVTIMSKDKPTA

SEQ ID NO: 1090 - protein, 16 aa, synthetic construct
  source 1..16: note = AAV2 R588A/589
AVLEGDSCVTIMSKDA

SEQ ID NO: 1091 - protein, 16 aa, synthetic construct
  source 1..16: note = AAV2 R588A/589
AELPGFLQSGKDKDAA

SEQ ID NO: 1092 - protein, 9 aa, synthetic construct
  source 1..9: note = AAV9 587/590
QQMSSHQPM

SEQ ID NO: 1093 - protein, 9 aa, synthetic construct
  source 1..9: note = AAV9 587/590
AYHEREQGQ

SEQ ID NO: 1094 - protein, 9 aa, synthetic construct
  source 1..9: note = AAV9 587/590
PKHERVGDL

SEQ ID NO: 1095 - protein, 9 aa, synthetic construct
  source 1..9: note = AAV9 587/590
MENADVNDG

SEQ ID NO: 1096 - protein, 9 aa, synthetic construct
  source 1..9: note = AAV9 587/590
IIEHVSYQY

SEQ ID NO: 1097 - protein, 9 aa, synthetic construct
  source 1..9: note = AAV9 587/590
NQEKQQSNT

SEQ ID NO: 1098 - protein, 9 aa, synthetic construct
  source 1..9: note = AAV9 587/590
KVIHMDDSY

SEQ ID NO: 1099 - protein, 9 aa, synthetic construct
  source 1..9: note = AAV9 587/590
NITINQREH

SEQ ID NO: 1100 - protein, 16 aa, synthetic construct
  source 1..16: note = AAV2 R588A/589
AGGCVTITAEGKPSMA

SEQ ID NO: 1101 - protein, 9 aa, synthetic construct
  source 1..9: note = AAV9 587/590
QVKQSSQLN

SEQ ID NO: 1102 - protein, 9 aa, synthetic construct
  source 1..9: note = AAV2 R588A/589
AADRSSPGA

SEQ ID NO: 1103 - protein, 9 aa, synthetic construct
  source 1..9: note = AAV9 587/590
NAPRGYMPD

SEQ ID NO: 1104 - protein, 9 aa, synthetic construct
  source 1..9: note = AAV9 587/590
IATPLQKQM

SEQ ID NO: 1105 - protein, 9 aa, synthetic construct
  source 1..9: note = AAV9 587/590
DVHHQYSQI

SEQ ID NO: 1106 - protein, 9 aa, synthetic construct
  source 1..9: note = AAV9 587/590
HHRDNSQSN

SEQ ID NO: 1107 - protein, 16 aa, synthetic construct
  source 1..16: note = AAV2 R588A/589
AGHGECVETINNYTCA

SEQ ID NO: 1108 - protein, 9 aa, synthetic construct
  source 1..9: note = AAV9 587/590
FQWQEETDT

SEQ ID NO: 1109 - protein, 16 aa, synthetic construct
  source 1..16: note = AAV2 R588A/589
AHAHRSDALQLGLGKA

SEQ ID NO: 1110 - protein, 9 aa, synthetic construct
  source 1..9: note = AAV9 587/590
MTTPPQSSP

SEQ ID NO: 1111 - protein, 16 aa, synthetic construct
  source 1..16: note = AAV2 R588A/589
ANSTWRTVDRLSATAA

SEQ ID NO: 1112 - protein, 9 aa, synthetic construct
  source 1..9: note = AAV9 587/590
TEGRQPVGF

SEQ ID NO: 1113 - protein, 9 aa, synthetic construct
  source 1..9: note = AAV9 587/590
PQEEGPSQF

SEQ ID NO: 1114 - protein, 9 aa, synthetic construct
  source 1..9: note = AAV9 587/590
PMNYSHGAL

SEQ ID NO: 1115 - protein, 16 aa, synthetic construct
  source 1..16: note = AAV2 R588A/589
ASSSERAHQVLRIRKA

SEQ ID NO: 1116 - protein, 16 aa, synthetic construct
  source 1..16: note = AAV2 R588A/589
AILRQQQHLFGSNVTA

SEQ ID NO: 1117 - protein, 9 aa, synthetic construct
  source 1..9: note = AAV9 587/590
QKPQPQQDP

SEQ ID NO: 1118 - protein, 9 aa, synthetic construct
  source 1..9: note = AAV9 587/590
HTAATSTKD

SEQ ID NO: 1119 - protein, 9 aa, synthetic construct
  source 1..9: note = AAV9 587/590
SVKAVMPFP

SEQ ID NO: 1120 - protein, 9 aa, synthetic construct
  source 1..9: note = AAV9 587/590
ALIFRHHRQ

SEQ ID NO: 1121 - protein, 9 aa, synthetic construct
  source 1..9: note = AAV9 587/590
PMEPEHMWG

SEQ ID NO: 1122 - protein, 9 aa, synthetic construct
  source 1..9: note = AAV9 587/590
EKDVMRVVQ

SEQ ID NO: 1123 - protein, 16 aa, synthetic construct
  source 1..16: note = AAV2 R588A/589
ARCRAIDGDVTFCRPA

SEQ ID NO: 1124 - protein, 16 aa, synthetic construct
  source 1..16: note = AAV2 R588A/589
ASVGGVFNSLGKGIHA

SEQ ID NO: 1125 - protein, 9 aa, synthetic construct
  source 1..9: note = AAV9 587/590
VASHHFQPQ

SEQ ID NO: 1126 - protein, 9 aa, synthetic construct
  source 1..9: note = AAV9 587/590
TNKQMAYST

SEQ ID NO: 1127 - protein, 16 aa, synthetic construct
  source 1..16: note = AAV2 R588A/589
ATPSLPTPPTREPKKA

SEQ ID NO: 1128 - protein, 9 aa, synthetic construct
  source 1..9: note = AAV9 587/590
YMEDLDPES

SEQ ID NO: 1129 - protein, 9 aa, synthetic construct
  source 1..9: note = AAV9 587/590
TMIQAKQLP

SEQ ID NO: 1130 - protein, 9 aa, synthetic construct
  source 1..9: note = AAV9 587/590
PPRVNDHAT

SEQ ID NO: 1131 - protein, 16 aa, synthetic construct
  source 1..16: note = AAV2 R588A/589
AMFDSSNYHGQDLLFA

SEQ ID NO: 1132 - protein, 9 aa, synthetic construct
  source 1..9: note = AAV9 587/590
HPTVQTHSQ

SEQ ID NO: 1133 - protein, 9 aa, synthetic construct
  source 1..9: note = AAV9 587/590
LGSSAGVSF

SEQ ID NO: 1134 - protein, 16 aa, synthetic construct
  source 1..16: note = AAV2 R588A/589
AGLTTRPGSGLTNIKA

SEQ ID NO: 1135 - protein, 9 aa, synthetic construct
  source 1..9: note = AAV2 R588A/589
AGHFSGSGA

SEQ ID NO: 1136 - protein, 9 aa, synthetic construct
  source 1..9: note = AAV9 587/590
RVQVGPVSS

SEQ ID NO: 1137 - protein, 9 aa, synthetic construct
  source 1..9: note = AAV9 587/590
SKINPQTPL

SEQ ID NO: 1138 - protein, 9 aa, synthetic construct
  source 1..9: note = AAV9 587/590
QRDQRVTPQ

SEQ ID NO: 1139 - protein, 9 aa, synthetic construct
  source 1..9: note = AAV9 587/590
SPAGYQANK

SEQ ID NO: 1140 - protein, 9 aa, synthetic construct
  source 1..9: note = AAV9 587/590
MTHAQQLPQ

SEQ ID NO: 1141 - protein, 16 aa, synthetic construct
  source 1..16: note = AAV2 R588A/589
AFKDSAIGFSRVPPRA

SEQ ID NO: 1142 - protein, 9 aa, synthetic construct
  source 1..9: note = AAV9 587/590
MTLKREIMS

SEQ ID NO: 1143 - protein, 9 aa, synthetic construct
  source 1..9: note = AAV9 587/590
IPMQGEQMR

SEQ ID NO: 1144 - protein, 9 aa, synthetic construct
  source 1..9: note = AAV9 587/590
AQETHYMTR

SEQ ID NO: 1145 - protein, 9 aa, synthetic construct
  source 1..9: note = AAV9 588/589
AVRHSSDFA

SEQ ID NO: 1146 - protein, 9 aa, synthetic construct
  source 1..9: note = AAV2 R588A/589
ALSSSMSMA

SEQ ID NO: 1147 - protein, 16 aa, synthetic construct
  source 1..16: note = AAV2 R588A/589
ANPRFNENNRRVIVCA

SEQ ID NO: 1148 - protein, 16 aa, synthetic construct
  source 1..16: note = AAV9 588/589
AAIENAVRSSLKVATA

SEQ ID NO: 1149 - protein, 9 aa, synthetic construct
  source 1..9: note = AAV9 587/590
RQGGNIMEK

SEQ ID NO: 1150 - protein, 9 aa, synthetic construct
  source 1..9: note = AAV9 587/590
PTSQLSAAH

SEQ ID NO: 1151 - protein, 9 aa, synthetic construct
  source 1..9: note = AAV9 587/590
KPSQTHNDM

SEQ ID NO: 1152 - protein, 16 aa, synthetic construct
  source 1..16: note = AAV2 R588A/589
AKVAVVRTPPKSPSSA

SEQ ID NO: 1153 - protein, 9 aa, synthetic construct
  source 1..9: note = AAV9 587/590
HDQGHAAQK

SEQ ID NO: 1154 - protein, 9 aa, synthetic construct
  source 1..9: note = AAV9 587/590
AMAAAMGNS

SEQ ID NO: 1155 - protein, 9 aa, synthetic construct
  source 1..9: note = AAV9 587/590
GDTQPRMQY

SEQ ID NO: 1156 - protein, 9 aa, synthetic construct
  source 1..9: note = AAV9 587/590
EYPSEIVYS

SEQ ID NO: 1157 - protein, 9 aa, synthetic construct
  source 1..9: note = AAV9 587/590
TWQHTTETS

SEQ ID NO: 1158 - protein, 9 aa, synthetic construct
  source 1..9: note = AAV9 587/590
TGGAYPVIV

SEQ ID NO: 1159 - protein, 16 aa, synthetic construct
  source 1..16: note = AAV2 R588A/589
AGYGSSSRRAPQTGIA

SEQ ID NO: 1160 - protein, 9 aa, synthetic construct
  source 1..9: note = AAV2 R588A/589
AVADSRSPA

SEQ ID NO: 1161 - protein, 16 aa, synthetic construct
  source 1..16: note = AAV2 R588A/589
AGIVSWGRGCALKDKA

SEQ ID NO: 1162 - protein, 9 aa, synthetic construct
  source 1..9: note = AAV2 R588A/589
AWESDDFTA

SEQ ID NO: 1163 - protein, 9 aa, synthetic construct
  source 1..9: note = AAV9 587/590
SVDQSHDPN

SEQ ID NO: 1164 - protein, 9 aa, synthetic construct
  source 1..9: note = AAV2 R588A/589
ARPNLPLPA

SEQ ID NO: 1165 - protein, 9 aa, synthetic construct
  source 1..9: note = AAV9 588/589
ARPNLPLPA

SEQ ID NO: 1166 - protein, 9 aa, synthetic construct
  source 1..9: note = AAV9 587/590
TDLADSEWR

SEQ ID NO: 1167 - protein, 16 aa, synthetic construct
  source 1..16: note = AAV2 R588A/589
AAFTTTLRGAQRLAAA

SEQ ID NO: 1168 - protein, 9 aa, synthetic construct
  source 1..9: note = AAV9 587/590
VSNPQPPSR

SEQ ID NO: 1169 - protein, 16 aa, synthetic construct
  source 1..16: note = AAV2 R588A/589
APSLPTPPTREPKKVA

SEQ ID NO: 1170 - protein, 9 aa, synthetic construct
  source 1..9: note = AAV9 587/590
PTHTQGRES

SEQ ID NO: 1171 - protein, 16 aa, synthetic construct
  source 1..16: note = AAV2 R588A/589
AQNAKNGNLPEFGDAA

SEQ ID NO: 1172 - protein, 9 aa, synthetic construct
  source 1..9: note = AAV9 587/590
KNAVMAPGN

SEQ ID NO: 1173 - protein, 16 aa, synthetic construct
  source 1..16: note = AAV9 588/589
ALVPIATQTYEAWLGA

SEQ ID NO: 1174 - protein, 9 aa, synthetic construct
  source 1..9: note = AAV9 587/590
IPMRIETDP

SEQ ID NO: 1175 - protein, 9 aa, synthetic construct
  source 1..9: note = AAV9 587/590
QARDSPKGW

SEQ ID NO: 1176 - protein, 16 aa, synthetic construct
  source 1..16: note = AAV2 R588A/589
AHRERMSQVMREWEEA

SEQ ID NO: 1177 - protein, 9 aa, synthetic construct
  source 1..9: note = AAV9 587/590
SRVSQNEHP

SEQ ID NO: 1178 - protein, 9 aa, synthetic construct
  source 1..9: note = AAV9 587/590
QRNEKSEFI

SEQ ID NO: 1179 - protein, 9 aa, synthetic construct
  source 1..9: note = AAV9 587/590
QTLPEVTKW

SEQ ID NO: 1180 - protein, 9 aa, synthetic construct
  source 1..9: note = AAV9 587/590
VKGWQVTER

SEQ ID NO: 1181 - protein, 16 aa, synthetic construct
  source 1..16: note = AAV2 R588A/589
APTTVESHGNYSTQIA

SEQ ID NO: 1182 - protein, 9 aa, synthetic construct
  source 1..9: note = AAV9 587/590
PRQGPSTNL

SEQ ID NO: 1183 - protein, 16 aa, synthetic construct
  source 1..16: note = AAV2 R588A/589
AEATDGMLKIQFSAQA

SEQ ID NO: 1184 - protein, 16 aa, synthetic construct
  source 1..16: note = AAV9 588/589
ALYRQKVEPLRAELQA

SEQ ID NO: 1185 - protein, 9 aa, synthetic construct
  source 1..9: note = AAV9 587/590
RPLTTEAIV

SEQ ID NO: 1186 - protein, 9 aa, synthetic construct
  source 1..9: note = AAV9 587/590
KPGSQQIMD

SEQ ID NO: 1187 - protein, 16 aa, synthetic construct
  source 1..16: note = AAV2 R588A/589
AHQLLGDLHQEGPPLA

SEQ ID NO: 1188 - protein, 9 aa, synthetic construct
  source 1..9: note = AAV9 587/590
MEQLVTFKT

SEQ ID NO: 1189 - protein, 9 aa, synthetic construct
  source 1..9: note = AAV9 587/590
GASMQTALW

SEQ ID NO: 1190 - protein, 16 aa, synthetic construct
  source 1..16: note = AAV2 R588A/589
AAFRCLAENAGDVAFA

SEQ ID NO: 1191 - protein, 9 aa, synthetic construct
  source 1..9: note = AAV9 587/590
IQTQLPPGK

SEQ ID NO: 1192 - protein, 9 aa, synthetic construct
  source 1..9: note = AAV9 587/590
NTVHQAQIM

SEQ ID NO: 1193 - protein, 9 aa, synthetic construct
  source 1..9: note = AAV9 587/590
PISNVNQSR

SEQ ID NO: 1194 - protein, 16 aa, synthetic construct
  source 1..16: note = AAV2 R588A/589
APDLVQEACESELNEA

SEQ ID NO: 1195 - protein, 16 aa, synthetic construct
  source 1..16: note = AAV2 R588A/589
AKIQVLVEPDHFKVAA

SEQ ID NO: 1196 - protein, 16 aa, synthetic construct
  source 1..16: note = AAV2 R588A/589
AREWFMDLNLPWSSAA

SEQ ID NO: 1197 - protein, 16 aa, synthetic construct
  source 1..16: note = AAV9 588/589
AEFSSNTVKLTSGHLA

SEQ ID NO: 1198 - protein, 16 aa, synthetic construct
  source 1..16: note = AAV2 R588A/589
AFTYKSSCAFSCEEGA

SEQ ID NO: 1199 - protein, 9 aa, synthetic construct
  source 1..9: note = AAV9 587/590
PYPTQASMK

SEQ ID NO: 1200 - protein, 9 aa, synthetic construct
  source 1..9: note = AAV9 587/590
KPTNSGQEH

SEQ ID NO: 1201 - protein, 9 aa, synthetic construct
  source 1..9: note = AAV9 587/590
LSTGPPEKI

SEQ ID NO: 1202 - protein, 9 aa, synthetic construct
  source 1..9: note = AAV9 587/590
APQLHVMSA

SEQ ID NO: 1203 - protein, 9 aa, synthetic construct
  source 1..9: note = AAV9 587/590
TASKMQDSK

SEQ ID NO: 1204 - protein, 16 aa, synthetic construct
  source 1..16: note = AAV2 R588A/589
AFTSSSGQKAMNGSRA

SEQ ID NO: 1205 - protein, 16 aa, synthetic construct
  source 1..16: note = AAV2 R588A/589
AVAEFSEATAELTVSA

SEQ ID NO: 1206 - protein, 9 aa, synthetic construct
  source 1..9: note = AAV9 587/590
INQNKAHDA

SEQ ID NO: 1207 - protein, 16 aa, synthetic construct
  source 1..16: note = AAV2 R588A/589
AARLSKELQAAQARLA

SEQ ID NO: 1208 - protein, 16 aa, synthetic construct
  source 1..16: note = AAV2 R588A/589
ATKPGYVDSIQKGIQA

SEQ ID NO: 1209 - protein, 9 aa, synthetic construct
  source 1..9: note = AAV9 587/590
PTTAPSMVL

SEQ ID NO: 1210 - protein, 16 aa, synthetic construct
  source 1..16: note = AAV2 R588A/589
AGGFVYIAGKCGLVPA

SEQ ID NO: 1211 - protein, 16 aa, synthetic construct
  source 1..16: note = AAV2 R588A/589
ANPTAKRQFVQSAKEA

SEQ ID NO: 1212 - protein, 16 aa, synthetic construct
  source 1..16: note = AAV2 R588A/589
ALALGRFWDYLRWVQA

SEQ ID NO: 1213 - protein, 16 aa, synthetic construct
  source 1..16: note = AAV2 R588A/589
ALRVIYERMNQSLSLA

SEQ ID NO: 1214 - protein, 9 aa, synthetic construct
  source 1..9: note = AAV9 587/590
VTMTQATNR

SEQ ID NO: 1215 - protein, 16 aa, synthetic construct
  source 1..16: note = AAV2 R588A/589
ALLQYNHRVKKLNEIA

SEQ ID NO: 1216 - protein, 16 aa, synthetic construct
  source 1..16: note = AAV2 R588A/589
ALLGKINLKALAALAA

SEQ ID NO: 1217 - protein, 9 aa, synthetic construct
  source 1..9: note = AAV9 587/590
RQRVSYMDD

SEQ ID NO: 1218 - protein, 9 aa, synthetic construct
  source 1..9: note = AAV9 587/590
ADMGQQRYD

SEQ ID NO: 1219 - protein, 16 aa, synthetic construct
  source 1..16: note = AAV2 R588A/589
ATGVLLRALAGVPVAA

SEQ ID NO: 1220 - protein, 9 aa, synthetic construct
  source 1..9: note = AAV9 587/590
SIKKDPPTV

SEQ ID NO: 1221 - protein, 9 aa, synthetic construct
  source 1..9: note = AAV9 587/590
PGKTMLGNH

SEQ ID NO: 1222 - protein, 9 aa, synthetic construct
  source 1..9: note = AAV9 587/590
HQGDTLQYV

SEQ ID NO: 1223 - protein, 16 aa, synthetic construct
  source 1..16: note = AAV2 R588A/589
ALGLGKHNYCRNPDGA

SEQ ID NO: 1224 - protein, 9 aa, synthetic construct
  source 1..9: note = AAV9 587/590
QERQANKSY

SEQ ID NO: 1225 - protein, 9 aa, synthetic construct
  source 1..9: note = AAV9 587/590
QQTRGVELN

SEQ ID NO: 1226 - protein, 9 aa, synthetic construct
  source 1..9: note = AAV2 R588A/589
ASMRVVPMA

SEQ ID NO: 1227 - protein, 9 aa, synthetic construct
  source 1..9: note = AAV2 R588A/589
ASSTDKRDA

SEQ ID NO: 1228 - protein, 9 aa, synthetic construct
  source 1..9: note = AAV2 R588A/589
ALIGGSIVA

SEQ ID NO: 1229 - protein, 9 aa, synthetic construct
  source 1..9: note = AAV9 587/590
LLHTGTSSH

SEQ ID NO: 1230 - protein, 9 aa, synthetic construct
  source 1..9: note = AAV9 587/590
TQHQAQVPR

SEQ ID NO: 1231 - protein, 16 aa, synthetic construct
  source 1..16: note = AAV2 R588A/589
AAGLFGAAEGQAFHLA

SEQ ID NO: 1232 - protein, 16 aa, synthetic construct
  source 1..16: note = AAV2 R588A/589
AENEHAHFQKAKERLA

SEQ ID NO: 1233 - protein, 735 aa, synthetic construct [sequence omitted]

SEQ ID NO: 1234 - protein, 736 aa, synthetic construct
  source 1..736: note = AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 1235 - protein, 743 aa, synthetic construct
  source 1..743: note = CNSRCV300
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAYVNIMDDMDQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 1236 - protein, 17 aa, synthetic construct
  source 1..17: note = AAV9 588/589
QALVPIATQTYEAWLGA